SEQUENCE LISTING (111 sequences)

SEQ ID NO: 1 - DNA, 2796 bp, African swine fever virus
atgatatctatcatggaccgttctgagattgttgcacgggagaacccggtgattacccaacgagttacaaatctcctacaaaccaatgctcctctactattcatgcccattgatatccatgaagtacgatatggagcctacacacttttcatgtatggttccctcgaaaacggttacaaagcagaagtaaggattgaaaacatcccagttttctttgacgtacagattgagttcaatgatacaaaccagctttttttaaagtcgctactgacggctgaaaatattgcgtatgaacggctggagacgctcacccagcgtcctgtaatggggtaccgcgagaaggaaaaagagtttgcaccatacattcgaatattttttaaaagcctgtatgagcaacgaaaagccattacttacttgaataatatgggttacaacaccgccgcggacgacacaacctgttactaccgaatggtttcccgagagctaaaactgcctcttacaagttggatacagcttcagcactattcctacgagcctcgcggcttggtacacaggttttccgtaacccccgaggatcttgtttcctatcaggatgatggccccacagaccacagcatcgttatggcctacgatatagagacctatagccctgttaagggaaccgttccggacccaaatcaggcaaacgacgtggtgttcatgatatgcatgcgcattttttggattcactccacagagcctctagcgagcacgtgcatcactatggcaccagcctctcgggatgaggcaaaaagcctcatggccaagggtgaatctcttcactacgtctcctttcactttaacaatcgtctcgtggaaggatggtttgtgcgacataataacgttcctgataaaatgggattatacccaaaagtactcatcgatctacttaacaaacgaaccgcccttaaacaagagcttaaaaaactaggtgagaaaaaagaatgtatccatgaatcccatcctgggtttaaggaactacagtttcgccatgccatggtagacgcgaagcaaaaggcgttgaaaattttcatgaacacgttttacggcgaggcaggtaacaatttgtcgcccttctttctgcttcctctagccggaggagtcaccagttcgggtcaatataatcttaaactcgtctataactttgttatcaataaaggttacggcatcaagtacggtgacaccgactcattatacattacatgcccagatagtctttatacagaggtaacagacgcatatttaaatagccaaaaaacaataaaacattatgagcaactctgccacgaaaaagtgcttctgtctatgaaagccatgtctacactatgcgccgaggtgaatgaatacctgcggcaagataatggcaccagttacctacgtatggcctacgaggaagtactctttcctgtgtgctttacaggcaagaaaaagtattacggtattgctcatgtaaacacacccaattttaatacaaaagaattattcatccgcggaatagatatcattaagcagggtcaaacaaaactcaccaaaacgataggtacgcgaattatggaagaatccatgaaactgcgccgccctgaggaccatcgcccccctcttattgaaatcgttaaaacggttttgaaggatgctgtggttaacatgaagcagtggaattttgaagacttcatccaaacagatgcgtggagaccggacaaagacaacaaagcagtccaaatctttatgtctcgcatgcacgctcggcgtgagcaactaaaaaaacacggcgccgcagcatcgcaatttgctgagcctgagccgggagaacgcttctcctacgttatcgtggaaaaacaagtacagtttgatattcagggccaccgcacagattcctccagaaagggggacaagatggaatacgtctctgaagcaaaggctaaaaatcttccaattgatatattgttttatatcaacaactatgttctaggcttgtgcgcgagattcattaatgaaaatgaagaatttcaaccccctgacaatgtcagcaataaggatgaatacgctcagcgccgagccaaatcctacctacaaaaattcgtacaatccattcaccctaaagacaagtctgtcattaagcaaggcattgttcatcgacagtgctacaaatacgttcaccaagaaattaaaaaaaaaataggcatctttgccgacctttataaggaattttttaacaacaccacaaaccccatcgaaagctttattcaaagcgctcggtttatgatacaatactctgatggagaacaaaaagtaaaccattctatgaaaaaaatggttgaacagcgtgctactttggcaagtaagcccgctggtaagcccgctggtaatccagctggcaacccagccggcaatgcgctgatgcgggctatatttacgcagctgattacggaagaaaaaaaaattgtacaagccttatacaataagggggatgcaatacacgatcttctcacctatatcattaacaatataaattacaaaattgccacgtttcagacgaaacagatgttgacgttcgagttttctagtactcatgtagaactgctattaaagctgaataagacgtggcttattttggctggaattcatgtggcgaaaaaacatctgcaagctcttttggattcatataataatgaaccaccgtctagaacattcattcagcaggctatagaggaagaatgtggcagtattaaaccatcttgctacgactttatttcctaa

SEQ ID NO: 2 - DNA, 3299 bp, African swine fever virus
aaaacgatggcccgggaatccccattgcaaagcatgagcaggccagtcttatcgccaagcgcgatgtgtatgttcccgaggtggcttcatgcttctttctagccggaacgaacatcaataaggccaaggactgtatcaaggggggaaccaacggcgtcgggctgaagctcgccatggtgcattcgcagtgggccattcttaccaccgccgacggcgcgcaaaagtatgttcaacaaatcaaccagcgcctagatatcattgagcctcctaccattacaccctccagggaaatgtttacacgtatcgagctcatgcccgtataccaggaactagggtacgcggagcctctgtctgaaacggagcaagcagatctttccgcctggatttatcttcgcgcctgccaatgcgcggcctacgtgggaaaaggcaccaccatttattacaatgataagccttgccgcacgggctctgtgatggcgctggccaaaatgtacaccctgttgagcgcgcctaatagcacgatacatacggcgaccattaaggccgacgcaaaaccctatagcctgcaccctctgcaggttgcggcggtcgtgtcccccaagtttaaaaaatttgaacacgtgtccattatcaacggggtaaattgtgtaaaaggagaacatgttacctttttgaaaaagaccattaatgaaatggtcattaaaaaatttcaacagacgattaaagataaaaaccgcaaaacaacattacgtgacagctgttcaaacatctttgtcgttatagtgggttccattccaggcatagaatggaccggccagcggaaggatgaacttagcatcgcagaaaatgtttttaaaacgcattactccatcccttctagttttttaacaagcatgacaaggtctatcgtggatattcttctgcaatccatttctaaaaaagataaccataaacaggtcgacgtagacaaatatacgcgtgcccgcaatgcgggagggaaaagggcgcaggactgcatgctactcgcggcggaaggggatagcgcactttccctgttgcgcacgggactgaccctgggaaagtccaacccaagcgggccctcctttgacttctgcggcatgatctccctgggaggggtcatcatgaatgcctgcaaaaaggtgacaaacattacaacggactctggagaaaccatcatggtgcgcaacgaacagcttaccaataataaagtgttgcagggaattgtgcaggtattgggtctagacttcaactgccattacaaaacgcaggaagagcgagcaaagctgagatacggctgcattgttgcgtgcgttgatcaagatctggatgggtgtggaaaaatccttggactgctgctggcctactttcacctgttttggcctcagcttattatccatggtttcgtaaaacgactgcttaccccgctgatacgtgtgtacgaaaagggcaagactatgcccgtagaattttactatgaacaggagtttgatgcctgggcaaaaaagcagaccagcttagtcaatcatactgtaaaatattacaagggattggcggcgcatgacacccatgaagtaaaaagcatgttcaaacattttgacaacatggtgtacacgtttaccctggatgactcggcaaaggagttgtttcatatttattttggcggggagtcggagttgcgaaaaagagagctttgcaccggcgtggtgccgctcactgaaacccagacgcagtccattcatagtgtccgacgaattccttgcagcctgcatctgcaggtagataccaaggcttacaagctggatgccatcgagcggcagattcccaacttcttagacggaatgacgcgggcgcggcgcaaaattttagccgggggggtgaaatgcttcgcttccaacaaccgtgaacgaaaggtttttcagttcgggggctacgttgcggatcacatgttttatcaccatggcgacatgtcgttaaacacaagtattataaaagccgcccagtattacccgggctcctcccacctctatccagtattcataggcataggaagcttcggctccaggcacctgggaggaaaggatgcaggatccccaagatacatcagtgtgcagcttgcgtctgaatttattaaaacaatgttccccgcggaggactcatggcttctcccctacgtctttgaggacggccagcgggcggaaccagagtactacgtgcctgtattgccgcttgctattatggagtacggcgccaacccatcggagggctggaagtacaccacttgggcccggcaactggaagacattttggccttggtgagggcctacgtcgacaaagacaacccaaaacacgagctactgcactatgcaataaaacataagattactatactcccgctgcggccctccaattacaatttcaagggccatttgaagcggtttggccaatactactacagctacggcacgtacgacatctcagagcagcgaaatataattactattacggagcttcctctgcgtgttcctacggttgcatatatcgaaagtataaaaaaatcgagtaaccgcatgacatttattgaagaaatcatcgactacagtagttcagaaaccattgaaattctggtgaaactaaagccaaatagtctcaaccgtatcgtggaagaatttaaggagactgaagagcaagattccatagaaaattttctgcgcctgcgcaattgtttacattcgcatctaaactttgtaaaacctaaaggtggtattatcgagtttaactcatattatgaaattttatatgcgtggctaccttacaggcgtgagctttaccaaaagcgtcttatgcgtgagcacgcggtgcttaagctgcgcattatcatggaaactgctattgtacgctacatcaatgagtctgcagagctaaatctttcccattatgaggatgaaaaggaggcaagccgcattctaagcgagcatggatttcccccgctgaaccacacgctgatcatttcccctgagtttgcctctatagaggaactcaatcaaaaagcgctgcagggctgttatacctatatactatctttgcaggctcgagaattgcttatcgcagccaaaactcgtcgggtggaaaaaataaaaaaaatgcaagctcgtcttgataaggttgagcagcttttgcaggagtctccctttcccggcgccagcgtatggctggaggaaattgatgcggtggaaaaggctattataaaaggaagaaatactcagtggaaatttcattaa

SEQ ID NO: 3 - DNA, 1530 bp, African swine fever virus
atggaggccattatatcctttgctggaataggaataaattataagaagctacaaagtaaattacaacatgatttcgggcgccttcttaaggcgctcaccgttacggcgcgggcattgcctgggcagccaaagcacatagccataagacaggaaactgccttcacgctgcagggggaatacatttattttcccatattgctgcgaaagcagtttgaaatgtttaacatggtttacacgacgcgccccgtgtcgctgcgggccctcccatgcgttgaaacagaatttccactatttaactaccagcaagaaatggtcgataagattcataaaaagctcctgtccccctatgggcgcttttacctacatctaaataccggtttggggaaaacgcgtattgcgatcagcattattcaaaaacttttgtaccctaccctggtcatcgtgcccaccaaggcgattcaaatacagtggatcgacgagctaacattgctcctgccccacctacgtgtagctgcttacaataatgcagcgtgcaagaaaaaggacatgacgagcaaagagtacgacgtcatcgtgggaatcattaataccctgcgcaagaagcctgagcagttctttgagccctttggtctagtcgtgttagatgaggcacatgaattacactcgccggagaattacaaaattttttggaaaatacaacttagtcggatattaggactgtccgccacacccctggaccggcccgatggtatggacaagattattattcaccatctaggacagccccagaggactgtaagtcccaccacaaccttttccgggtacgtgagggaaatcgaatatcagggacatcctgacttcgttagccctgtgtgtattaatgaaaaggtatcggccattgccaccattgataaactacttcaagatccttcgcgtatacaacttgtcgtaaatgaggcaaagcggctttactccctgcataccgctgagcctcacaaatgggggaccgatgagccgtatggcatcatcattttcgtggaatttcgcaaacttttagaaattttttatcaggcgctttccaaagaattcaaagatgttcaaattgtcgttccggaggtggcgctcctatgcggcggggtttcaaataccgctctttctcaggcacacagcgcttccattatcttgctgacctatggctacgggcgtagaggcatttccttcaagcatatgacatcgatcatcatggcaacgccccgcagaaacaacatggagcaaatcttgggacgtattacccggcagggatcggatgaaaaaaaggtacgcatcgtcgtggacattaaagatacactaagcccgctttctagccaggtctacgacaggcaccggatttacaagaaaaagggctaccccatttttaagtgcagcgctagctatcagcagccctattcttctaatgaagttttaatatgggatccttataacgagtcatgtcttgcgtgcacaacaacacctccttccccgtccaaatag

SEQ ID NO: 4 - DNA, 10772 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgattgttgcacgggagaaccgttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgactttggcaagtaagcccgcgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaaagaaaaagagaaaagtatacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttaacaatcgtctcgtggagttttagagctagaaatagcaagttaaaataaggctagtccgttatcaacttgaaaaagtggcaccgagtcggtgcttttttct

SEQ ID NO: 5 - DNA, 10771 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaccaagatctggacgggtggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgggtgtatgacacgttgtcggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaaagaaaaagagaaaagtatacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtgtttaacgacatatcgccagttttagagctagaaatagcaagttaaaataaggctagtccgttatcaacttgaaaaagtggcaccgagtcggtgcttttttct

SEQ ID NO: 6 - DNA, 813 bp, African swine fever virus
atgcttggtctgcaaatattcaccctactatctattccaactcttctttatacatatgagatagagcccctggaacgaacaagtaccccacctgaaaaggagcttggatactggtgcacttatgcaaaccattgtagattctgttgggactgtcaagatggtatctgtaggaacaaggcttttaaaaaccattctcccattcttgaaaatgactatatagctaactgcagtatttatcgtcgcaatgatttctgtatctactacataacttctataaagcctcataaaacgtaccgaacagaatgtccacaacacataaaccatgaaaggcatgaggctgatatacgaaaatggcagaaattgttaacctatggattttatcttgcgggatgtattttagctgtgaattacattcgcaaacgtagtttacagactgttatgtatttgctggtcttcctggtaatctcctttctgctttcccaactgatgctgtatggagagttagaagataaaaaacataaaattggcagcattcctcccaaaagagagcttgaacattggtgtactcatggaaaatattgtaacttttgctgggactgtcaaaatggcatctgtaagaataaggcttttaagaatcatccccccataggtgaaaatgattttattagatatgattgttggacaacacatctgccaaataaatgttcctatgaaaaaatatataaacactttaatacccatattatggaatgttcccaacctacacactttaaatggtatgataatttgatgaagaaacaagatattatgtag

SEQ ID NO: 7 - DNA, 315 bp, African swine fever virus
atgaggttctttagttacctcggcttgctgctagctggtctaactagtctacagggtttttcgaccgacaatctcctggaagaggagctaagatactggtgtcaatatgtgaaaaattgtcggttttgctggacttgtcaagatggtctttgtaagaataaagttcttaaagatatgtcttctgtacaggagcatagctatcccatggaacactgtatgattcaccgtcagtataaatatattagagatgggcccattttccaagtagaatgcacgatgcagacatctgatgccactcatttaataaatgcttga

SEQ ID NO: 8 - DNA, 375 bp, African swine fever virus
atgttggtgatcttcttgggaattcttggcctgctggccaatcaggtcttaggactacctactcaggcaggagggcatcttcgttcaacggataatcctccacaagaagaacttggatactggtgtacttacatggaaagctgcaagttttgctgggaatgtgcacatggaatttgcaagaacaaggtgaatgagagcatgccattgattattgagaacagttatttgacatcttgtgaggtttctcgctggtataaccagtgcacatatagtgaaggaaatgggcattaccatgttatggattgttctaatccagtacctcacaatcgtccacaccgattgggaaggaaaatttatgaaaaggaagatctgtga

SEQ ID NO: 9 - DNA, 249 bp, African swine fever virus
atgttagtaatcttcttgggaattcttggccttctggccaaccaggtctcaagccagctcgttggacaacttcatccaacggaaaatccttcagagaatgaacttgaatattggtgcacttacatggaatgttgccagttttgctgggactgtcaaaatggcctttgtgtgaataagttgggaaatacaacaattcttgaaaatgagtatgtgcatccatgtatagtttcccgctggctaaataaataa

SEQ ID NO: 10 - DNA, 357 bp, African swine fever virus
atgttggtgatctttttgggaattcttggccttctggccagccaggtttcaagtcaactcgttggacaacttcgaccaacagaggatcctccagaggaagaactcgaatactggtgcgcctacatggaaagttgtcaattttgctgggactgccaagatggcacttgtataaacaaaatagatgggtcggccatttataagaatgagtatgtgaaagcatgtctggtgtcccgttggctggataaatgtatgtatgatttagataaaggtatctaccataccatgaattgttctcagccatggtcttggaatccttacaaatacttcaggaaggaatggaaaaaagatgaactctag

SEQ ID NO: 11 - DNA, 20 bp, African swine fever virus
ggttctcccgtgcaacaatc

SEQ ID NO: 12 - DNA, 20 bp, African swine fever virus
tccacgagacgattgttaaa

SEQ ID NO: 13 - DNA, 20 bp, African swine fever virus
gcgggcttacttgccaaagt

SEQ ID NO: 14 - DNA, 20 bp, African swine fever virus
cgacaacgtgtcatacaccc

SEQ ID NO: 15 - DNA, 20 bp, African swine fever virus
cacccgtccagatcttggtc

SEQ ID NO: 16 - DNA, 20 bp, African swine fever virus
tggcgatatgtcgttaaaca

SEQ ID NO: 17 - DNA, 20 bp, African swine fever virus
cgtgttcgaaggaccccttt

SEQ ID NO: 18 - DNA, 20 bp, African swine fever virus
actatgtgcgttcccgtatg

SEQ ID NO: 19 - DNA, 20 bp, African swine fever virus
cttgtaaaagccgaagtaaa

SEQ ID NO: 20 - DNA, 20 bp, African swine fever virus
attcttgaaaatgactatat

SEQ ID NO: 21 - DNA, 20 bp, African swine fever virus
ttcttgaaaatgactatata

SEQ ID NO: 22 - DNA, 20 bp, African swine fever virus
ttgtagattctgttgggact

SEQ ID NO: 23 - DNA, 20 bp, Artificial Sequence
  Description of Artificial Sequence: Synthetic oligonucleotide
gattgttgcacgggagaacc

SEQ ID NO: 24 - DNA, 20 bp, Artificial Sequence
  Description of Artificial Sequence: Synthetic oligonucleotide
tttaacaatcgtctcgtgga

SEQ ID NO: 25 - DNA, 20 bp, Artificial Sequence
  Description of Artificial Sequence: Synthetic oligonucleotide
actttggcaagtaagcccgc

SEQ ID NO: 26 - DNA, 20 bp, Artificial Sequence
  Description of Artificial Sequence: Synthetic oligonucleotide
gggtgtatgacacgttgtcg

SEQ ID NO: 27 - DNA, 20 bp, Artificial Sequence
  Description of Artificial Sequence: Synthetic oligonucleotide
gaccaagatctggacgggtg

SEQ ID NO: 28 - DNA, 20 bp, Artificial Sequence
  Description of Artificial Sequence: Synthetic oligonucleotide
tgtttaacgacatatcgcca

SEQ ID NO: 29 - DNA, 20 bp, Artificial Sequence
  Description of Artificial Sequence: Synthetic oligonucleotide
aaaggggtccttcgaacacg

SEQ ID NO: 30 - DNA, 20 bp, Artificial Sequence
  Description of Artificial Sequence: Synthetic oligonucleotide
catacgggaacgcacatagt

SEQ ID NO: 31 - DNA, 20 bp, Artificial Sequence
  Description of Artificial Sequence: Synthetic oligonucleotide
tttacttcggcttttacaag

SEQ ID NO: 32 - DNA, 20 bp, Artificial Sequence
  Description of Artificial Sequence: Synthetic oligonucleotide
atatagtcattttcaagaat

SEQ ID NO: 33 - DNA, 20 bp, Artificial Sequence
  Description of Artificial Sequence: Synthetic oligonucleotide
tatatagtcattttcaagaa

SEQ ID NO: 34 - DNA, 20 bp, Artificial Sequence
  Description of Artificial Sequence: Synthetic oligonucleotide
agtcccaacagaatctacaa

SEQ ID NO: 35-36 - skipped (2 entries)

SEQ ID NO: 37 - DNA, 508 bp, Artificial Sequence
  Description of Artificial Sequence: Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 38 - DNA, 43 bp, African swine fever virus
tatttaataaaaacaataaattatttttataacattatatatg

SEQ ID NO: 39 - DNA, 175 bp, African swine fever virus
tttgtactttgccgcggataaattgccaagcatacataagttgttgataatttctaataaatctggatcgtgctgctgcagccatacagaaatcttgcaaaactgtttcatattagagggcatcttcttattattttataattttaaaattgaatggattttattttaaatatat

SEQ ID NO: 40 - DNA, 67 bp, African swine fever virus
tacccggtatagaaaataaaatttaaaataaaaaacggatgatatctattcatggaccgttctgaga

SEQ ID NO: 41 - DNA, 10776 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcaactcatagagagttagcggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcgtggtttagagaagcgcacgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaagcgccggcagcagcggcgtatacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcctctgaccttaattatagggttttagagctagaaatagcaagttaaaataaggctagtccgttatcaacttgaaaaagtggcaccgagtcggtgcttttttct

SEQ ID NO: 42 - DNA, 9873 bp, Artificial Sequence
  Description of Artificial Sequence: Synthetic polynucleotide
agacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaagcgccggcagcagcggcgtatacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccggagaagcttctctgttttagagctagaaatagcaagttaaaataaggctagtccgttatcaacttgaaaaagtggcaccgagtcggtgcttttttct

SEQ ID NO: 43 - DNA, 10774 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaccaagatctggacgggtggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgggtgtatgacacgttgtcggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaagcgccggcagcagcggcgtatacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtgtttaacgacatatcgccagttttagagctagaaatagcaagttaaaataaggctagtccgttatcaacttgaaaaagtggcaccgagtcggtgcttttttct

SEQ ID NO: 44 - DNA, 10773 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttacttcggcttttacaaggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaaaggggtccttcgaacacggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaagcgccggcagcagcggctacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcatacgggaacgcacatagtgttttagagctagaaatagcaagttaaaataaggctagtccgttatcaacttgaaaaagtggcaccgagtcggtgcttttttct

SEQ ID NO: 45 - DNA, 10775 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgattgttgcacgggagaaccgttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgactttggcaagtaagcccgcgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaagcgccggcagcagcggcgtatacccctacgacgtgcccgactacgccctcgaggagggcagaggaagtcttctaacatgcggtgacgtggaggagaatcccggccctatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaggcggccgcagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttaacaatcgtctcgtggagttttagagctagaaatagcaagttaaaataaggctagtccgttatcaacttgaaaaagtggcaccgagtcggtgcttttttct

SEQ ID NO: 46 - DNA, 8604 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcaactcatagagagttagcggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcgtggtttagagaagcgcacgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcctctgaccttaattatagggttttagagctagaaatagcaagttaaaataaggctagtccgttatcaacttgaaaaagtggcaccgagtcggtgcttttttct

SEQ ID NO: 47 - DNA, 8987 bp, Artificial Sequence
  Description of Artificial Sequence: Synthetic polynucleotide
agacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgacagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccggagaagcttctctgttttagagctagaaatagcaagttaaaataaggctagtccgttatcaacttgaaaaagtggcaccgagtcggtgcttttttct

SEQ ID NO: 48 - DNA, 9888 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaccaagatctggacgggtggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgggtgtatgacacgttgtcggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgacagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtgtttaacgacatatcgccagttttagagctagaaatagcaagttaaaataaggctagtccgttatcaacttgaaaaagtggcaccgagtcggtgcttttttct

SEQ ID NO: 49 - DNA, 8604 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttacttcggcttttacaaggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaaaggggtccttcgaacacggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcatacgggaacgcacatagtgttttagagctagaaatagcaagttaaaataaggctagtccgttatcaacttgaaaaagtggcaccgagtcggtgcttttttct

SEQ ID NO: 50 - DNA, 9889 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgattgttgcacgggagaaccgttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgactttggcaagtaagcccgcgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgacagatgggggtcctgggccccagggtgtgcagccactgacttggggactgctggtggggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattggagatccaaaccaaggcgcgcgctagcgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagcagtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcggtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttaacaatcgtctcgtggagttttagagctagaaatagcaagttaaaataaggctagtccgttatcaacttgaaaaagtggcaccgagtcggtgcttttttct

SEQ ID NO: 51 - DNA, 8604 bp, Artificial Sequence; identical to SEQ ID NO: 46
  Description of Artificial Sequence: Synthetic polynucleotide

SEQ ID NO: 52 - DNA, 7701 bp, Artificial Sequence
  Description of Artificial Sequence: Synthetic polynucleotide
agacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccggagaagcttctctgttttagagctagaaatagcaagttaaaataaggctagtccgttatcaacttgaaaaagtggcaccgagtcggtgcttttttct

SEQ ID NO: 53 - DNA, 8602 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaccaagatctggacgggtggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgggtgtatgacacgttgtcggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtgtttaacgacatatcgccagttttagagctagaaatagcaagttaaaataaggctagtccgttatcaacttgaaaaagtggcaccgagtcggtgcttttttct

SEQ ID NO: 54 - DNA, 8604 bp, Artificial Sequence; identical to SEQ ID NO: 49
  Description of Artificial Sequence: Synthetic polynucleotide

SEQ ID NO: 55 - DNA, 8603 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgattgttgcacgggagaaccgttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgactttggcaagtaagcccgcgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttaacaatcgtctcgtggagttttagagctagaaatagcaagttaaaataaggctagtccgttatcaacttgaaaaagtggcaccgagtcggtgcttttttct

SEQ ID NO: 56 - DNA, 8209 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcaactcatagagagttagcggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcgtggtttagagaagcgcacgttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggccttgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcctctgaccttaattatagggttttagagctagaaatagcaagttaaaataaggctagtccgttatcaacttgaaaaagtggcaccgagtcggtgcttttttct

SEQ ID NO: 57 - DNA, 7306 bp, Artificial Sequence
  Description of Artificial Sequence: Synthetic polynucleotide
agacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggccttgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccggagaagcttctctgttttagagctagaaatagcaagttaaaataaggctagtccgttatcaacttgaaaaagtggcaccgagtcggtgcttttttct

SEQ ID NO: 58 - DNA, 8207 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaccaagatctggacgggtggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgggtgtatgacacgttgtcggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggccttgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtgtttaacgacatatcgccagttttagagctagaaatagcaagttaaaataaggctagtccgttatcaacttgaaaaagtggcaccgagtcggtgcttttttct

SEQ ID NO: 59 - DNA, 8209 bp, Artificial Sequence
  Description of Artificial Sequence: Synthetic polynucleotide
agacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgtttacttcggcttttacaaggttttagagctagaaatagcaagttaaaataaggctagtccgttatcaacttgaaaaagtggcaccgagtcggtgcttttttctagacacaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgaaaggggtccttcgaacacggttttagagctagaaatagcaagttaaaataaggctagtccgttatcaacttgaaaaagtggcaccgagtcggtgcttttttctagacacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccaccatggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactgatgaccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaaggtaagcctgaatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttttttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggccttgcaacgatttaggtgacactatagaagagaaggaattaatacgactcactatagggagagagagagaattaccctcactaaagggaggagaagcatgaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgcatacgggaacgcacatagtgttttagagctagaaatagcaagttaaaataaggctagtccgttatcaacttgaaaaagtggcaccgagtcggtgcttttttct

SEQ ID NO: 60 - DNA, 8209 bp, Artificial Sequence; identical to SEQ ID NO: 59
  Description of Artificial Sequence: Synthetic polynucleotide

SEQ ID NO: 61 - DNA, 20 bp, African swine fever virus
agcaaaattgacaactttcc

SEQ ID NO: 62 - DNA, 20 bp, African swine fever virus
gcaaaattgacaactttcca

SEQ ID NO: 63 - DNA, 20 bp, African swine fever virus
tcttggcagtcccagcaaaa

SEQ ID NO: 64 - DNA, 20 bp, African swine fever virus
acagaggatcctccagagga

SEQ ID NO: 65 - DNA, 20 bp, Artificial Sequence
  Description of Artificial Sequence: Synthetic oligonucleotide
ggaaagttgtcaattttgct

SEQ ID NO: 66 - DNA, 20 bp, Artificial Sequence
  Description of Artificial Sequence: Synthetic oligonucleotide
tggaaagttgtcaattttgc

SEQ ID NO: 67 - DNA, 20 bp, Artificial Sequence
  Description of Artificial Sequence: Synthetic oligonucleotide
ttttgctgggactgccaaga

SEQ ID NO: 68 - DNA, 20 bp, Artificial Sequence
  Description of Artificial Sequence: Synthetic oligonucleotide
tcctctggaggatcctctgt

SEQ ID NO: 69 - DNA, 2525 bp, Unknown
  Description of Unknown: Procine pancreaticn amylase promoter sequence
ctgacataagctgaaccaatgccttgcataatacctgcaatttagagtctataagtaaaaaccacttattgatcacatgagccatcgtgctgtttttttgctaggaatattaactatgaaatctgctcttaataaggtttatccagaatgacagtcatgtaaatccttattttttataacattaatccaatatcacttaataacaacccggaggttaaaacctgccatacagaggagtacataactatggctgggaatatcaatataagtttcataaaggtatttttccaactgcatatgaaagtaggagtagttactagctattgaagggtgatacaagaaagaagaaaagccctggaaagtcatgaaagaataaaattgttgtcaaatacgcaaaatgtttattttttdcgggagatggatattggggactctgcacttgttgttccgcccctctaacaatttgaaatattgaacttaactccaatgtatgcgattaggctgtggggtctttgggaacaacttaggtcaaagtgacatcatgagagtggaggccccatgatgggttagtgtcctttacgaagagaaagagaatcaggatctctgagctcacactcgtgaggatacaagaagcttgctgtctgtgaacatggaatggggctttgcaagacactggagctgctgatagtgtagtcttgggtttccagcctctagaaatgtgagaaagaaatatttgttgctaagccatccagcctatatggcattcttgttacagcagctggaactgaatgagaaaaataggacacggagtatgttcacgatgtgggctggaggagggaccgaaggagagtgttgggattcacagagtgctctcggaccccctccacaaagctagtacttcctcacttttcctcatcttagtaaatggtgtcatcagatacctgtttcctcaatttttctctttcccccagtcttcggtgctaatctatcgataaaccgattgcttcgccacctctgagatatattctatcagggccctagagcagccactttcctctttcgtggaccactacaaaagcctacctgatctcttggcccccagtcgtgtcctcctataatccggtttttcacagcagagcaagaatggttttcttggaaaggaaatcagaatctcttcactcatcttctttcagcctcaaaagccctctctttccttatgttctacaaggttctacatgatctggcctacctctctgatttcatctcattttactcttccctttgtcactcacacatgtttagctgcactgatgttgaaagtttgttcagtgtcacttgagtatcccacggttgttcctaccttgggcttttgctattgcactttcctctatggagactgcttttcctctgatcttcaaataagtgggtccttctactccttccagttctggctgacaatcactccctctgaaacagctttcctgactatttccagtctaaaatatcctgaaaaattcagtccttttccctttaactgcaccgtgggttcatgctagttctcactgctctctttaacttagtatcgttgttgttatcattccatcttgctatattttccttaccttcccctagaatgtaggctgagaacaagagtcttgtctgtcttgttcatccttgtatcctgagtatcatgccggcatttagcaaaagcactcggccactacctgttggatgaatggattaggtttttcccacctgtacggttatgtctttactaggatttcttgtaccttacgaaggaaaatagatgtggattcattaacttagtgttttagcacatataagggactttttgctagaaggagaaaaaaaaaagtccattctttcctgctacagccagtgcattttcacatgcgttaatgtaagcgtggggaaaaaaaaatctgacacctaaagtcgtggtcatttcacttccggataacttcctaaatcttagtggagaatctcaagtatctaacaactagggtaggaggtaccaactgaactgagttgaataacatgtgtcttcttacaatggaaacattgcacgtgtttacagacagttagggcaccattgtgactgtgaattcagttggctctaattccgcctctgtcagtgaaggacttcagaaataaaatctaatcctacctaaacaatacatgattaagacctttctgtagataacatgccagatgtttcaaaacttgctgttccctcagtaaggaaaacattgtctgagaaggtcatttagatagtattcctgggagattttcgggatgttcctcacctgtttagtgtaattatcaatagttatttttggagtatgcattcacggtttgtgctctaagtatttattcatgtcaatatttgctttgtaaaatatgcttcttgcaggattataaatacttgccgggaagaccgttgacaacctcagagcaaaatgaagttgtttctgctgctttcagccattgggttctgctgggcc

SEQ ID NO: 70 - DNA, 413 bp, Unknown
  Description of Unknown: human and rat aquaporin-5 sequence
aagggaaccccggcctgggagagggcgcctccggggatccgttgcctagtccaggtactgcccagctaccgggcgtcgaggattgcgaacggtcggggcaggctggcacggtgcccacttttcccaaaactccagccttccaagcccagaagctcgcccggcccaggccgagctggccacgtcggacggccggaccgccctgcaggacccagcccggccgggggcccccgccggcggtgagggaggtgagcggcgccgacctgcgggacgagcaccccggcctcactccgacccagccgggggtgaggcgggtcaggatgctccggtcgcaggaggaaaaggaggagctggaccaaaagcccgaagagaagaaaaggggaaggccgcgcacggagcgcggtaaaggccggcgg

SEQ ID NO: 71 - DNA, 753 bp, Human alphaherpesvirus 1
aaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaa

SEQ ID NO: 72 - DNA, 358 bp, Macaca mulatta polyomavirus 1
ctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 73 - DNA, 508 bp, Human betaherpesvirus 5; identical to SEQ ID NO: 37

SEQ ID NO: 74 - DNA, 263 bp, Rous sarcoma virus
aatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattgcgcattgcagagataattgtatttaagtgcctagctcgatacaataaacgccatttgaccattcaccacattggtgtgcacc

SEQ ID NO: 75 - DNA, 625 bp, Murine leukemia virus
ccctcccccatatgtttacctactgaacatcacttggggttgtagaaactattgggaacttgtcctggagaaaattagtgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgcgctcaataaaagagcccacaacccctcactcggggcgccagtcctccgattgactgagtcgcccgggtacccgtgtatccaataaaccctcttgcagttgcatccgacttgtggtctcgctgttccttgggcgggtctcctctgagtgattgactacccgtcagcgggggtctttcatttgggggctcgtccgggatcgggagacccct

SEQ ID NO: 76 - DNA, 1182 bp, Unknown
  Description of Unknown: mammalian elongation factor 1alpha
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 77 - DNA, 165 bp, Unknown
  Description of Unknown: cytokeratin 18 seuqence
cccggggcggagcggcccggggcggaggggcgcgggctccgagccgtccacctgtggctccggcttccgaagcggctccggggcgggggcggggcctcactctgcgatataactcgggtcgcgcggctcgcgcaggccgccaccgtcgtccgcaaagcctgagtc

SEQ ID NO: 78 - DNA, 398 bp, Unknown
  Description of Unknown: kallikrein sequence
ctactgctggtttctagggtaaccttggactagaggtatatgaccttgtttagagcagtggattgggggtcactggctcctccaccttcctttcacaccccttcaccattgcccctgaccttgccatctgccttaggtcaccataacactaacagctccaggagcaacaggacctgccccacccaatctcagaccttggaaggtatccaaggtgacctagggcccacaggagagcaggtgcaacagggccctcccctcccacagccatgagggtgggagaagggagtacaactctgtcaggaagggcagggctttggccaacatctggctgccaacacggcaggggtggggctgtgggggagaatgagggtttttaaaggctccccaggagcctctac

SEQ ID NO: 79 - protein, 4 aa, Unknown
  Description of Unknown: “KDEL” motif
KDEL

SEQ ID NO: 80 - DNA, 219 bp, African swine fever virus
caagaagaacttggatactggtgcacttacatggaaagctgcaagttttgctgggaatgtgaacatggaatttgcaagaacaaggtgaataggagcatgccatggattattgagaacagttatttgacatcttgtgaggtttctcgctggtataaccagtgtacatatgatgaaggaaatggacattaccatgtaatggattgttctaatccagtacct

SEQ ID NO: 81 - DNA, 219 bp, African swine fever virus
caagaagaacttggatactggtgtacttacatggaaagctgcaagttttgctgggaatgtgcacatggaatttgcaagaacaaggtgaatgagagcatgccattgattattgagaacagttatttgacatcttgtgaggtttctcgctggtataaccagtgcacatatagtgaaggaaatgggcattaccatgttatggattgttctgatccagtacct

SEQ ID NO: 82 - DNA, 225 bp, African swine fever virus
aagggagagcttgagtattggtgcacctatgcaaaatcgtgcgacttctgctggaattgtcgacacgggatttgcaaaaataaggtttttgaaaaacatcctctcatcaaaaaaaatgattacatacaaatatgtagggtttcccgctataataatagatgtagctactttacagactctaggatacgccgctttcacatcatgagctgtacaaatcccacatat

SEQ ID NO: 83 - DNA, 228 bp, African swine fever virus
aagagtgagctttcatattggtgtacgtatgtaccacaatgcgacttctgttgggactgtcaagatggcatttgcaaaaataagataacggaatctcgtttcattgactccaatcattctattgtaaactgtagagtttttcgtgattccaagactcaatcctgtttatatgaaatatcagttaaaatacctaatcatcatagcatggaatgttcacatcctagaccc

SEQ ID NO: 84 - DNA, 225 bp, African swine fever virus
ataagagagcttgaatactggtgcacgtatggaaaacactgtgatttctgctgggaatgtaggaatggtatttgtaaaaataaagtttgggatgatatgcctcttattaaacaaaatgattatattagtcaatgtagtattgcccgctattttgaccgttgtatatattttattaaacccaaatcaccctatattcattatatggattgttctcaacctacagca

SEQ ID NO: 85 - DNA, 228 bp, African swine fever virus
gaaaaggagtttggatactggtgcacttatgcaaaccattgtagattctgttgggactgtcaagatggtatctgtaggaacaaggcttttaaaaaccattctcccattcttgaaaatgactatatagctaactgcagtatttatcgtcgcaatgatttctgtatctaccacataacttctataaagcctcataaaacgtaccgaacagaatgtccacaacacataaac

SEQ ID NO: 86 - DNA, 224 bp, African swine fever virus
gaagaggagctaagatactggtgtcaatatgtgaaaaattgtcggttttgctggacttgtcaagatggtctttgtaagaataaagttcttaaagatatgtcttctgtacaggagcatagctatcccatgaaacactgtatgattcaccgtcagtgtaaatatattagagatgggcccattttccaagtagaatgcacgatacagacatctgatgccactcattt

SEQ ID NO: 87 - DNA, 357 bp, Artificial Sequence
  Description of Artificial Sequence: Synthetic polynucleotide
  modified_base (6)..(6): a, c, t, g, unknown or other
  modified_base (9)..(9): a, c, t, g, unknown or other
  modified_base (11)..(11): a, c, t, g, unknown or other
  modified_base (15)..(15): a, c, t, g, unknown or other
  modified_base (19)..(20): a, c, t, g, unknown or other
  modified_base (24)..(25): a, c, t, g, unknown or other
  modified_base (27)..(29): a, c, t, g, unknown or other
  modified_base (33)..(33): a, c, t, g, unknown or other
  modified_base (36)..(36): a, c, t, g, unknown or other
  modified_base (39)..(39): a, c, t, g, unknown or other
  modified_base (41)..(42): a, c, t, g, unknown or other
  modified_base (44)..(54): a, c, t, g, unknown or other
  modified_base (60)..(61): a, c, t, g, unknown or other
  modified_base (63)..(63): a, c, t, g, unknown or other
  modified_base (65)..(66): a, c, t, g, unknown or other
  modified_base (69)..(69): a, c, t, g, unknown or other
  modified_base (73)..(76): a, c, t, g, unknown or other
  modified_base (81)..(81): a, c, t, g, unknown or other
  modified_base (84)..(84): a, c, t, g, unknown or other
  modified_base (90)..(90): a, c, t, g, unknown or other
  modified_base (93)..(93): a, c, t, g, unknown or other
  modified_base (96)..(97): a, c, t, g, unknown or other
  modified_base (99)..(99): a, c, t, g, unknown or other
  modified_base (102)..(102): a, c, t, g, unknown or other
  modified_base (105)..(105): a, c, t, g, unknown or other
  modified_base (107)..(107): a, c, t, g, unknown or other
  modified_base (117)..(118): a, c, t, g, unknown or other
  modified_base (120)..(120): a, c, t, g, unknown or other
  modified_base (123)..(124): a, c, t, g, unknown or other
  modified_base (127)..(127): a, c, t, g, unknown or other
  modified_base (130)..(131): a, c, t, g, unknown or other
  modified_base (135)..(138): a, c, t, g, unknown or other
  modified_base (150)..(150): a, c, t, g, unknown or other
  modified_base (157)..(157): a, c, t, g, unknown or other
  modified_base (162)..(163): a, c, t, g, unknown or other
  modified_base (170)..(170): a, c, t, g, unknown or other
  modified_base (174)..(174): a, c, t, g, unknown or other
  modified_base (177)..(178): a, c, t, g, unknown or other
  modified_base (180)..(193): a, c, t, g, unknown or other
  modified_base (195)..(196): a, c, t, g, unknown or other
  modified_base (198)..(198): a, c, t, g, unknown or other
  modified_base (202)..(203): a, c, t, g, unknown or other
  modified_base (207)..(207): a, c, t, g, unknown or other
  modified_base (211)..(213): a, c, t, g, unknown or other
  modified_base (217)..(217): a, c, t, g, unknown or other
  modified_base (220)..(223): a, c, t, g, unknown or other
  modified_base (225)..(225): a, c, t, g, unknown or other
  modified_base (229)..(229): a, c, t, g, unknown or other
  modified_base (233)..(234): a, c, t, g, unknown or other
  modified_base (236)..(237): a, c, t, g, unknown or other
  modified_base (244)..(248): a, c, t, g, unknown or other
  modified_base (250)..(250): a, c, t, g, unknown or other
  modified_base (253)..(259): a, c, t, g, unknown or other
  modified_base (262)..(266): a, c, t, g, unknown or other
  modified_base (268)..(270): a, c, t, g, unknown or other
  modified_base (272)..(280): a, c, t, g, unknown or other
  modified_base (282)..(282): a, c, t, g, unknown or other
  modified_base (286)..(288): a, c, t, g, unknown or other
  modified_base (292)..(330): a, c, t, g, unknown or other
  modified_base (333)..(336): a, c, t, g, unknown or other
  modified_base (338)..(354): a, c, t, g, unknown or other
  modified_base (356)..(357): a, c, t, g, unknown or other
atgttngtnancttnttgnnaatnntnnncctnctngcnanncnnnnnnnnnnncaactnntngnncancttnnnncaacnganaatccnccngannanganctngnatactggtgnncntanntgnaannttgnnnnttttgctgggantgtcaanatggnntttgtangaanaanntnnnnnnnnnnnnnncnncnattnntganaatnnntatntgnnnncntgtntggnntnncgctggnnnnntnaannnnnnnatnnnnnannnannnnnnnnnancatnnnatgnnnnnnnnnnnnnnnnnnnnnnnnnnnnnnnnnnnnnnnttnnnnannnnnnnnnnnnnnnnntnn

SEQ ID NO: 88 - DNA, 357 bp, African swine fever virus; identical to SEQ ID NO: 10

SEQ ID NO: 89 - DNA, 249 bp, African swine fever virus; identical to SEQ ID NO: 9

SEQ ID NO: 90 - DNA, 375 bp, African swine fever virus; identical to SEQ ID NO: 8

SEQ ID NO: 91 - DNA, 315 bp, African swine fever virus
atgaggttctttagttacctcggcttgctgctagctggtctaactagtctacagggtttttcgaccgacaatctcctggaagaggagctaagatactggtgtcaatatgtgaaaaattgtcggttttgctggacttgtcaagatggtctttgtaagaataaagttcttaaagatatgtcttctgtacaggagcatagctatcccatggaacactgtatgattcaccgtcagtgtaaatatattagagatgggcccattttccaagtagaatgcacgatgcagacatctgatgccactcatttaataaatgcttga

SEQ ID NO: 92 - DNA, 813 bp, African swine fever virus
atgcttggtctgcaaatattcaccctactatctattccaactcttctttatacatatgagatagagcccctggaacgaacaagtaccccacctgaaaaggagcttggatactggtgcacttatgcaaaccattgtagattctgttgggactgtcaagatggtatctgtaggaacaaggcttttaaaaaccattctcccattcttgaaaataactatatagctaactgcagtatttatcgtcgcaatgatttctgtatctactacataacttctataaagcctcataaaacgtaccgaacagaatgtccacaacacataaaccatgaaaggcatgaggctgatatacgaaaatggcagaaattgttaacctatggattttatcttgcgggatgtattttagctgtgaattacattcgcaaacgtagtttacagactgttatgtatttgctggtcttcctggtaatctcctttctgctttcccaactgatgctgtatggagagttagaagataaaaaacataaaattggcagcattcctcccaaaagagagcttgaacattggtgtactcatggaaaatattgtaacttttgctgggactgtcaaaatggcatctgtaagaataaggcttttaagaatcatccccccataggtgaaaatgattttattagatatgattgttggacaacacatctgccaaataaatgttcctatgaaaaaatatataaacactttaatacccatattatggaatgttcccaacctacacactttaaatggtatgataatttgatgaagaaacaagatattatgtag

SEQ ID NO: 93 - DNA, 60 bp, Artificial Sequence
  Description of Artificial Sequence: Synthetic oligonucleotide
  modified_base (5)..(5): a, c, t, g, unknown or other
aagangaacttggatactggtgcacttacatggaaagttgtcagttttgctgggactgtc

SEQ ID NO: 94 - DNA, 60 bp, African swine fever virus
aggaagaactcgaatactggtgcgcctacatggaaagttgtcaattttgctgggactgcc

SEQ ID NO: 95 - DNA, 60 bp, African swine fever virus
agaatgaacttgaatattggtgcacttacatggaatgttgccagttttgctgggactgtc

SEQ ID NO: 96 - DNA, 60 bp, African swine fever virus
aagaagaacttggatactggtgtacttacatggaaagctgcaagttttgctgggaatgtg

SEQ ID NO: 97 - DNA, 60 bp, African swine fever virus
aagaggagctaagatactggtgtcaatatgtgaaaaattgtcggttttgctggacttgtc

SEQ ID NO: 98 - DNA, 60 bp, African swine fever virus
aaaaggagcttggatactggtgcacttatgcaaaccattgtagattctgttgggactgtc

SEQ ID NO: 99 - DNA, 120 bp, Artificial Sequence
  Description of Artificial Sequence: Synthetic polynucleotide
  modified_base (5)..(5): a, c, t, g, unknown or other
  modified_base (68)..(68): a, c, t, g, unknown or other
  modified_base (76)..(76): a, c, t, g, unknown or other
  modified_base (86)..(88): a, c, t, g, unknown or other
  modified_base (90)..(90): a, c, t, g, unknown or other
  modified_base (95)..(95): a, c, t, g, unknown or other
  modified_base (97)..(97): a, c, t, g, unknown or other
  modified_base (101)..(102): a, c, t, g, unknown or other
  modified_base (104)..(104): a, c, t, g, unknown or other
  modified_base (119)..(119): a, c, t, g, unknown or other
aagangaacttggatactggtgcacttacatggaaagttgtcagttttgctgggactgtcaagatggnatttgtangaacaaggtnnntnaaganangtcnncnattcttgagaataant

SEQ ID NO: 100 - DNA, 114 bp, African swine fever virus
aggaagaactcgaatactggtgcgcctacatggaaagttgtcaattttgctgggactgccaagatggcacttgtataaacaaaatagatgggtcggccatttataagaatgagt

SEQ ID NO: 101 - DNA, 114 bp, African swine fever virus
agaatgaacttgaatattggtgcacttacatggaatgttgccagttttgctgggactgtcaaaatggcctttgtgtgaataagttgggaaatacaacaattcttgaaaatgagt

SEQ ID NO: 102 - DNA, 120 bp, African swine fever virus
aagaagaacttggatactggtgtacttacatggaaagctgcaagttttgctgggaatgtgcacatggaatttgcaagaacaaggtgaatgagagcatgccattgattattgagaacagtt

SEQ ID NO: 103 - DNA, 120 bp, African swine fever virus
aagaggagctaagatactggtgtcaatatgtgaaaaattgtcggttttgctggacttgtcaagatggtctttgtaagaataaagttcttaaagatatgtcttctgtacaggagcatagct

SEQ ID NO: 104 - DNA, 120 bp, African swine fever virus
aaaaggagcttggatactggtgcacttatgcaaaccattgtagattctgttgggactgtcaagatggtatctgtaggaacaaggcttttaaaaaccattctcccattcttgaaaataact

SEQ ID NO: 105 - DNA, 103 bp, Artificial Sequence
  Description of Artificial Sequence: Synthetic polynucleotide
  modified_base (1)..(1): a, c, t, g, unknown or other
  modified_base (3)..(3): a, c, t, g, unknown or other
  modified_base (18)..(18): a, c, t, g, unknown or other
  modified_base (23)..(23): a, c, t, g, unknown or other
  modified_base (30)..(30): a, c, t, g, unknown or other
  modified_base (33)..(33): a, c, t, g, unknown or other
  modified_base (48)..(48): a, c, t, g, unknown or other
ngncaactcgttggacancttcntccaacnganaatcctccagaagangaacttggatactggtgcacttacatggaaagttgtcagttttgctgggactgtc

SEQ ID NO: 106 - DNA, 103 bp, African swine fever virus
agtcaactcgttggacaacttcgaccaacagaggatcctccagaggaagaactcgaatactggtgcgcctacatggaaagttgtcaattttgctgggactgcc

SEQ ID NO: 107 - DNA, 103 bp, African swine fever virus
agccagctcgttggacaacttcatccaacggaaaatccttcagagaatgaacttgaatattggtgcacttacatggaatgttgccagttttgctgggactgtc

SEQ ID NO: 108 - DNA, 112 bp, African swine fever virus
ggactacctactcaggcaggagggcatcttcgttcaacggataatcctccacaagaagaacttggatactggtgtacttacatggaaagctgcaagttttgctgggaatgtg

SEQ ID NO: 109 - DNA, 99 bp, Artificial Sequence
  Description of Artificial Sequence: Synthetic oligonucleotide
taactagtctacagggtttttcgaccgacaatctcctggaagaggagctaagatactggtgtcaatatgtgaaaaattgtcggttttgctggacttgtc

SEQ ID NO: 110 - DNA, 117 bp, African swine fever virus
caactcttctttatacatatgagatagagcccctggaacgaacaagtaccccacctgaaaaggagcttggatactggtgcacttatgcaaaccattgtagattctgttgggactgtc

SEQ ID NO: 111 - DNA, 60 bp, Unknown
  Description of Unknown: cytokeratin 19 sequence
gatacccgccccttcaacatctccatccccctatgggcggggaagttgtagaggtagggg

(19) United States
(12) Patent Application Publication
(10) Pub. No.: US 2023/0310555 A1
(43) Pub. Date: Oct. 5, 2023

(54) COMPOSITIONS FOR GENOME EDITING AND METHODS OF USE THEREOF

(71) Applicant: CIBUS BIOTECHNOLOGIES, INC., Salt Lake City, UT (US)

(72) Inventor: F.C. Thomas ALLNUTT, Glenwood, MD (US)

(21) Appl. No.: 18/003,835

(22) PCT Filed: Jun. 30, 2021
(86) PCT No.: PCT/US2021/039947

§ 371 Date: Dec. 29, 2022

Related U.S. Application Data
(60) Provisional application No. 63/046,565, filed on Jun. 30, 2020.

(51) Int. Cl. A61K 38/46; C12N 15/85; C12N 15/11; C12N 9/22; C12N 15/90; C12N 9/12; C12N 9/90; A61K 31/7088; A61K 48/00; A61P 31/20

(52) U.S. Cl. CPC A61K 38/465; C12N 15/85; C12N 15/11; C12N 15/111; C12N 9/22; C12N 15/907; C12N 9/1252; C12N 9/90; C12Y 599/01003; A61K 31/7088; A61K 48/0066; A61P 31/20; C12N 2800/107; C12N 2310/20; C12N 2800/80; C12N 2800/22

(57) ABSTRACT

The present disclosure concerns methods and compositions for inhibiting replication of viruses in mammalian cells. In some cases the virus can be African Swine Fever virus, or related viruses. The methods described herein can make use of programmable nucleases.

### CROSS-REFERENCE

This application claims the benefit of U.S. Provisional Application No. 63/046,565, entitled “COMPOSITIONS FOR GENOME EDITING AND METHODS OF USE THEREOF”, filed on Jun. 30, 2020, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jun. 29, 2021, is named 58557-702 601 SL.txt and is 318,217 bytes in size.

### BACKGROUND

In the US and elsewhere, many of the approaches to control viral diseases rely on biosecurity to prevent viral disease agents from entering the food production system. Biosecurity is coupled with vaccination for prevention of disease should the viral agent be introduced. However, biosecurity is not absolute and many of the most important viral animal diseases either: (a) have no available and effective therapies or vaccines or (b) regulatory authorities and stakeholders prefer not to use vaccines to ease tracking of such viral diseases by serology. One such critical viral pathogen is an Asfarviridae such as African swine fever virus (ASFV), the causative agent of African Swine Fever (ASF). ASF is a lethal viral hemorrhagic disease of swine that has devastated and continues to devastate pig production in Africa and Asia, while posing a global threat to pork production (e.g., its recent foothold in Poland on the edge of Germany—a huge pork producer). In addition to infecting domestic swine (Sus scrofa ss. domesticus), the ASFV infects wild boar (Sus scrofa), which are playing a role in ASF's rapid spread around the world. Dispersal of the ASFV can occur through contact with infected animals (domestic or wild), while longer distance transmission can be through pork products, materials, and feeds contaminated with ASFV in which the virus has been shown to survive for months or even years depending on how the materials were stored.

### SUMMARY

Provided herein are methods, compositions, and systems for targeting viral genes in mammalian cells and preventing or reducing infection of mammalian cells by viruses. In some aspects, the present disclosure provides for a method for inhibiting infection of or reducing replication of a virus in an animal in need thereof, comprising introducing to a cell of said animal a nuclease comprising a gene-binding moiety, wherein said gene binding moiety is configured to bind at least one essential gene of said virus, wherein said virus belongs to the family Asfarviridae. In some embodiments, said one or more essential genes of said virus encode DNA polymerase or a fragment thereof, Topoisomerase II or a fragment thereof, RNA helicase or a fragment thereof, an MGF family member or a fragment thereof, or any combination thereof. In some embodiments, the DNA polymerase is G1211R or a fragment thereof. In some embodiments, the Topoisomerase II is p1192R or a fragment thereof. In some embodiments, the RNA helicase is QP509L, A859L, F105L, B92L, D1133LK, or Q706L. In some embodiments, said MGF family member belongs to the MGF-100, MGF-110, MGF-300, MGF-360, or MGF-505 families. In some embodiments, said gene-binding moiety is configured to bind more than one gene within a single MGF family. In some embodiments, the MGF-110 family member is MGF-110-L. In some embodiments, the gene-binding moiety binds more than one gene within the MGF-110 family. In some embodiments, said animal is a mammal. In some embodiments, said mammal is a porcine mammal. In some embodiments, said porcine mammal is Sus scrofa, Sus ahenobarbus, Sus barbatus, Sus cebrifons, Sus celebensis, Sus oliveri, Sus philippensis, or Sus verrucosus. In some embodiments, said virus belongs to the genus Asfivirus. In some embodiments, said virus is African swine fever virus (ASFV). In some embodiments, said gene-binding moiety is configured to bind a plurality of different portions of said one or more genes of said virus. In some embodiments, said gene-binding moiety is configured to bind a combination of at least two, at least three, or all four of DNA polymerase, Topoisomerase II, RNA helicase, a MGF-110 family member, or any combination thereof. In some embodiments, said nuclease is a programmable nuclease comprising at least one of a CRISPR-associated (Cas) polypeptide, a zinc finger nuclease (ZFN), a transcription activator-like effector nuclease (TALEN), or a combination thereof. In some embodiments, said nuclease is configured to bind at least 5 consecutive nucleotides at least one sequence selected from SEQ ID NOs: 1-10, 11-34, 61-69, any of the sequences in Table 3, any of the sequences in Table 4, any of the sequences in Table 3, or any of the genes in Tables 1-2 or a variant having at least 80%, 90%, 95%, or 99% identity thereto. In some embodiments, said nuclease is a programmable nuclease comprising a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide. In some embodiments, said gene-binding moiety of said nuclease comprises a heterologous RNA polynucleotide configured to hybridize to said one or more genes of said virus. In some embodiments, said heterologous RNA polynucleotide is configured to hybridize to DNA encoding one or more genes of said virus. In some embodiments, said heterologous RNA polynucleotide is configured to hybridize to mRNA encoding one or more genes of said virus. In some embodiments, said heterologous RNA polynucleotide comprises at least one, at least two, or at least three targeting sequences, wherein said targeting sequence comprises at least 17 consecutive nucleotides of at least one sequence selected from SEQ ID NOs: 11-34, 61-69, any of the sequences in Table 3, any of the sequences in Table 4 or a variant having at least 80%, 90%, 95%, or 99% identity thereto. In some embodiments, introducing a nuclease comprising a gene-binding moiety to said cell of said animal comprises contacting said cell with said nuclease. In some embodiments, said nuclease comprises a ribonucleoprotein complex comprising a Cas polypeptide and at least one, at least two, or at least three (e.g. up to 10, 20, 50, 100, or more) heterologous RNA polynucleotides configured to hybridize to said one or more genes of said virus. In some embodiments, introducing a nuclease comprising a gene-binding moiety to said cell of said animal comprises contacting said cell with a capped mRNA comprising a sequence encoding said nuclease. In some embodiments, said nuclease comprises a Cas polypeptide, wherein introducing a nuclease comprising a gene-binding moiety to said cell of said animal further comprises contacting said cell with at least one, at least two, or at least three heterologous RNA polynucleotides configured to hybridize to said one or more genes of said virus. In some embodiments, said capped mRNA and said heterologous RNA polynucleotide are separate RNAs. In some embodiments, introducing a nuclease comprising a gene-binding moiety to said cell of said animal comprises contacting said cell with a vector comprising a sequence encoding said nuclease. In some embodiments, said nuclease comprises a Cas polypeptide, wherein said vector further encodes at least one, at least two, or at least three heterologous RNA polynucleotides configured to hybridize to said one or more genes of said virus. In some embodiments, said vector is a plasmid, a minicircle, or a viral vector. In some embodiments, said vector is a viral vector, wherein said viral vector is a retroviral vector, an adenoviral vector, an adeno-associated viral vector (AAV), a lentiviral vector, a pox vector, a parvoviral vector, a measles viral vector, betaarterivirus vector, pseudorabies vector, or a herpes simplex virus vector (HSV). In some embodiments, said vector is a lentiviral vector. In some embodiments, said sequence encoding said nuclease is codon-optimized for expression in said animal. In some embodiments, said introducing occurs in vivo, ex vivo, or in vitro. In some embodiments, said nuclease cleaves viral DNA encoding said one or more genes of said virus within said cell of said animal. In some embodiments, said nuclease cleaves mRNA transcribed from viral DNA encoding one or more genes of said virus within said cell of said animal. In some embodiments, said method results in delay of mortality of said animal upon infection with said virus belonging to the family Asfarviridae, cure of viral infection upon infection with some virus, or immunity to infection from said virus. In some embodiments, said method results in reduced mortality of said animal upon infection with said virus belonging to the family Asfarviridae. In some embodiments, introducing to a cell of said animal said nuclease comprises injecting said animal with, administering orally to said animal, or administering nasally to said animal said nuclease or a vector encoding said nuclease.

In some aspects, the present disclosure provides for a vector comprising a sequence encoding at least one programmable nuclease configured to bind at least one essential viral gene of a virus from the family Asfarviridae. In some embodiments, said vector is a plasmid, a minicircle, or a viral vector. In some embodiments, said vector is a viral vector, wherein said viral vector is a retroviral vector, an adenoviral vector, an adeno-associated viral vector (AAV), a lentiviral vector, a pox vector, a parvoviral vector, a measles viral vector, a betaarterivirus vector, a pseudorabies vector or a herpes simplex virus vector (HSV). In some embodiments, said nuclease is a programmable nuclease comprising at least one of a CRISPR-associated (Cas) polypeptide, a zinc finger nuclease (ZFN), a transcription activator-like effector nuclease (TALEN), or a combination thereof. In some embodiments, said programmable nuclease is configured to bind a plurality of different portions of said one or more genes of said virus. In some embodiments, said one or more essential genes of said virus encode DNA polymerase or a fragment thereof, Topoisomerase II or a fragment thereof, RNA helicase or a fragment thereof, an MGF family member or a fragment thereof, or any combination thereof. In some embodiments, said programmable nuclease is configured to bind a combination of at least two, at least three, or all four of DNA polymerase, Topoisomerase II, RNA helicase, or an MGF family member. In some embodiments, said MGF family member belongs to the MGF-100, MGF-110, MGF-300, MGF-360, or MGF-505 families. In some embodiments, said gene-binding moiety is configured to bind more than one gene within a single MGF family. In some embodiments, the MGF family member is MGF-110L. In some embodiments, the gene-binding moiety binds more than one gene within the MGF-110 family. In some embodiments, said nuclease is configured to bind at least 5 consecutive nucleotides at least one sequence selected from SEQ ID NOs: 11-34, 61-69, any of the sequences in Table 3, any of the sequences in Table 4or a variant having at least 80%, 90%, 95%, or 99% identity thereto. In some embodiments, said programmable nuclease comprises a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide. In some embodiments, said vector further comprises a second sequence encoding at least one, at least two, or at least three heterologous RNA polynucleotides configured to hybridize to said one or more genes of said virus. In some embodiments, said heterologous RNA polynucleotide comprises at least one, at least two, or at least three targeting sequences, wherein said targeting sequence comprises at least 17 consecutive nucleotides of at least one sequence selected from SEQ ID NOs: 11-34, 61-69, any of the sequences in Table 3, any of the sequences in Table 4, a variant having at least 80%, 90%, 95%, or 99% identity thereto, or a variant substantially identical thereto. In some embodiments, said sequence encoding said heterologous RNA polynucleotide is operably linked to a sequence comprising a U6 or an ASFV p30 promoter. In some embodiments, said sequence encoding said heterologous RNA polynucleotide is operably linked to a sequence comprising at least 43 consecutive nucleotides of an ASFV p30 promoter, a variant having at least 80%, at least 90%, at least 95%, at least 99% identity thereto, or a variant substantially identical thereto. In some embodiments, said programmable nuclease is operably linked to a sequence comprising a CMV promoter or an ASFV p72 promoter. In some embodiments, said programmable nuclease is operably linked to a sequence comprising at least 43 consecutive nucleotides of an ASFV p72 promoter, a variant having at least 80%, at least 90%, at least 95%, at least 99% identity thereto, or a variant substantially identical thereto. In some embodiments, said sequence encoding said programmable nuclease is codon-optimized for expression in said animal. In some embodiments, said animal is a mammal. In some embodiments, said animal is a mammal and said mammal is a porcine mammal.

In some aspects, the present disclosure provides for a pharmaceutically-acceptable composition, comprising any of the vectors described herein and a pharmaceutically-acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 depicts a schematic of a CRISPR vector construct, based on GeneCopoeia vector pCRISPR-CG04. The vector carries a CMV promoter-driven, mammalian-optimized Cas9 nuclease gene, and 3 sgRNAs designed to target the ASFV DNA polymerase gene, each driven by the U6 promoter. The sgRNAs for each target are provided in the table below the map. Figure discloses SEQ ID NOS 26-28, respectively, in order of appearance.

FIG. 2 depicts a schematic of a CRISPR vector construct, based on GeneCopoeia vector pCRISPR-CG04. The vector carries a CMV promoter-driven, mammalian-optimized Cas9 nuclease gene, and 3 sgRNAs designed to target the ASFV topoisomerase II gene, each driven by the U6 promoter. The sgRNAs for each target are provided in the table below the map. FIG. 2 discloses SEQ ID NOS 23-25, respectively, in order of appearance.

FIG. 3 depicts a schematic of a CRISPR vector construct, based on GeneCopoeia vector pCRISPR-CG04. The vector carries a CMV promoter-driven, mammalian-optimized Cas9 nuclease gene, and 3 sgRNAs designed to target an ASFV RNA helicase gene, each driven by the U6 promoter. The sgRNAs for each target are provided in the table below the map. FIG. 3 discloses SEQ ID NOS 23-25, respectively, in order of appearance.

FIGS. 4, 4A, and 4B depict alignments of MGF multigene families in ASFV. FIG. 4 depicts an alignment of multigene family (MGF) 110 ASFV genes from the OURT 88/3 genome (NC 044957.1) using MAFFT v7.452. The table below depicts three sgRNAs targeting a region of the MGF 110-1R sequence that is highly conserved in other members of the MGF 110 family in ASFV. FIG. 4 discloses SEQ ID NOS 80-86 and 32-34, respectively, in order of appearance. FIG. 4A depicts an alignment of MGF 110 proteins L270L, U104L, XP124L, V82L and Y1118L showing conserved regions. FIG. 4A discloses SEQ ID NOS 87-92, respectively, in order of appearance. FIG. 4B shows conservation of targeted regions between MGF 110 proteins L270L, U104L, XP124L, V82L and Y1118L. FIG. 4B discloses SEQ ID NOS 93-110, respectively, in order of appearance.

FIG. 5 depicts a schematic of a CRISPR vector construct as in FIGS. 1-3 with replacement of the U6 and CMV promoters with early promoters from ASFV (the p30 and DNA polymerase promoters).

FIG. 6 depicts a western blot performed as in EXAMPLE 4 showing expression of Cas endonuclease (e.g. Cas9) from vectors of the current disclosure in mammalian cells.

FIG. 7 depicts a heteroduplex formation assay as described in EXAMPLE 5 demonstrating that the sgRNAs included in vectors according to the current disclosure are effective at targeting ASFV genes.

FIGS. 8, 9, 10, 11, 12, 13, 14, 15, 16, 17, 18, 19, 20, 21, 22, 23, 24, 25, 26, 27, 28, 29, 30, 31, and 32 depict vector maps of ASFV gene targeting vectors as described herein (e.g. described in Table 5). FIGS. 8-12 depict SEQ ID NOs: 71-75; FIGS. 13-18 depict SEQ ID NOs: 46-50; FIGS. 19-23 depict SEQ ID NOs: 51-55; FIGS. 24-28 depict SEQ ID NOs: 56-60; and FIGS. 29-33 present an alternative depiction of SEQ ID NOs: 71-75.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoan cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, crustacean, etc.), a cell from a vertebrate animal (e.g., fish, amphibian, reptile, bird, mammal, etc.), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates such as adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase).

The term “essential viral gene” or grammatical equivalents thereof generally refers to a viral gene required for an essential function of the virus, such as replication or viral particle integrity. Abrogation of function of essential viral genes prevents replication and/or infection with the virus.

The terms “pig”, “swine”, and “porcine” are used herein interchangeably to generally refer to anything related to pigs, including the various breeds of domestic pig, species Sus scrofa.

The terms “treatment,” “treating,” “alleviation” and the like, when used in the context of a disease, injury or disorder, are generally used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a pig, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refers to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors (including baculoviral vectors), liposomes, and other gene delivery vehicles. The vector generally comprises genetic elements, e.g., regulatory elements, operatively linked to a gene to facilitate expression of the gene in a target.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss water/nucleotide.html, optionally with default settings). Optimal alignment can be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” can be used to describe an event that takes place in a subject's body.

As used herein, the term “ex vivo” can be used to describe an event that takes place outside of a subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

The term “pharmaceutically acceptable carrier,” “pharmaceutically acceptable excipient,” “physiologically acceptable carrier,” or “physiologically acceptable excipient” generally refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, P A, 2005; Handbook of Pharmaceutical Excipients, 5th Edition”; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, F L, 2004).

The term “pharmaceutical composition” generally refers to a mixture of a compound (e.g. a polypeptide or polynucleotide) disclosed herein with other chemical components, such as diluents or carriers. The pharmaceutical composition can facilitate administration of the compound to an organism. Multiple techniques of administering a compound exist in the art including, but not limited to, oral, injection, nasal, aerosol, parenteral, and topical administration.

The term “vector” generally refers to an element for introducing a heterologous expressable gene into a cell (e.g. a eukaryotic, prokaryotic, mammalian, or porcine cell). Example vectors include viral (e.g. lentiviral, adenoviral, adeno-associated viral) and non-viral (e.g. plasmid or minicircle) vectors.

### Overview

There is need for improved methods and compositions for control of Asfarviridae (such as African swine flu virus) in porcine animals. Accordingly, provided herein are methods and protein and nucleic acid compositions for nuclease-based targeting of Asfarviridae.

### Antiviral Methods

In one aspect, the present disclosure provides for a method for inhibiting infection of or reducing replication of a virus in an animal in need thereof, comprising introducing to a cell of said animal a nuclease comprising a specific gene-binding moiety.

In some cases, the animal is a porcine animal or another mammal susceptible to infection by Asfarviridae. Exemplary mammals include livestock (including cattle, pigs, etc.), companion animals (e. g., dogs, cats, etc.) and rodents. (e.g., mice and rats). Exemplary porcine mammals include Sus scrofa, Sus ahenobarbus, Sus barbatus, Sus cebrifons, Sus celebensis, Sus oliveri, Sus philippensis, or Sus verrucosus.

In some cases, the virus is a member of the family Asfarviridae. Asfarviridae include members of the genus Asfivirus such as African swine flu virus (ASFV). Asfarviridae are double-stranded DNA viruses and are thus susceptible to genome targeting by nucleases such as Cas endonucleases, zinc-finger nucleases, and TALEN nucleases.

In some cases, the gene binding moiety is configured to bind at least one essential gene of said virus.

The one or more essential genes can include DNA polymerase or a fragment thereof, Topoisomerase II or a fragment thereof, RNA helicase or a fragment thereof, an MGF family member or a fragment thereof, or any combination thereof (e.g. any two of the preceding, any three of the preceding, any four of the preceding). The DNA polymerase can be G1211R or a fragment thereof. The Topoisomerase II can be p1192R or a fragment thereof. The RNA helicase can be at least one of QP509L, A859L, F105L, B92L, D1133LK, or Q706L, or a fragment thereof. The MGF family member can include a member of the MGF-100, MGF-110, MGF-300, MGF-360, or MGF-505 families. In some cases, the genes can include more than one gene within a single MGF family (e.g. by providing moieties that target regions conserved among multiple members of a single MGF family). In some cases, the MGF family is MGF-110 and the family member is MGF-110L. The genes can include Ep152R (see e.g., Borca, M. V., V. O'Donnell, L. G. Holinka, D. K. Rai, B. Sanford, M. Alfano, J. Carlson, P. A. Azzinaro, C. Alonso and D. P. Gladue (2016). “The Ep152R ORF of African swine fever virus strain Georgia encodes for an essential gene that interacts with host protein BAG6.” Virus Res 223: 181-18), I215L E2 ubiquitin-conjugating enzyme (see e.g., de Freitas, F. (2019). Functional characterization of unassigned African swine fever virus proteins putatively involved in transcription and replication towards an efficient vaccine design. PhD, University of Lisbon), Thymidine kinase A240L (see e.g., Moore, D. M., L. Zsak, J. G. Neilan, Z. Lu and D. L. Rock (1998). “The African swine fever virus thymidine kinase gene is required for efficient replication in swine macrophages and for virulence in swine.” J Virol 72(12): 10310-1031), structural protein P54 (see e.g., Rodriguez, F., V. Ley, P. Gomez-Puertas, R. Garcia, J. Rodriguez and J. Escribano (1996). “The structural protein p54 is essential for African swine fever virus viability.” Virus Research 40(2): 161-167), IL19IL, L19KL, L19LL (see e.g., Roberts, P. C., Z. Lu, G. F. Kutish and D. L. Rock (1993). “Three adjacent genes of African swine fever virus with similarity to essential poxvirus genes.” Arch Virol 132(3-4): 331-342), any of the genes described in Table 1 or Table 2 below, or a combination thereof. A fragment that is bound by the gene-binding moiety can include a sequence of a length sufficient to drive binding of the nuclease. Such sequence lengths can generally include from at least about 9 nucleotides to about 20 nucleotides, including at least 10, at least 11, at least 12, at least 13, at least 14, at least 15, at least 16, at least 17, at least 18, at least 19, at least 20, at most 20, at most 19, at most 18, at most 17, at most 16, at most 15, at most 14, at most 13, at most 12 nucleotides, at most 11 nucleotides, at most 10 nucleotides, or at most 9 nucleotides. The gene-binding moiety can be configured to bind a plurality of different (e.g. non-contiguous) portions of said one or more genes of said virus, such as at least 1 portion, at least 2 portions, at least 3 portions, at least 4 portions, at least 5 portions, or more. The gene binding moiety can be configured to bind a combination of at least two, at least three, or all four of DNA polymerase or a fragment thereof, Topoisomerase II or a fragment thereof, RNA helicase or a fragment thereof, an MGF-110 family member or a fragment thereof, or any combination thereof (e.g. any two of the preceding, any three of the preceding, any four of the preceding).

In some cases, the gene-binding moiety is configured to bind a specific sequence within the viral gene targeted. The gene-binding moiety can be configured to bind at least 5 consecutive nucleotides of at least one sequence selected from SEQ ID NOs: 1-10, 11-34, 61-69, any of the sequences in Table 3, any of the sequences in Table 4, any of the sequences in Table 3, or any of the genes in Tables 1-2. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of at least one sequence selected from SEQ ID NOs: 23-34, 65-68 or a reverse complement thereof. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of a variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 11-22 or 61-64 or a reverse complement thereof, or a variant being substantially identical to any one of SEQ ID NOs: 11-22 or 61-64or a reverse complement thereof.

In some cases, the nuclease comprising a gene-binding moiety can comprise a programmable nuclease. Programmable nucleases include at least one of a CRISPR-associated (Cas) polypeptide, a zinc finger nuclease (ZFN), a transcription activator-like effector nuclease (TALEN), or a combination thereof.

Cas polypeptides can include Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or functional fragments thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cash, Cas6e, Cas6f, Cas7, Cas8a, Cas8al, Cas8a2, Cas8b, Cas8c, Csn1, Csx12, Cas10, Cas10d, CasF, CasG, CasH, Csyl, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966. Cas13 can include Cas13a, Cas13b, Cas13c, and Cas 13d (e.g., CasRx). Cas can be DNA (e.g. Cpf1, Cas9) and/or RNA cleaving (e.g. Cas13 members such as Cas13a, Cas13b, Cas13c, or Cas13d).

In some embodiments, the nuclease disclosed herein can be a protein that lacks nucleic acid cleavage activity. In some cases, the Cas protein is a dead Cas protein. A dead Cas protein can be a protein that lacks nucleic acid cleavage activity, which can comprise a modified (e.g. mutated) form of a wild type Cas protein. The modified form of the wild type Cas protein can comprise an amino acid change (e.g., deletion, insertion, or substitution) that reduces the nucleic acid-cleaving activity of the Cas protein. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

In some embodiments, a dCas (e.g., dCas9) polypeptide can associate with a single guide RNA (sgRNA) to repress transcription of target DNA (e.g. when the nuclease further comprises a protein acting as a genetic repressor).

In some cases, the gene binding moiety of the nuclease can comprise a heterologous RNA polynucleotide configured to hybridize to said one or more genes of said virus (e.g. when the nuclease is a Cas polypeptide). The heterologous RNA can be a guide RNA, comprising both a targeting sequence directed against a particular gene sequence, and a scaffold sequence binding to a Cas polypeptide.

The heterologous RNA polynucleotide can comprise at least one (e.g. at least two, at least three) targeting sequences. The targeting sequences can comprise at least 17 (e.g. at least 18, at least 19, at least 20, at least 21, at least 22, at most 22, at most 20, at most 19, at most 18, or at most 17) consecutive nucleotides of at least one sequence selected from SEQ ID NOs: 23-34, 65-68, any of the sequences in Table 4 or a reverse complement thereof. The targeting sequences can comprise at least 17 (e.g. at least 18, at least 19, at least 20, at least 21, at least 22, at most 22, at most 20, at most 19, at most 18, or at most 17) consecutive nucleotides of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 23-34, 65-68, any of the sequences in Table 4 or a reverse complement thereof, or a sequence variant substantially identical to any one of SEQ ID NOs: SEQ ID NOs: 23-34, 65-68, any of the sequences in Table 4 or a reverse complement thereof.

In some cases, introducing a nuclease comprising a gene-binding moiety to said cell of said animal comprises contacting said cell with the nuclease. The nuclease can be a polypeptide alone (e.g. a zinc-finger or TALEN nuclease) or a ribonucleoprotein complex with a heterologous RNA (e.g. when the nuclease comprises a Cas protein). The nuclease can be contacted to the cell in the presence of a transfection agent and/or with the aid of a physical stimulus promoting entry of macromolecules into cells (e.g. electroporation, heat). Example transfection agents include lipid-based systems (e.g., oil-in-water emulsions, micelles, mixed micelles, and liposomes) or nanoparticle systems. Nanoparticle-based systems can comprise e.g., compounds such as chitosan, alginate, carbon nanotubes (see e.g., Zhu, B., G.-L. Liu, Y.-X. Gong, F. Ling and G.-X. Wang (2015). “Protective immunity of grass carp immunized with DNA vaccine encoding the vp7 gene of grass carp reovirus using carbon nanotubes as a carrier molecule.” Fish & Shellfish Immunology 42(2): 325-334), poly lactic acid (PLA see e.g., Betancourt, T., J. D. Byrne, N. Sunaryo, S. W. Crowder, M. Kadapakkam, S. Patel, S. Casciato and L. Brannon-Peppas (2009). “PEGylation strategies for active targeting of PLA/PLGA nanoparticles.” J Biomed Mater Res A 91(1): 263-276.), poly lactic-co-glycolic acid (PLGA, see e.g., Dubey, S., K. Avadhani, S. Mutalik, S. M. Sivadasan, B. Maiti, J. Paul, S. K. Girisha, M. N. Venugopal, S. Mutoloki, O. Evensen, I. Karunasagar and H. M. Munang′andu (2016)), or solid lipids (see e.g., Harde, H., M. Das and S. Jain (2011). “Solid lipid nanoparticles: an oral bioavailability enhancer vehicle.” Expert Opin Drug Deliv 8(11): 1407-1424). General background on construction of nanoparticles for delivery can be found in e.g., Tatiparti, K., S. Sau, S. K. Kashaw and A. K. Iyer (2017). “siRNA Delivery Strategies: A Comprehensive Review of Recent Developments.” Nanomaterials (Basel) 7(4).

In some embodiments, nanoparticles are modified to add targeting moieties to their surface. In some embodiments, the targeting moieties serve to direct the nanoparticles to a particular cell type, such as a macrophage. Such modifications can include addition of e.g., mannose containing compounds, ubiquitinated proteins, targeting aptamers or antibodies, or other cell-specific targeting moieties (see e.g., Hu, G., M. Guo, J. Xu, F. Wu, J. Fan, Q. Huang, G. Yang, Z. Lv, X. Wang and Y. Jin (2019). “Nanoparticles Targeting Macrophages as Potential Clinical Therapeutic Agents Against Cancer and Inflammation.” Frontiers in immunology 10: 1998-1998 for examples).

In some embodiments, nanoparticles are modified by addition of one or more chemical agent to alter release properties in the cell (see e.g., Tatiparti, K., S. Sau, S. K. Kashaw and A. K. Iyer (2017). “siRNA Delivery Strategies: A Comprehensive Review of Recent Developments.” Nanomaterials (Basel) 7(4).). Addition of such agents (e.g. cationic polymers to increase denosomolysis, neutrally charged ionizable lipids that become charged in the endosome to cause endosomal lysis) may enhance delivery of nucleic acids to the cytoplasm instead of endosomal/lysosomal compartments.

The ribonucleoprotein complex can comprise at least one Cas enzyme together with at least one (e.g. at least one, two, three, or more) heterologous RNA polynucleotides targeted against different regions of a same viral gene or different genes.

In some cases, introducing a nuclease comprising a gene-binding moiety to the cell of the animal comprises contacting said cell with a mRNA comprising a sequence encoding the nuclease. Such capped mRNAs can be chemically synthesized or in-vitro transcribed by a variety of suitable methods. Suitable systems for in-vitro transcription of mRNAs include systems based on e.g. rabbit reticulocyte lysate, wheat germ extract, and E. coli lysate. The mRNA can be contacted to the cell in the presence of a transfection agent (e.g. various lipid-based systems including oil-in-water emulsions, micelles, mixed micelles, and liposomes) and/or with the aid of a physical stimulus promoting entry of macromolecules into cells (e.g. electroporation, heat). The mRNA can also be contacted to the cell in the presence of at least one (e.g. at least two, at least three) heterologous RNA polynucleotides directed against one or more region of a viral gene, or one or more viral genes. In some embodiments, the mRNA is a 5′-capped mRNA. Suitable procedures for mRNA capping can be found in e.g., Fechter, P.; Brownlee, G. G. “Recognition of mRNA cap structures by viral and cellular proteins” J. Gen. Virology 2005, 86, 1239-1249; European patent publication 2 010 659 A2; U.S. Pat. No. 6,312,926. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. In some embodiments, the mRNA comprises a poly-A tail. Poly A tails can be added using a variety of procedures including but not limited to: (1) contacting transcribed with poly A polymerase (see e.g., Yokoe, el al. Nature Biotechnology. 1996; 14: 1252-1256), (2) encoding long poly A tails within the DNA used to transcribe the mRNA, (3) transcription directly from PCR products, or (4) ligating a poly A tail to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition).

### Vectors

In some cases, introducing a nuclease comprising a gene-binding moiety to a cell of the animal comprises contacting the cell with a vector comprising a sequence encoding the nuclease.

The vector can be a plasmid, a minicircle (see e.g., U.S. Ser. No. 10/612,030B2, which describes methods of producing minicircles), or a viral vector. Exemplary viral vectors include retroviral vectors, adenoviral vectors, adeno-associated viral vectors (AAVs), pox vectors, parvoviral vectors, baculovirus vectors, measles viral vectors, betaarterivirus vectors, pseudorabies vectors, or herpes simplex virus vectors (HSVs). In some instances, the retroviral vectors include gamma-retroviral vectors such as vectors derived from the Moloney Murine Leukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In some instances, the viral vector is a recombinant viral vector.

In some cases, the vector is a porcine-tropic viral vector. Several porcine viruses have been shown to be amenable to transgene element insertion. In some embodiments, the porcine-tropic viral vector is based on reproductive and respiratory syndrome virus (PRRSV) or pseudorabies virus (PRV). In some cases, the porcine-tropic viral vector is a modified live virus (MLV) or inactivated variant of PRRSV or PRV.

In some cases, the porcine tropic viral vector is a variant of PRRSV. Porcine reproductive and respiratory syndrome virus (PRRSV, also known as Betaarterivirus suid 1) is a single stranded, plus sense RNA virus with a genome of about 15 kb. The USDA has approved a live vaccine derived from PRRSV that has mycoplasma antigens engineered into it (49R8.21, FLEXMycoPRRS™) and is used as a live modified virus vaccine (MLV). Two other USDA approved vaccines are also modified live viral vaccines derived from PRRSV (49K9.RO & 1951.22). PRRSV (specifically, the PRRSV Suvaxyn MLV strain) has also been genetically modified to express interlukin-15 as an immunomodulator transgene (see e.g., Cao, Ni et al. J Virol 92:e00007-18 (2018), which is incorporated by reference herein for the purpose of PRRSV vector design). In some embodiments, the viral vector is a PRRSV Suvaxyn MLV variant. In some embodiments of a PRRSV Suvaxyn MLV variant, one or more of the Cas enzymes and/or sgRNA coding sequences described herein are introduced between ORF1b and ORF2a.

In some cases, the porcine-tropic viral vector is a variant of PRV. Pseudorabies virus (PRV) is a linear 150 kb DNA virus in the alpha herpes viruses. It has been genetically manipulated to remove the virulence genes in order to produce a live modified viral vaccine as well as to express a foreign gene from hog cholera to provide protection against that disease (see e.g. van Zijl, Wensvoort et al. J Virol. 1991 May; 65(5): 2761-2765, which is incorporated herein for the purpose of PRV vector design). The USDA has approved at least four PRV-based vaccines (1981.20, 1891.22, 1891.23 and 1891.24). In some cases, the porcine-tropic viral vector is a deletion variant of PRV strain NIA-3. In some cases, the porcine-tropic viral vector is a deletion variant of PRV in which the gI gene and part of the 11K gene are deleted. In some cases, the porcine-tropic viral vector is a variant of PRV in which a transgene is inserted to replace at least part of the nonessential glycoprotein gX (e.g. the BafI-NdeI fragment of gX). In some cases, the porcine-tropic viral vector is a variant of PRV in which a transgene is inserted to replace at least part of a nonessential gene (e.g. the TK, PK, gE, gI or gG gene). A recent study by Zheng and colleagues provides detailed methods for production of a live attenuated recombinant PRV expressing the porcine parvovirus structural protein VP2 as well as the porcine IL-6 protein (see e.g. Zheng et al. “Characterization of a recombinant pseudorabies virus expressing porcine parvovirus VP2 protein and porcine IL-6”. Virology Journal. 17(19) (2020), which is incorporated herein for the purpose of PRV vaccine design).

In some cases, engineered PRRSV or RSV vectors expressing the CRISPR/Cas nucleases and/or sgRNAs described herein are used as live modified viruses for delivery of therapeutic protection against ASFV and other diseases of swine. In some cases, such a vector has enhanced biosafety features or a lower regulatory approval burden due to the already understood features of such vectors.

The nuclease can comprise any of the nucleases comprising gene-binding moieties described herein, including a CRISPR-associated (Cas) polypeptide, a zinc finger nuclease (ZFN), a transcription activator-like effector nuclease (TALEN). Cas polypeptides can include Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or functional fragments thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cash, Cas6e, Cas6f, Cas7, Cas8a, Cas8al, Cas8a2, Cas8b, Cas8c, Csn1, Csx12, Cas10, Cas10d, Cas10, Cas10d, CasF, CasG, CasH, Csyl, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966. Cas13 can include Cas13a, Cas13b, Cas13c, and Cas 13d (e.g., CasRx). Cas can be DNA (e.g. Cpf1, Cas9) and/or RNA cleaving (e.g. Cas13).

The vector can comprise a sequence encoding the nuclease (e.g. a programmable nuclease, a Cas polypeptide, or any of the other nucleases comprising gene-binding moieties described herein) under the control of or operably linked to a promoter sequence suitable for the animal into which the vector is being introduced. In the case of porcine animals, exemplary promoter sequences include a CMV promoter or a functional fragment thereof or an ASFV p72 promoter or a functional fragment thereof. Such a functional ASFV p72 or CMV promoter can comprise at least 43 or at least 100 consecutive nucleotides (e.g. at least 150, at least 200, at least 250, at least 300, at least 400, at least 500, at least 750, at least 1000, at most 1000, at most 750, at most 500, at most 400, at most 300, at most 250, at most 200, at most 150, or at most 100) of an ASFV p72 or CMV promoter, or at least 43 or 100 consecutive nucleotides (e.g. at least 150, at least 200, at least 250, at least 300, at least 400, at least 500, at least 750, at least 1000, at most 1000, at most 750, at most 500, at most 400, at most 300, at most 250, at most 200, at most 150, or at most 100) of a sequence variant having at least 80%, at least 85%, at least 90%, at least 91%, at least 92%, at least 93%, at least 94%, at least 95%, at least 96%, at least 97%, at least 98%, at least 99% identity to an ASFV p72 or CMV promoter, or a sequence variant substantially identical to an ASFV p72 or CMV promoter.

In some cases, the programmable nuclease is configured to bind at least one essential gene of said virus.

The one or more genes can include DNA polymerase or a fragment thereof, Topoisomerase II or a fragment thereof, RNA helicase or a fragment thereof, an MGF family member or a fragment thereof, or any combination thereof (e.g. any two of the preceding, any three of the preceding, any four of the preceding). The DNA polymerase can be G1211R or a fragment thereof. The Topoisomerase II can be p1192R or a fragment thereof. The RNA helicase can be at least one of QP509L, A859L, F105L, B92L, D1133LK, or Q706L, or a fragment thereof. The MGF family member can include a member of the MGF-100, MGF-110, MGF-300, MGF-360, or MGF-505 families. In some cases, the genes can include more than one gene within a single MGF family (e.g. by providing moieties that target regions conserved among multiple members of a single MGF family). In some cases, the MGF family is MGF-110 and the family member is MGF-110L. The genes can include Ep152R (see e.g., Borca, M. V., V. O'Donnell, L. G. Holinka, D. K. Rai, B. Sanford, M. Alfano, J. Carlson, P. A. Azzinaro, C. Alonso and D. P. Gladue (2016). “The Ep152R ORF of African swine fever virus strain Georgia encodes for an essential gene that interacts with host protein BAG6.” Virus Res 223: 181-18), I215L E2 ubiquitin-conjugating enzyme (see e.g., de Freitas, F. (2019). Functional characterization of unassigned African swine fever virus proteins putatively involved in transcription and replication towards an efficient vaccine design. PhD, University of Lisbon), Thymidine kinase A240L (see e.g., Moore, D. M., L. Zsak, J. G. Neilan, Z. Lu and D. L. Rock (1998). “The African swine fever virus thymidine kinase gene is required for efficient replication in swine macrophages and for virulence in swine.” J Virol 72(12): 10310-1031), structural protein P54 (see e.g., Rodriguez, F., V. Ley, P. Gomez-Puertas, R. Garcia, J. Rodriguez and J. Escribano (1996). “The structural protein p54 is essential for African swine fever virus viability.” Virus Research 40(2): 161-167), IL19IL, L19KL, L19LL (see e.g., Roberts, P. C., Z. Lu, G. F. Kutish and D. L. Rock (1993). “Three adjacent genes of African swine fever virus with similarity to essential poxvirus genes.” Arch Virol 132(3-4): 331-342), any of the genes described in Table 1 or Table 2 below, or a combination thereof. A fragment that is bound by the gene-binding moiety can include a sequence of a length sufficient to drive binding of the nuclease. Such sequence lengths can generally include from at least about 9 nucleotides to about 20 nucleotides, including at least 10, at least 11, at least 12, at least 13, at least 14, at least 15, at least 16, at least 17, at least 18, at least 19, at least 20, at most 20, at most 19, at most 18, at most 17, at most 16, at most 15, at most 14, at most 13, at most 12 nucleotides, at most 11 nucleotides, at most 10 nucleotides, or at most 9 nucleotides. The gene-binding moiety can be configured to bind a plurality of different (e.g. non-contiguous) portions of said one or more genes of said virus, such as at least 1 portion, at least 2 portions, at least 3 portions, at least 4 portions, at least 5 portions, or more. The gene binding moiety can be configured to bind a combination of at least two, at least three, or all four of DNA polymerase or a fragment thereof, Topoisomerase II or a fragment thereof, RNA helicase or a fragment thereof, an MGF-110 family member or a fragment thereof, or any combination thereof (e.g. any two of the preceding, any three of the preceding, any four of the preceding).

In some cases, the programmable nuclease is directed against a specific sequence within the viral gene targeted. The gene-binding moiety can be configured to bind at least 5 consecutive nucleotides of at least one sequence selected from SEQ ID NOs: 1-10, 11-34, 61-69, any of the sequences in Table 3, any of the sequences in Table 4, any of the sequences in Table 3, or any of the genes in Tables 1-2 or a reverse complement thereof. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of at least one sequence selected from SEQ ID NOs: 22-82 or a reverse complement thereof. The programmable nuclease can be configured to bind at least 6, at least 7, at least 8, at least 9, at least 10, at least 11, at least 12, at least 13, at least 14, at least 15, at least 16, at least 17, at least 18, at least 19, or at least 20 nucleotides of a variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 11-22, 61-64, any of the sequences in Table 4, or a reverse complement thereof, or a variant being substantially identical to any one of SEQ ID NOs: 11-22, 61-64, any of the sequences in Table 4, or a reverse complement thereof.

In some cases (e.g. when the nuclease is a Cas polypeptide) the vector can comprise at least one (e.g. at least two, at least three) sequence encoding heterologous RNA polynucleotides comprising targeting sequences against at least one viral gene. The targeting sequences can comprise at least 17 (e.g. at least 18, at least 19, at least 20, at least 21, at least 22, at most 22, at most 20, at most 19, at most 18, or at most 17) consecutive nucleotides of at least one sequence selected from SEQ ID NOs: 23-34, 65-68, any of the sequences in Table 4 or a reverse complement thereof. The targeting sequences can comprise at least 17 (e.g. at least 18, at least 19, at least 20, at least 21, at least 22, at most 22, at most 20, at most 19, at most 18, or at most 17) consecutive nucleotides of a sequence variant having at least 80%, at least 85%, at least 90%, at least 91%, at least 92%, at least 93%, at least 94%, at least 95%, at least 96%, at least 97%, at least 98%, at least 99% identity to any one of SEQ ID NOs: 23-34, 65-68, any of the sequences in Table 4, or a reverse complement thereof, or a sequence variant substantially identical to any one of SEQ ID NOs: SEQ ID NOs: 25-36 or a reverse complement thereof.

In some cases, the at least one (e.g. at least two, at least three) sequence encoding heterologous RNA polynucleotides can be under the control of or operably linked to a viral promoter sequence or a mammalian or eukaryotic promoter sequence. An example eukaryotic promoter can be a U6 promoter. Alternatively or additionally, an exemplary viral promoter is the p30 promoter of ASFV, or a functional fragment thereof. Such a promoter sequence can comprise at least 43 or at least 100 (e.g. at least 150, at least 200, at least 250, at least 300, at least 400, at least 500, at least 750, at least 1000, at most 1000, at most 750, at most 500, at most 400, at most 300, at most 250, at most 200, at most 150, or at most 100) consecutive nucleotides of the p30 promoter of ASFV or a mammalian U6 promoter, or at least 43 or 100 consecutive nucleotides (e.g. at least 150, at least 200, at least 250, at least 300, at least 400, at least 500, at least 750, at least 1000, at most 1000, at most 750, at most 500, at most 400, at most 300, at most 250, at most 200, at most 150, or at most 100) of a sequence variant having at least 80%, at least 85%, at least 90%, at least 91%, at least 92%, at least 93%, at least 94%, at least 95%, at least 96%, at least 97%, at least 98%, at least 99% identity to the p30 promoter of ASFV or a mammalian U6 promoter, or a sequence variant substantially identical to the p30 promoter of ASFV or a mammalian U6 promoter.

TABLE 1
Expression of genes early in the infection of ASFV (Early Genes)
that were found to be highly expressed at five hours post infection
(5 hpi) that can be targeted by nucleases described herein.

| Gene | | |
|---|---|---|
| Name | Action/Notes | Activity |
| A151R | CXXC-motif | Top 20 exp in Early |
| Y118L | MGF 110-6L | Top 20 exp in Early; |
| | | potential action in viral |
| | | factory formation in ER |
| 173R | Uncharacterized | Top 20 exp in Early |
| DP141L | MGF 100-2L | |
| XP124L | MGF 110-3L | Top 20 exp in Early; |
| | | potential action in viral |
| | | factory formation in ER |
| pNG1 | uncharacterized | |
| CP201L | phosphoprotein | |
| pNG2 | uncharacterized | |
| K205R | Uncharacterized | Top 20 exp in Early |
| E165R | dUTPase | Top 20 exp in Early |
| DP238L | uncharacterized | |
| A179L | Bcl-2-bax homolog | Putative Apoptosis regulator |
| pNG3 | uncharacterized | |
| U104L | MGF 110-2L | |
| D205R | 8-hydroxy- | |
| | dGTpase Nudix | |
| A276R | MGF 360-15R | Top 20 exp in Early |
| DP96R | Uncharacterized | |
| pNG4 | uncharacterized | |
| A280R | MGF 505-3R | |
| A240L | Thymidylate kinase | Early expression; required |
| | | for replication in swine |
| | | macrophages |

TABLE 2
Expression of genes later in the ASFV infection (Late
Genes) at sixteen hours post-infection (16 hpi) that
can be targeted by nucleases described herein.

| Gene | Function | Function & Comments |
|---|---|---|
| A137R | p11.5 | |
| K78R | p10 | |
| E184L | TR (transmembrane) | |
| O61R | p12 | |
| A104R | Histone-like | |
| B646L | p72 | Top 20 late gene |
| E120R | DpNG-binding p14.5 | |
| D117L | p17 | |
| L57L | Uncharacterized | |
| CP312R | Uncharacterized | Top 20 late gene |
| K145R | Uncharacterized | |
| A151R | CXXC-containing protein | |
| K205R | Uncharacterized | |
| Y118L | MGF 110-6L | Top 20 late gene |
| pNG1 | Uncharacterized | |
| H171R | Uncharacterized | |
| B119L | FAD-dependent thiol oxidase | |
| 173R | Uncharacterized | |
| A224L | IAP homolog | Top 20 late gene |
| C84L | Putative signal peptide | |

TABLE 3
Sequences of genes that can be targeted by nuclease systems
described herein and designed vectors targeting said genes

| SEQ | | |
|---|---|---|
| ID | | |
| NO: | GENE | SEQUENCE |
| 1 | DNA | ATGATATCTATCATGGACCGTTCTGAGATTGTTGCACGGGA |
| | polymerase | GAACCCGGTGATTACCCAACGAGTTACAAATCTCCTACAAA |
| | G1211R | CCAATGCTCCTCTACTATTCATGCCCATTGATATCCATGAAG |
| | | TACGATATGGAGCCTACACACTTTTCATGTATGGTTCCCTCG |
| | | AAAACGGTTACAAAGCAGAAGTAAGGATTGAAAACATCCC |
| | | AGTTTTCTTTGACGTACAGATTGAGTTCAATGATACAAACC |
| | | AGCTTTTTTTAAAGTCGCTACTGACGGCTGAAAATATTGCGT |
| | | ATGAACGGCTGGAGACGCTCACCCAGCGTCCTGTAATGGGG |
| | | TACCGCGAGAAGGAAAAAGAGTTTGCACCATACATTCGAAT |
| | | ATTTTTTAAAAGCCTGTATGAGCAACGAAAAGCCATTACTT |
| | | ACTTGAATAATATGGGTTACAACACCGCCGCGGACGACACA |
| | | ACCTGTTACTACCGAATGGTTTCCCGAGAGCTAAAACTGCC |
| | | TCTTACAAGTTGGATACAGCTTCAGCACTATTCCTACGAGCC |
| | | TCGCGGCTTGGTACACAGGTTTTCCGTAACCCCCGAGGATC |
| | | TTGTTTCCTATCAGGATGATGGCCCCACAGACCACAGCATC |
| | | GTTATGGCCTACGATATAGAGACCTATAGCCCTGTTAAGGG |
| | | AACCGTTCCGGACCCAAATCAGGCAAACGACGTGGTGTTCA |
| | | TGATATGCATGCGCATTTTTTGGATTCACTCCACAGAGCCTC |
| | | TAGCGAGCACGTGCATCACTATGGCACCAGCCTCTCGGGAT |
| | | GAGGCAAAAAGCCTCATGGCCAAGGGTGAATCTCTTCACTA |
| | | CGTCTCCTTTCACTTTAACAATCGTCTCGTGGAAGGATGGTT |
| | | TGTGCGACATAATAACGTTCCTGATAAAATGGGATTATACC |
| | | CAAAAGTACTCATCGATCTACTTAACAAACGAACCGCCCTT |
| | | AAACAAGAGCTTAAAAAACTAGGTGAGAAAAAAGAATGTA |
| | | TCCATGAATCCCATCCTGGGTTTAAGGAACTACAGTTTCGCC |
| | | ATGCCATGGTAGACGCGAAGCAAAAGGCGTTGAAAATTTTC |
| | | ATGAACACGTTTTACGGCGAGGCAGGTAACAATTTGTCGCC |
| | | CTTCTTTCTGCTTCCTCTAGCCGGAGGAGTCACCAGTTCGGG |
| | | TCAATATAATCTTAAACTCGTCTATAACTTTGTTATCAATAA |
| | | AGGTTACGGCATCAAGTACGGTGACACCGACTCATTATACA |
| | | TTACATGCCCAGATAGTCTTTATACAGAGGTAACAGACGCA |
| | | TATTTAAATAGCCAAAAAACAATAAAACATTATGAGCAACT |
| | | CTGCCACGAAAAAGTGCTTCTGTCTATGAAAGCCATGTCTA |
| | | CACTATGCGCCGAGGTGAATGAATACCTGCGGCAAGATAAT |
| | | GGCACCAGTTACCTACGTATGGCCTACGAGGAAGTACTCTT |
| | | TCCTGTGTGCTTTACAGGCAAGAAAAAGTATTACGGTATTG |
| | | CTCATGTAAACACACCCAATTTTAATACAAAAGAATTATTC |
| | | ATCCGCGGAATAGATATCATTAAGCAGGGTCAAACAAAACT |
| | | CACCAAAACGATAGGTACGCGAATTATGGAAGAATCCATG |
| | | AAACTGCGCCGCCCTGAGGACCATCGCCCCCCTCTTATTGA |
| | | AATCGTTAAAACGGTTTTGAAGGATGCTGTGGTTAACATGA |
| | | AGCAGTGGAATTTTGAAGACTTCATCCAAACAGATGCGTGG |
| | | AGACCGGACAAAGACAACAAAGCAGTCCAAATCTTTATGTC |
| | | TCGCATGCACGCTCGGCGTGAGCAACTAAAAAAACACGGC |
| | | GCCGCAGCATCGCAATTTGCTGAGCCTGAGCCGGGAGAACG |
| | | CTTCTCCTACGTTATCGTGGAAAAACAAGTACAGTTTGATAT |
| | | TCAGGGCCACCGCACAGATTCCTCCAGAAAGGGGGACAAG |
| | | ATGGAATACGTCTCTGAAGCAAAGGCTAAAAATCTTCCAAT |
| | | TGATATATTGTTTTATATCAACAACTATGTTCTAGGCTTGTG |
| | | CGCGAGATTCATTAATGAAAATGAAGAATTTCAACCCCCTG |
| | | ACAATGTCAGCAATAAGGATGAATACGCTCAGCGCCGAGCC |
| | | AAATCCTACCTACAAAAATTCGTACAATCCATTCACCCTAA |
| | | AGACAAGTCTGTCATTAAGCAAGGCATTGTTCATCGACAGT |
| | | GCTACAAATACGTTCACCAAGAAATTAAAAAAAAAATAGG |
| | | CATCTTTGCCGACCTTTATAAGGAATTTTTTAACAACACCAC |
| | | AAACCCCATCGAAAGCTTTATTCAAAGCGCTCGGTTTATGA |
| | | TACAATACTCTGATGGAGAACAAAAAGTAAACCATTCTATG |
| | | AAAAAAATGGTTGAACAGCGTGCTACTTTGGCAAGTAAGCC |
| | | CGCTGGTAAGCCCGCTGGTAATCCAGCTGGCAACCCAGCCG |
| | | GCAATGCGCTGATGCGGGCTATATTTACGCAGCTGATTACG |
| | | GAAGAAAAAAAAATTGTACAAGCCTTATACAATAAGGGGG |
| | | ATGCAATACACGATCTTCTCACCTATATCATTAACAATATAA |
| | | ATTACAAAATTGCCACGTTTCAGACGAAACAGATGTTGACG |
| | | TTCGAGTTTTCTAGTACTCATGTAGAACTGCTATTAAAGCTG |
| | | AATAAGACGTGGCTTATTTTGGCTGGAATTCATGTGGCGAA |
| | | AAAACATCTGCAAGCTCTTTTGGATTCATATAATAATGAAC |
| | | CACCGTCTAGAACATTCATTCAGCAGGCTATAGAGGAAGAA |
| | | TGTGGCAGTATTAAACCATCTTGCTACGACTTTATTTCCTAA |
| 2 | P1192R | AAAACGATGGCCCGGGAATCCCCATTGCAAAGCATGAGCA |
| | Topoisomerase | GGCCAGTCTTATCGCCAAGCGCGATGTGTATGTTCCCGAGG |
| | II | TGGCTTCATGCTTCTTTCTAGCCGGAACGAACATCAATAAG |
| | | GCCAAGGACTGTATCAAGGGGGGAACCAACGGCGTCGGGC |
| | | TGAAGCTCGCCATGGTGCATTCGCAGTGGGCCATTCTTACC |
| | | ACCGCCGACGGCGCGCAAAAGTATGTTCAACAAATCAACCA |
| | | GCGCCTAGATATCATTGAGCCTCCTACCATTACACCCTCCAG |
| | | GGAAATGTTTACACGTATCGAGCTCATGCCCGTATACCAGG |
| | | AACTAGGGTACGCGGAGCCTCTGTCTGAAACGGAGCAAGC |
| | | AGATCTTTCCGCCTGGATTTATCTTCGCGCCTGCCAATGCGC |
| | | GGCCTACGTGGGAAAAGGCACCACCATTTATTACAATGATA |
| | | AGCCTTGCCGCACGGGCTCTGTGATGGCGCTGGCCAAAATG |
| | | TACACCCTGTTGAGCGCGCCTAATAGCACGATACATACGGC |
| | | GACCATTAAGGCCGACGCAAAACCCTATAGCCTGCACCCTC |
| | | TGCAGGTTGCGGCGGTCGTGTCCCCCAAGTTTAAAAAATTT |
| | | GAACACGTGTCCATTATCAACGGGGTAAATTGTGTAAAAGG |
| | | AGAACATGTTACCTTTTTGAAAAAGACCATTAATGAAATGG |
| | | TCATTAAAAAATTTCAACAGACGATTAAAGATAAAAACCGC |
| | | AAAACAACATTACGTGACAGCTGTTCAAACATCTTTGTCGT |
| | | TATAGTGGGTTCCATTCCAGGCATAGAATGGACCGGCCAGC |
| | | GGAAGGATGAACTTAGCATCGCAGAAAATGTTTTTAAAACG |
| | | CATTACTCCATCCCTTCTAGTTTTTTAACAAGCATGACAAGG |
| | | TCTATCGTGGATATTCTTCTGCAATCCATTTCTAAAAAAGAT |
| | | AACCATAAACAGGTCGACGTAGACAAATATACGCGTGCCCG |
| | | CAATGCGGGAGGGAAAAGGGCGCAGGACTGCATGCTACTC |
| | | GCGGCGGAAGGGGATAGCGCACTTTCCCTGTTGCGCACGGG |
| | | ACTGACCCTGGGAAAGTCCAACCCAAGCGGGCCCTCCTTTG |
| | | ACTTCTGCGGCATGATCTCCCTGGGAGGGGTCATCATGAAT |
| | | GCCTGCAAAAAGGTGACAAACATTACAACGGACTCTGGAG |
| | | AAACCATCATGGTGCGCAACGAACAGCTTACCAATAATAAA |
| | | GTGTTGCAGGGAATTGTGCAGGTATTGGGTCTAGACTTCAA |
| | | CTGCCATTACAAAACGCAGGAAGAGCGAGCAAAGCTGAGA |
| | | TACGGCTGCATTGTTGCGTGCGTTGATCAAGATCTGGATGG |
| | | GTGTGGAAAAATCCTTGGACTGCTGCTGGCCTACTTTCACCT |
| | | GTTTTGGCCTCAGCTTATTATCCATGGTTTCGTAAAACGACT |
| | | GCTTACCCCGCTGATACGTGTGTACGAAAAGGGCAAGACTA |
| | | TGCCCGTAGAATTTTACTATGAACAGGAGTTTGATGCCTGG |
| | | GCAAAAAAGCAGACCAGCTTAGTCAATCATACTGTAAAATA |
| | | TTACAAGGGATTGGCGGCGCATGACACCCATGAAGTAAAA |
| | | AGCATGTTCAAACATTTTGACAACATGGTGTACACGTTTAC |
| | | CCTGGATGACTCGGCAAAGGAGTTGTTTCATATTTATTTTGG |
| | | CGGGGAGTCGGAGTTGCGAAAAAGAGAGCTTTGCACCGGC |
| | | GTGGTGCCGCTCACTGAAACCCAGACGCAGTCCATTCATAG |
| | | TGTCCGACGAATTCCTTGCAGCCTGCATCTGCAGGTAGATA |
| | | CCAAGGCTTACAAGCTGGATGCCATCGAGCGGCAGATTCCC |
| | | AACTTCTTAGACGGAATGACGCGGGCGCGGCGCAAAATTTT |
| | | AGCCGGGGGGGTGAAATGCTTCGCTTCCAACAACCGTGAAC |
| | | GAAAGGTTTTTCAGTTCGGGGGCTACGTTGCGGATCACATG |
| | | TTTTATCACCATGGCGACATGTCGTTAAACACAAGTATTATA |
| | | AAAGCCGCCCAGTATTACCCGGGCTCCTCCCACCTCTATCC |
| | | AGTATTCATAGGCATAGGAAGCTTCGGCTCCAGGCACCTGG |
| | | GAGGAAAGGATGCAGGATCCCCAAGATACATCAGTGTGCA |
| | | GCTTGCGTCTGAATTTATTAAAACAATGTTCCCCGCGGAGG |
| | | ACTCATGGCTTCTCCCCTACGTCTTTGAGGACGGCCAGCGG |
| | | GCGGAACCAGAGTACTACGTGCCTGTATTGCCGCTTGCTAT |
| | | TATGGAGTACGGCGCCAACCCATCGGAGGGCTGGAAGTAC |
| | | ACCACTTGGGCCCGGCAACTGGAAGACATTTTGGCCTTGGT |
| | | GAGGGCCTACGTCGACAAAGACAACCCAAAACACGAGCTA |
| | | CTGCACTATGCAATAAAACATAAGATTACTATACTCCCGCT |
| | | GCGGCCCTCCAATTACAATTTCAAGGGCCATTTGAAGCGGT |
| | | TTGGCCAATACTACTACAGCTACGGCACGTACGACATCTCA |
| | | GAGCAGCGAAATATAATTACTATTACGGAGCTTCCTCTGCG |
| | | TGTTCCTACGGTTGCATATATCGAAAGTATAAAAAAATCGA |
| | | GTAACCGCATGACATTTATTGAAGAAATCATCGACTACAGT |
| | | AGTTCAGAAACCATTGAAATTCTGGTGAAACTAAAGCCAAA |
| | | TAGTCTCAACCGTATCGTGGAAGAATTTAAGGAGACTGAAG |
| | | AGCAAGATTCCATAGAAAATTTTCTGCGCCTGCGCAATTGT |
| | | TTACATTCGCATCTAAACTTTGTAAAACCTAAAGGTGGTATT |
| | | ATCGAGTTTAACTCATATTATGAAATTTTATATGCGTGGCTA |
| | | CCTTACAGGCGTGAGCTTTACCAAAAGCGTCTTATGCGTGA |
| | | GCACGCGGTGCTTAAGCTGCGCATTATCATGGAAACTGCTA |
| | | TTGTACGCTACATCAATGAGTCTGCAGAGCTAAATCTTTCCC |
| | | ATTATGAGGATGAAAAGGAGGCAAGCCGCATTCTAAGCGA |
| | | GCATGGATTTCCCCCGCTGAACCACACGCTGATCATTTCCCC |
| | | TGAGTTTGCCTCTATAGAGGAACTCAATCAAAAAGCGCTGC |
| | | AGGGCTGTTATACCTATATACTATCTTTGCAGGCTCGAGAAT |
| | | TGCTTATCGCAGCCAAAACTCGTCGGGTGGAAAAAATAAAA |
| | | AAAATGCAAGCTCGTCTTGATAAGGTTGAGCAGCTTTTGCA |
| | | GGAGTCTCCCTTTCCCGGCGCCAGCGTATGGCTGGAGGAAA |
| | | TTGATGCGGTGGAAAAGGCTATTATAAAAGGAAGAAATACT |
| | | CAGTGGAAATTTCATTAA |
| 3 | RNA | ATGGAGGCCATTATATCCTTTGCTGGAATAGGAATAAATTA |
| | helicase | TAAGAAGCTACAAAGTAAATTACAACATGATTTCGGGCGCC |
| | (QP509L) | TTCTTAAGGCGCTCACCGTTACGGCGCGGGCATTGCCTGGG |
| | | CAGCCAAAGCACATAGCCATAAGACAGGAAACTGCCTTCAC |
| | | GCTGCAGGGGGAATACATTTATTTTCCCATATTGCTGCGAA |
| | | AGCAGTTTGAAATGTTTAACATGGTTTACACGACGCGCCCC |
| | | GTGTCGCTGCGGGCCCTCCCATGCGTTGAAACAGAATTTCC |
| | | ACTATTTAACTACCAGCAAGAAATGGTCGATAAGATTCATA |
| | | AAAAGCTCCTGTCCCCCTATGGGCGCTTTTACCTACATCTAA |
| | | ATACCGGTTTGGGGAAAACGCGTATTGCGATCAGCATTATT |
| | | CAAAAACTTTTGTACCCTACCCTGGTCATCGTGCCCACCAA |
| | | GGCGATTCAAATACAGTGGATCGACGAGCTAACATTGCTCC |
| | | TGCCCCACCTACGTGTAGCTGCTTACAATAATGCAGCGTGC |
| | | AAGAAAAAGGACATGACGAGCAAAGAGTACGACGTCATCG |
| | | TGGGAATCATTAATACCCTGCGCAAGAAGCCTGAGCAGTTC |
| | | TTTGAGCCCTTTGGTCTAGTCGTGTTAGATGAGGCACATGA |
| | | ATTACACTCGCCGGAGAATTACAAAATTTTTTGGAAAATAC |
| | | AACTTAGTCGGATATTAGGACTGTCCGCCACACCCCTGGAC |
| | | CGGCCCGATGGTATGGACAAGATTATTATTCACCATCTAGG |
| | | ACAGCCCCAGAGGACTGTAAGTCCCACCACAACCTTTTCCG |
| | | GGTACGTGAGGGAAATCGAATATCAGGGACATCCTGACTTC |
| | | GTTAGCCCTGTGTGTATTAATGAAAAGGTATCGGCCATTGC |
| | | CACCATTGATAAACTACTTCAAGATCCTTCGCGTATACAACT |
| | | TGTCGTAAATGAGGCAAAGCGGCTTTACTCCCTGCATACCG |
| | | CTGAGCCTCACAAATGGGGGACCGATGAGCCGTATGGCATC |
| | | ATCATTTTCGTGGAATTTCGCAAACTTTTAGAAATTTTTTAT |
| | | CAGGCGCTTTCCAAAGAATTCAAAGATGTTCAAATTGTCGT |
| | | TCCGGAGGTGGCGCTCCTATGCGGCGGGGTTTCAAATACCG |
| | | CTCTTTCTCAGGCACACAGCGCTTCCATTATCTTGCTGACCT |
| | | ATGGCTACGGGCGTAGAGGCATTTCCTTCAAGCATATGACA |
| | | TCGATCATCATGGCAACGCCCCGCAGAAACAACATGGAGCA |
| | | AATCTTGGGACGTATTACCCGGCAGGGATCGGATGAAAAAA |
| | | AGGTACGCATCGTCGTGGACATTAAAGATACACTAAGCCCG |
| | | CTTTCTAGCCAGGTCTACGACAGGCACCGGATTTACAAGAA |
| | | AAAGGGCTACCCCATTTTTAAGTGCAGCGCTAGCTATCAGC |
| | | AGCCCTATTCTTCTAATGAAGTTTTAATATGGGATCCTTATA |
| | | ACGAGTCATGTCTTGCGTGCACAACAACACCTCCTTCCCCGT |
| | | CCAAATAG |
| 4 | DNA | agacacaattccccagtggaaagacgcgcaggcaaaacgcaccacgtgacggagcgtgaccgc |
| | polymerase | gcgccgagcgcgcgccaaggtcgggcaggaagagggcctatttcccatgattccttcatatttgca |
| | multiplexed | tatacgatacaaggctgttagagagataattagaattaatttgactgtaaacacaaagatattagtaca |
| | sgRNA | aaatacgtgacgtagaaagtaataatttcttgggtagtttgcagttttaaaattatgttttaaaatggacta |
| | plasmid | tcatatgcttaccgtaacttgaaagtatttcgatttcttgggtttatatatcttgtggaaaggacgaggat |
| | vector | ccGATTGTTGCACGGGAGAACCgttttagagctagaaatagcaagttaaaataag |
| | | gctagtccgttatcaacttgaaaaagtggcaccgagtcggtgcttttttctagacacaattccccagtg |
| | | gaaagacgcgcaggcaaaacgcaccacgtgacggagcgtgaccgcgcgccgagcgcgcgcc |
| | | aaggtcgggcaggaagagggcctatttcccatgattccttcatatttgcatatacgatacaaggctgtt |
| | | agagagataattagaattaatttgactgtaaacacaaagatattagtacaaaatacgtgacgtagaaa |
| | | gtaataatttcttgggtagtttgcagttttaaaattatgttttaaaatggactatcatatgcttaccgtaac |
| | | ttgaaagtatttcgatttcttgggtttatatatcttgtggaaaggacgaggatccGACTTTGGCA |
| | | AGTAAGCCCGCgttttagagctagaaatagcaagttaaaataaggctagtccgttatcaact |
| | | tgaaaaagtggcaccgagtcggtgcttttttctagacacaattgcatgaagaatctgcttagggttag |
| | | gcgttttgcgctgcttcgcgatgtacgggccagatatacgcgttgacattgattattgactagttattaa |
| | | tagtaatcaattacggggtcattagttcatagcccatatatggagttccgcgttacataacttacggtaa |
| | | atggcccgcctggctgaccgcccaacgacccccgcccattgacgtcaataatgacgtatgttccca |
| | | tagtaacgccaatagggactttccattgacgtcaatgggtggaGtatttacggtaaactgcccacttg |
| | | gcagtacatcaagtgtatcatatgccaagtacgccccctattgacgtcaatgacggtaaatggcccg |
| | | cctggcattatgcccagtacatgaccttatgggactttcctacttggcagtacatctacgtattagtcat |
| | | cgctattaccatggtgatgcggttttggcagtacatcaatgggcgtggatagcggtttgactcacggg |
| | | gatttccaagtctccaccccattgacgtcaatgggagtttgttttggcaccaaaatcaacgggactttc |
| | | caaaatgtcgtaacaactccgccccattgacgcaaatgggcggtaggcgtgtacggtgggaggtct |
| | | atataagcagagctctctggctaactagagaacccactgcttactggcttatcgaaattaatacgactc |
| | | actatagggagacccaagcttgccaccatggacaagaagtacagcatcggcctggacatcggtac |
| | | caacagcgtgggctgggccgtgatcaccgacgagtacaaggtgcccagcaagaagttcaaggtg |
| | | ctgggcaacaccgaccgccacagcatcaagaagaacctgatcggcgccctgctgttcgacagcg |
| | | gcgagaccgccgaggccacccgcctgaagcgcaccgcccgccgccgctacacccgccgcaag |
| | | aaccgcatctgctacctgcaggagatcttcagcaacgagatggccaaggtggacgacagcttcttc |
| | | caccgcctggaggagagcttcctggtggaggaggacaagaagcacgagcgccaccccatcttcg |
| | | gcaacatcgtggacgaggtggcctaccacgagaagtaccccaccatctaccacctgcgcaagaa |
| | | gctggtggacagcaccgacaaggccgacctgcgcctgatctacctggccctggcccacatgatca |
| | | agttccgcggccacttcctgatcgagggcgacctgaaccccgacaacagcgacgtggacaagct |
| | | gttcatccagctggtgcagacctacaaccagctgttcgaggagaaccccatcaacgccagcggcg |
| | | tggacgccaaggccatcctgagcgcccgcctgagcaagagccgccgcctggagaacctgatcg |
| | | cccagctgcccggcgagaagaagaacggcctgttcggcaacctgatcgccctgagcctgggcct |
| | | gacccccaacttcaagagcaacttcgacctggccgaggacgccaagctgcagctgagcaaggac |
| | | acctacgacgacgacctggacaacctgctggcccagatcggcgaccagtacgccgacctgttcct |
| | | ggccgccaagaacctgagcgacgccatcctgctgagcgacatcctgcgcgtgaacaccgagatc |
| | | accaaggcccccctgagcgccagcatgatcaagcgctacgacgagcaccaccaggacctgacc |
| | | ctgctgaaggccctggtgcgccagcagctgcccgagaagtacaaggagatcttcttcgaccagag |
| | | caagaacggctacgccggctacatcgacggcggcgccagccaggaggagttctacaagttcatc |
| | | aagcccatcctggagaagatggacggcaccgaggagctgctggtgaagctgaaccgcgaggac |
| | | ctgctgcgcaagcagcgcaccttcgacaacggcagcatcccccaccagatccacctgggcgagc |
| | | tgcacgccatcctgcgccgccaggaggacttctaccccttcctgaaggacaaccgcgagaagatc |
| | | gagaagatcctgaccttccgcatcccctactacgtgggccccctggcccgcggcaacagccgctt |
| | | cgcctggatgacccgcaagagcgaggagaccatcaccccctggaacttcgaggaggtggtggac |
| | | aagggcgccagcgcccagagcttcatcgagcgcatgaccaacttcgacaagaacctgcccaacg |
| | | agaaggtgctgcccaagcacagcctgctgtacgagtacttcaccgtgtacaacgagctgaccaag |
| | | gtgaagtacgtgaccgagggcatgcgcaagcccgccttcctgagcggcgagcagaagaaggcc |
| | | atcgtggacctgctgttcaagaccaaccgcaaggtgaccgtgaagcagctgaaggaggactactt |
| | | caagaagatcgagtgcttcgacagcgtggagatcagcggcgtggaggaccgcttcaacgccagc |
| | | ctgggcacctaccacgacctgctgaagatcatcaaggacaaggacttcctggacaacgaggagaa |
| | | cgaggacatcctggaggacatcgtgctgaccctgaccctgttcgaggaccgcgagatgatcgagg |
| | | agcgcctgaagacctacgcccacctgttcgacgacaaggtgatgaagcagctgaagcgccgccg |
| | | ctacaccggctggggccgcctgagccgcaagcttatcaacggcatccgcgacaagcagagcgg |
| | | caagaccatcctggacttcctgaagagcgacggcttcgccaaccgcaacttcatgcagctgatcca |
| | | cgacgacagcctgaccttcaaggaggacatccagaaggcccaggtgagcggccagggcgaca |
| | | gcctgcacgagcacatcgccaacctggccggcagccccgccatcaagaagggcatcctgcaga |
| | | ccgtgaaggtggtggacgagctggtgaaggtgatgggccgccacaagcccgagaacatcgtgat |
| | | cgagatggcccgcgagaaccagaccacccagaagggccagaagaacagccgcgagcgcatga |
| | | agcgcatcgaggagggcatcaaggagctgggcagccagatcctgaaggagcaccccgtggaga |
| | | acacccagctgcagaacgagaagctgtacctgtactacctgcagaacggccgcgacatgtacgtg |
| | | gaccaggagctggacatcaaccgcctgagcgactacgacgtggaccacatcgtgccccagagct |
| | | tcctgaaggacgacagcatcgacaacaaggtgctgacccgcagcgacaagaaccgcggcaaga |
| | | gcgacaacgtgcccagcgaggaggtggtgaagaagatgaagaactactggcgccagctgctga |
| | | acgccaagctgatcacccagcgcaagttcgacaacctgaccaaggccgagcgcggcggcctga |
| | | gcgagctggacaaggccggcttcatcaagcgccagctggtggagacccgccagatcaccaagc |
| | | acgtggcccagatcctggacagccgcatgaacaccaagtacgacgagaacgacaagctgatccg |
| | | cgaggtgaaggtgatcaccctgaagagcaagctggtgagcgacttccgcaaggacttccagttcta |
| | | caaggtgcgcgagatcaacaactaccaccacgcccacgacgcctacctgaacgccgtggtgggc |
| | | accgccctgatcaagaagtaccccaagctggagagcgagttcgtgtacggcgactacaaggtgta |
| | | cgacgtgcgcaagatgatcgccaagagcgagcaggagatcggcaaggccaccgccaagtactt |
| | | cttctacagcaacatcatgaacttcttcaagaccgagatcaccctggccaacggcgagatccgcaa |
| | | gogccccctgatcgagaccaacggcgagaccggcgagatcgtgtgggacaagggccgcgactt |
| | | cgccaccgtgcgcaaggtgctgagcatgccccaggtgaacatcgtgaagaagaccgaggtgcag |
| | | accggcggcttcagcaaggagagcatcctgcccaagcgcaacagcgacaagctgatcgcccgc |
| | | aagaaggactgggaccccaagaagtacggcggcttcgacagccccaccgtggcctacagcgtg |
| | | ctggtggtggccaaggtggagaagggcaagagcaagaagctgaagagcgtgaaggagctgctg |
| | | ggcatcaccatcatggagcgcagcagcttcgagaagaaccccatcgacttcctggaggccaagg |
| | | gctacaaggaggtgaagaaggacctgatcatcaagctgcccaagtacagcctgttcgagctggag |
| | | aacggccgcaagcgcatgctggccagcgccggcgagctgcagaagggcaacgagctggccct |
| | | gcccagcaagtacgtgaacttcctgtacctggccagccactacgagaagctgaagggcagcccc |
| | | gaggacaacgagcagaagcagctgttcgtggagcagcacaagcactacctggacgagatcatcg |
| | | agcagatcagcgagttcagcaagcgcgtgatcctggccgacgccaacctggacaaggtgctgag |
| | | cgcctacaacaagcaccgcgacaagcccatccgcgagcaggccgagaacatcatccacctgttc |
| | | accctgaccaacctgggcgcccccgccgccttcaagtacttcgacaccaccatcgaccgcaagcg |
| | | ctacaccagcaccaaggaggtgctggacgccaccctgatccaccagagcatcaccggtctgtacg |
| | | agacccgcatcgacctgagc |
| | | cagctgggcggcgacggcggctccggacctccaaagaaaaagagaaaagtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgcTAGg |
| | | cggccgcAgatgggggtcctgggccccagggtgtgcagccactgacttggggactgctggtgg |
| | | ggtagggatgagggagggaggggcattgtgatgtacagggctgctctgtgagatcaagggtctctt |
| | | aagggtgggagctggggcagggactacgagagcagccagatgggctgaaagtggaactcaag |
| | | gggtttctggcacctacctacctgcttcccgctggggggtggggagttggcccagagtcttaagatt |
| | | ggggcagggtggagaggtgggctcttcctgcttcccactcatcttatagctttctttccccagatccg |
| | | aattggagatccaaaccaaggcgcgcGCTAGCGCCACCatgggatcggccattgaaca |
| | | agatggattgcacgcaggttctccggccgcttgggtggagaggctattcggctatgactgggcaca |
| | | acagacaatcggctgctctgatgccgccgtgttccggctgtcagcgcaggggcgcccggttcttttt |
| | | gtcaagaccgacctgtccggtgccctgaatgaactgcaggacgaggcagcgcggctatcgtggct |
| | | ggccacgacgggcgttccttgcgcagcagtgctcgacgttgtcactgaagcgggaagggactgg |
| | | ctgctattgggcgaagtgccggggcaggatctcctgtcatctcaccttgctcctgccgagaaagtat |
| | | ccatcatggctgatgcaatgcggcggctgcatacgcttgatccggctacctgcccattcgaccacca |
| | | agcgaaacatcgcatcgagcgagcacgtactcggatggaagccggtcttgtcgatcaggatgatct |
| | | ggacgaagagcatcaggggctcgcgccagccgaactgttcgccaggctcaaggcgcgtatgccc |
| | | gacggogatgatctcgtcgtgactcatggcgatgcctgcttgccgaatatcatggtggaaaatggcc |
| | | gcttttctggattcatcgactgtggccggctgggtgtggcggaccgctatcaggacatagcgttggc |
| | | tacccgtgatattgctgaagagcttggcggcgaatgggctgaccgcttcctcgtgctttacggtatcg |
| | | ccgctcccgattcgcagcgcatcgccttctatcgccttcttgacgagttcttctgaacgcggtgctac |
| | | gagatttcgattccaccgccgccttctatgaaaggttgggcttcggaatcgttttccgggacgccggc |
| | | tggatgatcctccagcgcggggatctcatgctggagttcttcgcccaccccaacttgtttattgcagct |
| | | tataatggttacaaataaagcaatagcatcacaaatttcacaaataaagcatttttttcactgcattctag |
| | | ttgtggtttgtccaaactcatcaatgtatcttatcatgtctgtataccgtcgacctctagctagagcttgg |
| | | cgtaatcatggtcatagctgtttcctgtgtgaaattgttatccgctcacaattccacacaacatacgagc |
| | | cggaagcataaaggtaagcctgaatattgaaaaaggaagagtatgagtattcaacatttccgtgtcg |
| | | cccttattcccttttttgcggcattttgccttcctgtttttgctcacccagaaacgctggtgaaagtaaaa |
| | | gatgctgaagatcagttgggtgcacgagtgggttacatcgaactggatctcaacagcggtaagatc |
| | | cttgagagttttcgccccgaagaacgttttccaatgatgagcacttttaaagttctgctatgtggcgcg |
| | | gtattatcccgtattgacgccgggcaagagcaactcggtcgccgcatacactattctcagaatgactt |
| | | ggttgagtactcaccagtcacagaaaagcatcttacggatggcatgacagtaagagaattatgcagt |
| | | gctgccataaccatgagtgataacactgcggccaacttacttctgacaacgatcggaggaccgaag |
| | | gagctaaccgcttttttgcacaacatgggggatcatgtaactcgccttgatcgttgggaaccggagct |
| | | gaatgaagccataccaaacgacgagcgtgacaccacgatgcctgtagcaatggcaacaacgttgc |
| | | gcaaactattaactggcgaactacttactctagcttcccggcaacaattaatagactggatggaggc |
| | | ggataaagttgcaggaccacttctgcgctcggcccttccggctggctggtttattgctgataaatctg |
| | | gagccggtgagcgtgggtctcgcggtatcattgcagcactggggccagatggtaagccctcccgt |
| | | atcgtagttatctacacgacggggagtcaggcaactatggatgaacgaaatagacagatcgctgag |
| | | ataggtgcctcactgattaagcattggtaactgtcagaccaagtttactcatatatactttagattgattta |
| | | aaacttcatttttaatttaaaaggatctaggtgaagatcctttttgataatctcatgaccaaaatcccttaa |
| | | cgtgagttttcgttccactgagcgtcagaccccgtagaaaagatcaaaggatcttcttgagatccttttt |
| | | ttctgcgcgtaatctgctgcttgcaaacaaaaaaaccaccgctaccagcggtggtttttttgccggatc |
| | | aagagctaccaactctttttccgaaggtaactggcttcagcagagcgcagataccaaatactgttctt |
| | | ctagtgtagccgtagttaggccaccacttcaagaactctgtagcaccgcctacatacctcgctctgct |
| | | aatcctgttaccagtggctgctgccagtggcgataagtcgtgtcttaccgggttggactcaagacgat |
| | | agttaccggataaggcgcagcggtcgggctgaacggggggttcgtgcacacagcccagcttgga |
| | | gogaacgacctacaccgaactgagatacctacagcgtgagctatgagaaagcgccacgcttcccg |
| | | aagggagaaaggcggacaggtatccggtaagcggcagggtcggaacaggagagcgcacgag |
| | | ggagcttccagggggaaacgcctggtatctttatagtcctgtcgggtttcgccacctctgacttgagc |
| | | gtcgatttttgtgatgctcgtcaggggggcggagcctatggaaaaacgccagcaacgcggccttttt |
| | | acggttcctggccttttgctggccttttgctcacatgttctttcctgcgttatcccctgattctgtggataa |
| | | ccgtattaccgcctttgagtgagctgataccgctcgccgcagccgaacgaccgagcgcagcgagt |
| | | cagtgagcgaggaagcggaagagcgcccaatacgcaaaccgcctctccccgcgcgttggccga |
| | | ttcattaatgcagctggcacgacaggtttcccgactggaaagcgggcagtgagcgcaacgcaatta |
| | | atgtgagttagctcactcattaggcaccccaggctttacactttatgcttccggctcgtatgttgtgtgg |
| | | aattgtgagcggataacaatttcacacaggaaacagctatgacatgattacgaattgcaacgatttag |
| | | gtgacactatagaagagaaggaattaatacgactcactatagggagagagagagaattaccctcac |
| | | taaagggaggagaagcatgaattccccagtggaaagacgcgcaggcaaaacgcaccacgtgac |
| | | ggagcgtgaccgcgcgccgagcgcgcgccaaggtcgggcaggaagagggcctatttcccatga |
| | | ttccttcatatttgcatatacgatacaaggctgttagagagataattagaattaatttgactgtaaacaca |
| | | aagatattagtacaaaatacgtgacgtagaaagtaataatttcttgggtagtttgcagttttaaaattatg |
| | | ttttaaaatggactatcatatgcttaccgtaacttgaaagtatttcgatttcttgggtttatatatcttgtg |
| | | gaaaggacgaggatccGTTTAACAATCGTCTCGTGGAgttttagagctagaaatag |
| | | caagttaaaataaggctagtccgttatcaacttgaaaaagtggcaccgagtcggtgcttttttct |
| 5 | Topoisomerase | agacacaattccccagtggaaagacgcgcaggcaaaacgcaccacgtgacggagcgtgaccgc |
| | II (p1192R) | gcgccgagcgcgcgccaaggtcgggcaggaagagggcctatttcccatgattccttcatatttgca |
| | multiplexed | tatacgatacaaggctgttagagagataattagaattaatttgactgtaaacacaaagatattagtaca |
| | CRISPR/ | tcatatgcttaccgtaacttgaaagtatttcgatttcttgggtttatatatcttgtggaaaggacgaggat |
| | Cas9 vector | ccGACCAAGATCTGGACGGGTGgttttagagctagaaatagcaagttaaaataag |
| | | getagtccgttatcaacttgaaaaagtggcaccgagtcggtgcttttttctagacacaattccccagtg |
| | | gaaagacgcgcaggcaaaacgcaccacgtgacggagcgtgaccgcgcgccgagcgcgcgcc |
| | | aaggtcgggcaggaagagggcctatttcccatgattccttcatatttgcatatacgatacaaggctgtt |
| | | agagagataattagaattaatttgactgtaaacacaaagatattagtacaaaatacgtgacgtagaaa |
| | | gaaagtatttcgatttcttgggtttatatatcttgtggaaaggacgaggatccGGGTGTATGA |
| | | CACGTTGTCGgttttagagctagaaatagcaagttaaaataaggctagtccgttatcaacttg |
| | | aaaaagtggcaccgagtcggtgcttttttctagacacaattgcatgaagaatctgcttagggttaggc |
| | | gttttgcgctgcttcgcgatgtacgggccagatatacgcgttgacattgattattgactagttattaata |
| | | gtaatcaattacggggtcattagttcatagcccatatatggagttccgcgttacataacttacggtaaat |
| | | ggcccgcctggctgaccgcccaacgacccccgcccattgacgtcaataatgacgtatgttcccata |
| | | gtaacgccaatagggactttccattgacgtcaatgggtggaGtatttacggtaaactgcccacttgg |
| | | cagtacatcaagtgtatcatatgccaagtacgccccctattgacgtcaatgacggtaaatggcccgc |
| | | ctggcattatgcccagtacatgaccttatgggactttcctacttggcagtacatctacgtattagtcatc |
| | | gctattaccatggtgatgcggttttggcagtacatcaatgggcgtggatagcggtttgactcacggg |
| | | gatttccaagtctccaccccattgacgtcaatgggagtttgttttggcaccaaaatcaacgggactttc |
| | | caaaatgtcgtaacaactccgccccattgacgcaaatgggcggtaggcgtgtacggtgggaggtct |
| | | atataagcagagctctctggctaactagagaacccactgcttactggcttatcgaaattaatacgactc |
| | | actatagggagacccaagcttgccaccatggacaagaagtacagcatcggcctggacatcggtac |
| | | caacagcgtgggctgggccgtgatcaccgacgagtacaaggtgcccagcaagaagttcaaggtg |
| | | ctgggcaacaccgaccgccacagcatcaagaagaacctgatcggcgccctgctgttcgacagcg |
| | | gcgagaccgccgaggccacccgcctgaagcgcaccgcccgccgccgctacacccgccgcaag |
| | | aaccgcatctgctacctgcaggagatcttcagcaacgagatggccaaggtggacgacagcttcttc |
| | | caccgcctggaggagagcttcctggtggaggaggacaagaagcacgagcgccaccccatcttcg |
| | | gcaacatcgtggacgaggtggcctaccacgagaagtaccccaccatctaccacctgcgcaagaa |
| | | gctggtggacagcaccgacaaggccgacctgcgcctgatctacctggccctggcccacatgatca |
| | | agttccgcggccacttcctgatcgagggcgacctgaaccccgacaacagcgacgtggacaagct |
| | | gttcatccagctggtgcagacctacaaccagctgttcgaggagaaccccatcaacgccagcggcg |
| | | tggacgccaaggccatcctgagcgcccgcctgagcaagagccgccgcctggagaacctgatcg |
| | | cccagctgcccggcgagaagaagaacggcctgttcggcaacctgatcgccctgagcctgggcct |
| | | gacccccaacttcaagagcaacttcgacctggccgaggacgccaagctgcagctgagcaaggac |
| | | acctacgacgacgacctggacaacctgctggcccagatcggcgaccagtacgccgacctgttcct |
| | | ggccgccaagaacctgagcgacgccatcctgctgagcgacatcctgcgcgtgaacaccgagatc |
| | | accaaggcccccctgagcgccagcatgatcaagcgctacgacgagcaccaccaggacctgacc |
| | | ctgctgaaggccctggtgcgccagcagctgcccgagaagtacaaggagatcttcttcgaccagag |
| | | caagaacggctacgccggctacatcgacggcggcgccagccaggaggagttctacaagttcatc |
| | | aagcccatcctggagaagatggacggcaccgaggagctgctggtgaagctgaaccgcgaggac |
| | | ctgctgcgcaagcagcgcaccttcgacaacggcagcatcccccaccagatccacctgggcgagc |
| | | tgcacgccatcctgcgccgccaggaggacttctaccccttcctgaaggacaaccgcgagaagatc |
| | | gagaagatcctgaccttccgcatcccctactacgtgggccccctggcccgcggcaacagccgctt |
| | | cgcctggatgacccgcaagagcgaggagaccatcaccccctggaacttcgaggaggtggtggac |
| | | aagggcgccagcgcccagagcttcatcgagcgcatgaccaacttcgacaagaacctgcccaacg |
| | | agaaggtgctgcccaagcacagcctgctgtacgagtacttcaccgtgtacaacgagctgaccaag |
| | | gtgaagtacgtgaccgagggcatgcgcaagcccgccttcctgagcggcgagcagaagaaggcc |
| | | atcgtggacctgctgttcaagaccaaccgcaaggtgaccgtgaagcagctgaaggaggactactt |
| | | caagaagatcgagtgcttcgacagcgtggagatcagcggcgtggaggaccgcttcaacgccagc |
| | | ctgggcacctaccacgacctgctgaagatcatcaaggacaaggacttcctggacaacgaggagaa |
| | | cgaggacatcctggaggacatcgtgctgaccctgaccctgttcgaggaccgcgagatgatcgagg |
| | | agcgcctgaagacctacgcccacctgttcgacgacaaggtgatgaagcagctgaagcgccgccg |
| | | ctacaccggctggggccgcctgagccgcaagcttatcaacggcatccgcgacaagcagagcgg |
| | | caagaccatcctggacttcctgaagagcgacggcttcgccaaccgcaacttcatgcagctgatcca |
| | | cgacgacagcctgaccttcaaggaggacatccagaaggcccaggtgagcggccagggcgaca |
| | | gcctgcacgagcacatcgccaacctggccggcagccccgccatcaagaagggcatcctgcaga |
| | | ccgtgaaggtggtggacgagctggtgaaggtgatgggccgccacaagcccgagaacatcgtgat |
| | | cgagatggcccgcgagaaccagaccacccagaagggccagaagaacagccgcgagcgcatga |
| | | agcgcatcgaggagggcatcaaggagctgggcagccagatcctgaaggagcaccccgtggaga |
| | | acacccagctgcagaacgagaagctgtacctgtactacctgcagaacggccgcgacatgtacgtg |
| | | gaccaggagctggacatcaaccgcctgagcgactacgacgtggaccacatcgtgccccagagct |
| | | tcctgaaggacgacagcatcgacaacaaggtgctgacccgcagcgacaagaaccgcggcaaga |
| | | gcgacaacgtgcccagcgaggaggtggtgaagaagatgaagaactactggcgccagctgctga |
| | | acgccaagctgatcacccagcgcaagttcgacaacctgaccaaggccgagcgcggcggcctga |
| | | gcgagctggacaaggccggcttcatcaagcgccagctggtggagacccgccagatcaccaagc |
| | | acgtggcccagatcctggacagccgcatgaacaccaagtacgacgagaacgacaagctgatccg |
| | | cgaggtgaaggtgatcaccctgaagagcaagctggtgagcgacttccgcaaggacttccagttcta |
| | | caaggtgcgcgagatcaacaactaccaccacgcccacgacgcctacctgaacgccgtggtgggc |
| | | accgccctgatcaagaagtaccccaagctggagagcgagttcgtgtacggcgactacaaggtgta |
| | | cgacgtgcgcaagatgatcgccaagagcgagcaggagatcggcaaggccaccgccaagtactt |
| | | cttctacagcaacatcatgaacttcttcaagaccgagatcaccctggccaacggcgagatccgcaa |
| | | gogccccctgatcgagaccaacggcgagaccggcgagatcgtgtgggacaagggccgcgactt |
| | | cgccaccgtgcgcaaggtgctgagcatgccccaggtgaacatcgtgaagaagaccgaggtgcag |
| | | accggcggcttcagcaaggagagcatcctgcccaagcgcaacagcgacaagctgatcgcccgc |
| | | aagaaggactgggaccccaagaagtacggcggcttcgacagccccaccgtggcctacagcgtg |
| | | ctggtggtggccaaggtggagaagggcaagagcaagaagctgaagagcgtgaaggagctgctg |
| | | ggcatcaccatcatggagcgcagcagcttcgagaagaaccccatcgacttcctggaggccaagg |
| | | gctacaaggaggtgaagaaggacctgatcatcaagctgcccaagtacagcctgttcgagctggag |
| | | aacggccgcaagcgcatgctggccagcgccggcgagctgcagaagggcaacgagctggccct |
| | | gcccagcaagtacgtgaacttcctgtacctggccagccactacgagaagctgaagggcagcccc |
| | | gaggacaacgagcagaagcagctgttcgtggagcagcacaagcactacctggacgagatcatcg |
| | | agcagatcagcgagttcagcaagcgcgtgatcctggccgacgccaacctggacaaggtgctgag |
| | | cgcctacaacaagcaccgcgacaagcccatccgcgagcaggccgagaacatcatccacctgttc |
| | | accctgaccaacctgggcgcccccgccgccttcaagtacttcgacaccaccatcgaccgcaagcg |
| | | ctacaccagcaccaaggaggtgctggacgccaccctgatccaccagagcatcaccggtctgtacg |
| | | agacccgcatcgacctgagccagctgggcggcgacggcggctccggacctccaaagaaaaaga |
| | | gaaaagtatacccctacgacgtgcccgactacgccctcgaggagggcagaggaagtcttctaaca |
| | | tgcggtgacgtggaggagaatcccggccctatggagagcgacgagagcggcctgcccgccatg |
| | | gagatcgagtgccgcatcaccggcaccctgaacggcgtggagttcgagctggtgggcggcgga |
| | | gagggcacccccaagcagggccgcatgaccaacaagatgaagagcaccaaaggcgccctgac |
| | | cttcagcccctacctgctgagccacgtgatgggctacggcttctaccacttcggcacctaccccagc |
| | | ggctacgagaaccccttcctgcacgccatcaacaacggcggctacaccaacacccgcatcgaga |
| | | agtacgaggacggcggcgtgctgcacgtgagcttcagctaccgctacgaggccggccgcgtgat |
| | | cggcgacttcaaggtggtgggcaccggcttccccgaggacagcgtgatcttcaccgacaagatca |
| | | tccgcagcaacgccaccgtggagcacctgcaccccatgggcgataacgtgctggtgggcagcttc |
| | | gcccgcaccttcagcctgcgcgacggcggctactacagcttcgtggtggacagccacatgcacttc |
| | | aagagcgccatccaccccagcatcctgcagaacgggggccccatgttcgccttccgccgcgtgg |
| | | aggagctgcacagcaacaccgagctgggcatcgtggagtaccagcacgccttcaagacccccat |
| | | cgccttcgccagatcccgcgctcagtcgtccaattctgccgtggacggcaccgccggacccggct |
| | | ccaccggatctcgcTAGgcggccgcAgatgggggtcctgggccccagggtgtgcagccact |
| | | gacttggggactgctggtggggtagggatgagggagggaggggcattgtgatgtacagggctgc |
| | | tctgtgagatcaagggtctcttaagggtgggagctggggcagggactacgagagcagccagatg |
| | | ggctgaaagtggaactcaaggggtttctggcacctacctacctgcttcccgctggggggtgggga |
| | | gttggcccagagtcttaagattggggcagggtggagaggtgggctcttcctgcttcccactcatctta |
| | | tagctttctttccccagatccgaattggagatccaaaccaaggcgcgcGCTAGCGCCACC |
| | | atgggatcggccattgaacaagatggattgcacgcaggttctccggccgcttgggtggagaggcta |
| | | ttcggctatgactgggcacaacagacaatcggctgctctgatgccgccgtgttccggctgtcagcg |
| | | caggggcgcccggttctttttgtcaagaccgacctgtccggtgccctgaatgaactgcaggacgag |
| | | gcagcgcggctatcgtggctggccacgacgggcgttccttgcgcagcagtgctcgacgttgtcact |
| | | gaagcgggaagggactggctgctattgggcgaagtgccggggcaggatctcctgtcatctcacctt |
| | | gctcctgccgagaaagtatccatcatggctgatgcaatgcggcggctgcatacgcttgatccggcta |
| | | cctgcccattcgaccaccaagcgaaacatcgcatcgagcgagcacgtactcggatggaagccggt |
| | | cttgtcgatcaggatgatctggacgaagagcatcaggggctcgcgccagccgaactgttcgccag |
| | | gctcaaggcgcgtatgcccgacggcgatgatctcgtcgtgactcatggcgatgcctgcttgccgaa |
| | | tatcatggtggaaaatggccgcttttctggattcatcgactgtggccggctgggtgtggcggaccgc |
| | | tatcaggacatagcgttggctacccgtgatattgctgaagagcttggcggcgaatgggctgaccgct |
| | | tcctcgtgctttacggtatcgccgctcccgattcgcagcgcatcgccttctatcgccttcttgacgagtt |
| | | cttctgaacgcggtgctacgagatttcgattccaccgccgccttctatgaaaggttgggcttcggaat |
| | | cgttttccgggacgccggctggatgatcctccagcgcggggatctcatgctggagttcttcgcccac |
| | | catttttttcactgcattctagttgtggtttgtccaaactcatcaatgtatcttatcatgtctgtataccgt |
| | | cgacctctagctagagcttggcgtaatcatggtcatagctgtttcctgtgtgaaattgttatccgctcacaa |
| | | ttccacacaacatacgagccggaagcataaaggtaagcctgaatattgaaaaaggaagagtatga |
| | | gtattcaacatttccgtgtcgcccttattcccttttttgcggcattttgccttcctgtttttgctcacccag |
| | | aaacgctggtgaaagtaaaagatgctgaagatcagttgggtgcacgagtgggttacatcgaactggat |
| | | ctcaacagcggtaagatccttgagagttttcgccccgaagaacgttttccaatgatgagcacttttaaa |
| | | gttctgctatgtggcgcggtattatcccgtattgacgccgggcaagagcaactcggtcgccgcatac |
| | | actattctcagaatgacttggttgagtactcaccagtcacagaaaagcatcttacggatggcatgaca |
| | | gtaagagaattatgcagtgctgccataaccatgagtgataacactgcggccaacttacttctgacaac |
| | | gatcggaggaccgaaggagctaaccgcttttttgcacaacatgggggatcatgtaactcgccttgat |
| | | cgttgggaaccggagctgaatgaagccataccaaacgacgagcgtgacaccacgatgcctgtag |
| | | caatggcaacaacgttgcgcaaactattaactggcgaactacttactctagcttcccggcaacaatta |
| | | atagactggatggaggcggataaagttgcaggaccacttctgcgctcggcccttccggctggctgg |
| | | tttattgctgataaatctggagccggtgagcgtgggtctcgcggtatcattgcagcactggggccag |
| | | atggtaagccctcccgtatcgtagttatctacacgacggggagtcaggcaactatggatgaacgaa |
| | | atagacagatcgctgagataggtgcctcactgattaagcattggtaactgtcagaccaagtttactcat |
| | | atatactttagattgatttaaaacttcatttttaatttaaaaggatctaggtgaagatcctttttgataatc |
| | | tcatgaccaaaatcccttaacgtgagttttcgttccactgagcgtcagaccccgtagaaaagatcaaag |
| | | gatcttcttgagatcctttttttctgcgcgtaatctgctgcttgcaaacaaaaaaaccaccgctaccagc |
| | | ggtggtttttttgccggatcaagagctaccaactctttttccgaaggtaactggcttcagcagagcgca |
| | | gataccaaatactgttcttctagtgtagccgtagttaggccaccacttcaagaactctgtagcaccgc |
| | | ctacatacctcgctctgctaatcctgttaccagtggctgctgccagtggcgataagtcgtgtcttaccg |
| | | ggttggactcaagacgatagttaccggataaggcgcagcggtcgggctgaacggggggttcgtg |
| | | cacacagcccagcttggagcgaacgacctacaccgaactgagatacctacagcgtgagctatgag |
| | | aaagcgccacgcttcccgaagggagaaaggcggacaggtatccggtaagcggcagggtcggaa |
| | | caggagagcgcacgagggagcttccagggggaaacgcctggtatctttatagtcctgtcgggtttc |
| | | gccacctctgacttgagcgtcgatttttgtgatgctcgtcaggggggcggagcctatggaaaaacgc |
| | | cagcaacgcggcctttttacggttcctggccttttgctggccttttgctcacatgttctttcctgcgttatc |
| | | ccctgattctgtggataaccgtattaccgcctttgagtgagctgataccgctcgccgcagccgaacg |
| | | accgagcgcagcgagtcagtgagcgaggaagcggaagagcgcccaatacgcaaaccgcctctc |
| | | cccgcgcgttggccgattcattaatgcagctggcacgacaggtttcccgactggaaagcgggcagt |
| | | gagcgcaacgcaattaatgtgagttagctcactcattaggcaccccaggctttacactttatgcttccg |
| | | gctcgtatgttgtgtggaattgtgagcggataacaatttcacacaggaaacagctatgacatgattac |
| | | gaattgcaacgatttaggtgacactatagaagagaaggaattaatacgactcactatagggagaga |
| | | gagagaattaccctcactaaagggaggagaagcatgaattccccagtggaaagacgcgcaggca |
| | | aaacgcaccacgtgacggagcgtgaccgcgcgccgagcgcgcgccaaggtcgggcaggaag |
| | | agggcctatttcccatgattccttcatatttgcatatacgatacaaggctgttagagagataattagaatt |
| | | aatttgactgtaaacacaaagatattagtacaaaatacgtgacgtagaaagtaataatttcttgggtagt |
| | | ttgcagttttaaaattatgttttaaaatggactatcatatgcttaccgtaacttgaaagtatttcgatttct |
| | | tgggtttatatatcttgtggaaaggacgaggatccGTGTTTAACGACATATCGCCAg |
| | | ttttagagctagaaatagcaagttaaaataaggctagtccgttatcaacttgaaaaagtggcaccgag |
| | | tcggtgcttttttct |
| 6 | BA71- | ATGCTTGGTCTGCAAATATTCACCCTACTATCTATTCCAACT |
| | L270L | CTTCTTTATACATATGAGATAGAGCCCCTGGAACGAACAAG |
| | (MGF-110 | TACCCCACCTGAAAAGGAGCTTGGATACTGGTGCACTTATG |
| | family | CAAACCATTGTAGATTCTGTTGGGACTGTCAAGATGGTATC |
| | member) | TGTAGGAACAAGGCTTTTAAAAACCATTCTCCCATTCTTGA |
| | | AAATGACTATATAGCTAACTGCAGTATTTATCGTCGCAATG |
| | | ATTTCTGTATCTACTACATAACTTCTATAAAGCCTCATAAAA |
| | | CGTACCGAACAGAATGTCCACAACACATAAACCATGAAAG |
| | | GCATGAGGCTGATATACGAAAATGGCAGAAATTGTTAACCT |
| | | ATGGATTTTATCTTGCGGGATGTATTTTAGCTGTGAATTACA |
| | | TTCGCAAACGTAGTTTACAGACTGTTATGTATTTGCTGGTCT |
| | | TCCTGGTAATCTCCTTTCTGCTTTCCCAACTGATGCTGTATG |
| | | GAGAGTTAGAAGATAAAAAACATAAAATTGGCAGCATTCCT |
| | | CCCAAAAGAGAGCTTGAACATTGGTGTACTCATGGAAAATA |
| | | TTGTAACTTTTGCTGGGACTGTCAAAATGGCATCTGTAAGA |
| | | ATAAGGCTTTTAAGAATCATCCCCCCATAGGTGAAAATGAT |
| | | TTTATTAGATATGATTGTTGGACAACACATCTGCCAAATAA |
| | | ATGTTCCTATGAAAAAATATATAAACACTTTAATACCCATA |
| | | TTATGGAATGTTCCCAACCTACACACTTTAAATGGTATGATA |
| | | ATTTGATGAAGAAACAAGATATTATGTAG |
| 7 | BA71- | ATGAGGTTCTTTAGTTACCTCGGCTTGCTGCTAGCTGGTCTA |
| | U104L | ACTAGTCTACAGGGTTTTTCGACCGACAATCTCCTGGAAGA |
| | (MGF-110 | GGAGCTAAGATACTGGTGTCAATATGTGAAAAATTGTCGGT |
| | family | TTTGCTGGACTTGTCAAGATGGTCTTTGTAAGAATAAAGTTC |
| | member) | TTAAAGATATGTCTTCTGTACAGGAGCATAGCTATCCCATG |
| | | GAACACTGTATGATTCACCGTCAGTATAAATATATTAGAGA |
| | | TGGGCCCATTTTCCAAGTAGAATGCACGATGCAGACATCTG |
| | | ATGCCACTCATTTAATAAATGCTTGA |
| 8 | XP124L | ATGTTGGTGATCTTCTTGGGAATTCTTGGCCTGCTGGCCAAT |
| | (MGF-110 | CAGGTCTTAGGACTACCTACTCAGGCAGGAGGGCATCTTCG |
| | family | TTCAACGGATAATCCTCCACAAGAAGAACTTGGATACTGGT |
| | member) | GTACTTACATGGAAAGCTGCAAGTTTTGCTGGGAATGTGCA |
| | | CATGGAATTTGCAAGAACAAGGTGAATGAGAGCATGCCATT |
| | | GATTATTGAGAACAGTTATTTGACATCTTGTGAGGTTTCTCG |
| | | CTGGTATAACCAGTGCACATATAGTGAAGGAAATGGGCATT |
| | | ACCATGTTATGGATTGTTCTAATCCAGTACCTCACAATCGTC |
| | | CACACCGATTGGGAAGGAAAATTTATGAAAAGGAAGATCT |
| | | GTGA |
| 9 | V82L | ATGTTAGTAATCTTCTTGGGAATTCTTGGCCTTCTGGCCAAC |
| | (MGF-110 | CAGGTCTCAAGCCAGCTCGTTGGACAACTTCATCCAACGGA |
| | family | AAATCCTTCAGAGAATGAACTTGAATATTGGTGCACTTACA |
| | member) | TGGAATGTTGCCAGTTTTGCTGGGACTGTCAAAATGGCCTTT |
| | | GTGTGAATAAGTTGGGAAATACAACAATTCTTGAAAATGAG |
| | | TATGTGCATCCATGTATAGTTTCCCGCTGGCTAAATAAATAA |
| 10 | Y118L | ATGTTGGTGATCTTTTTGGGAATTCTTGGCCTTCTGGCCAGC |
| | (MGF-110 | CAGGTTTCAAGTCAACTCGTTGGACAACTTCGACCAACAGA |
| | family | GGATCCTCCAGAGGAAGAACTCGAATACTGGTGCGCCTACA |
| | member) | TGGAAAGTTGTCAATTTTGCTGGGACTGCCAAGATGGCACT |
| | | TGTATAAACAAAATAGATGGGTCGGCCATTTATAAGAATGA |
| | | GTATGTGAAAGCATGTCTGGTGTCCCGTTGGCTGGATAAAT |
| | | GTATGTATGATTTAGATAAAGGTATCTACCATACCATGAAT |
| | | TGTTCTCAGCCATGGTCTTGGAATCCTTACAAATACTTCAGG |
| | | AAGGAATGGAAAAAAGATGAACTCTAG |

TABLE 4
Nuclease Target viral gene sequences used in vectors and heterologous RNA
polynucleotides described herein

| | | | | Targeting | |
|---|---|---|---|---|---|
| | | | | sequence (reverse | |
| | | | SEQ ID | complement of | SEQ ID |
| # | Gene | Virus Sequence Targeted | NO: | virus sequence) | NO: |
| 1. | DNA | GGTTCTCCCGTGCAAC | 11. | GATTGTTGCAC | 23. |
| | polymerase | AATC | | GGGAGAACC | |
| | (G1211R) | | | | |
| 2. | DNA | TCCACGAGACGATTGT | 12. | TTTAACAATCG | 24. |
| | polymerase | TAAA | | TCTCGTGGA | |
| | (G1211R) | | | | |
| 3. | DNA | GCGGGCTTACTTGCCA | 13. | ACTTTGGCAAG | 25. |
| | polymerase | AAGT | | TAAGCCCGC | |
| | (G1211R) | | | | |
| 4. | Topoisomerase | CGACAACGTGTCATAC | 14. | GGGTGTATGAC | 26. |
| | II | ACCC | | ACGTTGTCG | |
| | (p1192R) | | | | |
| 5. | Topoisomerase | CACCCGTCCAGATCTT | 15. | GACCAAGATCT | 27. |
| | II | GGTC | | GGACGGGTG | |
| | (p1192R) | | | | |
| 6. | Topoisomerase | TGGCGATATGTCGTTA | 16. | TGTTTAACGAC | 28. |
| | II | AACA | | ATATCGCCA | |
| | (p1192R) | | | | |
| 7. | RNA | CGTGTTCGAAGGACCC | 17. | AAAGGGGTCCT | 29. |
| | Helicase | CTTT | | TCGAACACG | |
| | (QP509L) | | | | |
| 8. | RNA | ACTATGTGCGTTCCCG | 18. | CATACGGGAAC | 30. |
| | Helicase | TATG | | GCACATAGT | |
| | (QP509L) | | | | |
| 9. | RNA | CTTGTAAAAGCCGAAG | 19. | TTTACTTCGGC | 31. |
| | Helicase | TAAA | | TTTTACAAG | |
| | (QP509L) | | | | |
| 10. | MGF110- | ATTCTTGAAAATGACT | 20. | ATATAGTCATT | 32. |
| | 1L | ATAT | | TTCAAGAAT | |
| | (MGF110 | | | | |
| | family) | | | | |
| 11. | MGF110- | TTCTTGAAAATGACTA | 21. | TATATAGTCAT | 33. |
| | 1L | TATA | | TTTCAAGAA | |
| | (MGF110 | | | | |
| | family) | | | | |
| 12. | MGF110- | TTGTAGATTCTGTTGG | 22. | AGTCCCAACAG | 34. |
| | 1L | GACT | | AATCTACAA | |
| | (MGF110 | | | | |
| | family) | | | | |
| 13. | MGF110- | AGCAAAATTGACAACT | 61 | GGAAAGTTGTC | 65 |
| | 1L | TTCC | | AATTTTGCT | |
| | (MGF110 | | | | |
| | family) | | | | |
| 14. | MGF110- | GCAAAATTGACAACTT | 62 | TGGAAAGTTGT | 66 |
| | 1L | TCCA | | CAATTTTGC | |
| | (MGF110 | | | | |
| | family) | | | | |
| 15. | MGF110- | TCTTGGCAGTCCCAGC | 63 | TTTTGCTGGGA | 67 |
| | 1L | AAAA | | CTGCCAAGA | |
| | (MGF110 | | | | |
| | family) | | | | |
| 16. | MGF110- | ACAGAGGATCCTCCAG | 64 | TCCTCTGGAGG | 68 |
| | 1L | AGGA | | ATCCTCTGT | |
| | (MGF110 | | | | |
| | family) | | | | |

TABLE 5
Sequences of promoter elements that can be used to drive expression of nucleases and
heterologous RNA polynucleotides such as sgRNAs described herein

| SEQ | | |
|---|---|---|
| ID NO: | Promoter | SEQUENCE |
| 37 | CMV plus | CGTTACATAACTTACGGTAAATGGCCCGCCTGGCTGACCGC |
| | element | CCAACGACCCCCGCCCATTGACGTCAATAATGACGTATGTT |
| | (see e.g., | CCCATAGTAACGCCAATAGGGACTTTCCATTGACGTCAATG |
| | https://www. | GGTGGAGTATTTACGGTAAACTGCCCACTTGGCAGTACATC |
| | snapgene. | AAGTGTATCATATGCCAAGTACGCCCCCTATTGACGTCAAT |
| | com/ | GACGGTAAATGGCCCGCCTGGCATTATGCCCAGTACATGAC |
| | resources/ | CTTATGGGACTTTCCTACTTGGCAGTACATCTACGTATTAGT |
| | plasmid- | CATCGCTATTACCATGGTGATGCGGTTTTGGCAGTACATCA |
| | plafiles/?set = | ATGGGCGTGGATAGCGGTTTGACTCACGGGGATTTCCAAGT |
| | basic_cloning_ | CTCCACCCCATTGACGTCAATGGGAGTTTGTTTTGGCACCAA |
| | vectors & | AATCAACGGGACTTTCCAAAATGTCGTAACAACTCCGCCCC |
| | plasmid = | ATTGACGCAAATGGGCGGTAGGCGTGTACGGTGGGAGGTCT |
| | CMV_ | ATATAAGCAGAGCT |
| | promoter) | |
| | enhancer | |
| 38 | ASFV p72 | TATTTAATAAAAACAATAAATTATTTTTATAACATTATATAT |
| | promoter | G |
| | (see e.g., | |
| | Garcia- | |
| | Escudero | |
| | R, Viñuela | |
| | E. | |
| | Structure | |
| | of African | |
| | swine | |
| | fever virus | |
| | late | |
| | promoters: | |
| | requirement | |
| | of a | |
| | TATA | |
| | sequence | |
| | at the | |
| | initiation | |
| | region. J | |
| | Virol. | |
| | 2000; 74(17): | |
| | 8176- | |
| | 8182. | |
| | doi: 10.1128/ | |
| | jvi.74.17. | |
| | 8176- | |
| | 8182.2000) | |
| 39 | ASFV p30 | TTTGTACTTTGCCGCGGATAAATTGCCAAGCATACATAAGTT |
| | promoter | GTTGATAATTTCTAATAAATCTGGATCGTGCTGCTGCAGCCA |
| | (see e.g., | TACAGAAATCTTGCAAAACTGTTTCATATTAGAGGGCATCT |
| | Supplementary | TCTTATTATTTTATAATTTTAAAATTGAATGGATTTTATTTTA |
| | data. | AATATAT |
| | Hübner, | |
| | A., C. | |
| | Keβler, K. | |
| | Pannhorst, | |
| | J. H. | |
| | Forth, T. | |
| | Kabuuka, | |
| | A. Karger, | |
| | T. C. | |
| | Mettenleiter | |
| | and W. | |
| | Fuchs | |
| | (2019). | |
| | “Identification | |
| | and | |
| | characterization | |
| | of | |
| | the 285L | |
| | and | |
| | K145R | |
| | proteins of | |
| | African | |
| | swine | |
| | fever | |
| | virus.” | |
| | Journal of | |
| | General | |
| | Virology | |
| | 100(9): | |
| | 1303- | |
| | 1314.) | |
| 40 | ASFV | TACCCGGTATAGAAAATAAAATTTAAAATAAAAAACGGAT |
| | DNA | GATATCTATTCATGGACCGTTCTGAGA |
| | polymerase | |
| | promoter | |
| | (see e.g., | |
| | Portugal | |
| | RS, Bauer | |
| | A, Keil | |
| | GM 2017 | |
| | Selection | |
| | of | |
| | differentially | |
| | temporally | |
| | regulated | |
| | African | |
| | swine | |
| | fever virus | |
| | promoter | |
| | with | |
| | variable | |
| | expression | |
| | activities | |
| | and their | |
| | application | |
| | for | |
| | transient | |
| | and | |
| | recombinant | |
| | virus | |
| | mediated | |
| | gene | |
| | expression | |
| | Virology | |
| | 508: 70-80 | |
| | (supplementary | |
| | materials)) | |
| 71 | HSV | AAATGAGTCTTCGGACCTCGCGGGGGCCGCTTAAGCGGTGG |
| | thymidine | TTAGGGTTTGTCTGACGCGGGGGGAGGGGGAAGGAACGAA |
| | kinase | ACACTCTCATTCGGAGGCGGCTCGGGGTTTGGTCTTGGTGG |
| | (Tk) | CCACGGGCACGCAGAAGAGCGCCGCGATCCTCTTAAGCACC |
| | promoter | CCCCCGCCCTCCGTGGAGGCGGGGGTTTGGTCGGCGGGTGG |
| | | TAACTGGCGGGCCGCTGACTCGGGCGGGTCGCGCGCCCCAG |
| | | AGTGTGACCTTTTCGGTCTGCTCGCAGACCCCCGGGCGGCG |
| | | CCGCCGCGGCGGCGACGGGCTCGCTGGGTCCTAGGCTCCAT |
| | | GGGGACCGTATACGTGGACAGGCTCTGGAGCATCCGCACGA |
| | | CTGCGGTGATATTACCGGAGACCTTCTGCGGGACGAGCCGG |
| | | GTCACGCGGCTGACGCGGAGCGTCCGTTGGGCGACAAACAC |
| | | CAGGACGGGGCACAGGTACACTATCTTGTCACCCGGAGGCG |
| | | CGAGGGACTGCAGGAGCTTCAGGGAGTGGCGCAGCTGCTTC |
| | | ATCCCCGTGGCCCGTTGCTCGCGTTTGCTGGCGGTGTCCCCG |
| | | GAAGAAATATATTTGCATGTCTTTAGTTCTATGATGACACA |
| | | AACCCCGCCCAGCGTCTTGTCATTGGCGAATTCGAACACGC |
| | | AGATGCAGTCGGGGCGGCGCGGTCCCAGGTCCACTTCGCAT |
| | | ATTAAGGTGACGCGTGTGGCCTCGAACACCGAGCGACCCTG |
| | | CAGCGACCCGCTTAA |
| 72 | SV40 | CTGAGGCGGAAAGAACCAGCTGTGGAATGTGTGTCAGTTAG |
| | promoter | GGTGTGGAAAGTCCCCAGGCTCCCCAGCAGGCAGAAGTATG |
| | | CAAAGCATGCATCTCAATTAGTCAGCAACCAGGTGTGGAAA |
| | | GTCCCCAGGCTCCCCAGCAGGCAGAAGTATGCAAAGCATGC |
| | | ATCTCAATTAGTCAGCAACCATAGTCCCGCCCCTAACTCCG |
| | | CCCATCCCGCCCCTAACTCCGCCCAGTTCCGCCCATTCTCCG |
| | | CCCCATGGCTGACTAATTTTTTTTATTTATGCAGAGGCCGAG |
| | | GCCGCCTCGGCCTCTGAGCTATTCCAGAAGTAGTGAGGAGG |
| | | CTTTTTTGGAGGCCTAGGCTTTTGCAAA |
| 73 | cytomegalovirus | CGTTACATAACTTACGGTAAATGGCCCGCCTGGCTGACCGC |
| | (CMV; | CCAACGACCCCCGCCCATTGACGTCAATAATGACGTATGTT |
| | human | CCCATAGTAACGCCAATAGGGACTTTCCATTGACGTCAATG |
| | immediate | GGTGGAGTATTTACGGTAAACTGCCCACTTGGCAGTACATC |
| | early) | AAGTGTATCATATGCCAAGTACGCCCCCTATTGACGTCAAT |
| | | GACGGTAAATGGCCCGCCTGGCATTATGCCCAGTACATGAC |
| | | CTTATGGGACTTTCCTACTTGGCAGTACATCTACGTATTAGT |
| | | CATCGCTATTACCATGGTGATGCGGTTTTGGCAGTACATCA |
| | | ATGGGCGTGGATAGCGGTTTGACTCACGGGGATTTCCAAGT |
| | | CTCCACCCCATTGACGTCAATGGGAGTTTGTTTTGGCACCAA |
| | | AATCAACGGGACTTTCCAAAATGTCGTAACAACTCCGCCCC |
| | | ATTGACGCAAATGGGCGGTAGGCGTGTACGGTGGGAGGTCT |
| | | ATATAAGCAGAGCT |
| 74 | Rous | AATGTAGTCTTATGCAATACACTTGTAGTCTTGCAACATGGT |
| | sarcoma | AACGATGAGTTAGCAACATGCCTTACAAGGAGAGAAAAAG |
| | virus | CACCGTGCATGCCGATTGGTGGAAGTAAGGTGGTACGATCG |
| | (RSV) | TGCCTTATTAGGAAGGCAACAGACAGGTCTGACATGGATTG |
| | promoter | GACGAACCACTGAATTGCGCATTGCAGAGATAATTGTATTT |
| | | AAGTGCCTAGCTCGATACAATAAACGCCATTTGACCATTCA |
| | | CCACATTGGTGTGCACC |
| 75 | Moloney | ccctcccccatatgtttacctactgaacatcacttggggttgtagaaactattgggaacttgtcctgga |
| | murine | gaaaattagtgaaagaccccacctgtaggtttggcaagctagcttaagtaacgccattttgcaaggc |
| | leukemia | atggaaaaatacataactgagaatagagaagttcagatcaaggtcaggaacagatggaacagctg |
| | virus long | aatatgggccaaacaggatatctgtggtaagcagttcctgccccggctcagggccaagaacagat |
| | terminal | ggtccccagatgcggtccagccctcagcagtttctagagaaccatcagatgtttccagggtgcccc |
| | repeat | aaggacctgaaatgaccctgtgccttatttgaactaaccaatcagttcgcttctcgcttctgttcgcgc |
| | | gcttctgctccccgcgctcaataaaagagcccacaacccctcactcggggcgccagtcctccgatt |
| | | gactgagtcgcccgggtacccgtgtatccaataaaccctcttgcagttgcatccgacttgtggtctcg |
| | | ctgttccttggggggtctcctctgagtgattgactacccgtcagcgggggtctttcatttgggggctc |
| | | gtccgggatcgggagacccct |
| 76 | mammalian | GCTCCGGTGCCCGTCAGTGGGCAGAGCGCACATCGCCCACA |
| | [elongation | GTCCCCGAGAAGTTGGGGGGAGGGGTCGGCAATTGAACCG |
| | factor 1α | GTGCCTAGAGAAGGTGGCGCGGGGTAAACTGGGAAAGTGA |
| | (EF1α) | TGTCGTGTACTGGCTCCGCCTTTTTCCCGAGGGTGGGGGAG |
| | | AACCGTATATAAGTGCAGTAGTCGCCGTGAACGTTCTTTTTC |
| | | GCAACGGGTTTGCCGCCAGAACACAGGTAAGTGCCGTGTGT |
| | | GGTTCCCGCGGGCCTGGCCTCTTTACGGGTTATGGCCCTTGC |
| | | GTGCCTTGAATTACTTCCACGCCCCTGGCTGCAGTACGTGAT |
| | | TCTTGATCCCGAGCTTCGGGTTGGAAGTGGGTGGGAGAGTT |
| | | CGAGGCCTTGCGCTTAAGGAGCCCCTTCGCCTCGTGCTTGA |
| | | GTTGAGGCCTGGCCTGGGCGCTGGGGCCGCCGCGTGCGAAT |
| | | CTGGTGGCACCTTCGCGCCTGTCTCGCTGCTTTCGATAAGTC |
| | | TCTAGCCATTTAAAATTTTTGATGACCTGCTGCGACGCTTTT |
| | | TTTCTGGCAAGATAGTCTTGTAAATGCGGGCCAAGATCTGC |
| | | ACACTGGTATTTCGGTTTTTGGGGCCGCGGGCGGCGACGGG |
| | | GCCCGTGCGTCCCAGCGCACATGTTCGGCGAGGCGGGGCCT |
| | | GCGAGCGCGGCCACCGAGAATCGGACGGGGGTAGTCTCAA |
| | | GCTGGCCGGCCTGCTCTGGTGCCTGGCCTCGCGCCGCCGTG |
| | | TATCGCCCCGCCCTGGGCGGCAAGGCTGGCCCGGTCGGCAC |
| | | CAGTTGCGTGAGCGGAAAGATGGCCGCTTCCCGGCCCTGCT |
| | | GCAGGGAGCTCAAAATGGAGGACGCGGCGCTCGGGAGAGC |
| | | GGGCGGGTGAGTCACCCACACAAAGGAAAAGGGCCTTTCC |
| | | GTCCTCAGCCGTCGCTTCATGTGACTCCACGGAGTACCGGG |
| | | CGCCGTCCAGGCACCTCGATTAGTTCTCGAGCTTTTGGAGTA |
| | | CGTCGTCTTTAGGTTGGGGGGAGGGGTTTTATGCGATGGAG |
| | | TTTCCCCACACTGAGTGGGTGGAGACTGAAGTTAGGCCAGC |
| | | TTGGCACTTGATGTAATTCTCCTTGGAATTTGCCCTTTTTGA |
| | | GTTTGGATCTTGGTTCATTCTCAAGCCTCAGACAGTGGTTCA |
| | | AAGTTTTTTTCTTCCATTTCAGGTGTCGTGA |
| 77 | cytokeratin | CCCGGGGCGGAGCGGCCCGGGGCGGAGGGGCGCGGGCTCC |
| | 18 | GAGCCGTCCACCTGTGGCTCCGGCTTCCGAAGCGGCTCCGG |
| | (K18) | GGCGGGGGCGGGGCCTCACTCTGCGATATAACTCGGGTCGC |
| | | GCGGCTCGCGCAGGCCGCCACCGTCGTCCGCAAAGCCTGAG |
| | | TC |
| 111 | cytokeratin | GATACCCGCCCCTTCAACATCTCCATCCCCCTATGGGCGGG |
| | 19 | GAAGTTGTAGAGGTAGGGG |
| | (K19) | |
| 78 | kallikrein | CTACTGCTGGTTTCTAGGGTAACCTTGGACTAGAGGTATAT |
| | (Kall) | GACCTTGTTTAGAGCAGTGGATTGGGGGTCACTGGCTCCTC |
| | | CACCTTCCTTTCACACCCCTTCACCATTGCCCCTGACCTTGC |
| | | CATCTGCCTTAGGTCACCATAACACTAACAGCTCCAGGAGC |
| | | AACAGGACCTGCCCCACCCAATCTCAGACCTTGGAAGGTAT |
| | | CCAAGGTGACCTAGGGCCCACAGGAGAGCAGGTGCAACAG |
| | | GGCCCTCCCCTCCCACAGCCATGAGGGTGGGAGAAGGGAGT |
| | | ACAACTCTGTCAGGAAGGGCAGGGCTTTGGCCAACATCTGG |
| | | CTGCCAACACGGCAGGGGTGGGGCTGTGGGGGAGAATGAG |
| | | GGTTTTTAAAGGCTCCCCAGGAGCCTCTAC |
| 69 | Procine | ctgacataagctgaaccaatgccttgcataatacctgcaatttagagtctataagtaaaaaccacttatt |
| | pancreatic | gatcacatgagccatcgtgctgtttttttgctaggaatattaactatgaaatctgctcttaataaggtttat |
| | n amylase | ccagaatgacagtcatgtaaatccttattttttataacattaatccaatatcacttaataacaacccggag |
| | promoter | gttaaaacctgccatacagaggagtacataactatggctgggaatatcaatataagtttcataaaggt |
| | (AMY) | atttttccaactgcatatgaaagtaggagtagttactagctattgaagggtgatacaagaaagaagaa |
| | | aagccctggaaagtcatgaaagaataaaattgttgtcaaatacgcaaaatgtttattttttdcgggaga |
| | | tggatattggggactctgcacttgttgttccgcccctctaacaatttgaaatattgaacttaactccaatg |
| | | tatgcgattaggctgtggggtctttgggaacaacttaggtcaaagtgacatcatgagagtggaggcc |
| | | ccatgatgggttagtgtcctttacgaagagaaagagaatcaggatctctgagctcacactcgtgagg |
| | | atacaagaagCttgctgtctgtgaacatggaatggggctttgcaagacactggagctgctgatagtg |
| | | tagtCttgggtttccagcctctagaaatgtgagaaagaaatatttgttgctaagccatccagcctatat |
| | | ggcattcttgttacagcagctggaactgaatgagaaaaataggacacggagtatgttcacgatgtgg |
| | | gctggaggagggaccgaaggagagtgttgggattcacagagtgctctcggaccccctccacaaa |
| | | gctagtacttcctcacttttcctcatcttagtaaatggtgtcatcagatacctgtttcctcaatttttctctttc |
| | | ccccagtcttcggtgctaatctatcgataaaccgattgcttcgccacctctgagatatattctatcaggg |
| | | ccctagagcagccactttcctctttcgtggaccactacaaaagcctacctgatctcttggcccccagt |
| | | cgtgtcctcctataatccggtttttcacagcagagcaagaatggttttcttggaaaggaaatcagaatc |
| | | tcttcactcatcttctttcagcctcaaaagccctctctttccttatgttctacaaggttctacatgatctggc |
| | | ctacctctctgatttcatctcattttactcttccctttgtcactcacacatgtttagctgcactgatgttgaaa |
| | | gtttgttcagtgtcacttgagtatcccacggttgttcctaccttgggcttttgctattgcactttcctctatg |
| | | gagactgcttttcctctgatcttcaaataagtgggtccttctactccttccagttctggctgacaatcact |
| | | ccctctgaaacagctttcctgactatttccagtctaaaatatcctgaaaaattcagtccttttccctttaac |
| | | tgcaccgtgggttcatgctagttctcactgctctctttaacttagtatcgttgttgttatcattccatcttgct |
| | | atattttccttaccttcccctagaatgtaggctgagaacaagagtcttgtctgtcttgttcatccttgtatc |
| | | ctgagtatcatgccggcatttagcaaaagcactcggccactacctgttggatgaatggattaggttttt |
| | | cccacctgtacggttatgtctttactaggatttcttgtaccttacgaaggaaaatagatgtggattcatta |
| | | acttagtgttttagcacatataagggactttttgctagaaggagaaaaaaaaaagtccattctttcctgc |
| | | tacagccagtgcattttcacatgcgttaatgtaagcgtggggaaaaaaaaatctgacacctaaagtc |
| | | gtggtcatttcacttccggataacttcctaaatcttagtggagaatctcaagtatctaacaactagggta |
| | | ggaggtaccaactgaactgagttgaataacatgtgtcttcttacaatggaaacattgcacgtgtttaca |
| | | gacagttagggcaccattgtgactgtgaattcagttggctctaattccgcctctgtcagtgaaggactt |
| | | cagaaataaaatctaatcctacctaaacaatacatgattaagacctttctgtagataacatgccagatg |
| | | tttcaaaacttgctgttccctcagtaaggaaaacattgtctgagaaggtcatttagatagtattcctggg |
| | | agattttcgggatgttcctcacctgtttagtgtaattatcaatagttatttttggagtatgcattcacggttt |
| | | gtgctctaagtatttattcatgtcaatatttgctttgtaaaatatgcttcttgcaggattataaatacttgcc |
| | | gggaagaccgttgacaacctcagagcaaaatgaagttgtttctgctgctttcagccattgggttctgct |
| | | gggcc |
| 70 | human, | AAGGGAACCCCGGCCTGGGAGAGGGCGCCTCCGGGGATCC |
| | and rat | GTTGCCTAGTCCAGGTACTGCCCAGCTACCGGGCGTCGAGG |
| | aquaporin- | ATTGCGAACGGTCGGGGCAGGCTGGCACGGTGCCCACTTTT |
| | 5 (rAQP5) | CCCAAAACTCCAGCCTTCCAAGCCCAGAAGCTCGCCCGGCC |
| | | CAGGCCGAGCTGGCCACGTCGGACGGCCGGACCGCCCTGCA |
| | | GGACCCAGCCCGGCCGGGGGCCCCCGCCGGCGGTGAGGGA |
| | | GGTGAGCGGCGCCGACCTGCGGGACGAGCAccccggccTCACT |
| | | CCGACCCAGCCGGGGGTGAGGCGGGTCAGGATGCTCCGGTC |
| | | GCAGGAGGAAAAGGAGGAGCTGGACCAAAAGCCCGAAGA |
| | | GAAGAAAAGGGGAAGGCCGCGCACGGAGCGCGGTAAAGGC |
| | | CGGCGG |

TABLE 6
Sequences of gene-targeting vectors according to the present disclosure

| SEQ | | |
|---|---|---|
| ID | | |
| NO: | Description | SEQUENCE |
| 41 | P18 vector | agacacaattccccagtggaaagacgcgcaggcaaaacgcaccacgtgacggagcgtgaccgc |
| | for targeting | gcgccgagcgcgcgccaaggtcgggcaggaagagggcctatttcccatgattccttcatatttgca |
| | ASFV genes | tatacgatacaaggctgttagagagataattagaattaatttgactgtaaacacaaagatattagtaca |
| | including | aaatacgtgacgtagaaagtaataatttcttgggtagtttgcagttttaaaattatgttttaaaatggacta |
| | sgRNAs | tcatatgcttaccgtaacttgaaagtatttcgatttcttgggtttatatatcttgtggaaaggacgaggat |
| | scaffolds | ccgcaactcatagagagttagcggttttagagctagaaatagcaagttaaaataaggctagtccgtta |
| | driven by | tcaacttgaaaaagtggcaccgagtcggtgcttttttctagacacaattccccagtggaaagacgcg |
| | SP6 and U6 | caggcaaaacgcaccacgtgacggagcgtgaccgcgcgccgagcgcgcgccaaggtcgggca |
| | promoters | ggaagagggcctatttcccatgattccttcatatttgcatatacgatacaaggctgttagagagataatt |
| | and | agaattaatttgactgtaaacacaaagatattagtacaaaatacgtgacgtagaaagtaataatttcttg |
| | NLS(SAGSSG)- | ggtagtttgcagttttaaaattatgttttaaaatggactatcatatgcttaccgtaacttgaaagtatttcga |
| | Cas9 | tttcttgggtttatatatcttgtggaaaggacgaggatccgcgtggtttagagaagcgcacgttttaga |
| | driven by | gctagaaatagcaagttaaaataaggctagtccgttatcaacttgaaaaagtggcaccgagtcggtg |
| | CMV | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCgtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgctaggcg |
| | | gccgcagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | acaaaatacgtgacgtagaaagtaataatttcttgggtagtttgcagttttaaaattatgttttaaaatgg |
| | | actatcatatgcttaccgtaacttgaaagtatttcgatttcttgggtttatatatcttgtggaaaggacgag |
| | | gatccgcctctgaccttaattatagggttttagagctagaaatagcaagttaaaataaggctagtccgt |
| | | tatcaacttgaaaaagtggcaccgagtcggtgcttttttct |
| 42 | P19 vector | agacacaattgcatgaagaatctgcttagggttaggcgttttgcgctgcttcgcgatgtacgggcca |
| | for targeting | gatatacgcgttgacattgattattgactagttattaatagtaatcaattacggggtcattagttcatagc |
| | ASFV genes | ccatatatggagttccgcgttacataacttacggtaaatggcccgcctggctgaccgcccaacgacc |
| | including | cccgcccattgacgtcaataatgacgtatgttcccatagtaacgccaatagggactttccattgacgt |
| | sgRNAs | caatgggtggagtatttacggtaaactgcccacttggcagtacatcaagtgtatcatatgccaagtac |
| | scaffolds | gccccctattgacgtcaatgacggtaaatggcccgcctggcattatgcccagtacatgaccttatgg |
| | driven by | gactttcctacttggcagtacatctacgtattagtcatcgctattaccatggtgatgcggttttggcagta |
| | SP6 and U6 | catcaatgggcgtggatagcggtttgactcacggggatttccaagtctccaccccattgacgtcaat |
| | promoters | gggagtttgttttggcaccaaaatcaacgggactttccaaaatgtcgtaacaactccgccccattgac |
| | and | gcaaatgggcggtaggcgtgtacggtgggaggtctatataagcagagctctctggctaactagaga |
| | NLS(SAGSSG)- | acccactgcttactggcttatcgaaattaatacgactcactatagggagacccaagcttgccaccatg |
| | Cas9 | gacaagaagtacagcatcggcctggacatcggtaccaacagcgtgggctgggccgtgatcaccg |
| | driven by | acgagtacaaggtgcccagcaagaagttcaaggtgctgggcaacaccgaccgccacagcatcaa |
| | CMV | gaagaacctgatcggcgccctgctgttcgacagcggcgagaccgccgaggccacccgcctgaa |
| | | gcgcaccgcccgccgccgctacacccgccgcaagaaccgcatctgctacctgcaggagatcttc |
| | | agcaacgagatggccaaggtggacgacagcttcttccaccgcctggaggagagcttcctggtgga |
| | | ggaggacaagaagcacgagcgccaccccatcttcggcaacatcgtggacgaggtggcctaccac |
| | | gagaagtaccccaccatctaccacctgcgcaagaagctggtggacagcaccgacaaggccgacc |
| | | tgcgcctgatctacctggccctggcccacatgatcaagttccgcggccacttcctgatcgagggcg |
| | | acctgaaccccgacaacagcgacgtggacaagctgttcatccagctggtgcagacctacaaccag |
| | | ctgttcgaggagaaccccatcaacgccagcggcgtggacgccaaggccatcctgagcgcccgcc |
| | | tgagcaagagccgccgcctggagaacctgatcgcccagctgcccggcgagaagaagaacggcc |
| | | tgttcggcaacctgatcgccctgagcctgggcctgacccccaacttcaagagcaacttcgacctgg |
| | | ccgaggacgccaagctgcagctgagcaaggacacctacgacgacgacctggacaacctgctgg |
| | | cccagatcggcgaccagtacgccgacctgttcctggccgccaagaacctgagcgacgccatcctg |
| | | ctgagcgacatcctgcgcgtgaacaccgagatcaccaaggcccccctgagcgccagcatgatca |
| | | agcgctacgacgagcaccaccaggacctgaccctgctgaaggccctggtgcgccagcagctgcc |
| | | cgagaagtacaaggagatcttcttcgaccagagcaagaacggctacgccggctacatcgacggc |
| | | ggcgccagccaggaggagttctacaagttcatcaagcccatcctggagaagatggacggcaccg |
| | | aggagctgctggtgaagctgaaccgcgaggacctgctgcgcaagcagcgcaccttcgacaacgg |
| | | cagcatcccccaccagatccacctgggcgagctgcacgccatcctgcgccgccaggaggacttct |
| | | accccttcctgaaggacaaccgcgagaagatcgagaagatcctgaccttccgcatcccctactacg |
| | | tgggccccctggcccgcggcaacagccgcttcgcctggatgacccgcaagagcgaggagacca |
| | | tcaccccctggaacttcgaggaggtggtggacaagggcgccagcgcccagagcttcatcgagcg |
| | | catgaccaacttcgacaagaacctgcccaacgagaaggtgctgcccaagcacagcctgctgtacg |
| | | agtacttcaccgtgtacaacgagctgaccaaggtgaagtacgtgaccgagggcatgcgcaagccc |
| | | gccttcctgagcggcgagcagaagaaggccatcgtggacctgctgttcaagaccaaccgcaaggt |
| | | gaccgtgaagcagctgaaggaggactacttcaagaagatcgagtgcttcgacagcgtggagatca |
| | | gcggcgtggaggaccgcttcaacgccagcctgggcacctaccacgacctgctgaagatcatcaa |
| | | ggacaaggacttcctggacaacgaggagaacgaggacatcctggaggacatcgtgctgaccctg |
| | | accctgttcgaggaccgcgagatgatcgaggagcgcctgaagacctacgcccacctgttcgacga |
| | | caaggtgatgaagcagctgaagcgccgccgctacaccggctggggccgcctgagccgcaagctt |
| | | atcaacggcatccgcgacaagcagagcggcaagaccatcctggacttcctgaagagcgacggct |
| | | tcgccaaccgcaacttcatgcagctgatccacgacgacagcctgaccttcaaggaggacatccag |
| | | aaggcccaggtgagcggccagggcgacagcctgcacgagcacatcgccaacctggccggcag |
| | | ccccgccatcaagaagggcatcctgcagaccgtgaaggtggtggacgagctggtgaaggtgatg |
| | | ggccgccacaagcccgagaacatcgtgatcgagatggcccgcgagaaccagaccacccagaag |
| | | ggccagaagaacagccgcgagcgcatgaagcgcatcgaggagggcatcaaggagctgggcag |
| | | ccagatcctgaaggagcaccccgtggagaacacccagctgcagaacgagaagctgtacctgtact |
| | | acctgcagaacggccgcgacatgtacgtggaccaggagctggacatcaaccgcctgagcgacta |
| | | cgacgtggaccacatcgtgccccagagcttcctgaaggacgacagcatcgacaacaaggtgctg |
| | | acccgcagcgacaagaaccgcggcaagagcgacaacgtgcccagcgaggaggtggtgaagaa |
| | | gatgaagaactactggcgccagctgctgaacgccaagctgatcacccagcgcaagttcgacaacc |
| | | tgaccaaggccgagcgcggcggcctgagcgagctggacaaggccggcttcatcaagcgccagc |
| | | tggtggagacccgccagatcaccaagcacgtggcccagatcctggacagccgcatgaacaccaa |
| | | gtacgacgagaacgacaagctgatccgcgaggtgaaggtgatcaccctgaagagcaagctggtg |
| | | agcgacttccgcaaggacttccagttctacaaggtgcgcgagatcaacaactaccaccacgccca |
| | | cgacgcctacctgaacgccgtggtgggcaccgccctgatcaagaagtaccccaagctggagagc |
| | | gagttcgtgtacggcgactacaaggtgtacgacgtgcgcaagatgatcgccaagagcgagcagg |
| | | agatcggcaaggccaccgccaagtacttcttctacagcaacatcatgaacttcttcaagaccgagat |
| | | caccctggccaacggcgagatccgcaagcgccccctgatcgagaccaacggcgagaccggcg |
| | | agatcgtgtgggacaagggccgcgacttcgccaccgtgcgcaaggtgctgagcatgccccaggt |
| | | gaacatcgtgaagaagaccgaggtgcagaccggcggcttcagcaaggagagcatcctgcccaa |
| | | gcgcaacagcgacaagctgatcgcccgcaagaaggactgggaccccaagaagtacggcggctt |
| | | cgacagccccaccgtggcctacagcgtgctggtggtggccaaggtggagaagggcaagagcaa |
| | | gaagctgaagagcgtgaaggagctgctgggcatcaccatcatggagcgcagcagcttcgagaag |
| | | aaccccatcgacttcctggaggccaagggctacaaggaggtgaagaaggacctgatcatcaagct |
| | | gcccaagtacagcctgttcgagctggagaacggccgcaagcgcatgctggccagcgccggcga |
| | | gctgcagaagggcaacgagctggccctgcccagcaagtacgtgaacttcctgtacctggccagcc |
| | | actacgagaagctgaagggcagccccgaggacaacgagcagaagcagctgttcgtggagcagc |
| | | acaagcactacctggacgagatcatcgagcagatcagcgagttcagcaagcgcgtgatcctggcc |
| | | gacgccaacctggacaaggtgctgagcgcctacaacaagcaccgcgacaagcccatccgcgag |
| | | caggccgagaacatcatccacctgttcaccctgaccaacctgggcgcccccgccgccttcaagta |
| | | cttcgacaccaccatcgaccgcaagcgctacaccagcaccaaggaggtgctggacgccaccctg |
| | | atccaccagagcatcaccggtctgtacgagacccgcatcgacctgagccagctgggcggcgacg |
| | | gcggctccggacctccaaGCGCCGGCAGCAGCGGCgtatacccctacgacgtgcc |
| | | cgactacgccctcgaggagggcagaggaagtcttctaacatgcggtgacgtggaggagaatccc |
| | | ggccctatggagagcgacgagagcggcctgcccgccatggagatcgagtgccgcatcaccggc |
| | | accctgaacggcgtggagttcgagctggtgggcggcggagagggcacccccaagcagggccg |
| | | catgaccaacaagatgaagagcaccaaaggcgccctgaccttcagcccctacctgctgagccacg |
| | | tgatgggctacggcttctaccacttcggcacctaccccagcggctacgagaaccccttcctgcacg |
| | | ccatcaacaacggcggctacaccaacacccgcatcgagaagtacgaggacggcggcgtgctgc |
| | | acgtgagcttcagctaccgctacgaggccggccgcgtgatcggcgacttcaaggtggtgggcacc |
| | | ggcttccccgaggacagcgtgatcttcaccgacaagatcatccgcagcaacgccaccgtggagca |
| | | cctgcaccccatgggcgataacgtgctggtgggcagcttcgcccgcaccttcagcctgcgcgacg |
| | | gcggctactacagcttcgtggtggacagccacatgcacttcaagagcgccatccaccccagcatcc |
| | | tgcagaacgggggccccatgttcgccttccgccgcgtggaggagctgcacagcaacaccgagct |
| | | gggcatcgtggagtaccagcacgccttcaagacccccatcgccttcgccagatccegcgctcagt |
| | | cgtccaattctgccgtggacggcaccgccggacccggctccaccggatctcgctaggcggccgc |
| | | agatgggggtcctgggccccagggtgtgcagccactgacttggggactgctggtggggtaggga |
| | | tgagggagggaggggcattgtgatgtacagggctgctctgtgagatcaagggtctcttaagggtgg |
| | | gagctggggcagggactacgagagcagccagatgggctgaaagtggaactcaaggggtttctgg |
| | | cacctacctacctgcttcccgctggggggtggggagttggcccagagtcttaagattggggcagg |
| | | gtggagaggtgggctcttcctgcttcccactcatcttatagctttctttccccagatccgaattggagat |
| | | ccaaaccaaggcgcgcgctagcgccaccatgggatcggccattgaacaagatggattgcacgca |
| | | ggttctccggccgcttgggtggagaggctattcggctatgactgggcacaacagacaatcggctgc |
| | | tctgatgccgccgtgttccggctgtcagcgcaggggcgcccggttctttttgtcaagaccgacctgt |
| | | ccggtgccctgaatgaactgcaggacgaggcagcgcggctatcgtggctggccacgacgggcgt |
| | | tccttgcgcagcagtgctcgacgttgtcactgaagcgggaagggactggctgctattgggcgaagt |
| | | gccggggcaggatctcctgtcatctcaccttgctcctgccgagaaagtatccatcatggctgatgca |
| | | atgcggcggctgcatacgcttgatccggctacctgcccattcgaccaccaagcgaaacatcgcatc |
| | | gagcgagcacgtactcggatggaagccggtcttgtcgatcaggatgatctggacgaagagcatca |
| | | ggggctcgcgccagccgaactgttcgccaggctcaaggcgcgtatgcccgacggcgatgatctc |
| | | gtcgtgactcatggcgatgcctgcttgccgaatatcatggtggaaaatggccgcttttctggattcatc |
| | | gactgtggccggctgggtgtggcggaccgctatcaggacatagcgttggctacccgtgatattgct |
| | | gaagagcttggcggcgaatgggctgaccgcttcctcgtgctttacggtatcgccgctcccgattcgc |
| | | agcgcatcgccttctatcgccttcttgacgagttcttctgaacgcggtgctacgagatttcgattccac |
| | | cgccgccttctatgaaaggttgggcttcggaatcgttttccgggacgccggctggatgatcctccag |
| | | cgcggggatctcatgctggagttcttcgcccaccccaacttgtttattgcagcttataatggttacaaat |
| | | aaagcaatagcatcacaaatttcacaaataaagcatttttttcactgcattctagttgtggtttgtccaaa |
| | | ctcatcaatgtatcttatcatgtctgtataccgtcgacctctagctagagcttggcgtaatcatggtcata |
| | | gctgtttcctgtgtgaaattgttatccgctcacaattccacacaacatacgagccggaagcataaagg |
| | | taagcctgaatattgaaaaaggaagagtatgagtattcaacatttccgtgtcgcccttattcccttttttg |
| | | cggcattttgccttcctgtttttgctcacccagaaacgctggtgaaagtaaaagatgctgaagatcagt |
| | | tgggtgcacgagtgggttacatcgaactggatctcaacagcggtaagatccttgagagttttcgccc |
| | | cgaagaacgttttccaatgatgagcacttttaaagttctgctatgtggcgcggtattatcccgtattgac |
| | | gccgggcaagagcaactcggtcgccgcatacactattctcagaatgacttggttgagtactcacca |
| | | gtcacagaaaagcatcttacggatggcatgacagtaagagaattatgcagtgctgccataaccatga |
| | | gtgataacactgcggccaacttacttctgacaacgatcggaggaccgaaggagctaaccgctttttt |
| | | gcacaacatgggggatcatgtaactcgccttgatcgttgggaaccggagctgaatgaagccatacc |
| | | aaacgacgagcgtgacaccacgatgcctgtagcaatggcaacaacgttgcgcaaactattaactg |
| | | gcgaactacttactctagcttcccggcaacaattaatagactggatggaggcggataaagttgcagg |
| | | accacttctgcgctcggcccttccggctggctggtttattgctgataaatctggagccggtgagcgtg |
| | | ggtctcgcggtatcattgcagcactggggccagatggtaagccctcccgtatcgtagttatctacac |
| | | gacggggagtcaggcaactatggatgaacgaaatagacagatcgctgagataggtgcctcactga |
| | | ttaagcattggtaactgtcagaccaagtttactcatatatactttagattgatttaaaacttcatttttaattt |
| | | aaaaggatctaggtgaagatcctttttgataatctcatgaccaaaatcccttaacgtgagttttcgttcc |
| | | actgagcgtcagaccccgtagaaaagatcaaaggatcttcttgagatcctttttttctgcgcgtaatct |
| | | gctgcttgcaaacaaaaaaaccaccgctaccagcggtggtttttttgccggatcaagagctaccaac |
| | | tctttttccgaaggtaactggcttcagcagagcgcagataccaaatactgttcttctagtgtagccgta |
| | | gttaggccaccacttcaagaactctgtagcaccgcctacatacctcgctctgctaatcctgttaccagt |
| | | ggctgctgccagtggcgataagtcgtgtcttaccgggttggactcaagacgatagttaccggataag |
| | | gcgcagcggtcgggctgaacggggggttcgtgcacacagcccagcttggagcgaacgacctac |
| | | accgaactgagatacctacagcgtgagctatgagaaagcgccacgcttcccgaagggagaaagg |
| | | cggacaggtatccggtaagcggcagggtcggaacaggagagcgcacgagggagcttccaggg |
| | | ggaaacgcctggtatctttatagtcctgtcgggtttcgccacctctgacttgagcgtcgatttttgtgat |
| | | gctcgtcaggggggcggagcctatggaaaaacgccagcaacgcggcctttttacggttcctggcc |
| | | ttttgctggccttttgctcacatgttctttcctgcgttatcccctgattctgtggataaccgtattaccgcct |
| | | ttgagtgagctgataccgctcgccgcagccgaacgaccgagcgcagcgagtcagtgagcgagg |
| | | aagcggaagagcgcccaatacgcaaaccgcctctccccgcgcgttggccgattcattaatgcagc |
| | | tggcacgacaggtttcccgactggaaagcgggcagtgagcgcaacgcaattaatgtgagttagctc |
| | | actcattaggcaccccaggctttacactttatgcttccggctcgtatgttgtgtggaattgtgagcggat |
| | | aacaatttcacacaggaaacagctatgacatgattacgaattgcaacgatttaggtgacactatagaa |
| | | gagaaggaattaatacgactcactatagggagagagagagaattaccctcactaaagggaggaga |
| | | agcatgaattccccagtggaaagacgcgcaggcaaaacgcaccacgtgacggagcgtgaccgc |
| | | gcgccgagcgcgcgccaaggtcgggcaggaagagggcctatttcccatgattccttcatatttgca |
| | | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccggagaagcttctctgttttagagctagaaatagcaagttaaaataaggctagtccgttatcaacttg |
| | | aaaaagtggcaccgagtcggtgcttttttct |
| 43 | P20 vector | agacacaattccccagtggaaagacgcgcaggcaaaacgcaccacgtgacggagcgtgaccgc |
| | for targeting | gcgccgagcgcgcgccaaggtcgggcaggaagagggcctatttcccatgattccttcatatttgca |
| | ASFV genes | tatacgatacaaggctgttagagagataattagaattaatttgactgtaaacacaaagatattagtaca |
| | including | aaatacgtgacgtagaaagtaataatttcttgggtagtttgcagttttaaaattatgttttaaaatggacta |
| | sgRNAs | tcatatgcttaccgtaacttgaaagtatttcgatttcttgggtttatatatcttgtggaaaggacgaggat |
| | scaffolds | ccgaccaagatctggacgggtggttttagagctagaaatagcaagttaaaataaggctagtccgtta |
| | driven by | tcaacttgaaaaagtggcaccgagtcggtgcttttttctagacacaattccccagtggaaagacgcg |
| | SP6 and U6 | caggcaaaacgcaccacgtgacggagcgtgaccgcgcgccgagcgcgcgccaaggtcgggca |
| | promoters | ggaagagggcctatttcccatgattccttcatatttgcatatacgatacaaggctgttagagagataatt |
| | and | agaattaatttgactgtaaacacaaagatattagtacaaaatacgtgacgtagaaagtaataatttcttg |
| | NLS(SAGSSG)- | ggtagtttgcagttttaaaattatgttttaaaatggactatcatatgcttaccgtaacttgaaagtatttcga |
| | Cas9 | tttcttgggtttatatatcttgtggaaaggacgaggatccgggtgtatgacacgttgtcggttttagagc |
| | driven by | tagaaatagcaagttaaaataaggctagtccgttatcaacttgaaaaagtggcaccgagtcggtgctt |
| | CMV | ttttctagacacaattgcatgaagaatctgcttagggttaggcgttttgcgctgcttcgcgatgtacgg |
| | | gccagatatacgcgttgacattgattattgactagttattaatagtaatcaattacggggtcattagttca |
| | | tagcccatatatggagttccgcgttacataacttacggtaaatggcccgcctggctgaccgcccaac |
| | | gacccccgcccattgacgtcaataatgacgtatgttcccatagtaacgccaatagggactttccattg |
| | | acgtcaatgggtggagtatttacggtaaactgcccacttggcagtacatcaagtgtatcatatgccaa |
| | | gtacgccccctattgacgtcaatgacggtaaatggcccgcctggcattatgcccagtacatgacctt |
| | | atgggactttcctacttggcagtacatctacgtattagtcatcgctattaccatggtgatgcggttttggc |
| | | agtacatcaatgggcgtggatagcggtttgactcacggggatttccaagtctccaccccattgacgt |
| | | caatgggagtttgttttggcaccaaaatcaacgggactttccaaaatgtcgtaacaactccgccccat |
| | | tgacgcaaatgggcggtaggcgtgtacggtgggaggtctatataagcagagctctctggctaacta |
| | | gagaacccactgcttactggcttatcgaaattaatacgactcactatagggagacccaagcttgcca |
| | | ccatggacaagaagtacagcatcggcctggacatcggtaccaacagcgtgggctgggccgtgat |
| | | caccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCgtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgctaggcg |
| | | gccgcagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | actatcatatgcttaccgtaacttgaaagtatttcgatttcttgggtttatatatcttgtggaaaggacgag |
| | | gatccgtgtttaacgacatatcgccagttttagagctagaaatagcaagttaaaataaggctagtccgt |
| | | tatcaacttgaaaaagtggcaccgagtcggtgcttttttct |
| 44 | P21 vector | agacacaattccccagtggaaagacgcgcaggcaaaacgcaccacgtgacggagcgtgaccgc |
| | for targeting | gcgccgagcgcgcgccaaggtcgggcaggaagagggcctatttcccatgattccttcatatttgca |
| | ASFV genes | tatacgatacaaggctgttagagagataattagaattaatttgactgtaaacacaaagatattagtaca |
| | including | aaatacgtgacgtagaaagtaataatttcttgggtagtttgcagttttaaaattatgttttaaaatggacta |
| | sgRNAs | tcatatgcttaccgtaacttgaaagtatttcgatttcttgggtttatatatcttgtggaaaggacgaggat |
| | scaffolds | ccgtttacttcggcttttacaaggttttagagctagaaatagcaagttaaaataaggctagtccgttatc |
| | driven by | aacttgaaaaagtggcaccgagtcggtgcttttttctagacacaattccccagtggaaagacgcgca |
| | SP6 and U6 | ggcaaaacgcaccacgtgacggagcgtgaccgcgcgccgagcgcgcgccaaggtcgggcag |
| | promoters | gaagagggcctatttcccatgattccttcatatttgcatatacgatacaaggctgttagagagataatta |
| | and | gaattaatttgactgtaaacacaaagatattagtacaaaatacgtgacgtagaaagtaataatttcttgg |
| | NLS(SAGSSG)- | gtagtttgcagttttaaaattatgttttaaaatggactatcatatgcttaccgtaacttgaaagtatttcgat |
| | Cas9 | ttcttgggtttatatatcttgtggaaaggacgaggatccgaaaggggtccttcgaacacggttttagag |
| | driven by | ctagaaatagcaagttaaaataaggctagtccgttatcaacttgaaaaagtggcaccgagtcggtgc |
| | CMV | ttttttctagacacaattgcatgaagaatctgcttagggttaggcgttttgcgctgcttcgcgatgtacg |
| | | ggccagatatacgcgttgacattgattattgactagttattaatagtaatcaattacggggtcattagttc |
| | | atagcccatatatggagttccgcgttacataacttacggtaaatggcccgcctggctgaccgcccaa |
| | | cgacccccgcccattgacgtcaataatgacgtatgttcccatagtaacgccaatagggactttccatt |
| | | gacgtcaatgggtggagtatttacggtaaactgcccacttggcagtacatcaagtgtatcatatgcca |
| | | agtacgccccctattgacgtcaatgacggtaaatggcccgcctggcattatgcccagtacatgacct |
| | | tatgggactttcctacttggcagtacatctacgtattagtcatcgctattaccatggtgatgcggttttgg |
| | | cagtacatcaatgggcgtggatagcggtttgactcacggggatttccaagtctccaccccattgacg |
| | | tcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaact |
| | | agagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCtacccctacgacgtg |
| | | cccgactacgccctcgaggagggcagaggaagtcttctaacatgcggtgacgtggaggagaatc |
| | | ccggccctatggagagcgacgagagcggcctgcccgccatggagatcgagtgccgcatcaccg |
| | | gcaccctgaacggcgtggagttcgagctggtgggcggcggagagggcacccccaagcagggc |
| | | cgcatgaccaacaagatgaagagcaccaaaggcgccctgaccttcagcccctacctgctgagcca |
| | | cgtgatgggctacggcttctaccacttcggcacctaccccagcggctacgagaaccccttcctgca |
| | | cgccatcaacaacggcggctacaccaacacccgcatcgagaagtacgaggacggcggcgtgct |
| | | gcacgtgagcttcagctaccgctacgaggccggccgcgtgatcggcgacttcaaggtggtgggc |
| | | accggcttccccgaggacagcgtgatcttcaccgacaagatcatccgcagcaacgccaccgtgga |
| | | gcacctgcaccccatgggcgataacgtgctggtgggcagcttcgcccgcaccttcagcctgcgcg |
| | | acggcggctactacagcttcgtggtggacagccacatgcacttcaagagcgccatccaccccagc |
| | | atcctgcagaacgggggccccatgttcgccttccgccgcgtggaggagctgcacagcaacaccg |
| | | agctgggcatcgtggagtaccagcacgccttcaagacccccatcgccttcgccagatcccgcgct |
| | | cagtcgtccaattctgccgtggacggcaccgccggacccggctccaccggatctcgctaggcggc |
| | | cgcagatgggggtcctgggccccagggtgtgcagccactgacttggggactgctggtggggtag |
| | | ggatgagggagggaggggcattgtgatgtacagggctgctctgtgagatcaagggtctcttaagg |
| | | gtgggagctggggcagggactacgagagcagccagatgggctgaaagtggaactcaaggggttt |
| | | ctggcacctacctacctgcttcccgctggggggtggggagttggcccagagtcttaagattggggc |
| | | agggtggagaggtgggctcttcctgcttcccactcatcttatagctttctttccccagatccgaattgg |
| | | agatccaaaccaaggcgcgcgctagcgccaccatgggatcggccattgaacaagatggattgca |
| | | cgcaggttctccggccgcttgggtggagaggctattcggctatgactgggcacaacagacaatcg |
| | | gctgctctgatgccgccgtgttccggctgtcagcgcaggggcgcccggttctttttgtcaagaccga |
| | | cctgtccggtgccctgaatgaactgcaggacgaggcagcgcggctatcgtggctggccacgacg |
| | | ggcgttccttgcgcagcagtgctcgacgttgtcactgaagcgggaagggactggctgctattgggc |
| | | gaagtgccggggcaggatctcctgtcatctcaccttgctcctgccgagaaagtatccatcatggctg |
| | | atgcaatgcggcggctgcatacgcttgatccggctacctgcccattcgaccaccaagcgaaacatc |
| | | gcatcgagcgagcacgtactcggatggaagccggtcttgtcgatcaggatgatctggacgaagag |
| | | catcaggggctcgcgccagccgaactgttcgccaggctcaaggcgcgtatgcccgacggcgatg |
| | | atctcgtcgtgactcatggcgatgcctgcttgccgaatatcatggtggaaaatggccgcttttctggat |
| | | tcatcgactgtggccggctgggtgtggcggaccgctatcaggacatagcgttggctacccgtgata |
| | | ttgctgaagagcttggcggcgaatgggctgaccgcttcctcgtgctttacggtatcgccgctcccga |
| | | ttcgcagcgcatcgccttctatcgccttcttgacgagttcttctgaacgcggtgctacgagatttcgatt |
| | | ccaccgccgccttctatgaaaggttgggcttcggaatcgttttccgggacgccggctggatgatcct |
| | | ccagcgcggggatctcatgctggagttcttcgcccaccccaacttgtttattgcagcttataatggtta |
| | | caaataaagcaatagcatcacaaatttcacaaataaagcatttttttcactgcattctagttgtggtttgtc |
| | | caaactcatcaatgtatcttatcatgtctgtataccgtcgacctctagctagagcttggcgtaatcatgg |
| | | tcatagctgtttcctgtgtgaaattgttatccgctcacaattccacacaacatacgagccggaagcata |
| | | aaggtaagcctgaatattgaaaaaggaagagtatgagtattcaacatttccgtgtcgcccttattccct |
| | | tttttgcggcattttgccttcctgtttttgctcacccagaaacgctggtgaaagtaaaagatgctgaaga |
| | | tcagttgggtgcacgagtgggttacatcgaactggatctcaacagcggtaagatccttgagagttttc |
| | | gccccgaagaacgttttccaatgatgagcacttttaaagttctgctatgtggcgcggtattatcccgtat |
| | | tgacgccgggcaagagcaactcggtcgccgcatacactattctcagaatgacttggttgagtactca |
| | | ccagtcacagaaaagcatcttacggatggcatgacagtaagagaattatgcagtgctgccataacc |
| | | atgagtgataacactgcggccaacttacttctgacaacgatcggaggaccgaaggagctaaccgct |
| | | tttttgcacaacatgggggatcatgtaactcgccttgatcgttgggaaccggagctgaatgaagccat |
| | | accaaacgacgagcgtgacaccacgatgcctgtagcaatggcaacaacgttgcgcaaactattaa |
| | | ctggcgaactacttactctagcttcccggcaacaattaatagactggatggaggcggataaagttgc |
| | | aggaccacttctgcgctcggcccttccggctggctggtttattgctgataaatctggagccggtgag |
| | | cgtgggtctcgcggtatcattgcagcactggggccagatggtaagccctcccgtatcgtagttatcta |
| | | cacgacggggagtcaggcaactatggatgaacgaaatagacagatcgctgagataggtgcctcac |
| | | tgattaagcattggtaactgtcagaccaagtttactcatatatactttagattgatttaaaacttcattttta |
| | | atttaaaaggatctaggtgaagatcctttttgataatctcatgaccaaaatcccttaacgtgagttttcgtt |
| | | ccactgagcgtcagaccccgtagaaaagatcaaaggatcttcttgagatcctttttttctgcgcgtaat |
| | | ctgctgcttgcaaacaaaaaaaccaccgctaccagcggtggtttttttgccggatcaagagctacca |
| | | actctttttccgaaggtaactggcttcagcagagcgcagataccaaatactgttcttctagtgtagccg |
| | | tagttaggccaccacttcaagaactctgtagcaccgcctacatacctcgctctgctaatcctgttacca |
| | | gtggctgctgccagtggcgataagtcgtgtcttaccgggttggactcaagacgatagttaccggata |
| | | aggcgcagcggtcgggctgaacggggggttcgtgcacacagcccagcttggagcgaacgacct |
| | | acaccgaactgagatacctacagcgtgagctatgagaaagcgccacgcttcccgaagggagaaa |
| | | ggcggacaggtatccggtaagcggcagggtcggaacaggagagcgcacgagggagcttccag |
| | | ggggaaacgcctggtatctttatagtcctgtcgggtttcgccacctctgacttgagcgtcgatttttgtg |
| | | atgctcgtcaggggggcggagcctatggaaaaacgccagcaacgcggcctttttacggttcctgg |
| | | ccttttgctggccttttgctcacatgttctttcctgcgttatcccctgattctgtggataaccgtattaccgc |
| | | ctttgagtgagctgataccgctcgccgcagccgaacgaccgagcgcagcgagtcagtgagcgag |
| | | gaagcggaagagcgcccaatacgcaaaccgcctctccccgcgcgttggccgattcattaatgcag |
| | | ctggcacgacaggtttcccgactggaaagcgggcagtgagcgcaacgcaattaatgtgagttagct |
| | | cactcattaggcaccccaggctttacactttatgcttccggctcgtatgttgtgtggaattgtgagcgg |
| | | ataacaatttcacacaggaaacagctatgacatgattacgaattgcaacgatttaggtgacactatag |
| | | aagagaaggaattaatacgactcactatagggagagagagagaattaccctcactaaagggagga |
| | | gaagcatgaattccccagtggaaagacgcgcaggcaaaacgcaccacgtgacggagcgtgacc |
| | | gcgcgccgagcgcgcgccaaggtcgggcaggaagagggcctatttcccatgattccttcatatttg |
| | | catatacgatacaaggctgttagagagataattagaattaatttgactgtaaacacaaagatattagta |
| | | caaaatacgtgacgtagaaagtaataatttcttgggtagtttgcagttttaaaattatgttttaaaatgga |
| | | ctatcatatgcttaccgtaacttgaaagtatttcgatttcttgggtttatatatcttgtggaaaggacgag |
| | | gatccgcatacgggaacgcacatagtgttttagagctagaaatagcaagttaaaataaggctagtcc |
| | | gttatcaacttgaaaaagtggcaccgagtcggtgcttttttct |
| 45 | P22 vector | agacacaattccccagtggaaagacgcgcaggcaaaacgcaccacgtgacggagcgtgaccgc |
| | for targeting | gcgccgagcgcgcgccaaggtcgggcaggaagagggcctatttcccatgattccttcatatttgca |
| | ASFV genes | tatacgatacaaggctgttagagagataattagaattaatttgactgtaaacacaaagatattagtaca |
| | including | aaatacgtgacgtagaaagtaataatttcttgggtagtttgcagttttaaaattatgttttaaaatggacta |
| | sgRNAs | tcatatgcttaccgtaacttgaaagtatttcgatttcttgggtttatatatcttgtggaaaggacgaggat |
| | scaffolds | ccgattgttgcacgggagaaccgttttagagctagaaatagcaagttaaaataaggctagtccgttat |
| | driven by | caacttgaaaaagtggcaccgagtcggtgcttttttctagacacaattccccagtggaaagacgcgc |
| | SP6 and U6 | aggcaaaacgcaccacgtgacggagcgtgaccgcgcgccgagcgcgcgccaaggtcgggca |
| | promoters | ggaagagggcctatttcccatgattccttcatatttgcatatacgatacaaggctgttagagagataatt |
| | and | agaattaatttgactgtaaacacaaagatattagtacaaaatacgtgacgtagaaagtaataatttcttg |
| | NLS(SAGSSG)- | ggtagtttgcagttttaaaattatgttttaaaatggactatcatatgcttaccgtaacttgaaagtatttcga |
| | Cas9 | tttcttgggtttatatatcttgtggaaaggacgaggatccgactttggcaagtaagcccgcgttttaga |
| | driven by | gctagaaatagcaagttaaaataaggctagtccgttatcaacttgaaaaagtggcaccgagtcggtg |
| | CMV | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCgtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgctaggcg |
| | | gccgcagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | acaaaatacgtgacgtagaaagtaataatttcttgggtagtttgcagttttaaaattatgttttaaaatgg |
| | | actatcatatgcttaccgtaacttgaaagtatttcgatttcttgggtttatatatcttgtggaaaggacgag |
| | | gatccgtttaacaatcgtctcgtggagttttagagctagaaatagcaagttaaaataaggctagtccgt |
| | | tatcaacttgaaaaagtggcaccgagtcggtgcttttttct |
| 46 | p18 GFP_del | agacacaattccccagtggaaagacgcgcaggcaaaacgcaccacgtgacggagcgtgaccgc |
| | | gcgccgagcgcgcgccaaggtcgggcaggaagagggcctatttcccatgattccttcatatttgca |
| | | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgcaactcatagagagttagcggttttagagctagaaatagcaagttaaaataaggctagtccgtta |
| | | tcaacttgaaaaagtggcaccgagtcggtgcttttttctagacacaattccccagtggaaagacgcg |
| | | caggcaaaacgcaccacgtgacggagcgtgaccgcgcgccgagcgcgcgccaaggtcgggca |
| | | ggaagagggcctatttcccatgattccttcatatttgcatatacgatacaaggctgttagagagataatt |
| | | agaattaatttgactgtaaacacaaagatattagtacaaaatacgtgacgtagaaagtaataatttcttg |
| | | ggtagtttgcagttttaaaattatgttttaaaatggactatcatatgcttaccgtaacttgaaagtatttcga |
| | | tttcttgggtttatatatcttgtggaaaggacgaggatccgcgtggtttagagaagcgcacgttttaga |
| | | gctagaaatagcaagttaaaataaggctagtccgttatcaacttgaaaaagtggcaccgagtcggtg |
| | | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacTGATGAccaccccaacttgtttattgcagcttataatggttacaaataaagcaatagcatca |
| | | caaatttcacaaataaagcatttttttcactgcattctagttgtggtttgtccaaactcatcaatgtatcttat |
| | | catgtctgtataccgtcgacctctagctagagcttggcgtaatcatggtcatagctgtttcctgtgtgaa |
| | | attgttatccgctcacaattccacacaacatacgagccggaagcataaaggtaagcctgaatattga |
| | | aaaaggaagagtatgagtattcaacatttccgtgtcgcccttattcccttttttgcggcattttgccttcct |
| | | gtttttgctcacccagaaacgctggtgaaagtaaaagatgctgaagatcagttgggtgcacgagtgg |
| | | gttacatcgaactggatctcaacagcggtaagatccttgagagttttcgccccgaagaacgttttcca |
| | | atgatgagcacttttaaagttctgctatgtggcgcggtattatcccgtattgacgccgggcaagagca |
| | | actcggtcgccgcatacactattctcagaatgacttggttgagtactcaccagtcacagaaaagcatc |
| | | ttacggatggcatgacagtaagagaattatgcagtgctgccataaccatgagtgataacactgcggc |
| | | caacttacttctgacaacgatcggaggaccgaaggagctaaccgcttttttgcacaacatgggggat |
| | | catgtaactcgccttgatcgttgggaaccggagctgaatgaagccataccaaacgacgagcgtgac |
| | | accacgatgcctgtagcaatggcaacaacgttgcgcaaactattaactggcgaactacttactctag |
| | | cttcccggcaacaattaatagactggatggaggcggataaagttgcaggaccacttctgcgctcgg |
| | | cccttccggctggctggtttattgctgataaatctggagccggtgagcgtgggtctcgcggtatcatt |
| | | gcagcactggggccagatggtaagccctcccgtatcgtagttatctacacgacggggagtcaggc |
| | | aactatggatgaacgaaatagacagatcgctgagataggtgcctcactgattaagcattggtaactgt |
| | | cagaccaagtttactcatatatactttagattgatttaaaacttcatttttaatttaaaaggatctaggtgaa |
| | | gatcctttttgataatctcatgaccaaaatcccttaacgtgagttttcgttccactgagcgtcagacccc |
| | | gtagaaaagatcaaaggatcttcttgagatcctttttttctgcgcgtaatctgctgcttgcaaacaaaaa |
| | | aaccaccgctaccagcggtggtttttttgccggatcaagagctaccaactctttttccgaaggtaactg |
| | | gcttcagcagagcgcagataccaaatactgttcttctagtgtagccgtagttaggccaccacttcaag |
| | | aactctgtagcaccgcctacatacctcgctctgctaatcctgttaccagtggctgctgccagtggcga |
| | | taagtcgtgtcttaccgggttggactcaagacgatagttaccggataaggcgcagcggtcgggctg |
| | | aacggggggttcgtgcacacagcccagcttggagcgaacgacctacaccgaactgagataccta |
| | | cagcgtgagctatgagaaagcgccacgcttcccgaagggagaaaggcggacaggtatccggtaa |
| | | gcggcagggtcggaacaggagagcgcacgagggagcttccagggggaaacgcctggtatcttt |
| | | atagtcctgtcgggtttcgccacctctgacttgagcgtcgatttttgtgatgctcgtcaggggggcgg |
| | | agcctatggaaaaacgccagcaacgcggcctttttacggttcctggccttttgctggccttttgctcac |
| | | atgttctttcctgcgttatcccctgattctgtggataaccgtattaccgcctttgagtgagctgataccgc |
| | | tcgccgcagccgaacgaccgagcgcagcgagtcagtgagcgaggaagcggaagagcgcccaa |
| | | tacgcaaaccgcctctccccgcgcgttggccgattcattaatgcagctggcacgacaggtttcccga |
| | | ctggaaagcgggcagtgagcgcaacgcaattaatgtgagttagctcactcattaggcaccccagg |
| | | ctttacactttatgcttccggctcgtatgttgtgtggaattgtgagcggataacaatttcacacaggaaa |
| | | cagctatgacatgattacgaattgcaacgatttaggtgacactatagaagagaaggaattaatacga |
| | | ctcactatagggagagagagagaattaccctcactaaagggaggagaagcatgaattccccagtg |
| | | gaaagacgcgcaggcaaaacgcaccacgtgacggagcgtgaccgcgcgccgagcgcgcgcc |
| | | aaggtcgggcaggaagagggcctatttcccatgattccttcatatttgcatatacgatacaaggctgtt |
| | | agagagataattagaattaatttgactgtaaacacaaagatattagtacaaaatacgtgacgtagaaa |
| | | gtaataatttcttgggtagtttgcagttttaaaattatgttttaaaatggactatcatatgcttaccgtaactt |
| | | gaaagtatttcgatttcttgggtttatatatcttgtggaaaggacgaggatccgcctctgaccttaattat |
| | | agggttttagagctagaaatagcaagttaaaataaggctagtccgttatcaacttgaaaaagtggcac |
| | | cgagtcggtgcttttttct |
| 47 | p19 GFP | agacacaattgcatgaagaatctgcttagggttaggcgttttgcgctgcttcgcgatgtacgggcca |
| | deletion | gatatacgcgttgacattgattattgactagttattaatagtaatcaattacggggtcattagttcatagc |
| | | ccatatatggagttccgcgttacataacttacggtaaatggcccgcctggctgaccgcccaacgacc |
| | | cccgcccattgacgtcaataatgacgtatgttcccatagtaacgccaatagggactttccattgacgt |
| | | caatgggtggagtatttacggtaaactgcccacttggcagtacatcaagtgtatcatatgccaagtac |
| | | gccccctattgacgtcaatgacggtaaatggcccgcctggcattatgcccagtacatgaccttatgg |
| | | gactttcctacttggcagtacatctacgtattagtcatcgctattaccatggtgatgcggttttggcagta |
| | | catcaatgggcgtggatagcggtttgactcacggggatttccaagtctccaccccattgacgtcaat |
| | | gggagtttgttttggcaccaaaatcaacgggactttccaaaatgtcgtaacaactccgccccattgac |
| | | gcaaatgggcggtaggcgtgtacggtgggaggtctatataagcagagctctctggctaactagaga |
| | | acccactgcttactggcttatcgaaattaatacgactcactatagggagacccaagcttgccaccatg |
| | | gacaagaagtacagcatcggcctggacatcggtaccaacagcgtgggctgggccgtgatcaccg |
| | | acgagtacaaggtgcccagcaagaagttcaaggtgctgggcaacaccgaccgccacagcatcaa |
| | | gaagaacctgatcggcgccctgctgttcgacagcggcgagaccgccgaggccacccgcctgaa |
| | | gcgcaccgcccgccgccgctacacccgccgcaagaaccgcatctgctacctgcaggagatcttc |
| | | agcaacgagatggccaaggtggacgacagcttcttccaccgcctggaggagagcttcctggtgga |
| | | ggaggacaagaagcacgagcgccaccccatcttcggcaacatcgtggacgaggtggcctaccac |
| | | gagaagtaccccaccatctaccacctgcgcaagaagctggtggacagcaccgacaaggccgacc |
| | | tgcgcctgatctacctggccctggcccacatgatcaagttccgcggccacttcctgatcgagggcg |
| | | acctgaaccccgacaacagcgacgtggacaagctgttcatccagctggtgcagacctacaaccag |
| | | ctgttcgaggagaaccccatcaacgccagcggcgtggacgccaaggccatcctgagcgcccgcc |
| | | tgagcaagagccgccgcctggagaacctgatcgcccagctgcccggcgagaagaagaacggcc |
| | | tgttcggcaacctgatcgccctgagcctgggcctgacccccaacttcaagagcaacttcgacctgg |
| | | ccgaggacgccaagctgcagctgagcaaggacacctacgacgacgacctggacaacctgctgg |
| | | cccagatcggcgaccagtacgccgacctgttcctggccgccaagaacctgagcgacgccatcctg |
| | | ctgagcgacatcctgcgcgtgaacaccgagatcaccaaggcccccctgagcgccagcatgatca |
| | | agcgctacgacgagcaccaccaggacctgaccctgctgaaggccctggtgcgccagcagctgcc |
| | | cgagaagtacaaggagatcttcttcgaccagagcaagaacggctacgccggctacatcgacggc |
| | | ggcgccagccaggaggagttctacaagttcatcaagcccatcctggagaagatggacggcaccg |
| | | aggagctgctggtgaagctgaaccgcgaggacctgctgcgcaagcagcgcaccttcgacaacgg |
| | | cagcatcccccaccagatccacctgggcgagctgcacgccatcctgcgccgccaggaggacttct |
| | | accccttcctgaaggacaaccgcgagaagatcgagaagatcctgaccttccgcatcccctactacg |
| | | tgggccccctggcccgcggcaacagccgcttcgcctggatgacccgcaagagcgaggagacca |
| | | tcaccccctggaacttcgaggaggtggtggacaagggcgccagcgcccagagcttcatcgagcg |
| | | catgaccaacttcgacaagaacctgcccaacgagaaggtgctgcccaagcacagcctgctgtacg |
| | | agtacttcaccgtgtacaacgagctgaccaaggtgaagtacgtgaccgagggcatgcgcaagccc |
| | | gccttcctgagcggcgagcagaagaaggccatcgtggacctgctgttcaagaccaaccgcaaggt |
| | | gaccgtgaagcagctgaaggaggactacttcaagaagatcgagtgcttcgacagcgtggagatca |
| | | gcggcgtggaggaccgcttcaacgccagcctgggcacctaccacgacctgctgaagatcatcaa |
| | | ggacaaggacttcctggacaacgaggagaacgaggacatcctggaggacatcgtgctgaccctg |
| | | accctgttcgaggaccgcgagatgatcgaggagcgcctgaagacctacgcccacctgttcgacga |
| | | caaggtgatgaagcagctgaagcgccgccgctacaccggctggggccgcctgagccgcaagctt |
| | | atcaacggcatccgcgacaagcagagcggcaagaccatcctggacttcctgaagagcgacggct |
| | | tcgccaaccgcaacttcatgcagctgatccacgacgacagcctgaccttcaaggaggacatccag |
| | | aaggcccaggtgagcggccagggcgacagcctgcacgagcacatcgccaacctggccggcag |
| | | ccccgccatcaagaagggcatcctgcagaccgtgaaggtggtggacgagctggtgaaggtgatg |
| | | ggccgccacaagcccgagaacatcgtgatcgagatggcccgcgagaaccagaccacccagaag |
| | | ggccagaagaacagccgcgagcgcatgaagcgcatcgaggagggcatcaaggagctgggcag |
| | | ccagatcctgaaggagcaccccgtggagaacacccagctgcagaacgagaagctgtacctgtact |
| | | acctgcagaacggccgcgacatgtacgtggaccaggagctggacatcaaccgcctgagcgacta |
| | | cgacgtggaccacatcgtgccccagagcttcctgaaggacgacagcatcgacaacaaggtgctg |
| | | acccgcagcgacaagaaccgcggcaagagcgacaacgtgcccagcgaggaggtggtgaagaa |
| | | gatgaagaactactggcgccagctgctgaacgccaagctgatcacccagcgcaagttcgacaacc |
| | | tgaccaaggccgagcgcggcggcctgagcgagctggacaaggccggcttcatcaagcgccagc |
| | | tggtggagacccgccagatcaccaagcacgtggcccagatcctggacagccgcatgaacaccaa |
| | | gtacgacgagaacgacaagctgatccgcgaggtgaaggtgatcaccctgaagagcaagctggtg |
| | | agcgacttccgcaaggacttccagttctacaaggtgcgcgagatcaacaactaccaccacgccca |
| | | cgacgcctacctgaacgccgtggtgggcaccgccctgatcaagaagtaccccaagctggagagc |
| | | gagttcgtgtacggcgactacaaggtgtacgacgtgcgcaagatgatcgccaagagcgagcagg |
| | | agatcggcaaggccaccgccaagtacttcttctacagcaacatcatgaacttcttcaagaccgagat |
| | | caccctggccaacggcgagatccgcaagcgccccctgatcgagaccaacggcgagaccggcg |
| | | agatcgtgtgggacaagggccgcgacttcgccaccgtgcgcaaggtgctgagcatgccccaggt |
| | | gaacatcgtgaagaagaccgaggtgcagaccggcggcttcagcaaggagagcatcctgcccaa |
| | | gcgcaacagcgacaagctgatcgcccgcaagaaggactgggaccccaagaagtacggcggctt |
| | | cgacagccccaccgtggcctacagcgtgctggtggtggccaaggtggagaagggcaagagcaa |
| | | gaagctgaagagcgtgaaggagctgctgggcatcaccatcatggagcgcagcagcttcgagaag |
| | | aaccccatcgacttcctggaggccaagggctacaaggaggtgaagaaggacctgatcatcaagct |
| | | gcccaagtacagcctgttcgagctggagaacggccgcaagcgcatgctggccagcgccggcga |
| | | gctgcagaagggcaacgagctggccctgcccagcaagtacgtgaacttcctgtacctggccagcc |
| | | actacgagaagctgaagggcagccccgaggacaacgagcagaagcagctgttcgtggagcagc |
| | | acaagcactacctggacgagatcatcgagcagatcagcgagttcagcaagcgcgtgatcctggcc |
| | | gacgccaacctggacaaggtgctgagcgcctacaacaagcaccgcgacaagcccatccgcgag |
| | | caggccgagaacatcatccacctgttcaccctgaccaacctgggcgcccccgccgccttcaagta |
| | | cttcgacaccaccatcgaccgcaagcgctacaccagcaccaaggaggtgctggacgccaccctg |
| | | atccaccagagcatcaccggtctgtacgagacccgcatcgacctgagccagctgggcggcgact |
| | | gatgacagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | acaaaatacgtgacgtagaaagtaataatttcttgggtagtttgcagttttaaaattatgttttaaaatgg |
| | | actatcatatgcttaccgtaacttgaaagtatttcgatttcttgggtttatatatcttgtggaaaggacgag |
| | | gatccggagaagcttctctgttttagagctagaaatagcaagttaaaataaggctagtccgttatcaac |
| | | ttgaaaaagtggcaccgagtcggtgcttttttct |
| 48 | P20 GFP | agacacaattccccagtggaaagacgcgcaggcaaaacgcaccacgtgacggagcgtgaccgc |
| | deletion | gcgccgagcgcgcgccaaggtcgggcaggaagagggcctatttcccatgattccttcatatttgca |
| | | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgaccaagatctggacgggtggttttagagctagaaatagcaagttaaaataaggctagtccgtta |
| | | tcaacttgaaaaagtggcaccgagtcggtgcttttttctagacacaattccccagtggaaagacgcg |
| | | caggcaaaacgcaccacgtgacggagcgtgaccgcgcgccgagcgcgcgccaaggtcgggca |
| | | ggaagagggcctatttcccatgattccttcatatttgcatatacgatacaaggctgttagagagataatt |
| | | agaattaatttgactgtaaacacaaagatattagtacaaaatacgtgacgtagaaagtaataatttcttg |
| | | ggtagtttgcagttttaaaattatgttttaaaatggactatcatatgcttaccgtaacttgaaagtatttcga |
| | | tttcttgggtttatatatcttgtggaaaggacgaggatccgggtgtatgacacgttgtcggttttagagc |
| | | tagaaatagcaagttaaaataaggctagtccgttatcaacttgaaaaagtggcaccgagtcggtgctt |
| | | ttttctagacacaattgcatgaagaatctgcttagggttaggcgttttgcgctgcttcgcgatgtacgg |
| | | gccagatatacgcgttgacattgattattgactagttattaatagtaatcaattacggggtcattagttca |
| | | tagcccatatatggagttccgcgttacataacttacggtaaatggcccgcctggctgaccgcccaac |
| | | gacccccgcccattgacgtcaataatgacgtatgttcccatagtaacgccaatagggactttccattg |
| | | acgtcaatgggtggagtatttacggtaaactgcccacttggcagtacatcaagtgtatcatatgccaa |
| | | gtacgccccctattgacgtcaatgacggtaaatggcccgcctggcattatgcccagtacatgacctt |
| | | atgggactttcctacttggcagtacatctacgtattagtcatcgctattaccatggtgatgcggttttggc |
| | | agtacatcaatgggcgtggatagcggtttgactcacggggatttccaagtctccaccccattgacgt |
| | | caatgggagtttgttttggcaccaaaatcaacgggactttccaaaatgtcgtaacaactccgccccat |
| | | tgacgcaaatgggcggtaggcgtgtacggtgggaggtctatataagcagagctctctggctaacta |
| | | gagaacccactgcttactggcttatcgaaattaatacgactcactatagggagacccaagcttgcca |
| | | ccatggacaagaagtacagcatcggcctggacatcggtaccaacagcgtgggctgggccgtgat |
| | | caccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgacagatgggggtcctgggccccagggtgtgcagccactgacttggggactgctggtg |
| | | gggtagggatgagggagggaggggcattgtgatgtacagggctgctctgtgagatcaagggtctc |
| | | ttaagggtgggagctggggcagggactacgagagcagccagatgggctgaaagtggaactcaa |
| | | ggggtttctggcacctacctacctgcttcccgctggggggtggggagttggcccagagtcttaagat |
| | | tggggcagggtggagaggtgggctcttcctgcttcccactcatcttatagctttctttccccagatccg |
| | | aattggagatccaaaccaaggcgcgcgctagcgccaccatgggatcggccattgaacaagatgg |
| | | attgcacgcaggttctccggccgcttgggtggagaggctattcggctatgactgggcacaacagac |
| | | aatcggctgctctgatgccgccgtgttccggctgtcagcgcaggggcgcccggttctttttgtcaag |
| | | accgacctgtccggtgccctgaatgaactgcaggacgaggcagcgcggctatcgtggctggcca |
| | | cgacgggcgttccttgcgcagcagtgctcgacgttgtcactgaagcgggaagggactggctgctat |
| | | tgggcgaagtgccggggcaggatctcctgtcatctcaccttgctcctgccgagaaagtatccatcat |
| | | ggctgatgcaatgcggcggctgcatacgcttgatccggctacctgcccattcgaccaccaagcgaa |
| | | acatcgcatcgagcgagcacgtactcggatggaagccggtcttgtcgatcaggatgatctggacga |
| | | agagcatcaggggctcgcgccagccgaactgttcgccaggctcaaggcgcgtatgcccgacggc |
| | | gatgatctcgtcgtgactcatggcgatgcctgcttgccgaatatcatggtggaaaatggccgcttttct |
| | | ggattcatcgactgtggccggctgggtgtggcggaccgctatcaggacatagcgttggctacccgt |
| | | gatattgctgaagagcttggcggcgaatgggctgaccgcttcctcgtgctttacggtatcgccgctc |
| | | ccgattcgcagcgcatcgccttctatcgccttcttgacgagttcttctgaacgcggtgctacgagattt |
| | | cgattccaccgccgccttctatgaaaggttgggcttcggaatcgttttccgggacgccggctggatg |
| | | atcctccagcgcggggatctcatgctggagttcttcgcccaccccaacttgtttattgcagcttataat |
| | | ggttacaaataaagcaatagcatcacaaatttcacaaataaagcatttttttcactgcattctagttgtgg |
| | | tttgtccaaactcatcaatgtatcttatcatgtctgtataccgtcgacctctagctagagcttggcgtaat |
| | | catggtcatagctgtttcctgtgtgaaattgttatccgctcacaattccacacaacatacgagccggaa |
| | | gcataaaggtaagcctgaatattgaaaaaggaagagtatgagtattcaacatttccgtgtcgcccttat |
| | | tcccttttttgcggcattttgccttcctgtttttgctcacccagaaacgctggtgaaagtaaaagatgctg |
| | | aagatcagttgggtgcacgagtgggttacatcgaactggatctcaacagcggtaagatccttgaga |
| | | gttttcgccccgaagaacgttttccaatgatgagcacttttaaagttctgctatgtggcgcggtattatc |
| | | ccgtattgacgccgggcaagagcaactcggtcgccgcatacactattctcagaatgacttggttgag |
| | | tactcaccagtcacagaaaagcatcttacggatggcatgacagtaagagaattatgcagtgctgcca |
| | | taaccatgagtgataacactgcggccaacttacttctgacaacgatcggaggaccgaaggagctaa |
| | | ccgcttttttgcacaacatgggggatcatgtaactcgccttgatcgttgggaaccggagctgaatgaa |
| | | gccataccaaacgacgagcgtgacaccacgatgcctgtagcaatggcaacaacgttgcgcaaact |
| | | attaactggcgaactacttactctagcttcccggcaacaattaatagactggatggaggcggataaa |
| | | gttgcaggaccacttctgcgctcggcccttccggctggctggtttattgctgataaatctggagccgg |
| | | tgagcgtgggtctcgcggtatcattgcagcactggggccagatggtaagccctcccgtatcgtagtt |
| | | atctacacgacggggagtcaggcaactatggatgaacgaaatagacagatcgctgagataggtgc |
| | | ctcactgattaagcattggtaactgtcagaccaagtttactcatatatactttagattgatttaaaacttca |
| | | tttttaatttaaaaggatctaggtgaagatcctttttgataatctcatgaccaaaatcccttaacgtgagtt |
| | | ttcgttccactgagcgtcagaccccgtagaaaagatcaaaggatcttcttgagatcctttttttctgcgc |
| | | gtaatctgctgcttgcaaacaaaaaaaccaccgctaccagcggtggtttttttgccggatcaagagct |
| | | accaactctttttccgaaggtaactggcttcagcagagcgcagataccaaatactgttcttctagtgta |
| | | gccgtagttaggccaccacttcaagaactctgtagcaccgcctacatacctcgctctgctaatcctgtt |
| | | accagtggctgctgccagtggcgataagtcgtgtcttaccgggttggactcaagacgatagttaccg |
| | | gataaggcgcagcggtcgggctgaacggggggttcgtgcacacagcccagcttggagcgaacg |
| | | acctacaccgaactgagatacctacagcgtgagctatgagaaagcgccacgcttcccgaagggag |
| | | aaaggcggacaggtatccggtaagcggcagggtcggaacaggagagcgcacgagggagcttc |
| | | cagggggaaacgcctggtatctttatagtcctgtcgggtttcgccacctctgacttgagcgtcgattttt |
| | | gtgatgctcgtcaggggggcggagcctatggaaaaacgccagcaacgcggcctttttacggttcct |
| | | ggccttttgctggccttttgctcacatgttctttcctgcgttatcccctgattctgtggataaccgtattac |
| | | cgcctttgagtgagctgataccgctcgccgcagccgaacgaccgagcgcagcgagtcagtgagc |
| | | gaggaagcggaagagcgcccaatacgcaaaccgcctctccccgcgcgttggccgattcattaatg |
| | | cagctggcacgacaggtttcccgactggaaagcgggcagtgagcgcaacgcaattaatgtgagtt |
| | | agctcactcattaggcaccccaggctttacactttatgcttccggctcgtatgttgtgtggaattgtgag |
| | | cggataacaatttcacacaggaaacagctatgacatgattacgaattgcaacgatttaggtgacacta |
| | | tagaagagaaggaattaatacgactcactatagggagagagagagaattaccctcactaaaggga |
| | | ggagaagcatgaattccccagtggaaagacgcgcaggcaaaacgcaccacgtgacggagcgtg |
| | | accgcgcgccgagcgcgcgccaaggtcgggcaggaagagggcctatttcccatgattccttcata |
| | | tttgcatatacgatacaaggctgttagagagataattagaattaatttgactgtaaacacaaagatatta |
| | | gtacaaaatacgtgacgtagaaagtaataatttcttgggtagtttgcagttttaaaattatgttttaaaatg |
| | | gactatcatatgcttaccgtaacttgaaagtatttcgatttcttgggtttatatatcttgtggaaaggacga |
| | | ggatccgtgtttaacgacatatcgccagttttagagctagaaatagcaagttaaaataaggctagtcc |
| | | gttatcaacttgaaaaagtggcaccgagtcggtgcttttttct |
| 49 | P21 GFP | agacacaattccccagtggaaagacgcgcaggcaaaacgcaccacgtgacggagcgtgaccgc |
| | deletion | gcgccgagcgcgcgccaaggtcgggcaggaagagggcctatttcccatgattccttcatatttgca |
| | | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgtttacttcggcttttacaaggttttagagctagaaatagcaagttaaaataaggctagtccgttatc |
| | | aacttgaaaaagtggcaccgagtcggtgcttttttctagacacaattccccagtggaaagacgcgca |
| | | ggcaaaacgcaccacgtgacggagcgtgaccgcgcgccgagcgcgcgccaaggtcgggcag |
| | | gaagagggcctatttcccatgattccttcatatttgcatatacgatacaaggctgttagagagataatta |
| | | gaattaatttgactgtaaacacaaagatattagtacaaaatacgtgacgtagaaagtaataatttcttgg |
| | | gtagtttgcagttttaaaattatgttttaaaatggactatcatatgcttaccgtaacttgaaagtatttcgat |
| | | ttcttgggtttatatatcttgtggaaaggacgaggatccgaaaggggtccttcgaacacggttttagag |
| | | ctagaaatagcaagttaaaataaggctagtccgttatcaacttgaaaaagtggcaccgagtcggtgc |
| | | ttttttctagacacaattgcatgaagaatctgcttagggttaggcgttttgcgctgcttcgcgatgtacg |
| | | ggccagatatacgcgttgacattgattattgactagttattaatagtaatcaattacggggtcattagttc |
| | | atagcccatatatggagttccgcgttacataacttacggtaaatggcccgcctggctgaccgcccaa |
| | | cgacccccgcccattgacgtcaataatgacgtatgttcccatagtaacgccaatagggactttccatt |
| | | gacgtcaatgggtggagtatttacggtaaactgcccacttggcagtacatcaagtgtatcatatgcca |
| | | agtacgccccctattgacgtcaatgacggtaaatggcccgcctggcattatgcccagtacatgacct |
| | | tatgggactttcctacttggcagtacatctacgtattagtcatcgctattaccatggtgatgcggttttgg |
| | | cagtacatcaatgggcgtggatagcggtttgactcacggggatttccaagtctccaccccattgacg |
| | | tcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaact |
| | | agagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggcctttttacggttcctggccttttgctggccttttgctcacatgtt |
| | | ctttcctgcgttatcccctgattctgtggataaccgtattaccgcctttgagtgagctgataccgctcgc |
| | | cgcagccgaacgaccgagcgcagcgagtcagtgagcgaggaagcggaagagcgcccaatacg |
| | | caaaccgcctctccccgcgcgttggccgattcattaatgcagctggcacgacaggtttcccgactg |
| | | gaaagcgggcagtgagcgcaacgcaattaatgtgagttagctcactcattaggcaccccaggcttt |
| | | acactttatgcttccggctcgtatgttgtgtggaattgtgagcggataacaatttcacacaggaaacag |
| | | ctatgacatgattacgaattgcaacgatttaggtgacactatagaagagaaggaattaatacgactca |
| | | ctatagggagagagagagaattaccctcactaaagggaggagaagcatgaattccccagtggaaa |
| | | gacgcgcaggcaaaacgcaccacgtgacggagcgtgaccgcgcgccgagcgcgcgccaaggt |
| | | cgggcaggaagagggcctatttcccatgattccttcatatttgcatatacgatacaaggctgttagag |
| | | agataattagaattaatttgactgtaaacacaaagatattagtacaaaatacgtgacgtagaaagtaat |
| | | aatttcttgggtagtttgcagttttaaaattatgttttaaaatggactatcatatgcttaccgtaacttgaaa |
| | | gtatttcgatttcttgggtttatatatcttgtggaaaggacgaggatccgcatacgggaacgcacatag |
| | | tgttttagagctagaaatagcaagttaaaataaggctagtccgttatcaacttgaaaaagtggcaccg |
| | | agtcggtgcttttttct |
| 50 | p22 | agacacaattccccagtggaaagacgcgcaggcaaaacgcaccacgtgacggagcgtgaccgc |
| | GFP deletion | gcgccgagcgcgcgccaaggtcgggcaggaagagggcctatttcccatgattccttcatatttgca |
| | | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgattgttgcacgggagaaccgttttagagctagaaatagcaagttaaaataaggctagtccgttat |
| | | caacttgaaaaagtggcaccgagtcggtgcttttttctagacacaattccccagtggaaagacgcgc |
| | | aggcaaaacgcaccacgtgacggagcgtgaccgcgcgccgagcgcgcgccaaggtcgggca |
| | | ggaagagggcctatttcccatgattccttcatatttgcatatacgatacaaggctgttagagagataatt |
| | | agaattaatttgactgtaaacacaaagatattagtacaaaatacgtgacgtagaaagtaataatttcttg |
| | | ggtagtttgcagttttaaaattatgttttaaaatggactatcatatgcttaccgtaacttgaaagtatttcga |
| | | tttcttgggtttatatatcttgtggaaaggacgaggatccgactttggcaagtaagcccgcgttttaga |
| | | gctagaaatagcaagttaaaataaggctagtccgttatcaacttgaaaaagtggcaccgagtcggtg |
| | | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgacagatgggggtcctgggccccagggtgtgcagccactgacttggggactgctggtg |
| | | gggtagggatgagggagggaggggcattgtgatgtacagggctgctctgtgagatcaagggtctc |
| | | ttaagggtgggagctggggcagggactacgagagcagccagatgggctgaaagtggaactcaa |
| | | ggggtttctggcacctacctacctgcttcccgctggggggtggggagttggcccagagtcttaagat |
| | | tggggcagggtggagaggtgggctcttcctgcttcccactcatcttatagctttctttccccagatccg |
| | | aattggagatccaaaccaaggcgcgcgctagcgccaccatgggatcggccattgaacaagatgg |
| | | attgcacgcaggttctccggccgcttgggtggagaggctattcggctatgactgggcacaacagac |
| | | aatcggctgctctgatgccgccgtgttccggctgtcagcgcaggggcgcccggttctttttgtcaag |
| | | accgacctgtccggtgccctgaatgaactgcaggacgaggcagcgcggctatcgtggctggcca |
| | | cgacgggcgttccttgcgcagcagtgctcgacgttgtcactgaagcgggaagggactggctgctat |
| | | tgggcgaagtgccggggcaggatctcctgtcatctcaccttgctcctgccgagaaagtatccatcat |
| | | ggctgatgcaatgcggcggctgcatacgcttgatccggctacctgcccattcgaccaccaagcgaa |
| | | acatcgcatcgagcgagcacgtactcggatggaagccggtcttgtcgatcaggatgatctggacga |
| | | agagcatcaggggctcgcgccagccgaactgttcgccaggctcaaggcgcgtatgcccgacggc |
| | | gatgatctcgtcgtgactcatggcgatgcctgcttgccgaatatcatggtggaaaatggccgcttttct |
| | | ggattcatcgactgtggccggctgggtgtggcggaccgctatcaggacatagcgttggctacccgt |
| | | gatattgctgaagagcttggcggcgaatgggctgaccgcttcctcgtgctttacggtatcgccgctc |
| | | ccgattcgcagcgcatcgccttctatcgccttcttgacgagttcttctgaacgcggtgctacgagattt |
| | | cgattccaccgccgccttctatgaaaggttgggcttcggaatcgttttccgggacgccggctggatg |
| | | atcctccagcgcggggatctcatgctggagttcttcgcccaccccaacttgtttattgcagcttataat |
| | | ggttacaaataaagcaatagcatcacaaatttcacaaataaagcatttttttcactgcattctagttgtgg |
| | | tttgtccaaactcatcaatgtatcttatcatgtctgtataccgtcgacctctagctagagcttggcgtaat |
| | | catggtcatagctgtttcctgtgtgaaattgttatccgctcacaattccacacaacatacgagccggaa |
| | | gcataaaggtaagcctgaatattgaaaaaggaagagtatgagtattcaacatttccgtgtcgcccttat |
| | | tcccttttttgcggcattttgccttcctgtttttgctcacccagaaacgctggtgaaagtaaaagatgctg |
| | | aagatcagttgggtgcacgagtgggttacatcgaactggatctcaacagcggtaagatccttgaga |
| | | gttttcgccccgaagaacgttttccaatgatgagcacttttaaagttctgctatgtggcgcggtattatc |
| | | ccgtattgacgccgggcaagagcaactcggtcgccgcatacactattctcagaatgacttggttgag |
| | | tactcaccagtcacagaaaagcatcttacggatggcatgacagtaagagaattatgcagtgctgcca |
| | | taaccatgagtgataacactgcggccaacttacttctgacaacgatcggaggaccgaaggagctaa |
| | | ccgcttttttgcacaacatgggggatcatgtaactcgccttgatcgttgggaaccggagctgaatgaa |
| | | gccataccaaacgacgagcgtgacaccacgatgcctgtagcaatggcaacaacgttgcgcaaact |
| | | attaactggcgaactacttactctagcttcccggcaacaattaatagactggatggaggcggataaa |
| | | gttgcaggaccacttctgcgctcggcccttccggctggctggtttattgctgataaatctggagccgg |
| | | tgagcgtgggtctcgcggtatcattgcagcactggggccagatggtaagccctcccgtatcgtagtt |
| | | atctacacgacggggagtcaggcaactatggatgaacgaaatagacagatcgctgagataggtgc |
| | | ctcactgattaagcattggtaactgtcagaccaagtttactcatatatactttagattgatttaaaacttca |
| | | tttttaatttaaaaggatctaggtgaagatcctttttgataatctcatgaccaaaatcccttaacgtgagtt |
| | | ttcgttccactgagcgtcagaccccgtagaaaagatcaaaggatcttcttgagatcctttttttctgcgc |
| | | gtaatctgctgcttgcaaacaaaaaaaccaccgctaccagcggtggtttttttgccggatcaagagct |
| | | accaactctttttccgaaggtaactggcttcagcagagcgcagataccaaatactgttcttctagtgta |
| | | gccgtagttaggccaccacttcaagaactctgtagcaccgcctacatacctcgctctgctaatcctgtt |
| | | accagtggctgctgccagtggcgataagtcgtgtcttaccgggttggactcaagacgatagttaccg |
| | | gataaggcgcagcggtcgggctgaacggggggttcgtgcacacagcccagcttggagcgaacg |
| | | acctacaccgaactgagatacctacagcgtgagctatgagaaagcgccacgcttcccgaagggag |
| | | aaaggcggacaggtatccggtaagcggcagggtcggaacaggagagcgcacgagggagcttc |
| | | cagggggaaacgcctggtatctttatagtcctgtcgggtttcgccacctctgacttgagcgtcgattttt |
| | | gtgatgctcgtcaggggggcggagcctatggaaaaacgccagcaacgcggcctttttacggttcct |
| | | ggccttttgctggccttttgctcacatgttctttcctgcgttatcccctgattctgtggataaccgtattac |
| | | cgcctttgagtgagctgataccgctcgccgcagccgaacgaccgagcgcagcgagtcagtgagc |
| | | gaggaagcggaagagcgcccaatacgcaaaccgcctctccccgcgcgttggccgattcattaatg |
| | | cagctggcacgacaggtttcccgactggaaagcgggcagtgagcgcaacgcaattaatgtgagtt |
| | | agctcactcattaggcaccccaggctttacactttatgcttccggctcgtatgttgtgtggaattgtgag |
| | | cggataacaatttcacacaggaaacagctatgacatgattacgaattgcaacgatttaggtgacacta |
| | | tagaagagaaggaattaatacgactcactatagggagagagagagaattaccctcactaaaggga |
| | | ggagaagcatgaattccccagtggaaagacgcgcaggcaaaacgcaccacgtgacggagcgtg |
| | | accgcgcgccgagcgcgcgccaaggtcgggcaggaagagggcctatttcccatgattccttcata |
| | | tttgcatatacgatacaaggctgttagagagataattagaattaatttgactgtaaacacaaagatatta |
| | | gtacaaaatacgtgacgtagaaagtaataatttcttgggtagtttgcagttttaaaattatgttttaaaatg |
| | | gactatcatatgcttaccgtaacttgaaagtatttcgatttcttgggtttatatatcttgtggaaaggacga |
| | | ggatccgtttaacaatcgtctcgtggagttttagagctagaaatagcaagttaaaataaggctagtcc |
| | | gttatcaacttgaaaaagtggcaccgagtcggtgcttttttct |
| 51 | p18 GFP and | agacacaattccccagtggaaagacgcgcaggcaaaacgcaccacgtgacggagcgtgaccgc |
| | Neomycin | gcgccgagcgcgcgccaaggtcgggcaggaagagggcctatttcccatgattccttcatatttgca |
| | deletion | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgcaactcatagagagttagcggttttagagctagaaatagcaagttaaaataaggctagtccgtta |
| | | tcaacttgaaaaagtggcaccgagtcggtgcttttttctagacacaattccccagtggaaagacgcg |
| | | caggcaaaacgcaccacgtgacggagcgtgaccgcgcgccgagcgcgcgccaaggtcgggca |
| | | ggaagagggcctatttcccatgattccttcatatttgcatatacgatacaaggctgttagagagataatt |
| | | agaattaatttgactgtaaacacaaagatattagtacaaaatacgtgacgtagaaagtaataatttcttg |
| | | ggtagtttgcagttttaaaattatgttttaaaatggactatcatatgcttaccgtaacttgaaagtatttcga |
| | | tttcttgggtttatatatcttgtggaaaggacgaggatccgcgtggtttagagaagcgcacgttttaga |
| | | gctagaaatagcaagttaaaataaggctagtccgttatcaacttgaaaaagtggcaccgagtcggtg |
| | | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacTGATGAccaccccaacttgtttattgcagcttataatggttacaaataaagcaatagcatca |
| | | caaatttcacaaataaagcatttttttcactgcattctagttgtggtttgtccaaactcatcaatgtatcttat |
| | | catgtctgtataccgtcgacctctagctagagcttggcgtaatcatggtcatagctgtttcctgtgtgaa |
| | | attgttatccgctcacaattccacacaacatacgagccggaagcataaaggtaagcctgaatattga |
| | | aaaaggaagagtatgagtattcaacatttccgtgtcgcccttattcccttttttgcggcattttgccttcct |
| | | gtttttgctcacccagaaacgctggtgaaagtaaaagatgctgaagatcagttgggtgcacgagtgg |
| | | gttacatcgaactggatctcaacagcggtaagatccttgagagttttcgccccgaagaacgttttcca |
| | | atgatgagcacttttaaagttctgctatgtggcgcggtattatcccgtattgacgccgggcaagagca |
| | | actcggtcgccgcatacactattctcagaatgacttggttgagtactcaccagtcacagaaaagcatc |
| | | ttacggatggcatgacagtaagagaattatgcagtgctgccataaccatgagtgataacactgcggc |
| | | caacttacttctgacaacgatcggaggaccgaaggagctaaccgcttttttgcacaacatgggggat |
| | | catgtaactcgccttgatcgttgggaaccggagctgaatgaagccataccaaacgacgagcgtgac |
| | | accacgatgcctgtagcaatggcaacaacgttgcgcaaactattaactggcgaactacttactctag |
| | | cttcccggcaacaattaatagactggatggaggcggataaagttgcaggaccacttctgcgctcgg |
| | | cccttccggctggctggtttattgctgataaatctggagccggtgagcgtgggtctcgcggtatcatt |
| | | gcagcactggggccagatggtaagccctcccgtatcgtagttatctacacgacggggagtcaggc |
| | | aactatggatgaacgaaatagacagatcgctgagataggtgcctcactgattaagcattggtaactgt |
| | | cagaccaagtttactcatatatactttagattgatttaaaacttcatttttaatttaaaaggatctaggtgaa |
| | | gatcctttttgataatctcatgaccaaaatcccttaacgtgagttttcgttccactgagcgtcagacccc |
| | | gtagaaaagatcaaaggatcttcttgagatcctttttttctgcgcgtaatctgctgcttgcaaacaaaaa |
| | | aaccaccgctaccagcggtggtttttttgccggatcaagagctaccaactctttttccgaaggtaactg |
| | | gcttcagcagagcgcagataccaaatactgttcttctagtgtagccgtagttaggccaccacttcaag |
| | | aactctgtagcaccgcctacatacctcgctctgctaatcctgttaccagtggctgctgccagtggcga |
| | | taagtcgtgtcttaccgggttggactcaagacgatagttaccggataaggcgcagcggtcgggctg |
| | | aacggggggttcgtgcacacagcccagcttggagcgaacgacctacaccgaactgagataccta |
| | | cagcgtgagctatgagaaagcgccacgcttcccgaagggagaaaggcggacaggtatccggtaa |
| | | gcggcagggtcggaacaggagagcgcacgagggagcttccagggggaaacgcctggtatcttt |
| | | atagtcctgtcgggtttcgccacctctgacttgagcgtcgatttttgtgatgctcgtcaggggggcgg |
| | | agcctatggaaaaacgccagcaacgcggcctttttacggttcctggccttttgctggccttttgctcac |
| | | atgttctttcctgcgttatcccctgattctgtggataaccgtattaccgcctttgagtgagctgataccgc |
| | | tcgccgcagccgaacgaccgagcgcagcgagtcagtgagcgaggaagcggaagagcgcccaa |
| | | tacgcaaaccgcctctccccgcgcgttggccgattcattaatgcagctggcacgacaggtttcccga |
| | | ctggaaagcgggcagtgagcgcaacgcaattaatgtgagttagctcactcattaggcaccccagg |
| | | ctttacactttatgcttccggctcgtatgttgtgtggaattgtgagcggataacaatttcacacaggaaa |
| | | cagctatgacatgattacgaattgcaacgatttaggtgacactatagaagagaaggaattaatacga |
| | | ctcactatagggagagagagagaattaccctcactaaagggaggagaagcatgaattccccagtg |
| | | gaaagacgcgcaggcaaaacgcaccacgtgacggagcgtgaccgcgcgccgagcgcgcgcc |
| | | aaggtcgggcaggaagagggcctatttcccatgattccttcatatttgcatatacgatacaaggctgtt |
| | | agagagataattagaattaatttgactgtaaacacaaagatattagtacaaaatacgtgacgtagaaa |
| | | gtaataatttcttgggtagtttgcagttttaaaattatgttttaaaatggactatcatatgcttaccgtaactt |
| | | gaaagtatttcgatttcttgggtttatatatcttgtggaaaggacgaggatccgcctctgaccttaattat |
| | | agggttttagagctagaaatagcaagttaaaataaggctagtccgttatcaacttgaaaaagtggcac |
| | | cgagtcggtgcttttttct |
| 52 | p19 GFP and | agacacaattgcatgaagaatctgcttagggttaggcgttttgcgctgcttcgcgatgtacgggcca |
| | neomycin | gatatacgcgttgacattgattattgactagttattaatagtaatcaattacggggtcattagttcatagc |
| | deletion | ccatatatggagttccgcgttacataacttacggtaaatggcccgcctggctgaccgcccaacgacc |
| | | cccgcccattgacgtcaataatgacgtatgttcccatagtaacgccaatagggactttccattgacgt |
| | | caatgggtggagtatttacggtaaactgcccacttggcagtacatcaagtgtatcatatgccaagtac |
| | | gccccctattgacgtcaatgacggtaaatggcccgcctggcattatgcccagtacatgaccttatgg |
| | | gactttcctacttggcagtacatctacgtattagtcatcgctattaccatggtgatgcggttttggcagta |
| | | catcaatgggcgtggatagcggtttgactcacggggatttccaagtctccaccccattgacgtcaat |
| | | gggagtttgttttggcaccaaaatcaacgggactttccaaaatgtcgtaacaactccgccccattgac |
| | | gcaaatgggcggtaggcgtgtacggtgggaggtctatataagcagagctctctggctaactagaga |
| | | acccactgcttactggcttatcgaaattaatacgactcactatagggagacccaagcttgccaccatg |
| | | gacaagaagtacagcatcggcctggacatcggtaccaacagcgtgggctgggccgtgatcaccg |
| | | acgagtacaaggtgcccagcaagaagttcaaggtgctgggcaacaccgaccgccacagcatcaa |
| | | gaagaacctgatcggcgccctgctgttcgacagcggcgagaccgccgaggccacccgcctgaa |
| | | gcgcaccgcccgccgccgctacacccgccgcaagaaccgcatctgctacctgcaggagatcttc |
| | | agcaacgagatggccaaggtggacgacagcttcttccaccgcctggaggagagcttcctggtgga |
| | | ggaggacaagaagcacgagcgccaccccatcttcggcaacatcgtggacgaggtggcctaccac |
| | | gagaagtaccccaccatctaccacctgcgcaagaagctggtggacagcaccgacaaggccgacc |
| | | tgcgcctgatctacctggccctggcccacatgatcaagttccgcggccacttcctgatcgagggcg |
| | | acctgaaccccgacaacagcgacgtggacaagctgttcatccagctggtgcagacctacaaccag |
| | | ctgttcgaggagaaccccatcaacgccagcggcgtggacgccaaggccatcctgagcgcccgcc |
| | | tgagcaagagccgccgcctggagaacctgatcgcccagctgcccggcgagaagaagaacggcc |
| | | tgttcggcaacctgatcgccctgagcctgggcctgacccccaacttcaagagcaacttcgacctgg |
| | | ccgaggacgccaagctgcagctgagcaaggacacctacgacgacgacctggacaacctgctgg |
| | | cccagatcggcgaccagtacgccgacctgttcctggccgccaagaacctgagcgacgccatcctg |
| | | ctgagcgacatcctgcgcgtgaacaccgagatcaccaaggcccccctgagcgccagcatgatca |
| | | agcgctacgacgagcaccaccaggacctgaccctgctgaaggccctggtgcgccagcagctgcc |
| | | cgagaagtacaaggagatcttcttcgaccagagcaagaacggctacgccggctacatcgacggc |
| | | ggcgccagccaggaggagttctacaagttcatcaagcccatcctggagaagatggacggcaccg |
| | | aggagctgctggtgaagctgaaccgcgaggacctgctgcgcaagcagcgcaccttcgacaacgg |
| | | cagcatcccccaccagatccacctgggcgagctgcacgccatcctgcgccgccaggaggacttct |
| | | accccttcctgaaggacaaccgcgagaagatcgagaagatcctgaccttccgcatcccctactacg |
| | | tgggccccctggcccgcggcaacagccgcttcgcctggatgacccgcaagagcgaggagacca |
| | | tcaccccctggaacttcgaggaggtggtggacaagggcgccagcgcccagagcttcatcgagcg |
| | | catgaccaacttcgacaagaacctgcccaacgagaaggtgctgcccaagcacagcctgctgtacg |
| | | agtacttcaccgtgtacaacgagctgaccaaggtgaagtacgtgaccgagggcatgcgcaagccc |
| | | gccttcctgagcggcgagcagaagaaggccatcgtggacctgctgttcaagaccaaccgcaaggt |
| | | gaccgtgaagcagctgaaggaggactacttcaagaagatcgagtgcttcgacagcgtggagatca |
| | | gcggcgtggaggaccgcttcaacgccagcctgggcacctaccacgacctgctgaagatcatcaa |
| | | ggacaaggacttcctggacaacgaggagaacgaggacatcctggaggacatcgtgctgaccctg |
| | | accctgttcgaggaccgcgagatgatcgaggagcgcctgaagacctacgcccacctgttcgacga |
| | | caaggtgatgaagcagctgaagcgccgccgctacaccggctggggccgcctgagccgcaagctt |
| | | atcaacggcatccgcgacaagcagagcggcaagaccatcctggacttcctgaagagcgacggct |
| | | tcgccaaccgcaacttcatgcagctgatccacgacgacagcctgaccttcaaggaggacatccag |
| | | aaggcccaggtgagcggccagggcgacagcctgcacgagcacatcgccaacctggccggcag |
| | | ccccgccatcaagaagggcatcctgcagaccgtgaaggtggtggacgagctggtgaaggtgatg |
| | | ggccgccacaagcccgagaacatcgtgatcgagatggcccgcgagaaccagaccacccagaag |
| | | ggccagaagaacagccgcgagcgcatgaagcgcatcgaggagggcatcaaggagctgggcag |
| | | ccagatcctgaaggagcaccccgtggagaacacccagctgcagaacgagaagctgtacctgtact |
| | | acctgcagaacggccgcgacatgtacgtggaccaggagctggacatcaaccgcctgagcgacta |
| | | cgacgtggaccacatcgtgccccagagcttcctgaaggacgacagcatcgacaacaaggtgctg |
| | | acccgcagcgacaagaaccgcggcaagagcgacaacgtgcccagcgaggaggtggtgaagaa |
| | | gatgaagaactactggcgccagctgctgaacgccaagctgatcacccagcgcaagttcgacaacc |
| | | tgaccaaggccgagcgcggcggcctgagcgagctggacaaggccggcttcatcaagcgccagc |
| | | tggtggagacccgccagatcaccaagcacgtggcccagatcctggacagccgcatgaacaccaa |
| | | gtacgacgagaacgacaagctgatccgcgaggtgaaggtgatcaccctgaagagcaagctggtg |
| | | agcgacttccgcaaggacttccagttctacaaggtgcgcgagatcaacaactaccaccacgccca |
| | | cgacgcctacctgaacgccgtggtgggcaccgccctgatcaagaagtaccccaagctggagagc |
| | | gagttcgtgtacggcgactacaaggtgtacgacgtgcgcaagatgatcgccaagagcgagcagg |
| | | agatcggcaaggccaccgccaagtacttcttctacagcaacatcatgaacttcttcaagaccgagat |
| | | caccctggccaacggcgagatccgcaagcgccccctgatcgagaccaacggcgagaccggcg |
| | | agatcgtgtgggacaagggccgcgacttcgccaccgtgcgcaaggtgctgagcatgccccaggt |
| | | gaacatcgtgaagaagaccgaggtgcagaccggcggcttcagcaaggagagcatcctgcccaa |
| | | gcgcaacagcgacaagctgatcgcccgcaagaaggactgggaccccaagaagtacggcggctt |
| | | cgacagccccaccgtggcctacagcgtgctggtggtggccaaggtggagaagggcaagagcaa |
| | | gaagctgaagagcgtgaaggagctgctgggcatcaccatcatggagcgcagcagcttcgagaag |
| | | aaccccatcgacttcctggaggccaagggctacaaggaggtgaagaaggacctgatcatcaagct |
| | | gcccaagtacagcctgttcgagctggagaacggccgcaagcgcatgctggccagcgccggcga |
| | | gctgcagaagggcaacgagctggccctgcccagcaagtacgtgaacttcctgtacctggccagcc |
| | | actacgagaagctgaagggcagccccgaggacaacgagcagaagcagctgttcgtggagcagc |
| | | acaagcactacctggacgagatcatcgagcagatcagcgagttcagcaagcgcgtgatcctggcc |
| | | gacgccaacctggacaaggtgctgagcgcctacaacaagcaccgcgacaagcccatccgcgag |
| | | caggccgagaacatcatccacctgttcaccctgaccaacctgggcgcccccgccgccttcaagta |
| | | cttcgacaccaccatcgaccgcaagcgctacaccagcaccaaggaggtgctggacgccaccctg |
| | | atccaccagagcatcaccggtctgtacgagacccgcatcgacctgagccagctgggcggcgact |
| | | gatgaccaccccaacttgtttattgcagcttataatggttacaaataaagcaatagcatcacaaatttca |
| | | caaataaagcatttttttcactgcattctagttgtggtttgtccaaactcatcaatgtatcttatcatgtctgt |
| | | ataccgtcgacctctagctagagcttggcgtaatcatggtcatagctgtttcctgtgtgaaattgttatc |
| | | cgctcacaattccacacaacatacgagccggaagcataaaggtaagcctgaatattgaaaaagga |
| | | agagtatgagtattcaacatttccgtgtcgcccttattcccttttttgcggcattttgccttcctgtttttgct |
| | | cacccagaaacgctggtgaaagtaaaagatgctgaagatcagttgggtgcacgagtgggttacatc |
| | | gaactggatctcaacagcggtaagatccttgagagttttcgccccgaagaacgttttccaatgatgag |
| | | cacttttaaagttctgctatgtggcgcggtattatcccgtattgacgccgggcaagagcaactcggtc |
| | | gccgcatacactattctcagaatgacttggttgagtactcaccagtcacagaaaagcatcttacggat |
| | | ggcatgacagtaagagaattatgcagtgctgccataaccatgagtgataacactgcggccaacttac |
| | | ttctgacaacgatcggaggaccgaaggagctaaccgcttttttgcacaacatgggggatcatgtaac |
| | | tcgccttgatcgttgggaaccggagctgaatgaagccataccaaacgacgagcgtgacaccacga |
| | | tgcctgtagcaatggcaacaacgttgcgcaaactattaactggcgaactacttactctagcttcccgg |
| | | caacaattaatagactggatggaggcggataaagttgcaggaccacttctgcgctcggcccttccg |
| | | gctggctggtttattgctgataaatctggagccggtgagcgtgggtctcgcggtatcattgcagcact |
| | | ggggccagatggtaagccctcccgtatcgtagttatctacacgacggggagtcaggcaactatgga |
| | | tgaacgaaatagacagatcgctgagataggtgcctcactgattaagcattggtaactgtcagaccaa |
| | | gtttactcatatatactttagattgatttaaaacttcatttttaatttaaaaggatctaggtgaagatccttttt |
| | | gataatctcatgaccaaaatcccttaacgtgagttttcgttccactgagcgtcagaccccgtagaaaa |
| | | gatcaaaggatcttcttgagatcctttttttctgcgcgtaatctgctgcttgcaaacaaaaaaaccaccg |
| | | ctaccagcggtggtttttttgccggatcaagagctaccaactctttttccgaaggtaactggcttcagc |
| | | agagcgcagataccaaatactgttcttctagtgtagccgtagttaggccaccacttcaagaactctgt |
| | | agcaccgcctacatacctcgctctgctaatcctgttaccagtggctgctgccagtggcgataagtcgt |
| | | gtcttaccgggttggactcaagacgatagttaccggataaggcgcagcggtcgggctgaacgggg |
| | | ggttcgtgcacacagcccagcttggagcgaacgacctacaccgaactgagatacctacagcgtga |
| | | gctatgagaaagcgccacgcttcccgaagggagaaaggcggacaggtatccggtaagcggcag |
| | | ggtcggaacaggagagcgcacgagggagcttccagggggaaacgcctggtatctttatagtcctg |
| | | tcgggtttcgccacctctgacttgagcgtcgatttttgtgatgctcgtcaggggggcggagcctatgg |
| | | aaaaacgccagcaacgcggcctttttacggttcctggccttttgctggccttttgctcacatgttctttcc |
| | | tgcgttatcccctgattctgtggataaccgtattaccgcctttgagtgagctgataccgctcgccgcag |
| | | ccgaacgaccgagcgcagcgagtcagtgagcgaggaagcggaagagcgcccaatacgcaaac |
| | | cgcctctccccgcgcgttggccgattcattaatgcagctggcacgacaggtttcccgactggaaag |
| | | cgggcagtgagcgcaacgcaattaatgtgagttagctcactcattaggcaccccaggctttacacttt |
| | | atgcttccggctcgtatgttgtgtggaattgtgagcggataacaatttcacacaggaaacagctatga |
| | | catgattacgaattgcaacgatttaggtgacactatagaagagaaggaattaatacgactcactatag |
| | | ggagagagagagaattaccctcactaaagggaggagaagcatgaattccccagtggaaagacgc |
| | | gcaggcaaaacgcaccacgtgacggagcgtgaccgcgcgccgagcgcgcgccaaggtcggg |
| | | caggaagagggcctatttcccatgattccttcatatttgcatatacgatacaaggctgttagagagata |
| | | attagaattaatttgactgtaaacacaaagatattagtacaaaatacgtgacgtagaaagtaataatttc |
| | | ttgggtagtttgcagttttaaaattatgttttaaaatggactatcatatgcttaccgtaacttgaaagtattt |
| | | cgatttcttgggtttatatatcttgtggaaaggacgaggatccggagaagcttctctgttttagagctag |
| | | aaatagcaagttaaaataaggctagtccgttatcaacttgaaaaagtggcaccgagtcggtgctttttt |
| | | ct |
| 53 | p20 GFP and | agacacaattccccagtggaaagacgcgcaggcaaaacgcaccacgtgacggagcgtgaccgc |
| | Neomycin | gcgccgagcgcgcgccaaggtcgggcaggaagagggcctatttcccatgattccttcatatttgca |
| | deletion | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgaccaagatctggacgggtggttttagagctagaaatagcaagttaaaataaggctagtccgtta |
| | | tcaacttgaaaaagtggcaccgagtcggtgcttttttctagacacaattccccagtggaaagacgcg |
| | | caggcaaaacgcaccacgtgacggagcgtgaccgcgcgccgagcgcgcgccaaggtcgggca |
| | | ggaagagggcctatttcccatgattccttcatatttgcatatacgatacaaggctgttagagagataatt |
| | | agaattaatttgactgtaaacacaaagatattagtacaaaatacgtgacgtagaaagtaataatttcttg |
| | | ggtagtttgcagttttaaaattatgttttaaaatggactatcatatgcttaccgtaacttgaaagtatttcga |
| | | tttcttgggtttatatatcttgtggaaaggacgaggatccgggtgtatgacacgttgtcggttttagagc |
| | | tagaaatagcaagttaaaataaggctagtccgttatcaacttgaaaaagtggcaccgagtcggtgctt |
| | | ttttctagacacaattgcatgaagaatctgcttagggttaggcgttttgcgctgcttcgcgatgtacgg |
| | | gccagatatacgcgttgacattgattattgactagttattaatagtaatcaattacggggtcattagttca |
| | | tagcccatatatggagttccgcgttacataacttacggtaaatggcccgcctggctgaccgcccaac |
| | | gacccccgcccattgacgtcaataatgacgtatgttcccatagtaacgccaatagggactttccattg |
| | | acgtcaatgggtggagtatttacggtaaactgcccacttggcagtacatcaagtgtatcatatgccaa |
| | | gtacgccccctattgacgtcaatgacggtaaatggcccgcctggcattatgcccagtacatgacctt |
| | | atgggactttcctacttggcagtacatctacgtattagtcatcgctattaccatggtgatgcggttttggc |
| | | agtacatcaatgggcgtggatagcggtttgactcacggggatttccaagtctccaccccattgacgt |
| | | caatgggagtttgttttggcaccaaaatcaacgggactttccaaaatgtcgtaacaactccgccccat |
| | | tgacgcaaatgggcggtaggcgtgtacggtgggaggtctatataagcagagctctctggctaacta |
| | | gagaacccactgcttactggcttatcgaaattaatacgactcactatagggagacccaagcttgcca |
| | | ccatggacaagaagtacagcatcggcctggacatcggtaccaacagcgtgggctgggccgtgat |
| | | caccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggcctttttacggttcctggccttttgctggccttttgctcacatgtt |
| | | ctttcctgcgttatcccctgattctgtggataaccgtattaccgcctttgagtgagctgataccgctcgc |
| | | cgcagccgaacgaccgagcgcagcgagtcagtgagcgaggaagcggaagagcgcccaatacg |
| | | caaaccgcctctccccgcgcgttggccgattcattaatgcagctggcacgacaggtttcccgactg |
| | | gaaagcgggcagtgagcgcaacgcaattaatgtgagttagctcactcattaggcaccccaggcttt |
| | | acactttatgcttccggctcgtatgttgtgtggaattgtgagcggataacaatttcacacaggaaacag |
| | | ctatgacatgattacgaattgcaacgatttaggtgacactatagaagagaaggaattaatacgactca |
| | | ctatagggagagagagagaattaccctcactaaagggaggagaagcatgaattccccagtggaaa |
| | | gacgcgcaggcaaaacgcaccacgtgacggagcgtgaccgcgcgccgagcgcgcgccaaggt |
| | | cgggcaggaagagggcctatttcccatgattccttcatatttgcatatacgatacaaggctgttagag |
| | | agataattagaattaatttgactgtaaacacaaagatattagtacaaaatacgtgacgtagaaagtaat |
| | | aatttcttgggtagtttgcagttttaaaattatgttttaaaatggactatcatatgcttaccgtaacttgaaa |
| | | gtatttcgatttcttgggtttatatatcttgtggaaaggacgaggatccgtgtttaacgacatatcgcca |
| | | gttttagagctagaaatagcaagttaaaataaggctagtccgttatcaacttgaaaaagtggcaccga |
| | | gtcggtgcttttttct |
| 54 | p21 GFP and | agacacaattccccagtggaaagacgcgcaggcaaaacgcaccacgtgacggagcgtgaccgc |
| | Neomycin | gcgccgagcgcgcgccaaggtcgggcaggaagagggcctatttcccatgattccttcatatttgca |
| | deletion | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgtttacttcggcttttacaaggttttagagctagaaatagcaagttaaaataaggctagtccgttatc |
| | | aacttgaaaaagtggcaccgagtcggtgcttttttctagacacaattccccagtggaaagacgcgca |
| | | ggcaaaacgcaccacgtgacggagcgtgaccgcgcgccgagcgcgcgccaaggtcgggcag |
| | | gaagagggcctatttcccatgattccttcatatttgcatatacgatacaaggctgttagagagataatta |
| | | gaattaatttgactgtaaacacaaagatattagtacaaaatacgtgacgtagaaagtaataatttcttgg |
| | | gtagtttgcagttttaaaattatgttttaaaatggactatcatatgcttaccgtaacttgaaagtatttcgat |
| | | ttcttgggtttatatatcttgtggaaaggacgaggatccgaaaggggtccttcgaacacggttttagag |
| | | ctagaaatagcaagttaaaataaggctagtccgttatcaacttgaaaaagtggcaccgagtcggtgc |
| | | ttttttctagacacaattgcatgaagaatctgcttagggttaggcgttttgcgctgcttcgcgatgtacg |
| | | ggccagatatacgcgttgacattgattattgactagttattaatagtaatcaattacggggtcattagttc |
| | | atagcccatatatggagttccgcgttacataacttacggtaaatggcccgcctggctgaccgcccaa |
| | | cgacccccgcccattgacgtcaataatgacgtatgttcccatagtaacgccaatagggactttccatt |
| | | gacgtcaatgggtggagtatttacggtaaactgcccacttggcagtacatcaagtgtatcatatgcca |
| | | agtacgccccctattgacgtcaatgacggtaaatggcccgcctggcattatgcccagtacatgacct |
| | | tatgggactttcctacttggcagtacatctacgtattagtcatcgctattaccatggtgatgcggttttgg |
| | | cagtacatcaatgggcgtggatagcggtttgactcacggggatttccaagtctccaccccattgacg |
| | | tcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaact |
| | | agagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggcctttttacggttcctggccttttgctggccttttgctcacatgtt |
| | | ctttcctgcgttatcccctgattctgtggataaccgtattaccgcctttgagtgagctgataccgctcgc |
| | | cgcagccgaacgaccgagcgcagcgagtcagtgagcgaggaagcggaagagcgcccaatacg |
| | | caaaccgcctctccccgcgcgttggccgattcattaatgcagctggcacgacaggtttcccgactg |
| | | gaaagcgggcagtgagcgcaacgcaattaatgtgagttagctcactcattaggcaccccaggcttt |
| | | acactttatgcttccggctcgtatgttgtgtggaattgtgagcggataacaatttcacacaggaaacag |
| | | ctatgacatgattacgaattgcaacgatttaggtgacactatagaagagaaggaattaatacgactca |
| | | ctatagggagagagagagaattaccctcactaaagggaggagaagcatgaattccccagtggaaa |
| | | gacgcgcaggcaaaacgcaccacgtgacggagcgtgaccgcgcgccgagcgcgcgccaaggt |
| | | cgggcaggaagagggcctatttcccatgattccttcatatttgcatatacgatacaaggctgttagag |
| | | agataattagaattaatttgactgtaaacacaaagatattagtacaaaatacgtgacgtagaaagtaat |
| | | aatttcttgggtagtttgcagttttaaaattatgttttaaaatggactatcatatgcttaccgtaacttgaaa |
| | | gtatttcgatttcttgggtttatatatcttgtggaaaggacgaggatccgcatacgggaacgcacatag |
| | | tgttttagagctagaaatagcaagttaaaataaggctagtccgttatcaacttgaaaaagtggcaccg |
| | | agtcggtgcttttttct |
| 55 | p22 GFP and | agacacaattccccagtggaaagacgcgcaggcaaaacgcaccacgtgacggagcgtgaccgc |
| | Neomycin | gcgccgagcgcgcgccaaggtcgggcaggaagagggcctatttcccatgattccttcatatttgca |
| | deletion | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccgattgttgcacgggagaaccgttttagagctagaaatagcaagttaaaataaggctagtccgttat |
| | | caacttgaaaaagtggcaccgagtcggtgcttttttctagacacaattccccagtggaaagacgcgc |
| | | aggcaaaacgcaccacgtgacggagcgtgaccgcgcgccgagcgcgcgccaaggtcgggca |
| | | ggaagagggcctatttcccatgattccttcatatttgcatatacgatacaaggctgttagagagataatt |
| | | agaattaatttgactgtaaacacaaagatattagtacaaaatacgtgacgtagaaagtaataatttcttg |
| | | ggtagtttgcagttttaaaattatgttttaaaatggactatcatatgcttaccgtaacttgaaagtatttcga |
| | | tttcttgggtttatatatcttgtggaaaggacgaggatccgactttggcaagtaagcccgcgttttaga |
| | | gctagaaatagcaagttaaaataaggctagtccgttatcaacttgaaaaagtggcaccgagtcggtg |
| | | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggcctttttacggttcctggccttttgctggccttttgctcacatgtt |
| | | ctttcctgcgttatcccctgattctgtggataaccgtattaccgcctttgagtgagctgataccgctcgc |
| | | cgcagccgaacgaccgagcgcagcgagtcagtgagcgaggaagcggaagagcgcccaatacg |
| | | caaaccgcctctccccgcgcgttggccgattcattaatgcagctggcacgacaggtttcccgactg |
| | | gaaagcgggcagtgagcgcaacgcaattaatgtgagttagctcactcattaggcaccccaggcttt |
| | | acactttatgcttccggctcgtatgttgtgtggaattgtgagcggataacaatttcacacaggaaacag |
| | | ctatgacatgattacgaattgcaacgatttaggtgacactatagaagagaaggaattaatacgactca |
| | | ctatagggagagagagagaattaccctcactaaagggaggagaagcatgaattccccagtggaaa |
| | | gacgcgcaggcaaaacgcaccacgtgacggagcgtgaccgcgcgccgagcgcgcgccaaggt |
| | | cgggcaggaagagggcctatttcccatgattccttcatatttgcatatacgatacaaggctgttagag |
| | | agataattagaattaatttgactgtaaacacaaagatattagtacaaaatacgtgacgtagaaagtaat |
| | | aatttcttgggtagtttgcagttttaaaattatgttttaaaatggactatcatatgcttaccgtaacttgaaa |
| | | gtatttcgatttcttgggtttatatatcttgtggaaaggacgaggatccgtttaacaatcgtctcgtggag |
| | | ttttagagctagaaatagcaagttaaaataaggctagtccgttatcaacttgaaaaagtggcaccgag |
| | | tcggtgcttttttct |
| 56 | P18 smallest; | agacacaattccccagtggaaagacgcgcaggcaaaacgcaccacgtgacggagcgtgaccgc |
| | stop codon | gcgccgagcgcgcgccaaggtcgggcaggaagagggcctatttcccatgattccttcatatttgca |
| | was added | tatacgatacaaggctgttagagagataattagaattaatttgactgtaaacacaaagatattagtaca |
| | after the | aaatacgtgacgtagaaagtaataatttcttgggtagtttgcagttttaaaattatgttttaaaatggacta |
| | Cas9 operon | tcatatgcttaccgtaacttgaaagtatttcgatttcttgggtttatatatcttgtggaaaggacgaggat |
| | and the GFP | ccgcaactcatagagagttagcggttttagagctagaaatagcaagttaaaataaggctagtccgtta |
| | gene and | tcaacttgaaaaagtggcaccgagtcggtgcttttttctagacacaattccccagtggaaagacgcg |
| | neomycin | caggcaaaacgcaccacgtgacggagcgtgaccgcgcgccgagcgcgcgccaaggtcgggca |
| | genes | ggaagagggcctatttcccatgattccttcatatttgcatatacgatacaaggctgttagagagataatt |
| | sequences | agaattaatttgactgtaaacacaaagatattagtacaaaatacgtgacgtagaaagtaataatttcttg |
| | were | ggtagtttgcagttttaaaattatgttttaaaatggactatcatatgcttaccgtaacttgaaagtatttcga |
| | removed. | tttcttgggtttatatatcttgtggaaaggacgaggatccgcgtggtttagagaagcgcacgttttaga |
| | Additionally, | gctagaaatagcaagttaaaataaggctagtccgttatcaacttgaaaaagtggcaccgagtcggtg |
| | 395 bases | cttttttctagacacaattgcatgaagaatctgcttagggttaggcgttttgcgctgcttcgcgatgtac |
| | were | gggccagatatacgcgttgacattgattattgactagttattaatagtaatcaattacggggtcattagtt |
| | removed | catagcccatatatggagttccgcgttacataacttacggtaaatggcccgcctggctgaccgccca |
| | located | acgacccccgcccattgacgtcaataatgacgtatgttcccatagtaacgccaatagggactttccat |
| | between the | tgacgtcaatgggtggagtatttacggtaaactgcccacttggcagtacatcaagtgtatcatatgcc |
| | origin of | aagtacgccccctattgacgtcaatgacggtaaatggcccgcctggcattatgcccagtacatgacc |
| | replication | ttatgggactttcctacttggcagtacatctacgtattagtcatcgctattaccatggtgatgcggttttg |
| | and the sp6 | gcagtacatcaatgggcgtggatagcggtttgactcacggggatttccaagtctccaccccattgac |
| | promoter | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacTGATGAccaccccaacttgtttattgcagcttataatggttacaaataaagcaatagcatca |
| | | caaatttcacaaataaagcatttttttcactgcattctagttgtggtttgtccaaactcatcaatgtatcttat |
| | | catgtctgtataccgtcgacctctagctagagcttggcgtaatcatggtcatagctgtttcctgtgtgaa |
| | | attgttatccgctcacaattccacacaacatacgagccggaagcataaaggtaagcctgaatattga |
| | | aaaaggaagagtatgagtattcaacatttccgtgtcgcccttattcccttttttgcggcattttgccttcct |
| | | gtttttgctcacccagaaacgctggtgaaagtaaaagatgctgaagatcagttgggtgcacgagtgg |
| | | gttacatcgaactggatctcaacagcggtaagatccttgagagttttcgccccgaagaacgttttcca |
| | | atgatgagcacttttaaagttctgctatgtggcgcggtattatcccgtattgacgccgggcaagagca |
| | | actcggtcgccgcatacactattctcagaatgacttggttgagtactcaccagtcacagaaaagcatc |
| | | ttacggatggcatgacagtaagagaattatgcagtgctgccataaccatgagtgataacactgcggc |
| | | caacttacttctgacaacgatcggaggaccgaaggagctaaccgcttttttgcacaacatgggggat |
| | | catgtaactcgccttgatcgttgggaaccggagctgaatgaagccataccaaacgacgagcgtgac |
| | | accacgatgcctgtagcaatggcaacaacgttgcgcaaactattaactggcgaactacttactctag |
| | | cttcccggcaacaattaatagactggatggaggcggataaagttgcaggaccacttctgcgctcgg |
| | | cccttccggctggctggtttattgctgataaatctggagccggtgagcgtgggtctcgcggtatcatt |
| | | gcagcactggggccagatggtaagccctcccgtatcgtagttatctacacgacggggagtcaggc |
| | | aactatggatgaacgaaatagacagatcgctgagataggtgcctcactgattaagcattggtaactgt |
| | | cagaccaagtttactcatatatactttagattgatttaaaacttcatttttaatttaaaaggatctaggtgaa |
| | | gatcctttttgataatctcatgaccaaaatcccttaacgtgagttttcgttccactgagcgtcagacccc |
| | | gtagaaaagatcaaaggatcttcttgagatcctttttttctgcgcgtaatctgctgcttgcaaacaaaaa |
| | | aaccaccgctaccagcggtggtttttttgccggatcaagagctaccaactctttttccgaaggtaactg |
| | | gcttcagcagagcgcagataccaaatactgttcttctagtgtagccgtagttaggccaccacttcaag |
| | | aactctgtagcaccgcctacatacctcgctctgctaatcctgttaccagtggctgctgccagtggcga |
| | | taagtcgtgtcttaccgggttggactcaagacgatagttaccggataaggcgcagcggtcgggctg |
| | | aacggggggttcgtgcacacagcccagcttggagcgaacgacctacaccgaactgagataccta |
| | | cagcgtgagctatgagaaagcgccacgcttcccgaagggagaaaggcggacaggtatccggtaa |
| | | gcggcagggtcggaacaggagagcgcacgagggagcttccagggggaaacgcctggtatcttt |
| | | atagtcctgtcgggtttcgccacctctgacttgagcgtcgatttttgtgatgctcgtcaggggggcgg |
| | | agcctatggaaaaacgccagcaacgcggccttgcaacgatttaggtgacactatagaagagaagg |
| | | aattaatacgactcactatagggagagagagagaattaccctcactaaagggaggagaagcatga |
| | | attccccagtggaaagacgcgcaggcaaaacgcaccacgtgacggagcgtgaccgcgcgccga |
| | | gcgcgcgccaaggtcgggcaggaagagggcctatttcccatgattccttcatatttgcatatacgat |
| | | acaaggctgttagagagataattagaattaatttgactgtaaacacaaagatattagtacaaaatacgt |
| | | gacgtagaaagtaataatttcttgggtagtttgcagttttaaaattatgttttaaaatggactatcatatgc |
| | | ttaccgtaacttgaaagtatttcgatttcttgggtttatatatcttgtggaaaggacgaggatccgcctct |
| | | gaccttaattatagggttttagagctagaaatagcaagttaaaataaggctagtccgttatcaacttga |
| | | aaaagtggcaccgagtcggtgcttttttct |
| 57 | P19 smallest; | agacacaattgcatgaagaatctgcttagggttaggcgttttgcgctgcttcgcgatgtacgggcca |
| | stop codon | gatatacgcgttgacattgattattgactagttattaatagtaatcaattacggggtcattagttcatagc |
| | was added | ccatatatggagttccgcgttacataacttacggtaaatggcccgcctggctgaccgcccaacgacc |
| | after the | cccgcccattgacgtcaataatgacgtatgttcccatagtaacgccaatagggactttccattgacgt |
| | Cas9 operon | caatgggtggagtatttacggtaaactgcccacttggcagtacatcaagtgtatcatatgccaagtac |
| | and the GFP | gccccctattgacgtcaatgacggtaaatggcccgcctggcattatgcccagtacatgaccttatgg |
| | gene and | gactttcctacttggcagtacatctacgtattagtcatcgctattaccatggtgatgcggttttggcagta |
| | neomycin | catcaatgggcgtggatagcggtttgactcacggggatttccaagtctccaccccattgacgtcaat |
| | genes | gggagtttgttttggcaccaaaatcaacgggactttccaaaatgtcgtaacaactccgccccattgac |
| | sequences | gcaaatgggcggtaggcgtgtacggtgggaggtctatataagcagagctctctggctaactagaga |
| | were | acccactgcttactggcttatcgaaattaatacgactcactatagggagacccaagcttgccaccatg |
| | removed. | gacaagaagtacagcatcggcctggacatcggtaccaacagcgtgggctgggccgtgatcaccg |
| | Additionally, | acgagtacaaggtgcccagcaagaagttcaaggtgctgggcaacaccgaccgccacagcatcaa |
| | 395 bases | gaagaacctgatcggcgccctgctgttcgacagcggcgagaccgccgaggccacccgcctgaa |
| | were | gcgcaccgcccgccgccgctacacccgccgcaagaaccgcatctgctacctgcaggagatcttc |
| | removed | agcaacgagatggccaaggtggacgacagcttcttccaccgcctggaggagagcttcctggtgga |
| | located | ggaggacaagaagcacgagcgccaccccatcttcggcaacatcgtggacgaggtggcctaccac |
| | between the | gagaagtaccccaccatctaccacctgcgcaagaagctggtggacagcaccgacaaggccgacc |
| | origin of | tgcgcctgatctacctggccctggcccacatgatcaagttccgcggccacttcctgatcgagggcg |
| | replication | acctgaaccccgacaacagcgacgtggacaagctgttcatccagctggtgcagacctacaaccag |
| | and the sp6 | ctgttcgaggagaaccccatcaacgccagcggcgtggacgccaaggccatcctgagcgcccgcc |
| | promoter | tgagcaagagccgccgcctggagaacctgatcgcccagctgcccggcgagaagaagaacggcc |
| | | tgttcggcaacctgatcgccctgagcctgggcctgacccccaacttcaagagcaacttcgacctgg |
| | | ccgaggacgccaagctgcagctgagcaaggacacctacgacgacgacctggacaacctgctgg |
| | | cccagatcggcgaccagtacgccgacctgttcctggccgccaagaacctgagcgacgccatcctg |
| | | ctgagcgacatcctgcgcgtgaacaccgagatcaccaaggcccccctgagcgccagcatgatca |
| | | agcgctacgacgagcaccaccaggacctgaccctgctgaaggccctggtgcgccagcagctgcc |
| | | cgagaagtacaaggagatcttcttcgaccagagcaagaacggctacgccggctacatcgacggc |
| | | ggcgccagccaggaggagttctacaagttcatcaagcccatcctggagaagatggacggcaccg |
| | | aggagctgctggtgaagctgaaccgcgaggacctgctgcgcaagcagcgcaccttcgacaacgg |
| | | cagcatcccccaccagatccacctgggcgagctgcacgccatcctgcgccgccaggaggacttct |
| | | accccttcctgaaggacaaccgcgagaagatcgagaagatcctgaccttccgcatcccctactacg |
| | | tgggccccctggcccgcggcaacagccgcttcgcctggatgacccgcaagagcgaggagacca |
| | | tcaccccctggaacttcgaggaggtggtggacaagggcgccagcgcccagagcttcatcgagcg |
| | | catgaccaacttcgacaagaacctgcccaacgagaaggtgctgcccaagcacagcctgctgtacg |
| | | agtacttcaccgtgtacaacgagctgaccaaggtgaagtacgtgaccgagggcatgcgcaagccc |
| | | gccttcctgagcggcgagcagaagaaggccatcgtggacctgctgttcaagaccaaccgcaaggt |
| | | gaccgtgaagcagctgaaggaggactacttcaagaagatcgagtgcttcgacagcgtggagatca |
| | | gcggcgtggaggaccgcttcaacgccagcctgggcacctaccacgacctgctgaagatcatcaa |
| | | ggacaaggacttcctggacaacgaggagaacgaggacatcctggaggacatcgtgctgaccctg |
| | | accctgttcgaggaccgcgagatgatcgaggagcgcctgaagacctacgcccacctgttcgacga |
| | | caaggtgatgaagcagctgaagcgccgccgctacaccggctggggccgcctgagccgcaagctt |
| | | atcaacggcatccgcgacaagcagagcggcaagaccatcctggacttcctgaagagcgacggct |
| | | tcgccaaccgcaacttcatgcagctgatccacgacgacagcctgaccttcaaggaggacatccag |
| | | aaggcccaggtgagcggccagggcgacagcctgcacgagcacatcgccaacctggccggcag |
| | | ccccgccatcaagaagggcatcctgcagaccgtgaaggtggtggacgagctggtgaaggtgatg |
| | | ggccgccacaagcccgagaacatcgtgatcgagatggcccgcgagaaccagaccacccagaag |
| | | ggccagaagaacagccgcgagcgcatgaagcgcatcgaggagggcatcaaggagctgggcag |
| | | ccagatcctgaaggagcaccccgtggagaacacccagctgcagaacgagaagctgtacctgtact |
| | | acctgcagaacggccgcgacatgtacgtggaccaggagctggacatcaaccgcctgagcgacta |
| | | cgacgtggaccacatcgtgccccagagcttcctgaaggacgacagcatcgacaacaaggtgctg |
| | | acccgcagcgacaagaaccgcggcaagagcgacaacgtgcccagcgaggaggtggtgaagaa |
| | | gatgaagaactactggcgccagctgctgaacgccaagctgatcacccagcgcaagttcgacaacc |
| | | tgaccaaggccgagcgcggcggcctgagcgagctggacaaggccggcttcatcaagcgccagc |
| | | tggtggagacccgccagatcaccaagcacgtggcccagatcctggacagccgcatgaacaccaa |
| | | gtacgacgagaacgacaagctgatccgcgaggtgaaggtgatcaccctgaagagcaagctggtg |
| | | agcgacttccgcaaggacttccagttctacaaggtgcgcgagatcaacaactaccaccacgccca |
| | | cgacgcctacctgaacgccgtggtgggcaccgccctgatcaagaagtaccccaagctggagagc |
| | | gagttcgtgtacggcgactacaaggtgtacgacgtgcgcaagatgatcgccaagagcgagcagg |
| | | agatcggcaaggccaccgccaagtacttcttctacagcaacatcatgaacttcttcaagaccgagat |
| | | caccctggccaacggcgagatccgcaagcgccccctgatcgagaccaacggcgagaccggcg |
| | | agatcgtgtgggacaagggccgcgacttcgccaccgtgcgcaaggtgctgagcatgccccaggt |
| | | gaacatcgtgaagaagaccgaggtgcagaccggcggcttcagcaaggagagcatcctgcccaa |
| | | gcgcaacagcgacaagctgatcgcccgcaagaaggactgggaccccaagaagtacggcggctt |
| | | cgacagccccaccgtggcctacagcgtgctggtggtggccaaggtggagaagggcaagagcaa |
| | | gaagctgaagagcgtgaaggagctgctgggcatcaccatcatggagcgcagcagcttcgagaag |
| | | aaccccatcgacttcctggaggccaagggctacaaggaggtgaagaaggacctgatcatcaagct |
| | | gcccaagtacagcctgttcgagctggagaacggccgcaagcgcatgctggccagcgccggcga |
| | | gctgcagaagggcaacgagctggccctgcccagcaagtacgtgaacttcctgtacctggccagcc |
| | | actacgagaagctgaagggcagccccgaggacaacgagcagaagcagctgttcgtggagcagc |
| | | acaagcactacctggacgagatcatcgagcagatcagcgagttcagcaagcgcgtgatcctggcc |
| | | gacgccaacctggacaaggtgctgagcgcctacaacaagcaccgcgacaagcccatccgcgag |
| | | caggccgagaacatcatccacctgttcaccctgaccaacctgggcgcccccgccgccttcaagta |
| | | cttcgacaccaccatcgaccgcaagcgctacaccagcaccaaggaggtgctggacgccaccctg |
| | | atccaccagagcatcaccggtctgtacgagacccgcatcgacctgagccagctgggcggcgact |
| | | gatgaccaccccaacttgtttattgcagcttataatggttacaaataaagcaatagcatcacaaatttca |
| | | caaataaagcatttttttcactgcattctagttgtggtttgtccaaactcatcaatgtatcttatcatgtctgt |
| | | ataccgtcgacctctagctagagcttggcgtaatcatggtcatagctgtttcctgtgtgaaattgttatc |
| | | cgctcacaattccacacaacatacgagccggaagcataaaggtaagcctgaatattgaaaaagga |
| | | agagtatgagtattcaacatttccgtgtcgcccttattcccttttttgcggcattttgccttcctgtttttgct |
| | | cacccagaaacgctggtgaaagtaaaagatgctgaagatcagttgggtgcacgagtgggttacatc |
| | | gaactggatctcaacagcggtaagatccttgagagttttcgccccgaagaacgttttccaatgatgag |
| | | cacttttaaagttctgctatgtggcgcggtattatcccgtattgacgccgggcaagagcaactcggtc |
| | | gccgcatacactattctcagaatgacttggttgagtactcaccagtcacagaaaagcatcttacggat |
| | | ggcatgacagtaagagaattatgcagtgctgccataaccatgagtgataacactgcggccaacttac |
| | | ttctgacaacgatcggaggaccgaaggagctaaccgcttttttgcacaacatgggggatcatgtaac |
| | | tcgccttgatcgttgggaaccggagctgaatgaagccataccaaacgacgagcgtgacaccacga |
| | | tgcctgtagcaatggcaacaacgttgcgcaaactattaactggcgaactacttactctagcttcccgg |
| | | caacaattaatagactggatggaggcggataaagttgcaggaccacttctgcgctcggcccttccg |
| | | gctggctggtttattgctgataaatctggagccggtgagcgtgggtctcgcggtatcattgcagcact |
| | | ggggccagatggtaagccctcccgtatcgtagttatctacacgacggggagtcaggcaactatgga |
| | | tgaacgaaatagacagatcgctgagataggtgcctcactgattaagcattggtaactgtcagaccaa |
| | | gtttactcatatatactttagattgatttaaaacttcatttttaatttaaaaggatctaggtgaagatccttttt |
| | | gataatctcatgaccaaaatcccttaacgtgagttttcgttccactgagcgtcagaccccgtagaaaa |
| | | gatcaaaggatcttcttgagatcctttttttctgcgcgtaatctgctgcttgcaaacaaaaaaaccaccg |
| | | ctaccagcggtggtttttttgccggatcaagagctaccaactctttttccgaaggtaactggcttcagc |
| | | agagcgcagataccaaatactgttcttctagtgtagccgtagttaggccaccacttcaagaactctgt |
| | | agcaccgcctacatacctcgctctgctaatcctgttaccagtggctgctgccagtggcgataagtcgt |
| | | gtcttaccgggttggactcaagacgatagttaccggataaggcgcagcggtcgggctgaacgggg |
| | | ggttcgtgcacacagcccagcttggagcgaacgacctacaccgaactgagatacctacagcgtga |
| | | gctatgagaaagcgccacgcttcccgaagggagaaaggcggacaggtatccggtaagcggcag |
| | | ggtcggaacaggagagcgcacgagggagcttccagggggaaacgcctggtatctttatagtcctg |
| | | tcgggtttcgccacctctgacttgagcgtcgatttttgtgatgctcgtcaggggggcggagcctatgg |
| | | aaaaacgccagcaacgcggccttgcaacgatttaggtgacactatagaagagaaggaattaatac |
| | | gactcactatagggagagagagagaattaccctcactaaagggaggagaagcatgaattccccag |
| | | tggaaagacgcgcaggcaaaacgcaccacgtgacggagcgtgaccgcgcgccgagcgcgcgc |
| | | caaggtcgggcaggaagagggcctatttcccatgattccttcatatttgcatatacgatacaaggctg |
| | | ttagagagataattagaattaatttgactgtaaacacaaagatattagtacaaaatacgtgacgtagaa |
| | | agtaataatttcttgggtagtttgcagttttaaaattatgttttaaaatggactatcatatgcttaccgtaac |
| | | ttgaaagtatttcgatttcttgggtttatatatcttgtggaaaggacgaggatccggagaagcttctctgt |
| | | tttagagctagaaatagcaagttaaaataaggctagtccgttatcaacttgaaaaagtggcaccgagt |
| | | cggtgcttttttct |
| 58 | P20 smallest; | agacacaattccccagtggaaagacgcgcaggcaaaacgcaccacgtgacggagcgtgaccgc |
| | stop codon | gcgccgagcgcgcgccaaggtcgggcaggaagagggcctatttcccatgattccttcatatttgca |
| | was added | tatacgatacaaggctgttagagagataattagaattaatttgactgtaaacacaaagatattagtaca |
| | after the | aaatacgtgacgtagaaagtaataatttcttgggtagtttgcagttttaaaattatgttttaaaatggacta |
| | Cas9 operon | tcatatgcttaccgtaacttgaaagtatttcgatttcttgggtttatatatcttgtggaaaggacgaggat |
| | and the GFP | ccgaccaagatctggacgggtggttttagagctagaaatagcaagttaaaataaggctagtccgtta |
| | gene and | tcaacttgaaaaagtggcaccgagtcggtgcttttttctagacacaattccccagtggaaagacgcg |
| | neomycin | caggcaaaacgcaccacgtgacggagcgtgaccgcgcgccgagcgcgcgccaaggtcgggca |
| | genes | ggaagagggcctatttcccatgattccttcatatttgcatatacgatacaaggctgttagagagataatt |
| | sequences | agaattaatttgactgtaaacacaaagatattagtacaaaatacgtgacgtagaaagtaataatttcttg |
| | were | ggtagtttgcagttttaaaattatgttttaaaatggactatcatatgcttaccgtaacttgaaagtatttcga |
| | removed. | tttcttgggtttatatatcttgtggaaaggacgaggatccgggtgtatgacacgttgtcggttttagagc |
| | Additionally, | tagaaatagcaagttaaaataaggctagtccgttatcaacttgaaaaagtggcaccgagtcggtgctt |
| | 395 bases | ttttctagacacaattgcatgaagaatctgcttagggttaggcgttttgcgctgcttcgcgatgtacgg |
| | were | gccagatatacgcgttgacattgattattgactagttattaatagtaatcaattacggggtcattagttca |
| | removed | tagcccatatatggagttccgcgttacataacttacggtaaatggcccgcctggctgaccgcccaac |
| | located | gacccccgcccattgacgtcaataatgacgtatgttcccatagtaacgccaatagggactttccattg |
| | between the | acgtcaatgggtggagtatttacggtaaactgcccacttggcagtacatcaagtgtatcatatgccaa |
| | origin of | gtacgccccctattgacgtcaatgacggtaaatggcccgcctggcattatgcccagtacatgacctt |
| | replication | atgggactttcctacttggcagtacatctacgtattagtcatcgctattaccatggtgatgcggttttggc |
| | and the sp6 | agtacatcaatgggcgtggatagcggtttgactcacggggatttccaagtctccaccccattgacgt |
| | promoter | caatgggagtttgttttggcaccaaaatcaacgggactttccaaaatgtcgtaacaactccgccccat |
| | | tgacgcaaatgggcggtaggcgtgtacggtgggaggtctatataagcagagctctctggctaacta |
| | | gagaacccactgcttactggcttatcgaaattaatacgactcactatagggagacccaagcttgcca |
| | | ccatggacaagaagtacagcatcggcctggacatcggtaccaacagcgtgggctgggccgtgat |
| | | caccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggccttgcaacgatttaggtgacactatagaagagaaggaatta |
| | | atacgactcactatagggagagagagagaattaccctcactaaagggaggagaagcatgaattcc |
| | | ccagtggaaagacgcgcaggcaaaacgcaccacgtgacggagcgtgaccgcgcgccgagcgc |
| | | gcgccaaggtcgggcaggaagagggcctatttcccatgattccttcatatttgcatatacgatacaag |
| | | gctgttagagagataattagaattaatttgactgtaaacacaaagatattagtacaaaatacgtgacgt |
| | | agaaagtaataatttcttgggtagtttgcagttttaaaattatgttttaaaatggactatcatatgcttaccg |
| | | taacttgaaagtatttcgatttcttgggtttatatatcttgtggaaaggacgaggatccgtgtttaacgac |
| | | atatcgccagttttagagctagaaatagcaagttaaaataaggctagtccgttatcaacttgaaaaagt |
| | | ggcaccgagtcggtgcttttttct |
| 59 | P21 smallest; | agacacaattccccagtggaaagacgcgcaggcaaaacgcaccacgtgacggagcgtgaccgc |
| | stop codon | gcgccgagcgcgcgccaaggtcgggcaggaagagggcctatttcccatgattccttcatatttgca |
| | was added | tatacgatacaaggctgttagagagataattagaattaatttgactgtaaacacaaagatattagtaca |
| | after the | aaatacgtgacgtagaaagtaataatttcttgggtagtttgcagttttaaaattatgttttaaaatggacta |
| | Cas9 operon | tcatatgcttaccgtaacttgaaagtatttcgatttcttgggtttatatatcttgtggaaaggacgaggat |
| | and the GFP | ccgtttacttcggcttttacaaggttttagagctagaaatagcaagttaaaataaggctagtccgttatc |
| | gene and | aacttgaaaaagtggcaccgagtcggtgcttttttctagacacaattccccagtggaaagacgcgca |
| | neomycin | ggcaaaacgcaccacgtgacggagcgtgaccgcgcgccgagcgcgcgccaaggtcgggcag |
| | genes | gaagagggcctatttcccatgattccttcatatttgcatatacgatacaaggctgttagagagataatta |
| | sequences | gaattaatttgactgtaaacacaaagatattagtacaaaatacgtgacgtagaaagtaataatttcttgg |
| | were | gtagtttgcagttttaaaattatgttttaaaatggactatcatatgcttaccgtaacttgaaagtatttcgat |
| | removed. | ttcttgggtttatatatcttgtggaaaggacgaggatccgaaaggggtccttcgaacacggttttagag |
| | Additionally, | ctagaaatagcaagttaaaataaggctagtccgttatcaacttgaaaaagtggcaccgagtcggtgc |
| | 395 bases | ttttttctagacacaattgcatgaagaatctgcttagggttaggcgttttgcgctgcttcgcgatgtacg |
| | were | ggccagatatacgcgttgacattgattattgactagttattaatagtaatcaattacggggtcattagttc |
| | removed | atagcccatatatggagttccgcgttacataacttacggtaaatggcccgcctggctgaccgcccaa |
| | located | cgacccccgcccattgacgtcaataatgacgtatgttcccatagtaacgccaatagggactttccatt |
| | between the | gacgtcaatgggtggagtatttacggtaaactgcccacttggcagtacatcaagtgtatcatatgcca |
| | origin of | agtacgccccctattgacgtcaatgacggtaaatggcccgcctggcattatgcccagtacatgacct |
| | replication | tatgggactttcctacttggcagtacatctacgtattagtcatcgctattaccatggtgatgcggttttgg |
| | and the sp6 | cagtacatcaatgggcgtggatagcggtttgactcacggggatttccaagtctccaccccattgacg |
| | promoter | tcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaact |
| | | agagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggccttgcaacgatttaggtgacactatagaagagaaggaatta |
| | | atacgactcactatagggagagagagagaattaccctcactaaagggaggagaagcatgaattcc |
| | | ccagtggaaagacgcgcaggcaaaacgcaccacgtgacggagcgtgaccgcgcgccgagcgc |
| | | gcgccaaggtcgggcaggaagagggcctatttcccatgattccttcatatttgcatatacgatacaag |
| | | gctgttagagagataattagaattaatttgactgtaaacacaaagatattagtacaaaatacgtgacgt |
| | | agaaagtaataatttcttgggtagtttgcagttttaaaattatgttttaaaatggactatcatatgcttaccg |
| | | taacttgaaagtatttcgatttcttgggtttatatatcttgtggaaaggacgaggatccgcatacgggaa |
| | | cgcacatagtgttttagagctagaaatagcaagttaaaataaggctagtccgttatcaacttgaaaaa |
| | | gtggcaccgagtcggtgcttttttct |
| 60 | P22 smallest; | agacacaattccccagtggaaagacgcgcaggcaaaacgcaccacgtgacggagcgtgaccgc |
| | stop codon | gcgccgagcgcgcgccaaggtcgggcaggaagagggcctatttcccatgattccttcatatttgca |
| | was added | tatacgatacaaggctgttagagagataattagaattaatttgactgtaaacacaaagatattagtaca |
| | after the | aaatacgtgacgtagaaagtaataatttcttgggtagtttgcagttttaaaattatgttttaaaatggacta |
| | Cas9 operon | tcatatgcttaccgtaacttgaaagtatttcgatttcttgggtttatatatcttgtggaaaggacgaggat |
| | and the GFP | ccgtttacttcggcttttacaaggttttagagctagaaatagcaagttaaaataaggctagtccgttatc |
| | gene and | aacttgaaaaagtggcaccgagtcggtgcttttttctagacacaattccccagtggaaagacgcgca |
| | neomycin | ggcaaaacgcaccacgtgacggagcgtgaccgcgcgccgagcgcgcgccaaggtcgggcag |
| | genes | gaagagggcctatttcccatgattccttcatatttgcatatacgatacaaggctgttagagagataatta |
| | sequences | gaattaatttgactgtaaacacaaagatattagtacaaaatacgtgacgtagaaagtaataatttcttgg |
| | were | gtagtttgcagttttaaaattatgttttaaaatggactatcatatgcttaccgtaacttgaaagtatttcgat |
| | removed. | ttcttgggtttatatatcttgtggaaaggacgaggatccgaaaggggtccttcgaacacggttttagag |
| | Additionally, | ctagaaatagcaagttaaaataaggctagtccgttatcaacttgaaaaagtggcaccgagtcggtgc |
| | 395 bases | ttttttctagacacaattgcatgaagaatctgcttagggttaggcgttttgcgctgcttcgcgatgtacg |
| | were | ggccagatatacgcgttgacattgattattgactagttattaatagtaatcaattacggggtcattagttc |
| | removed | atagcccatatatggagttccgcgttacataacttacggtaaatggcccgcctggctgaccgcccaa |
| | located | cgacccccgcccattgacgtcaataatgacgtatgttcccatagtaacgccaatagggactttccatt |
| | between the | gacgtcaatgggtggagtatttacggtaaactgcccacttggcagtacatcaagtgtatcatatgcca |
| | origin of | agtacgccccctattgacgtcaatgacggtaaatggcccgcctggcattatgcccagtacatgacct |
| | replication | tatgggactttcctacttggcagtacatctacgtattagtcatcgctattaccatggtgatgcggttttgg |
| | and the sp6 | cagtacatcaatgggcgtggatagcggtttgactcacggggatttccaagtctccaccccattgacg |
| | promoter | tcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaact |
| | | agagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gactgatgaccaccccaacttgtttattgcagcttataatggttacaaataaagcaatagcatcacaaa |
| | | tttcacaaataaagcatttttttcactgcattctagttgtggtttgtccaaactcatcaatgtatcttatcatg |
| | | tctgtataccgtcgacctctagctagagcttggcgtaatcatggtcatagctgtttcctgtgtgaaattgt |
| | | tatccgctcacaattccacacaacatacgagccggaagcataaaggtaagcctgaatattgaaaaa |
| | | ggaagagtatgagtattcaacatttccgtgtcgcccttattcccttttttgcggcattttgccttcctgttttt |
| | | gctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgat |
| | | gagcacttttaaagttctgctatgtggcgcggtattatcccgtattgacgccgggcaagagcaactcg |
| | | gtcgccgcatacactattctcagaatgacttggttgagtactcaccagtcacagaaaagcatcttacg |
| | | gatggcatgacagtaagagaattatgcagtgctgccataaccatgagtgataacactgcggccaac |
| | | ttacttctgacaacgatcggaggaccgaaggagctaaccgcttttttgcacaacatgggggatcatg |
| | | taactcgccttgatcgttgggaaccggagctgaatgaagccataccaaacgacgagcgtgacacc |
| | | acgatgcctgtagcaatggcaacaacgttgcgcaaactattaactggcgaactacttactctagcttc |
| | | ccggcaacaattaatagactggatggaggcggataaagttgcaggaccacttctgcgctcggccct |
| | | tccggctggctggtttattgctgataaatctggagccggtgagcgtgggtctcgcggtatcattgcag |
| | | cactggggccagatggtaagccctcccgtatcgtagttatctacacgacggggagtcaggcaacta |
| | | tggatgaacgaaatagacagatcgctgagataggtgcctcactgattaagcattggtaactgtcaga |
| | | ccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtag |
| | | aaaagatcaaaggatcttcttgagatcctttttttctgcgcgtaatctgctgcttgcaaacaaaaaaacc |
| | | accgctaccagcggtggtttttttgccggatcaagagctaccaactctttttccgaaggtaactggctt |
| | | cagcagagcgcagataccaaatactgttcttctagtgtagccgtagttaggccaccacttcaagaact |
| | | ctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacg |
| | | gggggttcgtgcacacagcccagcttggagcgaacgacctacaccgaactgagatacctacagc |
| | | gtgagctatgagaaagcgccacgcttcccgaagggagaaaggcggacaggtatccggtaagcg |
| | | gcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcc |
| | | tatggaaaaacgccagcaacgcggccttgcaacgatttaggtgacactatagaagagaaggaatta |
| | | atacgactcactatagggagagagagagaattaccctcactaaagggaggagaagcatgaattcc |
| | | ccagtggaaagacgcgcaggcaaaacgcaccacgtgacggagcgtgaccgcgcgccgagcgc |
| | | gcgccaaggtcgggcaggaagagggcctatttcccatgattccttcatatttgcatatacgatacaag |
| | | gctgttagagagataattagaattaatttgactgtaaacacaaagatattagtacaaaatacgtgacgt |
| | | agaaagtaataatttcttgggtagtttgcagttttaaaattatgttttaaaatggactatcatatgcttaccg |
| | | taacttgaaagtatttcgatttcttgggtttatatatcttgtggaaaggacgaggatccgcatacgggaa |
| | | cgcacatagtgttttagagctagaaatagcaagttaaaataaggctagtccgttatcaacttgaaaaa |
| | | gtggcaccgagtcggtgcttttttct |
| 71 | P18 | agacacaattccccagtggaaagacgcgcaggcaaaacgcaccacgtgacggagcgtgaccgc |
| | NLS_removee | gcgccgagcgcgcgccaaggtcgggcaggaagagggcctatttcccatgattccttcatatttgca |
| | (the | tatacgatacaaggctgttagagagataattagaattaatttgactgtaaacacaaagatattagtaca |
| | Nuclear | aaatacgtgacgtagaaagtaataatttcttgggtagtttgcagttttaaaattatgttttaaaatggacta |
| | Localization | tcatatgcttaccgtaacttgaaagtatttcgatttcttgggtttatatatcttgtggaaaggacgaggat |
| | Sequence | ccgcaactcatagagagttagcggttttagagctagaaatagcaagttaaaataaggctagtccgtta |
| | after the | tcaacttgaaaaagtggcaccgagtcggtgcttttttctagacacaattccccagtggaaagacgcg |
| | Cas9 | caggcaaaacgcaccacgtgacggagcgtgaccgcgcgccgagcgcgcgccaaggtcgggca |
| | sequence was | ggaagagggcctatttcccatgattccttcatatttgcatatacgatacaaggctgttagagagataatt |
| | changed to a | agaattaatttgactgtaaacacaaagatattagtacaaaatacgtgacgtagaaagtaataatttcttg |
| | poly-serine | ggtagtttgcagttttaaaattatgttttaaaatggactatcatatgcttaccgtaacttgaaagtatttcga |
| | sequence | tttcttgggtttatatatcttgtggaaaggacgaggatccgcgtggtttagagaagcgcacgttttaga |
| | (i.e. | gctagaaatagcaagttaaaataaggctagtccgttatcaacttgaaaaagtggcaccgagtcggtg |
| | KKKRK → | cttttttctagacacaattgcatgaagaatctgcttagggttaggcgttttgcgctgcttcgcgatgtac |
| | SAGSSG) | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCgtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgctaggcg |
| | | gccgcagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | acaaaatacgtgacgtagaaagtaataatttcttgggtagtttgcagttttaaaattatgttttaaaatgg |
| | | actatcatatgcttaccgtaacttgaaagtatttcgatttcttgggtttatatatcttgtggaaaggacgag |
| | | gatccgcctctgaccttaattatagggttttagagctagaaatagcaagttaaaataaggctagtccgt |
| | | tatcaacttgaaaaagtggcaccgagtcggtgcttttttct |
| 72 | P19 | agacacaattgcatgaagaatctgcttagggttaggcgttttgcgctgcttcgcgatgtacgggcca |
| | NLS removed | gatatacgcgttgacattgattattgactagttattaatagtaatcaattacggggtcattagttcatagc |
| | (the | ccatatatggagttccgcgttacataacttacggtaaatggcccgcctggctgaccgcccaacgacc |
| | Nuclear | cccgcccattgacgtcaataatgacgtatgttcccatagtaacgccaatagggactttccattgacgt |
| | Localization | caatgggtggagtatttacggtaaactgcccacttggcagtacatcaagtgtatcatatgccaagtac |
| | Sequence | gccccctattgacgtcaatgacggtaaatggcccgcctggcattatgcccagtacatgaccttatgg |
| | after the | gactttcctacttggcagtacatctacgtattagtcatcgctattaccatggtgatgcggttttggcagta |
| | Cas9 | catcaatgggcgtggatagcggtttgactcacggggatttccaagtctccaccccattgacgtcaat |
| | sequence was | gggagtttgttttggcaccaaaatcaacgggactttccaaaatgtcgtaacaactccgccccattgac |
| | changed to a | gcaaatgggcggtaggcgtgtacggtgggaggtctatataagcagagctctctggctaactagaga |
| | poly-serine | acccactgcttactggcttatcgaaattaatacgactcactatagggagacccaagcttgccaccatg |
| | sequence | gacaagaagtacagcatcggcctggacatcggtaccaacagcgtgggctgggccgtgatcaccg |
| | (i.e. | acgagtacaaggtgcccagcaagaagttcaaggtgctgggcaacaccgaccgccacagcatcaa |
| | KKKRK → | gaagaacctgatcggcgccctgctgttcgacagcggcgagaccgccgaggccacccgcctgaa |
| | SAGSSG) | gcgcaccgcccgccgccgctacacccgccgcaagaaccgcatctgctacctgcaggagatcttc |
| | | agcaacgagatggccaaggtggacgacagcttcttccaccgcctggaggagagcttcctggtgga |
| | | ggaggacaagaagcacgagcgccaccccatcttcggcaacatcgtggacgaggtggcctaccac |
| | | gagaagtaccccaccatctaccacctgcgcaagaagctggtggacagcaccgacaaggccgacc |
| | | tgcgcctgatctacctggccctggcccacatgatcaagttccgcggccacttcctgatcgagggcg |
| | | acctgaaccccgacaacagcgacgtggacaagctgttcatccagctggtgcagacctacaaccag |
| | | ctgttcgaggagaaccccatcaacgccagcggcgtggacgccaaggccatcctgagcgcccgcc |
| | | tgagcaagagccgccgcctggagaacctgatcgcccagctgcccggcgagaagaagaacggcc |
| | | tgttcggcaacctgatcgccctgagcctgggcctgacccccaacttcaagagcaacttcgacctgg |
| | | ccgaggacgccaagctgcagctgagcaaggacacctacgacgacgacctggacaacctgctgg |
| | | cccagatcggcgaccagtacgccgacctgttcctggccgccaagaacctgagcgacgccatcctg |
| | | ctgagcgacatcctgcgcgtgaacaccgagatcaccaaggcccccctgagcgccagcatgatca |
| | | agcgctacgacgagcaccaccaggacctgaccctgctgaaggccctggtgcgccagcagctgcc |
| | | cgagaagtacaaggagatcttcttcgaccagagcaagaacggctacgccggctacatcgacggc |
| | | ggcgccagccaggaggagttctacaagttcatcaagcccatcctggagaagatggacggcaccg |
| | | aggagctgctggtgaagctgaaccgcgaggacctgctgcgcaagcagcgcaccttcgacaacgg |
| | | cagcatcccccaccagatccacctgggcgagctgcacgccatcctgcgccgccaggaggacttct |
| | | accccttcctgaaggacaaccgcgagaagatcgagaagatcctgaccttccgcatcccctactacg |
| | | tgggccccctggcccgcggcaacagccgcttcgcctggatgacccgcaagagcgaggagacca |
| | | tcaccccctggaacttcgaggaggtggtggacaagggcgccagcgcccagagcttcatcgagcg |
| | | catgaccaacttcgacaagaacctgcccaacgagaaggtgctgcccaagcacagcctgctgtacg |
| | | agtacttcaccgtgtacaacgagctgaccaaggtgaagtacgtgaccgagggcatgcgcaagccc |
| | | gccttcctgagcggcgagcagaagaaggccatcgtggacctgctgttcaagaccaaccgcaaggt |
| | | gaccgtgaagcagctgaaggaggactacttcaagaagatcgagtgcttcgacagcgtggagatca |
| | | gcggcgtggaggaccgcttcaacgccagcctgggcacctaccacgacctgctgaagatcatcaa |
| | | ggacaaggacttcctggacaacgaggagaacgaggacatcctggaggacatcgtgctgaccctg |
| | | accctgttcgaggaccgcgagatgatcgaggagcgcctgaagacctacgcccacctgttcgacga |
| | | caaggtgatgaagcagctgaagcgccgccgctacaccggctggggccgcctgagccgcaagctt |
| | | atcaacggcatccgcgacaagcagagcggcaagaccatcctggacttcctgaagagcgacggct |
| | | tcgccaaccgcaacttcatgcagctgatccacgacgacagcctgaccttcaaggaggacatccag |
| | | aaggcccaggtgagcggccagggcgacagcctgcacgagcacatcgccaacctggccggcag |
| | | ccccgccatcaagaagggcatcctgcagaccgtgaaggtggtggacgagctggtgaaggtgatg |
| | | ggccgccacaagcccgagaacatcgtgatcgagatggcccgcgagaaccagaccacccagaag |
| | | ggccagaagaacagccgcgagcgcatgaagcgcatcgaggagggcatcaaggagctgggcag |
| | | ccagatcctgaaggagcaccccgtggagaacacccagctgcagaacgagaagctgtacctgtact |
| | | acctgcagaacggccgcgacatgtacgtggaccaggagctggacatcaaccgcctgagcgacta |
| | | cgacgtggaccacatcgtgccccagagcttcctgaaggacgacagcatcgacaacaaggtgctg |
| | | acccgcagcgacaagaaccgcggcaagagcgacaacgtgcccagcgaggaggtggtgaagaa |
| | | gatgaagaactactggcgccagctgctgaacgccaagctgatcacccagcgcaagttcgacaacc |
| | | tgaccaaggccgagcgcggcggcctgagcgagctggacaaggccggcttcatcaagcgccagc |
| | | tggtggagacccgccagatcaccaagcacgtggcccagatcctggacagccgcatgaacaccaa |
| | | gtacgacgagaacgacaagctgatccgcgaggtgaaggtgatcaccctgaagagcaagctggtg |
| | | agcgacttccgcaaggacttccagttctacaaggtgcgcgagatcaacaactaccaccacgccca |
| | | cgacgcctacctgaacgccgtggtgggcaccgccctgatcaagaagtaccccaagctggagagc |
| | | gagttcgtgtacggcgactacaaggtgtacgacgtgcgcaagatgatcgccaagagcgagcagg |
| | | agatcggcaaggccaccgccaagtacttcttctacagcaacatcatgaacttcttcaagaccgagat |
| | | caccctggccaacggcgagatccgcaagcgccccctgatcgagaccaacggcgagaccggcg |
| | | agatcgtgtgggacaagggccgcgacttcgccaccgtgcgcaaggtgctgagcatgccccaggt |
| | | gaacatcgtgaagaagaccgaggtgcagaccggcggcttcagcaaggagagcatcctgcccaa |
| | | gcgcaacagcgacaagctgatcgcccgcaagaaggactgggaccccaagaagtacggcggctt |
| | | cgacagccccaccgtggcctacagcgtgctggtggtggccaaggtggagaagggcaagagcaa |
| | | gaagctgaagagcgtgaaggagctgctgggcatcaccatcatggagcgcagcagcttcgagaag |
| | | aaccccatcgacttcctggaggccaagggctacaaggaggtgaagaaggacctgatcatcaagct |
| | | gcccaagtacagcctgttcgagctggagaacggccgcaagcgcatgctggccagcgccggcga |
| | | gctgcagaagggcaacgagctggccctgcccagcaagtacgtgaacttcctgtacctggccagcc |
| | | actacgagaagctgaagggcagccccgaggacaacgagcagaagcagctgttcgtggagcagc |
| | | acaagcactacctggacgagatcatcgagcagatcagcgagttcagcaagcgcgtgatcctggcc |
| | | gacgccaacctggacaaggtgctgagcgcctacaacaagcaccgcgacaagcccatccgcgag |
| | | caggccgagaacatcatccacctgttcaccctgaccaacctgggcgcccccgccgccttcaagta |
| | | cttcgacaccaccatcgaccgcaagcgctacaccagcaccaaggaggtgctggacgccaccctg |
| | | atccaccagagcatcaccggtctgtacgagacccgcatcgacctgagccagctgggcggcgacg |
| | | gcggctccggacctccaaGCGCCGGCAGCAGCGGCgtatacccctacgacgtgcc |
| | | cgactacgccctcgaggagggcagaggaagtcttctaacatgcggtgacgtggaggagaatccc |
| | | ggccctatggagagcgacgagagcggcctgcccgccatggagatcgagtgccgcatcaccggc |
| | | accctgaacggcgtggagttcgagctggtgggcggcggagagggcacccccaagcagggccg |
| | | catgaccaacaagatgaagagcaccaaaggcgccctgaccttcagcccctacctgctgagccacg |
| | | tgatgggctacggcttctaccacttcggcacctaccccagcggctacgagaaccccttcctgcacg |
| | | ccatcaacaacggcggctacaccaacacccgcatcgagaagtacgaggacggcggcgtgctgc |
| | | acgtgagcttcagctaccgctacgaggccggccgcgtgatcggcgacttcaaggtggtgggcacc |
| | | ggcttccccgaggacagcgtgatcttcaccgacaagatcatccgcagcaacgccaccgtggagca |
| | | cctgcaccccatgggcgataacgtgctggtgggcagcttcgcccgcaccttcagcctgcgcgacg |
| | | gcggctactacagcttcgtggtggacagccacatgcacttcaagagcgccatccaccccagcatcc |
| | | tgcagaacgggggccccatgttcgccttccgccgcgtggaggagctgcacagcaacaccgagct |
| | | gggcatcgtggagtaccagcacgccttcaagacccccatcgccttcgccagatcccgcgctcagt |
| | | cgtccaattctgccgtggacggcaccgccggacccggctccaccggatctcgctaggcggccgc |
| | | agatgggggtcctgggccccagggtgtgcagccactgacttggggactgctggtggggtaggga |
| | | tgagggagggaggggcattgtgatgtacagggctgctctgtgagatcaagggtctcttaagggtgg |
| | | gagctggggcagggactacgagagcagccagatgggctgaaagtggaactcaaggggtttctgg |
| | | cacctacctacctgcttcccgctggggggtggggagttggcccagagtcttaagattggggcagg |
| | | gtggagaggtgggctcttcctgcttcccactcatcttatagctttctttccccagatccgaattggagat |
| | | ccaaaccaaggcgcgcgctagcgccaccatgggatcggccattgaacaagatggattgcacgca |
| | | ggttctccggccgcttgggtggagaggctattcggctatgactgggcacaacagacaatcggctgc |
| | | tctgatgccgccgtgttccggctgtcagcgcaggggcgcccggttctttttgtcaagaccgacctgt |
| | | ccggtgccctgaatgaactgcaggacgaggcagcgcggctatcgtggctggccacgacgggcgt |
| | | tccttgcgcagcagtgctcgacgttgtcactgaagcgggaagggactggctgctattgggcgaagt |
| | | gccggggcaggatctcctgtcatctcaccttgctcctgccgagaaagtatccatcatggctgatgca |
| | | atgcggcggctgcatacgcttgatccggctacctgcccattcgaccaccaagcgaaacatcgcatc |
| | | gagcgagcacgtactcggatggaagccggtcttgtcgatcaggatgatctggacgaagagcatca |
| | | ggggctcgcgccagccgaactgttcgccaggctcaaggcgcgtatgcccgacggcgatgatctc |
| | | gtcgtgactcatggcgatgcctgcttgccgaatatcatggtggaaaatggccgcttttctggattcatc |
| | | gactgtggccggctgggtgtggcggaccgctatcaggacatagcgttggctacccgtgatattgct |
| | | gaagagcttggcggcgaatgggctgaccgcttcctcgtgctttacggtatcgccgctcccgattcgc |
| | | agcgcatcgccttctatcgccttcttgacgagttcttctgaacgcggtgctacgagatttcgattccac |
| | | cgccgccttctatgaaaggttgggcttcggaatcgttttccgggacgccggctggatgatcctccag |
| | | cgcggggatctcatgctggagttcttcgcccaccccaacttgtttattgcagcttataatggttacaaat |
| | | aaagcaatagcatcacaaatttcacaaataaagcatttttttcactgcattctagttgtggtttgtccaaa |
| | | ctcatcaatgtatcttatcatgtctgtataccgtcgacctctagctagagcttggcgtaatcatggtcata |
| | | gctgtttcctgtgtgaaattgttatccgctcacaattccacacaacatacgagccggaagcataaagg |
| | | taagcctgaatattgaaaaaggaagagtatgagtattcaacatttccgtgtcgcccttattcccttttttg |
| | | cggcattttgccttcctgtttttgctcacccagaaacgctggtgaaagtaaaagatgctgaagatcagt |
| | | tgggtgcacgagtgggttacatcgaactggatctcaacagcggtaagatccttgagagttttcgccc |
| | | cgaagaacgttttccaatgatgagcacttttaaagttctgctatgtggcgcggtattatcccgtattgac |
| | | gccgggcaagagcaactcggtcgccgcatacactattctcagaatgacttggttgagtactcacca |
| | | gtcacagaaaagcatcttacggatggcatgacagtaagagaattatgcagtgctgccataaccatga |
| | | gtgataacactgcggccaacttacttctgacaacgatcggaggaccgaaggagctaaccgctttttt |
| | | gcacaacatgggggatcatgtaactcgccttgatcgttgggaaccggagctgaatgaagccatacc |
| | | aaacgacgagcgtgacaccacgatgcctgtagcaatggcaacaacgttgcgcaaactattaactg |
| | | gcgaactacttactctagcttcccggcaacaattaatagactggatggaggcggataaagttgcagg |
| | | accacttctgcgctcggcccttccggctggctggtttattgctgataaatctggagccggtgagcgtg |
| | | ggtctcgcggtatcattgcagcactggggccagatggtaagccctcccgtatcgtagttatctacac |
| | | gacggggagtcaggcaactatggatgaacgaaatagacagatcgctgagataggtgcctcactga |
| | | ttaagcattggtaactgtcagaccaagtttactcatatatactttagattgatttaaaacttcatttttaattt |
| | | aaaaggatctaggtgaagatcctttttgataatctcatgaccaaaatcccttaacgtgagttttcgttcc |
| | | actgagcgtcagaccccgtagaaaagatcaaaggatcttcttgagatcctttttttctgcgcgtaatct |
| | | gctgcttgcaaacaaaaaaaccaccgctaccagcggtggtttttttgccggatcaagagctaccaac |
| | | tctttttccgaaggtaactggcttcagcagagcgcagataccaaatactgttcttctagtgtagccgta |
| | | gttaggccaccacttcaagaactctgtagcaccgcctacatacctcgctctgctaatcctgttaccagt |
| | | ggctgctgccagtggcgataagtcgtgtcttaccgggttggactcaagacgatagttaccggataag |
| | | gcgcagcggtcgggctgaacggggggttcgtgcacacagcccagcttggagcgaacgacctac |
| | | accgaactgagatacctacagcgtgagctatgagaaagcgccacgcttcccgaagggagaaagg |
| | | cggacaggtatccggtaagcggcagggtcggaacaggagagcgcacgagggagcttccaggg |
| | | ggaaacgcctggtatctttatagtcctgtcgggtttcgccacctctgacttgagcgtcgatttttgtgat |
| | | gctcgtcaggggggcggagcctatggaaaaacgccagcaacgcggcctttttacggttcctggcc |
| | | ttttgctggccttttgctcacatgttctttcctgcgttatcccctgattctgtggataaccgtattaccgcct |
| | | ttgagtgagctgataccgctcgccgcagccgaacgaccgagcgcagcgagtcagtgagcgagg |
| | | aagcggaagagcgcccaatacgcaaaccgcctctccccgcgcgttggccgattcattaatgcagc |
| | | tggcacgacaggtttcccgactggaaagcgggcagtgagcgcaacgcaattaatgtgagttagctc |
| | | actcattaggcaccccaggctttacactttatgcttccggctcgtatgttgtgtggaattgtgagcggat |
| | | aacaatttcacacaggaaacagctatgacatgattacgaattgcaacgatttaggtgacactatagaa |
| | | gagaaggaattaatacgactcactatagggagagagagagaattaccctcactaaagggaggaga |
| | | agcatgaattccccagtggaaagacgcgcaggcaaaacgcaccacgtgacggagcgtgaccgc |
| | | gcgccgagcgcgcgccaaggtcgggcaggaagagggcctatttcccatgattccttcatatttgca |
| | | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | | tcatatgcttaccgtaacttgaaagtatttcgatttcttgggtttatatatcttgtggaaaggacgaggat |
| | | ccggagaagcttctctgttttagagctagaaatagcaagttaaaataaggctagtccgttatcaacttg |
| | | aaaaagtggcaccgagtcggtgcttttttct |
| 73 | P20 | agacacaattccccagtggaaagacgcgcaggcaaaacgcaccacgtgacggagcgtgaccgc |
| | NLS_removed | gcgccgagcgcgcgccaaggtcgggcaggaagagggcctatttcccatgattccttcatatttgca |
| | (the | tatacgatacaaggctgttagagagataattagaattaatttgactgtaaacacaaagatattagtaca |
| | | aaatacgtgacgtagaaagtaataatttcttgggtagtttgcagttttaaaattatgttttaaaatggacta |
| | Nuclear | tcatatgcttaccgtaacttgaaagtatttcgatttcttgggtttatatatcttgtggaaaggacgaggat |
| | Localization | ccgaccaagatctggacgggtggttttagagctagaaatagcaagttaaaataaggctagtccgtta |
| | Sequence | tcaacttgaaaaagtggcaccgagtcggtgcttttttctagacacaattccccagtggaaagacgcg |
| | after the | caggcaaaacgcaccacgtgacggagcgtgaccgcgcgccgagcgcgcgccaaggtcgggca |
| | Cas9 | ggaagagggcctatttcccatgattccttcatatttgcatatacgatacaaggctgttagagagataatt |
| | sequence was | agaattaatttgactgtaaacacaaagatattagtacaaaatacgtgacgtagaaagtaataatttcttg |
| | changed to a | ggtagtttgcagttttaaaattatgttttaaaatggactatcatatgcttaccgtaacttgaaagtatttcga |
| | poly-serine | tttcttgggtttatatatcttgtggaaaggacgaggatccgggtgtatgacacgttgtcggttttagagc |
| | sequence | tagaaatagcaagttaaaataaggctagtccgttatcaacttgaaaaagtggcaccgagtcggtgctt |
| | (i.e. | ttttctagacacaattgcatgaagaatctgcttagggttaggcgttttgcgctgcttcgcgatgtacgg |
| | KKKRK → | gccagatatacgcgttgacattgattattgactagttattaatagtaatcaattacggggtcattagttca |
| | SAGSSG) | tagcccatatatggagttccgcgttacataacttacggtaaatggcccgcctggctgaccgcccaac |
| | | gacccccgcccattgacgtcaataatgacgtatgttcccatagtaacgccaatagggactttccattg |
| | | acgtcaatgggtggagtatttacggtaaactgcccacttggcagtacatcaagtgtatcatatgccaa |
| | | gtacgccccctattgacgtcaatgacggtaaatggcccgcctggcattatgcccagtacatgacctt |
| | | atgggactttcctacttggcagtacatctacgtattagtcatcgctattaccatggtgatgcggttttggc |
| | | agtacatcaatgggcgtggatagcggtttgactcacggggatttccaagtctccaccccattgacgt |
| | | caatgggagtttgttttggcaccaaaatcaacgggactttccaaaatgtcgtaacaactccgccccat |
| | | tgacgcaaatgggcggtaggcgtgtacggtgggaggtctatataagcagagctctctggctaacta |
| | | gagaacccactgcttactggcttatcgaaattaatacgactcactatagggagacccaagcttgcca |
| | | ccatggacaagaagtacagcatcggcctggacatcggtaccaacagcgtgggctgggccgtgat |
| | | caccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCgtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgctaggcg |
| | | gccgcagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | acaaaatacgtgacgtagaaagtaataatttcttgggtagtttgcagttttaaaattatgttttaaaatgg |
| | | actatcatatgcttaccgtaacttgaaagtatttcgatttcttgggtttatatatcttgtggaaaggacgag |
| | | gatccgtgtttaacgacatatcgccagttttagagctagaaatagcaagttaaaataaggctagtccgt |
| | | tatcaacttgaaaaagtggcaccgagtcggtgcttttttct |
| 74 | P21 NLS | agacacaattccccagtggaaagacgcgcaggcaaaacgcaccacgtgacggagcgtgaccgc |
| | removed (the | gcgccgagcgcgcgccaaggtcgggcaggaagagggcctatttcccatgattccttcatatttgca |
| | Nuclear | tatacgatacaaggctgttagagagataattagaattaatttgactgtaaacacaaagatattagtaca |
| | Localization | aaatacgtgacgtagaaagtaataatttcttgggtagtttgcagttttaaaattatgttttaaaatggacta |
| | Sequence | tcatatgcttaccgtaacttgaaagtatttcgatttcttgggtttatatatcttgtggaaaggacgaggat |
| | after the | ccgtttacttcggcttttacaaggttttagagctagaaatagcaagttaaaataaggctagtccgttatc |
| | Cas9 | aacttgaaaaagtggcaccgagtcggtgcttttttctagacacaattccccagtggaaagacgcgca |
| | sequence was | ggcaaaacgcaccacgtgacggagcgtgaccgcgcgccgagcgcgcgccaaggtcgggcag |
| | changed to a | gaagagggcctatttcccatgattccttcatatttgcatatacgatacaaggctgttagagagataatta |
| | poly-serine | gaattaatttgactgtaaacacaaagatattagtacaaaatacgtgacgtagaaagtaataatttcttgg |
| | sequence | gtagtttgcagttttaaaattatgttttaaaatggactatcatatgcttaccgtaacttgaaagtatttcgat |
| | (i.e. | ttcttgggtttatatatcttgtggaaaggacgaggatccgaaaggggtccttcgaacacggttttagag |
| | KKKRK → | ctagaaatagcaagttaaaataaggctagtccgttatcaacttgaaaaagtggcaccgagtcggtgc |
| | SAGSSG) | ttttttctagacacaattgcatgaagaatctgcttagggttaggcgttttgcgctgcttcgcgatgtacg |
| | | ggccagatatacgcgttgacattgattattgactagttattaatagtaatcaattacggggtcattagttc |
| | | atagcccatatatggagttccgcgttacataacttacggtaaatggcccgcctggctgaccgcccaa |
| | | cgacccccgcccattgacgtcaataatgacgtatgttcccatagtaacgccaatagggactttccatt |
| | | gacgtcaatgggtggagtatttacggtaaactgcccacttggcagtacatcaagtgtatcatatgcca |
| | | agtacgccccctattgacgtcaatgacggtaaatggcccgcctggcattatgcccagtacatgacct |
| | | tatgggactttcctacttggcagtacatctacgtattagtcatcgctattaccatggtgatgcggttttgg |
| | | cagtacatcaatgggcgtggatagcggtttgactcacggggatttccaagtctccaccccattgacg |
| | | tcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaact |
| | | agagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCtacccctacgacgtg |
| | | cccgactacgccctcgaggagggcagaggaagtcttctaacatgcggtgacgtggaggagaatc |
| | | ccggccctatggagagcgacgagagcggcctgcccgccatggagatcgagtgccgcatcaccg |
| | | gcaccctgaacggcgtggagttcgagctggtgggcggcggagagggcacccccaagcagggc |
| | | cgcatgaccaacaagatgaagagcaccaaaggcgccctgaccttcagcccctacctgctgagcca |
| | | cgtgatgggctacggcttctaccacttcggcacctaccccagcggctacgagaaccccttcctgca |
| | | cgccatcaacaacggcggctacaccaacacccgcatcgagaagtacgaggacggcggcgtgct |
| | | gcacgtgagcttcagctaccgctacgaggccggccgcgtgatcggcgacttcaaggtggtgggc |
| | | accggcttccccgaggacagcgtgatcttcaccgacaagatcatccgcagcaacgccaccgtgga |
| | | gcacctgcaccccatgggcgataacgtgctggtgggcagcttcgcccgcaccttcagcctgcgcg |
| | | acggcggctactacagcttcgtggtggacagccacatgcacttcaagagcgccatccaccccagc |
| | | atcctgcagaacgggggccccatgttcgccttccgccgcgtggaggagctgcacagcaacaccg |
| | | agctgggcatcgtggagtaccagcacgccttcaagacccccatcgccttcgccagatcccgcgct |
| | | cagtcgtccaattctgccgtggacggcaccgccggacccggctccaccggatctcgctaggcggc |
| | | cgcagatgggggtcctgggccccagggtgtgcagccactgacttggggactgctggtggggtag |
| | | ggatgagggagggaggggcattgtgatgtacagggctgctctgtgagatcaagggtctcttaagg |
| | | gtgggagctggggcagggactacgagagcagccagatgggctgaaagtggaactcaaggggttt |
| | | ctggcacctacctacctgcttcccgctggggggtggggagttggcccagagtcttaagattggggc |
| | | agggtggagaggtgggctcttcctgcttcccactcatcttatagctttctttccccagatccgaattgg |
| | | agatccaaaccaaggcgcgcgctagcgccaccatgggatcggccattgaacaagatggattgca |
| | | cgcaggttctccggccgcttgggtggagaggctattcggctatgactgggcacaacagacaatcg |
| | | gctgctctgatgccgccgtgttccggctgtcagcgcaggggcgcccggttctttttgtcaagaccga |
| | | cctgtccggtgccctgaatgaactgcaggacgaggcagcgcggctatcgtggctggccacgacg |
| | | ggcgttccttgcgcagcagtgctcgacgttgtcactgaagcgggaagggactggctgctattgggc |
| | | gaagtgccggggcaggatctcctgtcatctcaccttgctcctgccgagaaagtatccatcatggctg |
| | | atgcaatgcggcggctgcatacgcttgatccggctacctgcccattcgaccaccaagcgaaacatc |
| | | gcatcgagcgagcacgtactcggatggaagccggtcttgtcgatcaggatgatctggacgaagag |
| | | catcaggggctcgcgccagccgaactgttcgccaggctcaaggcgcgtatgcccgacggcgatg |
| | | atctcgtcgtgactcatggcgatgcctgcttgccgaatatcatggtggaaaatggccgcttttctggat |
| | | tcatcgactgtggccggctgggtgtggcggaccgctatcaggacatagcgttggctacccgtgata |
| | | ttgctgaagagcttggcggcgaatgggctgaccgcttcctcgtgctttacggtatcgccgctcccga |
| | | ttcgcagcgcatcgccttctatcgccttcttgacgagttcttctgaacgcggtgctacgagatttcgatt |
| | | ccaccgccgccttctatgaaaggttgggcttcggaatcgttttccgggacgccggctggatgatcct |
| | | ccagcgcggggatctcatgctggagttcttcgcccaccccaacttgtttattgcagcttataatggtta |
| | | caaataaagcaatagcatcacaaatttcacaaataaagcatttttttcactgcattctagttgtggtttgtc |
| | | caaactcatcaatgtatcttatcatgtctgtataccgtcgacctctagctagagcttggcgtaatcatgg |
| | | tcatagctgtttcctgtgtgaaattgttatccgctcacaattccacacaacatacgagccggaagcata |
| | | aaggtaagcctgaatattgaaaaaggaagagtatgagtattcaacatttccgtgtcgcccttattccct |
| | | tttttgcggcattttgccttcctgtttttgctcacccagaaacgctggtgaaagtaaaagatgctgaaga |
| | | tcagttgggtgcacgagtgggttacatcgaactggatctcaacagcggtaagatccttgagagttttc |
| | | gccccgaagaacgttttccaatgatgagcacttttaaagttctgctatgtggcgcggtattatcccgtat |
| | | tgacgccgggcaagagcaactcggtcgccgcatacactattctcagaatgacttggttgagtactca |
| | | ccagtcacagaaaagcatcttacggatggcatgacagtaagagaattatgcagtgctgccataacc |
| | | atgagtgataacactgcggccaacttacttctgacaacgatcggaggaccgaaggagctaaccgct |
| | | tttttgcacaacatgggggatcatgtaactcgccttgatcgttgggaaccggagctgaatgaagccat |
| | | accaaacgacgagcgtgacaccacgatgcctgtagcaatggcaacaacgttgcgcaaactattaa |
| | | ctggcgaactacttactctagcttcccggcaacaattaatagactggatggaggcggataaagttgc |
| | | aggaccacttctgcgctcggcccttccggctggctggtttattgctgataaatctggagccggtgag |
| | | cgtgggtctcgcggtatcattgcagcactggggccagatggtaagccctcccgtatcgtagttatcta |
| | | cacgacggggagtcaggcaactatggatgaacgaaatagacagatcgctgagataggtgcctcac |
| | | tgattaagcattggtaactgtcagaccaagtttactcatatatactttagattgatttaaaacttcattttta |
| | | atttaaaaggatctaggtgaagatcctttttgataatctcatgaccaaaatcccttaacgtgagttttcgtt |
| | | ccactgagcgtcagaccccgtagaaaagatcaaaggatcttcttgagatcctttttttctgcgcgtaat |
| | | ctgctgcttgcaaacaaaaaaaccaccgctaccagcggtggtttttttgccggatcaagagctacca |
| | | actctttttccgaaggtaactggcttcagcagagcgcagataccaaatactgttcttctagtgtagccg |
| | | tagttaggccaccacttcaagaactctgtagcaccgcctacatacctcgctctgctaatcctgttacca |
| | | gtggctgctgccagtggcgataagtcgtgtcttaccgggttggactcaagacgatagttaccggata |
| | | aggcgcagcggtcgggctgaacggggggttcgtgcacacagcccagcttggagcgaacgacct |
| | | acaccgaactgagatacctacagcgtgagctatgagaaagcgccacgcttcccgaagggagaaa |
| | | ggcggacaggtatccggtaagcggcagggtcggaacaggagagcgcacgagggagcttccag |
| | | ggggaaacgcctggtatctttatagtcctgtcgggtttcgccacctctgacttgagcgtcgatttttgtg |
| | | atgctcgtcaggggggcggagcctatggaaaaacgccagcaacgcggcctttttacggttcctgg |
| | | ccttttgctggccttttgctcacatgttctttcctgcgttatcccctgattctgtggataaccgtattaccgc |
| | | ctttgagtgagctgataccgctcgccgcagccgaacgaccgagcgcagcgagtcagtgagcgag |
| | | gaagcggaagagcgcccaatacgcaaaccgcctctccccgcgcgttggccgattcattaatgcag |
| | | ctggcacgacaggtttcccgactggaaagcgggcagtgagcgcaacgcaattaatgtgagttagct |
| | | cactcattaggcaccccaggctttacactttatgcttccggctcgtatgttgtgtggaattgtgagcgg |
| | | ataacaatttcacacaggaaacagctatgacatgattacgaattgcaacgatttaggtgacactatag |
| | | aagagaaggaattaatacgactcactatagggagagagagagaattaccctcactaaagggagga |
| | | gaagcatgaattccccagtggaaagacgcgcaggcaaaacgcaccacgtgacggagcgtgacc |
| | | gcgcgccgagcgcgcgccaaggtcgggcaggaagagggcctatttcccatgattccttcatatttg |
| | | catatacgatacaaggctgttagagagataattagaattaatttgactgtaaacacaaagatattagta |
| | | caaaatacgtgacgtagaaagtaataatttcttgggtagtttgcagttttaaaattatgttttaaaatgga |
| | | ctatcatatgcttaccgtaacttgaaagtatttcgatttcttgggtttatatatcttgtggaaaggacgag |
| | | gatccgcatacgggaacgcacatagtgttttagagctagaaatagcaagttaaaataaggctagtcc |
| | | gttatcaacttgaaaaagtggcaccgagtcggtgcttttttct |
| 75 | P22_NLS_ | agacacaattccccagtggaaagacgcgcaggcaaaacgcaccacgtgacggagcgtgaccgc |
| | removed (the | gcgccgagcgcgcgccaaggtcgggcaggaagagggcctatttcccatgattccttcatatttgca |
| | Nuclear | tatacgatacaaggctgttagagagataattagaattaatttgactgtaaacacaaagatattagtaca |
| | Localization | aaatacgtgacgtagaaagtaataatttcttgggtagtttgcagttttaaaattatgttttaaaatggacta |
| | Sequence | tcatatgcttaccgtaacttgaaagtatttcgatttcttgggtttatatatcttgtggaaaggacgaggat |
| | after the | ccgattgttgcacgggagaaccgttttagagctagaaatagcaagttaaaataaggctagtccgttat |
| | Cas9 | caacttgaaaaagtggcaccgagtcggtgcttttttctagacacaattccccagtggaaagacgcgc |
| | sequence was | aggcaaaacgcaccacgtgacggagcgtgaccgcgcgccgagcgcgcgccaaggtcgggca |
| | changed to a | ggaagagggcctatttcccatgattccttcatatttgcatatacgatacaaggctgttagagagataatt |
| | poly-serine | agaattaatttgactgtaaacacaaagatattagtacaaaatacgtgacgtagaaagtaataatttcttg |
| | sequence | ggtagtttgcagttttaaaattatgttttaaaatggactatcatatgcttaccgtaacttgaaagtatttcga |
| | (i.e. | tttcttgggtttatatatcttgtggaaaggacgaggatccgactttggcaagtaagcccgcgttttaga |
| | KKKRK → | gctagaaatagcaagttaaaataaggctagtccgttatcaacttgaaaaagtggcaccgagtcggtg |
| | SAGSSG) | cttttttctagacacaattgcatgaagaatctgcttagggttaggcgttttgcgctgcttcgcgatgtac |
| | | gggccagatatacgcgttgacattgattattgactagttattaatagtaatcaattacggggtcattagtt |
| | | catagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcc |
| | | aagtacgccccctattgacgtcaatgacggtaaatggcccgcctggcattatgcccagtacatgacc |
| | | ttatgggactttcctacttggcagtacatctacgtattagtcatcgctattaccatggtgatgcggttttg |
| | | gcagtacatcaatgggcgtggatagcggtttgactcacggggatttccaagtctccaccccattgac |
| | | gtcaatgggagtttgttttggcaccaaaatcaacgggactttccaaaatgtcgtaacaactccgcccc |
| | | attgacgcaaatgggcggtaggcgtgtacggtgggaggtctatataagcagagctctctggctaac |
| | | tagagaacccactgcttactggcttatcgaaattaatacgactcactatagggagacccaagcttgcc |
| | | accatggacaagaagtacagcatcggcctggacatcggtaccaacagcgtgggctgggccgtga |
| | | tcaccgacgagtacaaggtgcccagcaagaagttcaaggtgctgggcaacaccgaccgccacag |
| | | catcaagaagaacctgatcggcgccctgctgttcgacagcggcgagaccgccgaggccacccgc |
| | | ctgaagcgcaccgcccgccgccgctacacccgccgcaagaaccgcatctgctacctgcaggaga |
| | | tcttcagcaacgagatggccaaggtggacgacagcttcttccaccgcctggaggagagcttcctgg |
| | | tggaggaggacaagaagcacgagcgccaccccatcttcggcaacatcgtggacgaggtggccta |
| | | ccacgagaagtaccccaccatctaccacctgcgcaagaagctggtggacagcaccgacaaggcc |
| | | gacctgcgcctgatctacctggccctggcccacatgatcaagttccgcggccacttcctgatcgag |
| | | ggcgacctgaaccccgacaacagcgacgtggacaagctgttcatccagctggtgcagacctacaa |
| | | ccagctgttcgaggagaaccccatcaacgccagcggcgtggacgccaaggccatcctgagcgcc |
| | | cgcctgagcaagagccgccgcctggagaacctgatcgcccagctgcccggcgagaagaagaac |
| | | ggcctgttcggcaacctgatcgccctgagcctgggcctgacccccaacttcaagagcaacttcgac |
| | | ctggccgaggacgccaagctgcagctgagcaaggacacctacgacgacgacctggacaacctg |
| | | ctggcccagatcggcgaccagtacgccgacctgttcctggccgccaagaacctgagcgacgcca |
| | | tcctgctgagcgacatcctgcgcgtgaacaccgagatcaccaaggcccccctgagcgccagcatg |
| | | atcaagcgctacgacgagcaccaccaggacctgaccctgctgaaggccctggtgcgccagcagc |
| | | tgcccgagaagtacaaggagatcttcttcgaccagagcaagaacggctacgccggctacatcgac |
| | | ggcggcgccagccaggaggagttctacaagttcatcaagcccatcctggagaagatggacggca |
| | | ccgaggagctgctggtgaagctgaaccgcgaggacctgctgcgcaagcagcgcaccttcgacaa |
| | | cggcagcatcccccaccagatccacctgggcgagctgcacgccatcctgcgccgccaggagga |
| | | cttctaccccttcctgaaggacaaccgcgagaagatcgagaagatcctgaccttccgcatcccctac |
| | | tacgtgggccccctggcccgcggcaacagccgcttcgcctggatgacccgcaagagcgaggag |
| | | accatcaccccctggaacttcgaggaggtggtggacaagggcgccagcgcccagagcttcatcg |
| | | agcgcatgaccaacttcgacaagaacctgcccaacgagaaggtgctgcccaagcacagcctgct |
| | | gtacgagtacttcaccgtgtacaacgagctgaccaaggtgaagtacgtgaccgagggcatgcgca |
| | | agcccgccttcctgagcggcgagcagaagaaggccatcgtggacctgctgttcaagaccaaccg |
| | | caaggtgaccgtgaagcagctgaaggaggactacttcaagaagatcgagtgcttcgacagcgtgg |
| | | agatcagcggcgtggaggaccgcttcaacgccagcctgggcacctaccacgacctgctgaagat |
| | | catcaaggacaaggacttcctggacaacgaggagaacgaggacatcctggaggacatcgtgctg |
| | | accctgaccctgttcgaggaccgcgagatgatcgaggagcgcctgaagacctacgcccacctgtt |
| | | cgacgacaaggtgatgaagcagctgaagcgccgccgctacaccggctggggccgcctgagccg |
| | | caagcttatcaacggcatccgcgacaagcagagcggcaagaccatcctggacttcctgaagagcg |
| | | acggcttcgccaaccgcaacttcatgcagctgatccacgacgacagcctgaccttcaaggaggac |
| | | atccagaaggcccaggtgagcggccagggcgacagcctgcacgagcacatcgccaacctggcc |
| | | ggcagccccgccatcaagaagggcatcctgcagaccgtgaaggtggtggacgagctggtgaag |
| | | gtgatgggccgccacaagcccgagaacatcgtgatcgagatggcccgcgagaaccagaccacc |
| | | cagaagggccagaagaacagccgcgagcgcatgaagcgcatcgaggagggcatcaaggagct |
| | | gggcagccagatcctgaaggagcaccccgtggagaacacccagctgcagaacgagaagctgta |
| | | cctgtactacctgcagaacggccgcgacatgtacgtggaccaggagctggacatcaaccgcctga |
| | | gcgactacgacgtggaccacatcgtgccccagagcttcctgaaggacgacagcatcgacaacaa |
| | | ggtgctgacccgcagcgacaagaaccgcggcaagagcgacaacgtgcccagcgaggaggtgg |
| | | tgaagaagatgaagaactactggcgccagctgctgaacgccaagctgatcacccagcgcaagttc |
| | | gacaacctgaccaaggccgagcgcggcggcctgagcgagctggacaaggccggcttcatcaag |
| | | cgccagctggtggagacccgccagatcaccaagcacgtggcccagatcctggacagccgcatga |
| | | acaccaagtacgacgagaacgacaagctgatccgcgaggtgaaggtgatcaccctgaagagcaa |
| | | gctggtgagcgacttccgcaaggacttccagttctacaaggtgcgcgagatcaacaactaccacca |
| | | cgcccacgacgcctacctgaacgccgtggtgggcaccgccctgatcaagaagtaccccaagctg |
| | | gagagcgagttcgtgtacggcgactacaaggtgtacgacgtgcgcaagatgatcgccaagagcg |
| | | agcaggagatcggcaaggccaccgccaagtacttcttctacagcaacatcatgaacttcttcaagac |
| | | cgagatcaccctggccaacggcgagatccgcaagcgccccctgatcgagaccaacggcgagac |
| | | cggcgagatcgtgtgggacaagggccgcgacttcgccaccgtgcgcaaggtgctgagcatgccc |
| | | caggtgaacatcgtgaagaagaccgaggtgcagaccggcggcttcagcaaggagagcatcctgc |
| | | ccaagcgcaacagcgacaagctgatcgcccgcaagaaggactgggaccccaagaagtacggc |
| | | ggcttcgacagccccaccgtggcctacagcgtgctggtggtggccaaggtggagaagggcaaga |
| | | gcaagaagctgaagagcgtgaaggagctgctgggcatcaccatcatggagcgcagcagcttcga |
| | | gaagaaccccatcgacttcctggaggccaagggctacaaggaggtgaagaaggacctgatcatc |
| | | aagctgcccaagtacagcctgttcgagctggagaacggccgcaagcgcatgctggccagcgccg |
| | | gcgagctgcagaagggcaacgagctggccctgcccagcaagtacgtgaacttcctgtacctggcc |
| | | agccactacgagaagctgaagggcagccccgaggacaacgagcagaagcagctgttcgtggag |
| | | cagcacaagcactacctggacgagatcatcgagcagatcagcgagttcagcaagcgcgtgatcct |
| | | ggccgacgccaacctggacaaggtgctgagcgcctacaacaagcaccgcgacaagcccatccg |
| | | cgagcaggccgagaacatcatccacctgttcaccctgaccaacctgggcgcccccgccgccttca |
| | | agtacttcgacaccaccatcgaccgcaagcgctacaccagcaccaaggaggtgctggacgccac |
| | | cctgatccaccagagcatcaccggtctgtacgagacccgcatcgacctgagccagctgggcggc |
| | | gacggcggctccggacctccaaGCGCCGGCAGCAGCGGCgtatacccctacgac |
| | | gtgcccgactacgccctcgaggagggcagaggaagtcttctaacatgcggtgacgtggaggaga |
| | | atcccggccctatggagagcgacgagagcggcctgcccgccatggagatcgagtgccgcatcac |
| | | cggcaccctgaacggcgtggagttcgagctggtgggcggcggagagggcacccccaagcagg |
| | | gccgcatgaccaacaagatgaagagcaccaaaggcgccctgaccttcagcccctacctgctgag |
| | | ccacgtgatgggctacggcttctaccacttcggcacctaccccagcggctacgagaaccccttcct |
| | | gcacgccatcaacaacggcggctacaccaacacccgcatcgagaagtacgaggacggcggcgt |
| | | gctgcacgtgagcttcagctaccgctacgaggccggccgcgtgatcggcgacttcaaggtggtgg |
| | | gcaccggcttccccgaggacagcgtgatcttcaccgacaagatcatccgcagcaacgccaccgtg |
| | | gagcacctgcaccccatgggcgataacgtgctggtgggcagcttcgcccgcaccttcagcctgcg |
| | | cgacggcggctactacagcttcgtggtggacagccacatgcacttcaagagcgccatccacccca |
| | | gcatcctgcagaacgggggccccatgttcgccttccgccgcgtggaggagctgcacagcaacac |
| | | cgagctgggcatcgtggagtaccagcacgccttcaagacccccatcgccttcgccagatcccgcg |
| | | ctcagtcgtccaattctgccgtggacggcaccgccggacccggctccaccggatctcgctaggcg |
| | | gccgcagatgggggtcctgggccccagggtgtgcagccactgacttggggactgctggtggggt |
| | | agggatgagggagggaggggcattgtgatgtacagggctgctctgtgagatcaagggtctcttaa |
| | | gggtgggagctggggcagggactacgagagcagccagatgggctgaaagtggaactcaaggg |
| | | gtttctggcacctacctacctgcttcccgctggggggtggggagttggcccagagtcttaagattgg |
| | | ggcagggtggagaggtgggctcttcctgcttcccactcatcttatagctttctttccccagatccgaat |
| | | tggagatccaaaccaaggcgcgcgctagcgccaccatgggatcggccattgaacaagatggattg |
| | | cacgcaggttctccggccgcttgggtggagaggctattcggctatgactgggcacaacagacaatc |
| | | ggctgctctgatgccgccgtgttccggctgtcagcgcaggggcgcccggttctttttgtcaagaccg |
| | | acctgtccggtgccctgaatgaactgcaggacgaggcagcgcggctatcgtggctggccacgac |
| | | gggcgttccttgcgcagcagtgctcgacgttgtcactgaagcgggaagggactggctgctattggg |
| | | cgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacat |
| | | cgcatcgagcgagcacgtactcggatggaagccggtcttgtcgatcaggatgatctggacgaaga |
| | | gcatcaggggctcgcgccagccgaactgttcgccaggctcaaggcgcgtatgcccgacggcgat |
| | | gatctcgtcgtgactcatggcgatgcctgcttgccgaatatcatggtggaaaatggccgcttttctgg |
| | | attcatcgactgtggccggctgggtgtggcggaccgctatcaggacatagcgttggctacccgtga |
| | | tattgctgaagagcttggcggcgaatgggctgaccgcttcctcgtgctttacggtatcgccgctccc |
| | | gattcgcagcgcatcgccttctatcgccttcttgacgagttcttctgaacgcggtgctacgagatttcg |
| | | attccaccgccgccttctatgaaaggttgggcttcggaatcgttttccgggacgccggctggatgatc |
| | | ctccagcgcggggatctcatgctggagttcttcgcccaccccaacttgtttattgcagcttataatggtt |
| | | acaaataaagcaatagcatcacaaatttcacaaataaagcatttttttcactgcattctagttgtggtttgt |
| | | ccaaactcatcaatgtatcttatcatgtctgtataccgtcgacctctagctagagcttggcgtaatcatg |
| | | gtcatagctgtttcctgtgtgaaattgttatccgctcacaattccacacaacatacgagccggaagcat |
| | | aaaggtaagcctgaatattgaaaaaggaagagtatgagtattcaacatttccgtgtcgcccttattccc |
| | | ttttttgcggcattttgccttcctgtttttgctcacccagaaacgctggtgaaagtaaaagatgctgaag |
| | | atcagttgggtgcacgagtgggttacatcgaactggatctcaacagcggtaagatccttgagagtttt |
| | | cgccccgaagaacgttttccaatgatgagcacttttaaagttctgctatgtggcgcggtattatcccgt |
| | | attgacgccgggcaagagcaactcggtcgccgcatacactattctcagaatgacttggttgagtact |
| | | caccagtcacagaaaagcatcttacggatggcatgacagtaagagaattatgcagtgctgccataa |
| | | ccatgagtgataacactgcggccaacttacttctgacaacgatcggaggaccgaaggagctaacc |
| | | gcttttttgcacaacatgggggatcatgtaactcgccttgatcgttgggaaccggagctgaatgaagc |
| | | cataccaaacgacgagcgtgacaccacgatgcctgtagcaatggcaacaacgttgcgcaaactatt |
| | | aactggcgaactacttactctagcttcccggcaacaattaatagactggatggaggcggataaagtt |
| | | gcaggaccacttctgcgctcggcccttccggctggctggtttattgctgataaatctggagccggtg |
| | | agcgtgggtctcgcggtatcattgcagcactggggccagatggtaagccctcccgtatcgtagttat |
| | | ctacacgacggggagtcaggcaactatggatgaacgaaatagacagatcgctgagataggtgcct |
| | | cactgattaagcattggtaactgtcagaccaagtttactcatatatactttagattgatttaaaacttcattt |
| | | ttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgt |
| | | aatctgctgcttgcaaacaaaaaaaccaccgctaccagcggtggtttttttgccggatcaagagctac |
| | | caactctttttccgaaggtaactggcttcagcagagcgcagataccaaatactgttcttctagtgtagc |
| | | cgtagttaggccaccacttcaagaactctgtagcaccgcctacatacctcgctctgctaatcctgttac |
| | | cagtggctgctgccagtggcgataagtcgtgtcttaccgggttggactcaagacgatagttaccgga |
| | | taaggcgcagcggtcgggctgaacggggggttcgtgcacacagcccagcttggagcgaacgac |
| | | ctacaccgaactgagatacctacagcgtgagctatgagaaagcgccacgcttcccgaagggagaa |
| | | aggcggacaggtatccggtaagcggcagggtcggaacaggagagcgcacgagggagcttcca |
| | | gggggaaacgcctggtatctttatagtcctgtcgggtttcgccacctctgacttgagcgtcgatttttgt |
| | | gatgctcgtcaggggggcggagcctatggaaaaacgccagcaacgcggcctttttacggttcctg |
| | | gccttttgctggccttttgctcacatgttctttcctgcgttatcccctgattctgtggataaccgtattacc |
| | | gcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgc |
| | | agctggcacgacaggtttcccgactggaaagcgggcagtgagcgcaacgcaattaatgtgagtta |
| | | gctcactcattaggcaccccaggctttacactttatgcttccggctcgtatgttgtgtggaattgtgagc |
| | | ggataacaatttcacacaggaaacagctatgacatgattacgaattgcaacgatttaggtgacactat |
| | | agaagagaaggaattaatacgactcactatagggagagagagagaattaccctcactaaagggag |
| | | gagaagcatgaattccccagtggaaagacgcgcaggcaaaacgcaccacgtgacggagcgtga |
| | | ccgcgcgccgagcgcgcgccaaggtcgggcaggaagagggcctatttcccatgattccttcatatt |
| | | tgcatatacgatacaaggctgttagagagataattagaattaatttgactgtaaacacaaagatattagt |
| | | acaaaatacgtgacgtagaaagtaataatttcttgggtagtttgcagttttaaaattatgttttaaaatgg |
| | | actatcatatgcttaccgtaacttgaaagtatttcgatttcttgggtttatatatcttgtggaaaggacgag |
| | | gatccgtttaacaatcgtctcgtggagttttagagctagaaatagcaagttaaaataaggctagtccgt |
| | | tatcaacttgaaaaagtggcaccgagtcggtgcttttttct |

### EXAMPLES

### Example 1. Control of ASF Through CRISPR/Cas Mediated Direct Editing of the ASFV Genome in the Animal Through Use of a DNA Based Vector

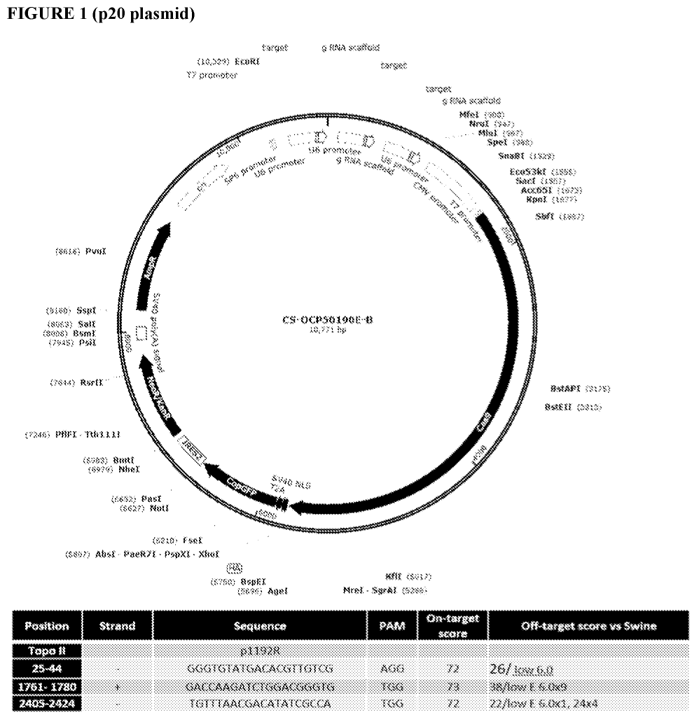

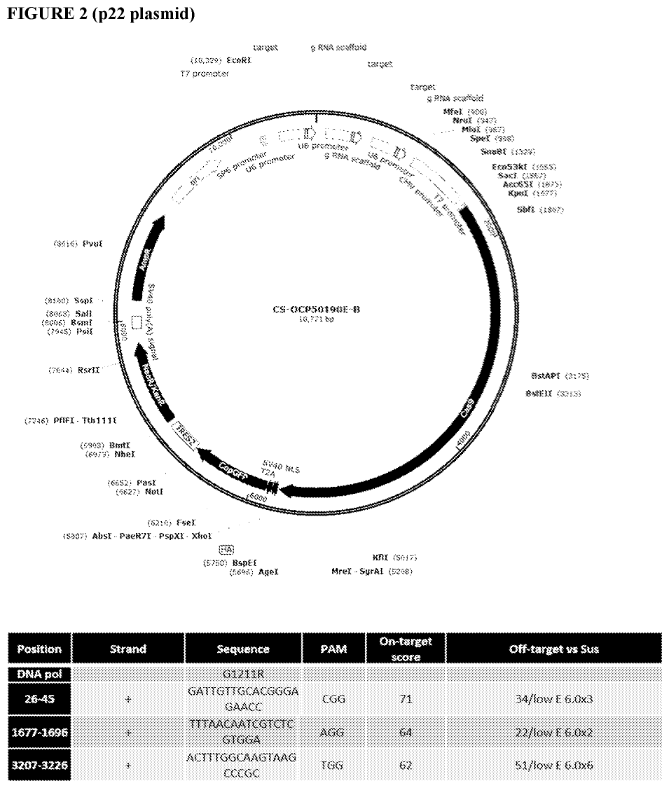

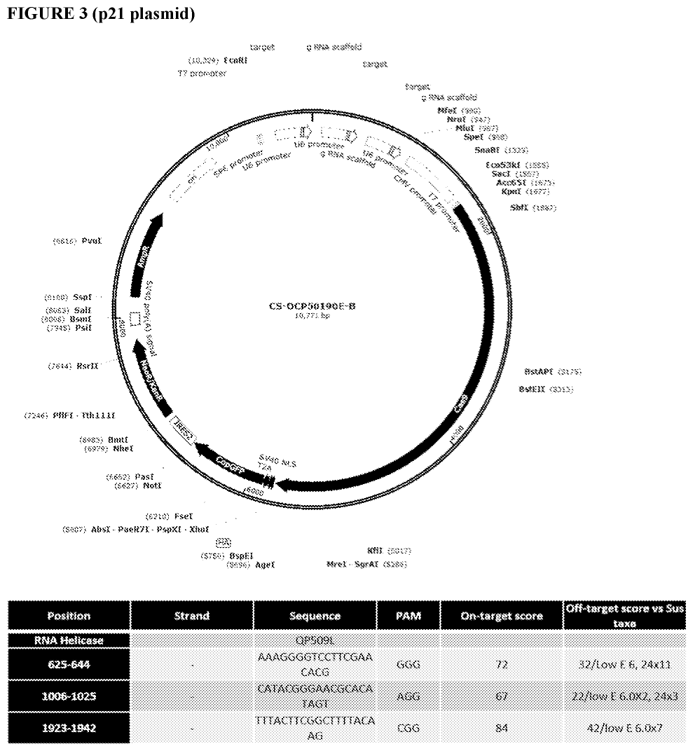

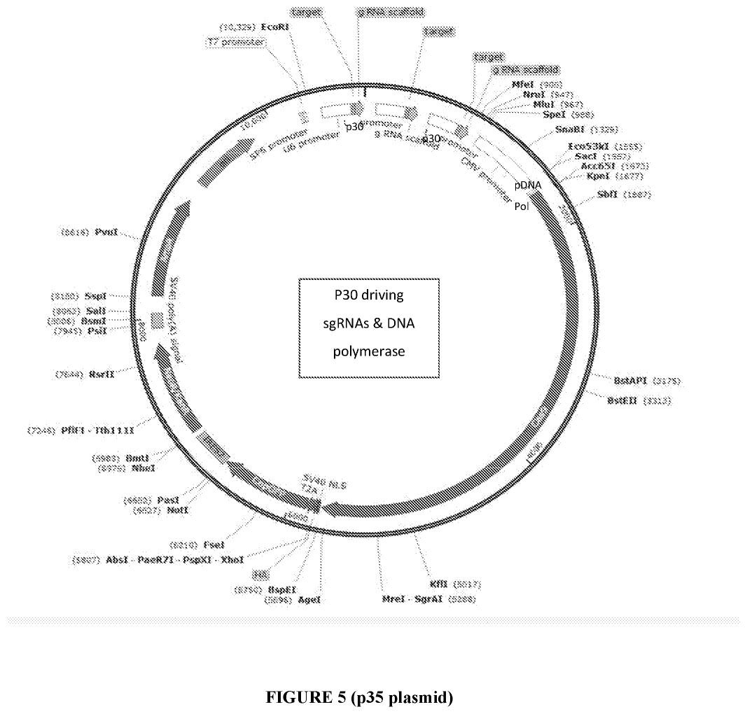

The direct prevention of ASFV using genome editing in the animal to target the virus early in its development of an infection can be accomplished by delivery of a DNA construct such as described in the specification as well as in FIG. 1, FIG. 2, FIG. 3 and FIG. 5. These constructs are designed to target genes for cleavage that are involved early in the replication cycle of ASFV and to disrupt the gene and stop replication. This is accomplished by having promoters drive expression of the caspase gene to produce the Cas9 endonuclease and the sgRNAs in the cell. The sgRNAs then recognize invading ASFV DNA, bind to the complementary sequences and Cas9 binds to form a complex that disrupts expression and function of these genes in the cell. The DNA vector(s) can be delivered via injection of a solution into the blood stream comprising the vector DNA protected in nanoparticles

### Example 2. Construction of a CRISPR/Cas9 Construct Targeting DNA Polymerase and Topoisomerase II of ASFV for Direct Gene Targeting

A construct was synthesized containing one or more specific guide RNAs that target the DNA polymerase of African Swine Fever Virus. Strain BA71V (NC_001695.2) of ASFV was used for the present example. TABLE 5 provides the sequences for polymerase, Topoisomerase II, and RNA helicase.

Using these sequences, sequences for potential specific guide RNAs were generated. sgRNAs were generated that had strong on-target effects for genome editing. Some of these potential sites are provided in Table 7 below.

TABLE 7
Example sgRNA target sequences for DNA polymerase

| | | | | | |
|---|---|---|---|---|---|
| | | | | On- | |
| | | | | target | Off-target |
| Position | Strand | Sequence | PAM | score | vs Sus |

DNA polymerase (G1211R)

| | | | | | |
|---|---|---|---|---|---|
| 26-45 | + | GATTGTTGCACGGG | CGG | 71 | 34/low |
| | | AGAACC | | | E 6.0x3 |
| | | (SEQ ID NO: 23) | | | |
| 1677- | + | TTTAACAATCGTCT | AGG | 64 | 22/low |
| 1696 | | CGTGGA | | | E 6.0x2 |
| | | (SEQ ID NO: 24) | | | |
| 3207- | + | ACTTTGGCAAGTAA | TGG | 62 | 51/low |
| 3226 | | GCCCGC | | | E 6.0x6 |
| | | (SEQ ID NO: 25) | | | |

Using a Cas9 gene that has been codon optimized for mammalian expression driven by a mammalian functional promoter (e.g., CMV or ASFV p72 promoter (see e.g., Garcia-Escudero, R., G. Andres, F. Almazán and E. Vinuela (1998). “Inducible Gene Expression from African Swine Fever Virus Recombinants: Analysis of the Major Capsid Protein p72.” Journal of Virology 72(4): 3185-3195, or Garcia-Escudero, R. and E. Vinuela (2000). “Structure of African swine fever virus late promoters: requirement of a TATA sequence at the initiation region.” J Virol 74(17): 8176-8182 or Rodriguez, J. M. and M. L. Salas (2013). “African swine fever virus transcription.” Virus Res 173(1): 15-28.) and the sgRNA cassette driven by another mammalian functional promoter(s) (e.g., U6 or ASFV p30) a circular plasmid vector that can be amplified in bacteria (or cell free) to produce DNA as well as able to express the cloned gene and sgRNA(s) in swine to produce the CRISPR/Cas9 elements needed to specifically edit the ASFV DNA polymerase adding insertions or deletions that will prevent expression of the gene was developed.

An example vector generated via this procedure is provided as SEQ ID NO: 4.

As with DNA polymerase above, the topoisomerase II gene p1192R was utilized to generate sgRNA targeting sequences for p1192R. Some of these potential sites are provided in Table 8 below. These sequences were used to generate a triple sgRNA targeting vector as above, which is provided as SEQ ID NO: 5.

TABLE 8
Example sgRNA target sequences for
Topoisomerase II

| | | | | | |
|---|---|---|---|---|---|
| | | | | | Off- |
| | | | | On- | target |
| | | | | target | score |
| Position | Strand | Sequence | PAM | score | vs Swine |

Topoisomerase II (p1192R)

| | | | | | |
|---|---|---|---|---|---|
| 25-44 | − | GGGTGTATGACACG | AGG | 72 | 26/E value |
| | | TTGTCG | | | low 6.0 |
| | | (SEQ ID NO: 26) | | | |
| 1761- | + | GACCAAGATCTGGA | TGG | 73 | 38/low |
| 1780 | | CGGGTG | | | E 6.0x9 |
| | | (SEQ ID NO: 27) | | | |
| 2405- | − | TGTTTAACGACATA | TGG | 72 | 22/low E |
| 2424 | | TCGCCA | | | 6.0x1, |
| | | (SEQ ID NO: 28) | | | 24x4 |

The same process was used to generate sgRNA target sequences for RNA helicase QP509L. Some of these potential sites are provided in Table 9, which can be used to generate a triple sgRNA targeting vector as above.

TABLE 9
Example sgRNA target sequences for RNA
helicase QP509L

| | | | | | |
|---|---|---|---|---|---|
| | | | | On- | Off-target |
| | | | | target | score vs |
| Position | Strand | Sequence | PAM | score | Sus taxa |

RNA Helicase (QP509L)

| | | | | | |
|---|---|---|---|---|---|
| 625-644 | − | AAAGGGGTCCTTCG | GGG | 72 | 32/Low |
| | | AACACG | | | E 6, |
| | | (SEQ ID NO: 29) | | | 24x11 |
| 1006- | − | CATACGGGAACGCA | AGG | 67 | 22/low E |
| 1025 | | CATAGT | | | 6.0x2, |
| | | (SEQ ID NO: 30) | | | 24x3 |
| 1923- | − | TTTACTTCGGCTTT | CGG | 84 | 42/low E |
| 1942 | | TACAAG | | | 6.0x7 |
| | | (SEQ ID NO: 31) | | | |

### Example 3. Selection of sgRNAs Capable of Targeting Multiple Genes in African Swine Fever Virus Genome Through the Targeting of Conserved Sites within Multigene Families (MGF)

ASFV is unique in having a large number of multigene families in its genome (e.g., MGF 100, 110, 305, 505/560 and p22). This provides an opportunity to target a number of genes simultaneously in the same MGF through genome editing to amplify the ability of the instant invention to stop ASFV replication and infection.

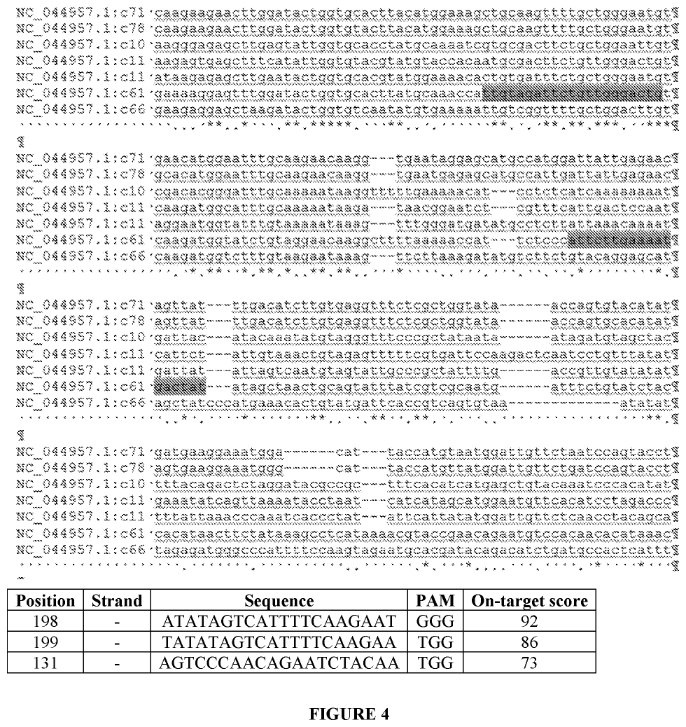

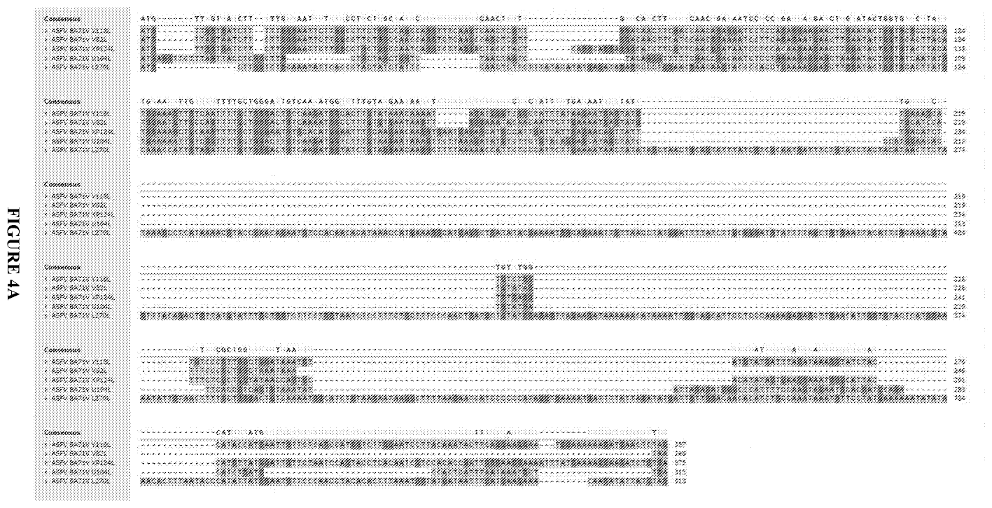

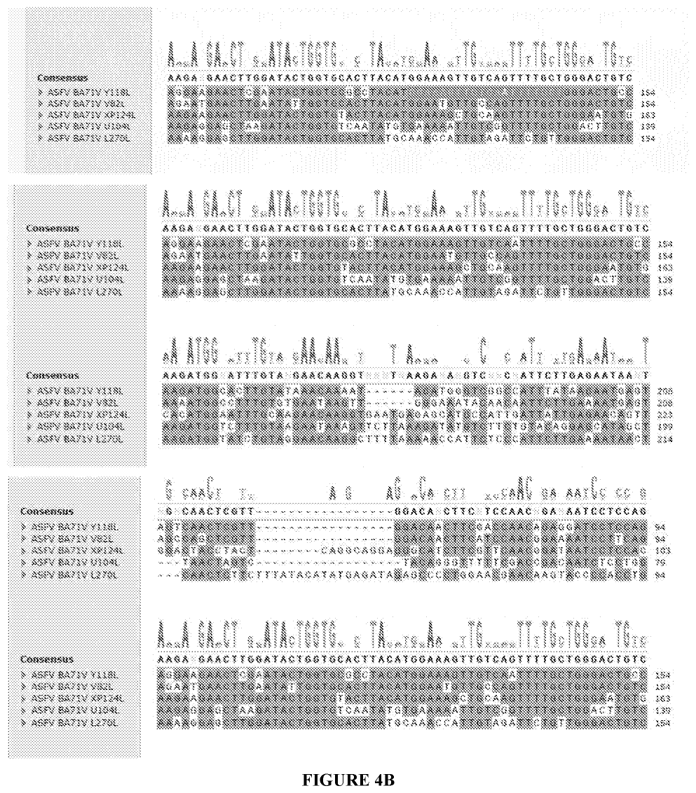

MGF 110-1L protein of ASFV can be used as an example of how this can be done. One does a multiple gene alignment of all members of MGF 110 (FIG. 4). T The OURT 88/3 genome (NC 044957.1) is used to pull out all of the MGF 110 genes and align using the Clustal alignment by MAFFT (v7.452) a portion of which is provided in FIG. 4. While some of the MGF 110 genes are relatively small and have minimal regions of high homology, most have areas of high homology that are targeted for genome editing by designing sgRNAs located in those regions. Three sgRNAs were designed using the MGF 110-1L gene and are located in a region of high homology with other members of the multigene family. The sequences of these sgRNAs are provided in Table 10 below. As in Example 2 above, such sequences can be inserted into a multi-sgRNA expression vector for targeting of MGF 110 family genes in ASFV.

TABLE 10
sgRNA targeting sequences (represented as
targeted DNA sequence for insertion into an
encoding vector) designed to target multiple
members of the MGF 110 gene family via
targeting of conserved regions

| | | | | On- | SEQ |
|---|---|---|---|---|---|
| | | | | target | ID |
| Position | Strand | Sequence | PAM | score | NO: |
| 198 | − | ATATAGTCATTTTCAAGAAT | GGG | 92 | 32 |
| 199 | − | TATATAGTCATTTTCAAGAA | TGG | 86 | 33 |
| 131 | − | AGTCCCAACAGAATCTACAA | TGG | 73 | 34 |

A similar procedure can be utilized to target MGF 110 proteins L270L, U104L, XP124L, V82L and Y1118L simultaneously. A sequence alignment of these members of the MGF 110 gene family from BA71V show regions of strong homology at the beginning and end of the genes (FIG. 4A).

Using the genome sequence of BA71V (NC 001659.2) and focusing on the 5′ end of the genes, one can design sgRNAs using an crRNA design tool after aligning the sequences of the MGF 110 genes and looking for regions of high identity to target. At least four different sgRNAs can be designed that retain the PAM sites and also have strong nucleotide identity with all of the MGF110 genes of BA71V (see Table 10A). Conservation of the sites targeted between MGF family members can be seen in FIG. 4B.

TABLE 10A
sgRNAs targeting sequences (represented as
targeted DNA sequence for insertion into an
encoding vector) designed to target multiple
members of the MGF 110 gene family via
targeting of conserved regions

| | | | | On- | SEQ |
|---|---|---|---|---|---|
| Starting | | | | Target | ID |
| Position | Strand | Sequence | PAM | Score | NO: |
| 125 | + | GGAAAGTTGTCAATTTTGCT | GGG | 72 | 65 |
| 124 | + | TGGAAAGTTGTCAATTTTGC | TGG | 70 | 66 |
| 138 | + | TTTTGCTGGGACTGCCAAGA | TGG | 70 | 67 |
| 78 | − | TCCTCTGGAGGATCCTCTGT | TGG | 69 | 68 |

### Example 4. Verification of Component Expression from Designed Vectors in Mammalian Cells

For each of the vectors designed and presented in Table 5, verification of Cas protein and guide RNA expression from vector promoters was verified. Briefly, each vector (p18, p19, p20, p21, and p23) was transfected into cultured HEK293 cells using the standard Lipofectamine 3000 protocol. 24 hours after transfection, expression of Cas endonuclease (e.g. Cas9) was verified by western blotting using mouse monoclonal anti-Cas9 antibody (Invitrogen clone 7A903A3) and RT-PCR using PowerUp SYBR green chemistry using the manufacturer's protocol with primers CCTGTTCGACGACAAGGTGA and CGTTGATAAGCTTGCGGCTC; sgRNA expression was verified by RT-PCR using the same protocol with primers for sgRNAs targeting DNA polymerase (TCGTCTCGTGGAGTTTTAGAGC & CGACTCGGTGCCACTTTTTC), RNA helicase (ACGGGAACGCACATAGTGTTTTA & CGACTCGGTGCCACTTTTT) and Topoisomerase II (CGACTCGGTGCCACTTTTT & TGGACGGGTGGTTTTAGAGC).

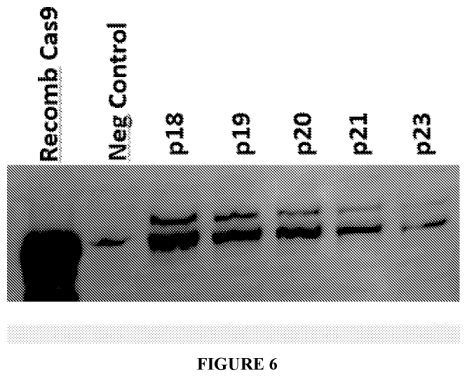

For Cas expression, example results are presented in FIG. 6. Each of the cell conditions transfected by vectors (e.g. p18, p19, p20, p21, and p23) showed increases in intensity of a band corresponding to Cas9.

For sgRNA expression, example results are presented in Table 11 below. For all sgRNAs induced by the vectors, fold increases versus scrambled control or no sgRNA controls was very large, indicating that these constructs successfully expressed guide RNAs needed for viral gene targeting in transfected cells.

TABLE 11

| | | Fold Increase vs. | Fold Increase vs. |
|---|---|---|---|
| Plasmid | sgRNA | scrambled control | no sgRNA control |
| Cas9/Topoisomerase II | Topo 1 | 2512 | 6994 |
| sgRNAs (p20 plasmid) | Topo 2 | 12353 | 9624 |
| Cas9/RNA helicase | RH1 | 3016 | 32899 |
| sgRNAs (p21 plasmid) | RH2 | 521 | 1460 |
| Cas9/DNA polymerase | Pol1 | 12360 | 13259 |
| sgRNAs (p22 plasmid) | Pol2 | 18195 | 21876 |

### Example 5. Verification of Viral Gene Targeting in Mammalian Cells by Designed Vectors

For each of the vectors designed and presented in Table 5, verification of targeting of viral genes in infected mammalian cells was next performed. For these experiments, each vector (p18, p19, p20, p21, and p23) was transfected into cultured HEK293 cells using Lipofectamine 3000 as directed by the manufacturer. As a proxy for viral infection, each targeted gene was inserted into a lentiviral vector (pLVX-AcGFP1-C1, Clonetech) by Genscript and transformed into E. coli for production of model plasmid DNA and lentiviruses bearing the corresponding genes were co-transfected alongside the targeting plasmids using 2 pg of DNA from each plasmid.

48 hours after co-transfection, viral gene targeting was assessed by a heteroduplex formation assay using the AltR heteroduplex kit from IDT Technologies. In this assay, editing of corresponding genes is assessed by annealing of PCR amplicons generated from primers the span the insertion site on the pLVX-AcGFP-C1 multicloning site (CCGGCCTGCTCTGGTG & CTCGAGATCTGAGTCCGGACT) to form a loop that is cut by an endonuclease in an AltR assay.

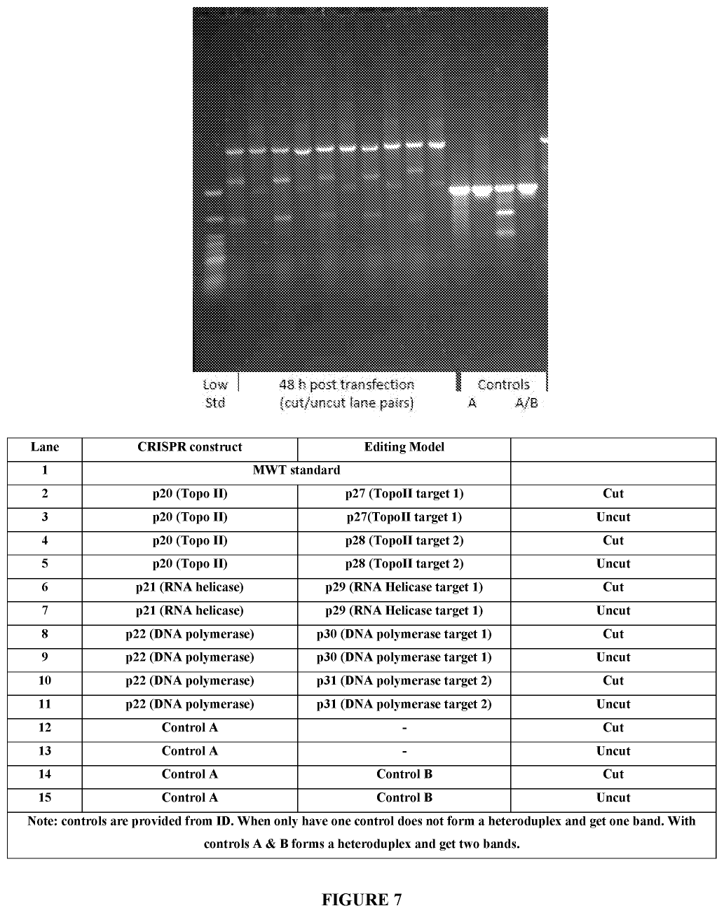

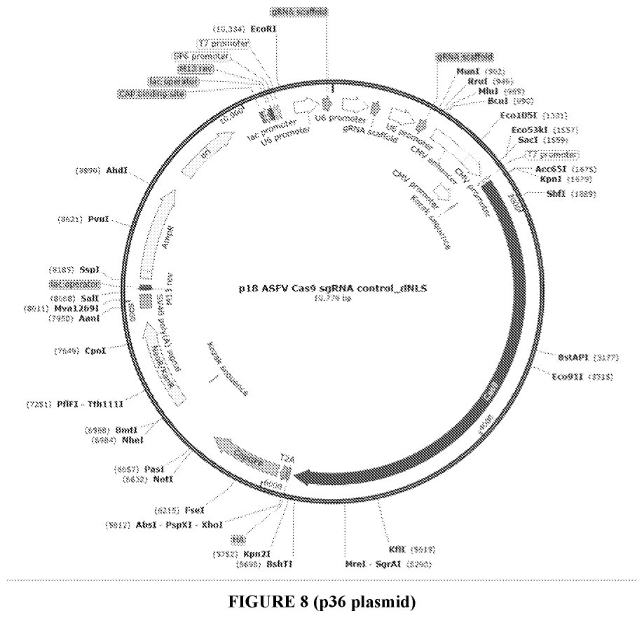

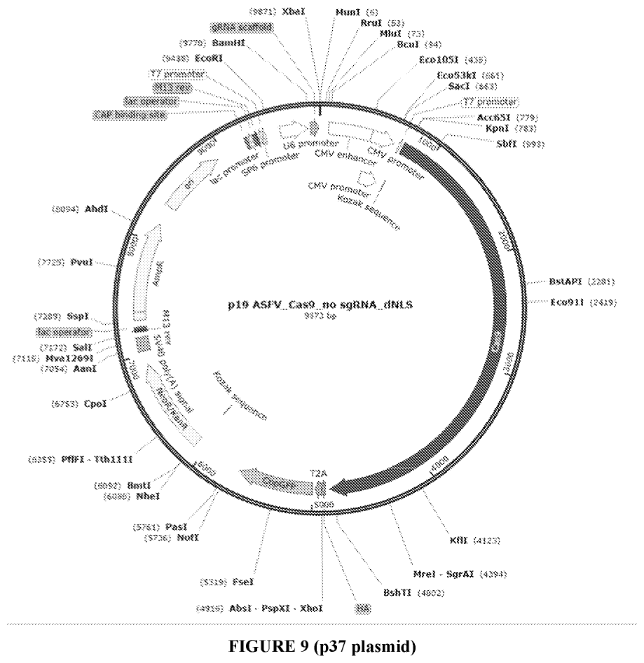

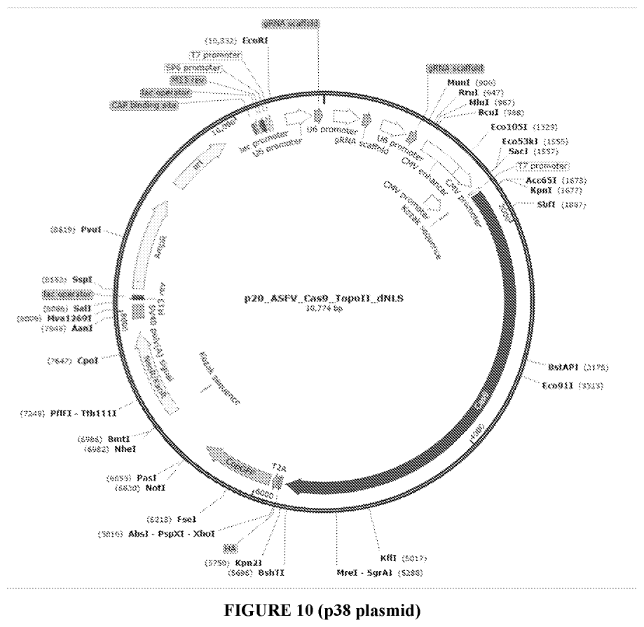

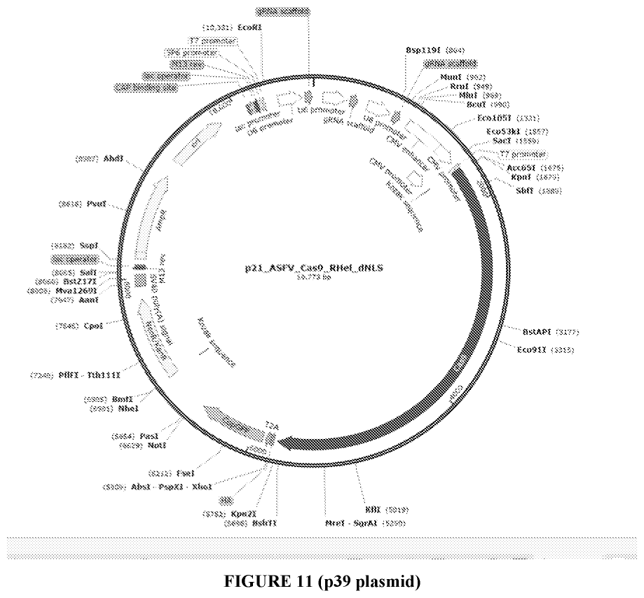

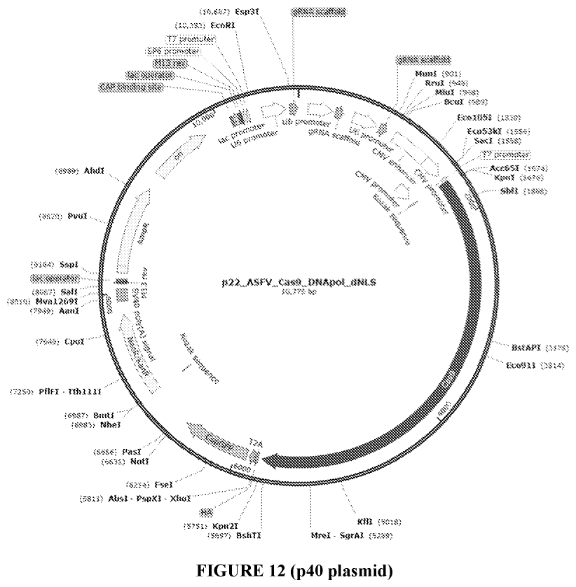

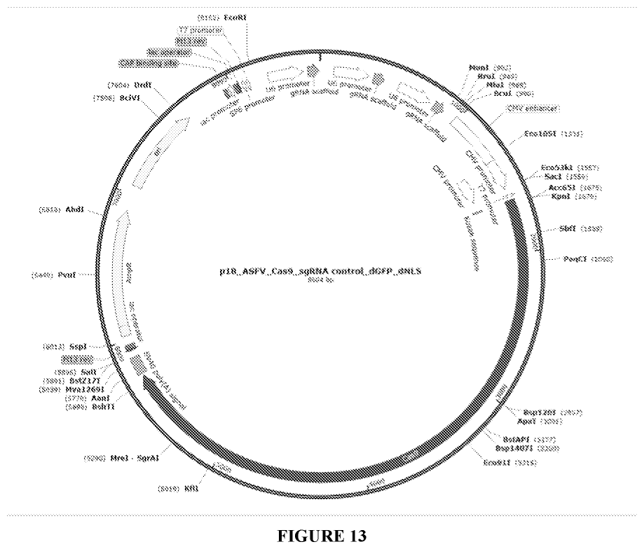

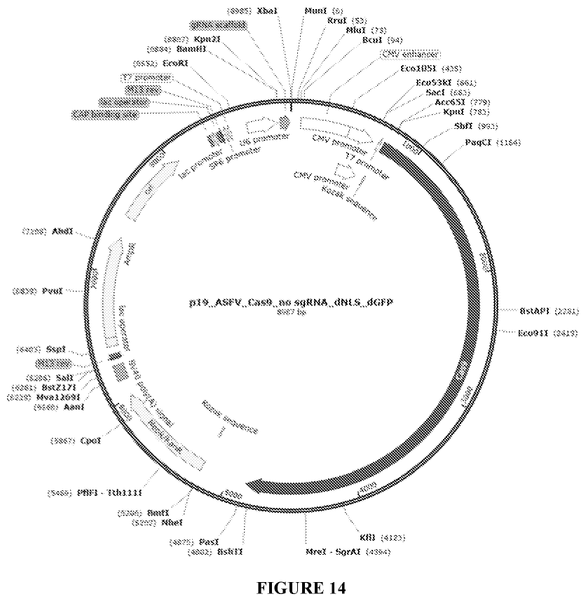

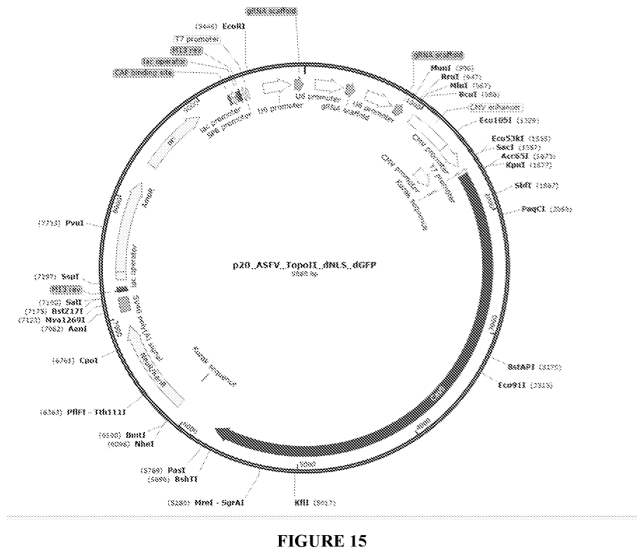

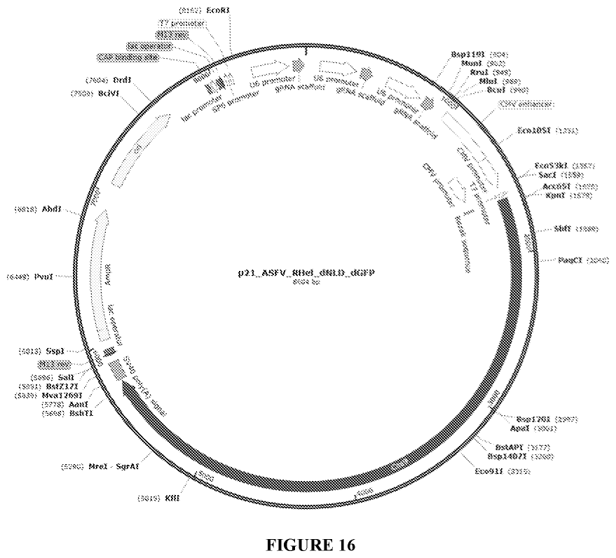

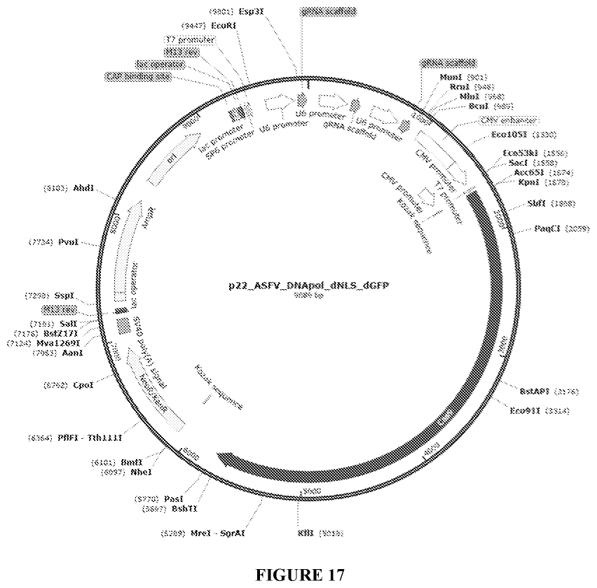

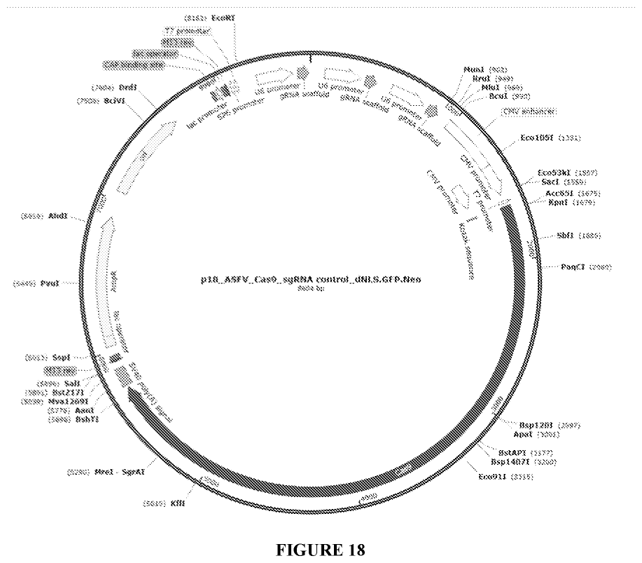

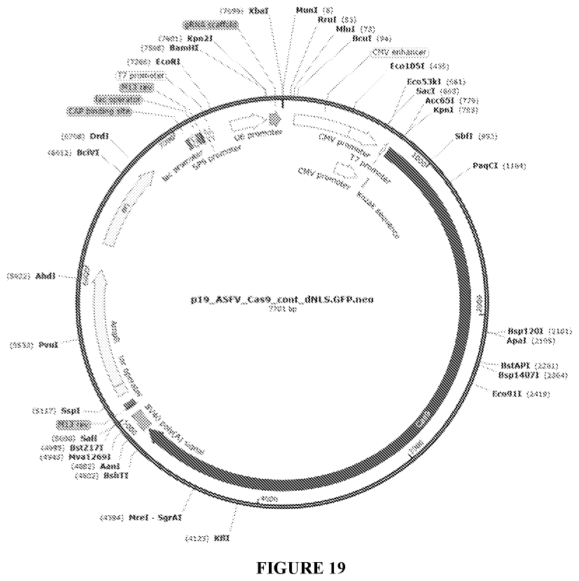

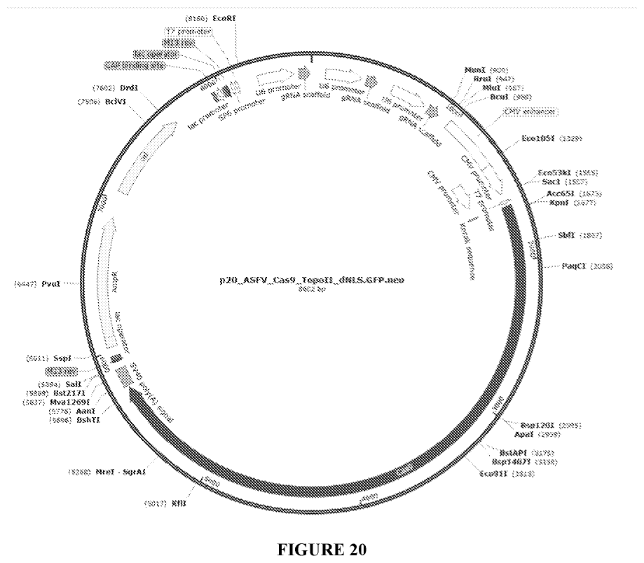

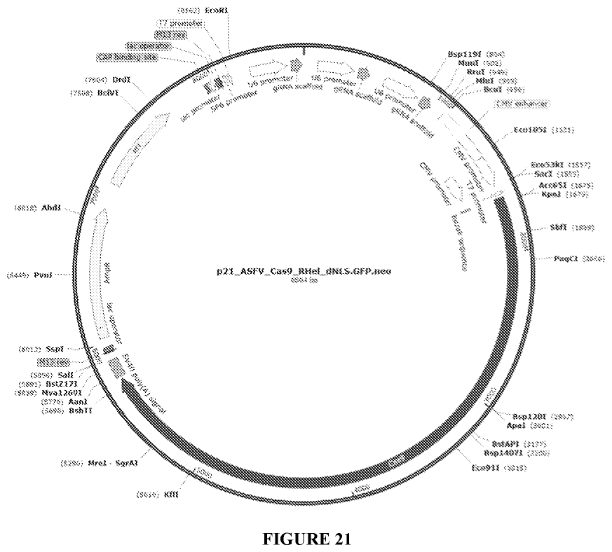

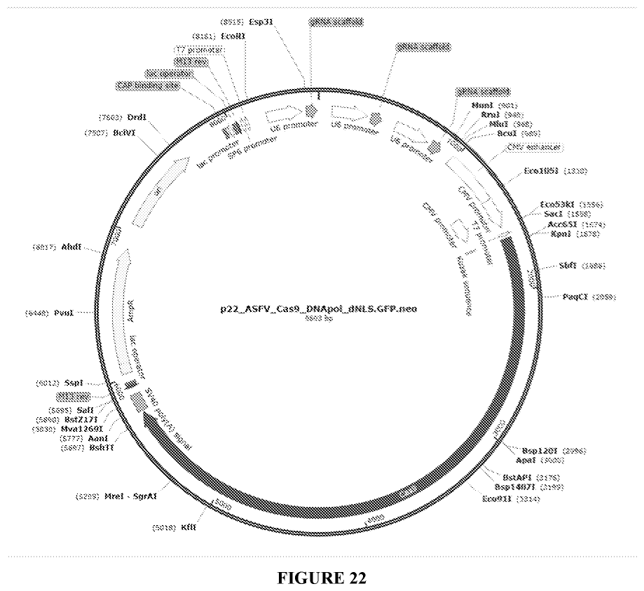

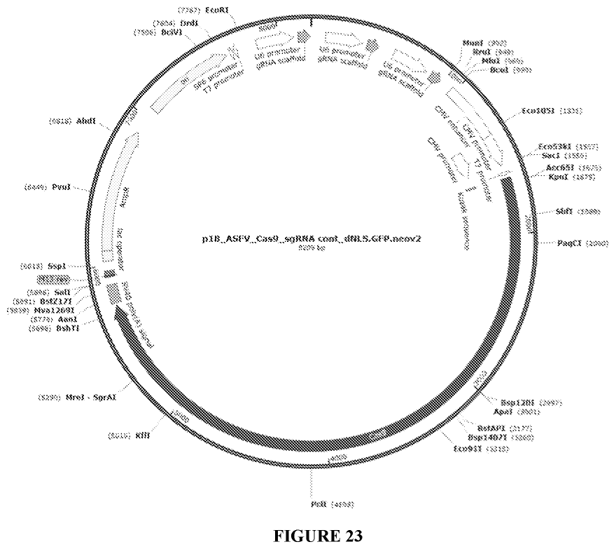

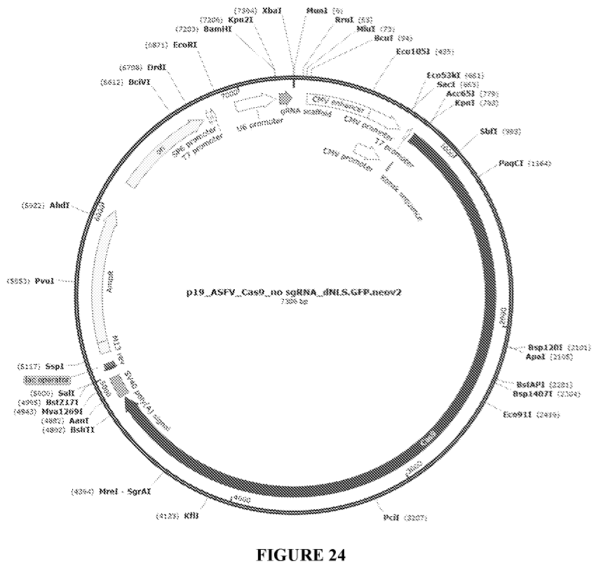

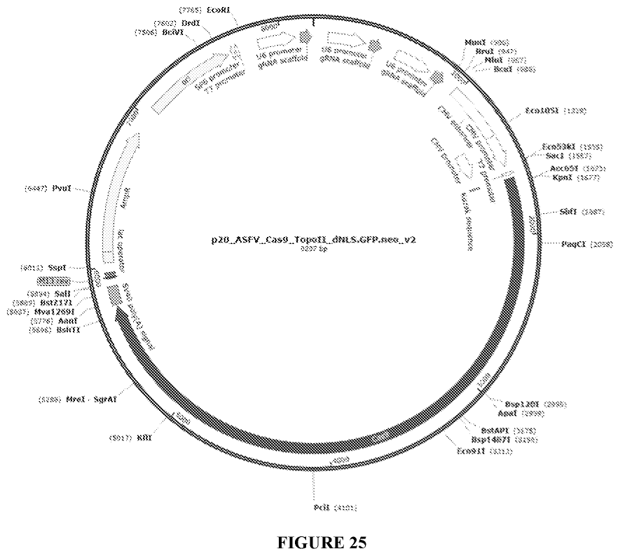

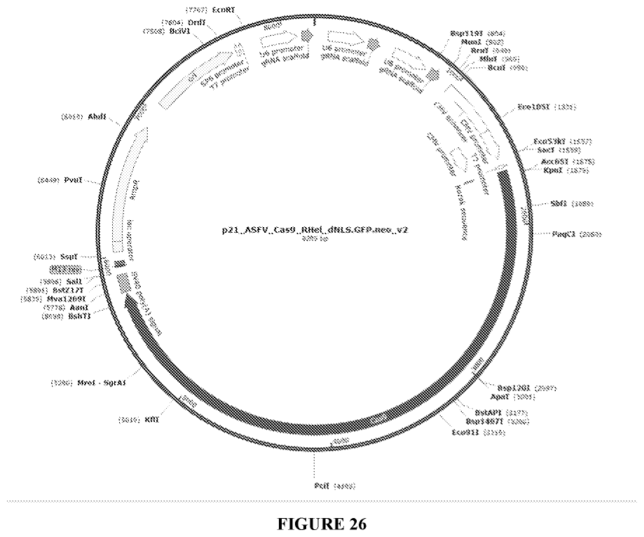

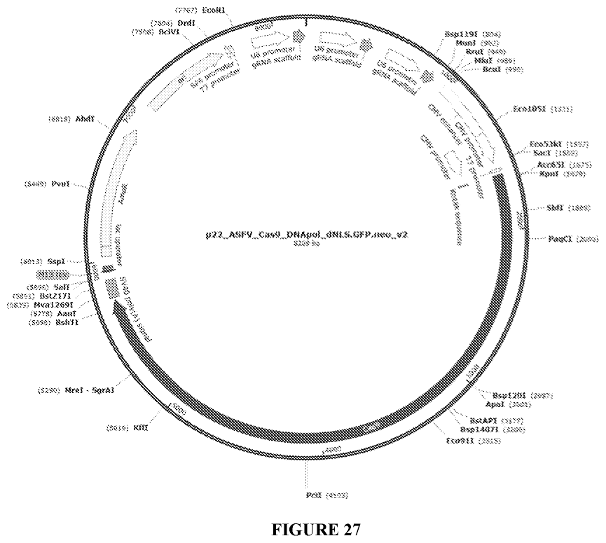

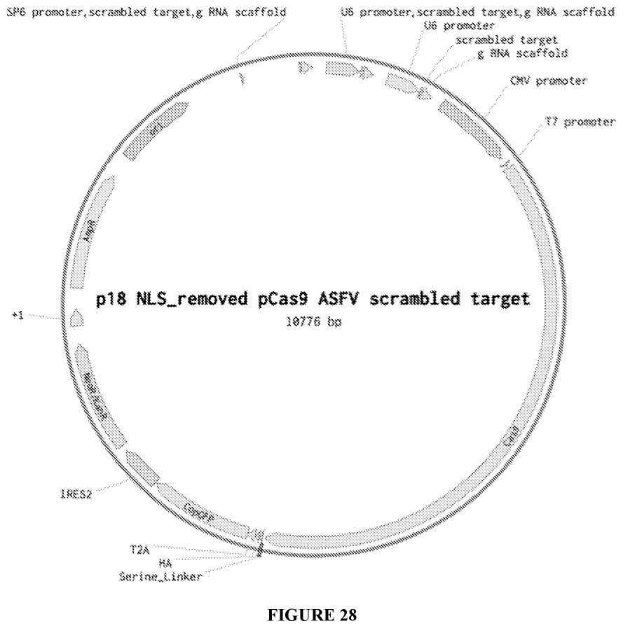

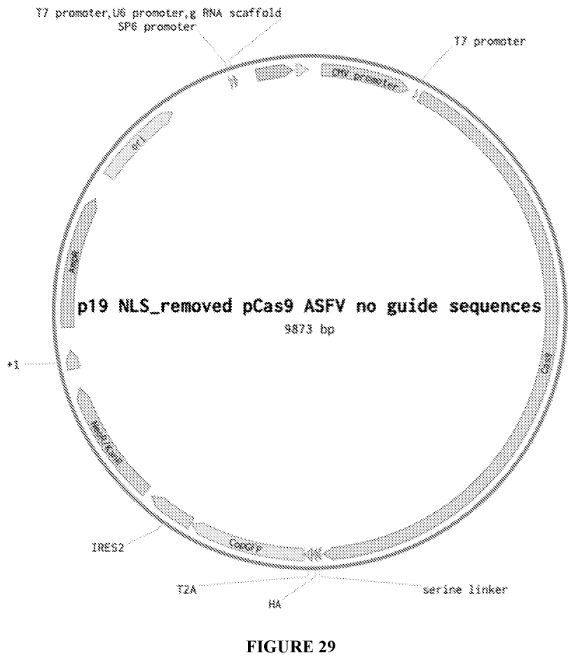

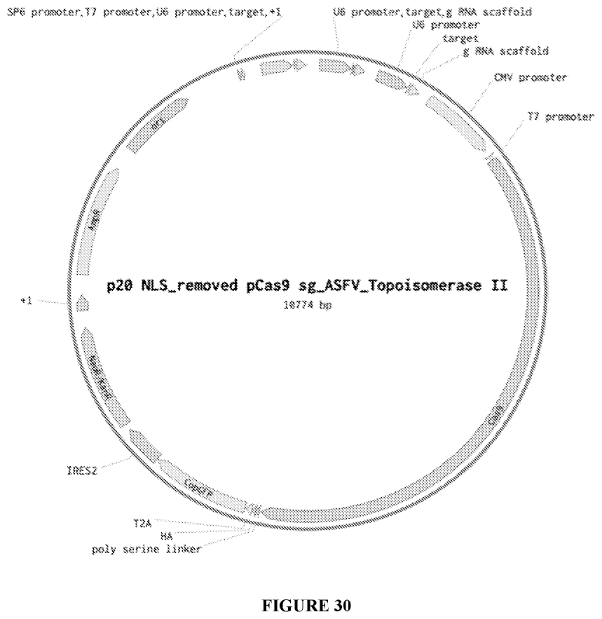

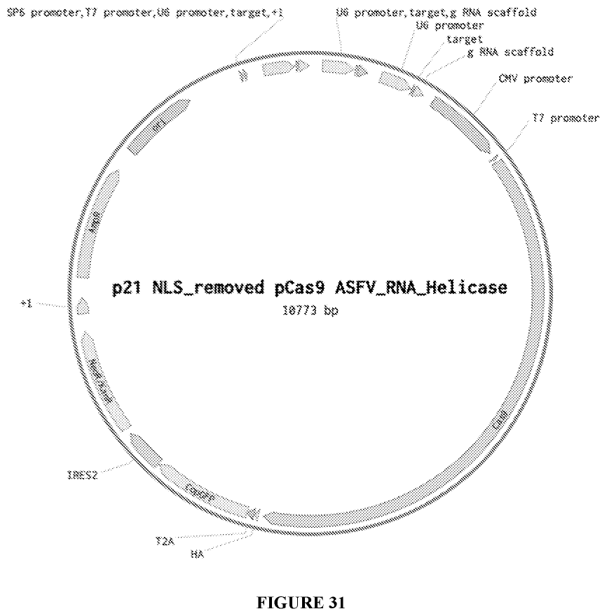

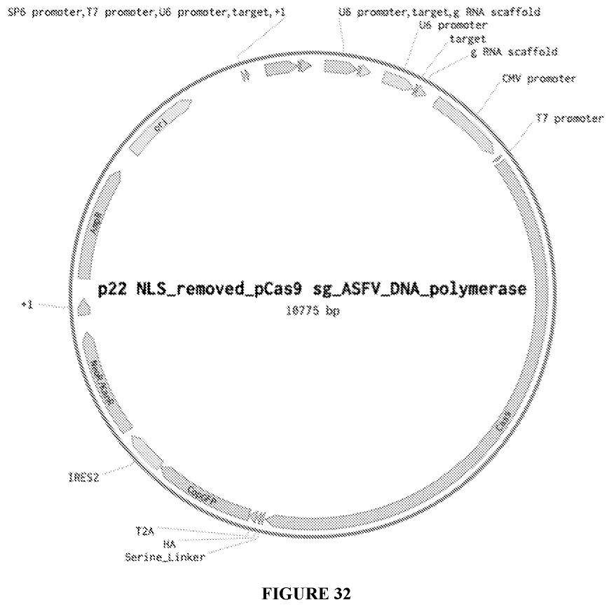

The results of this experiment are presented in FIG. 7. For each vector, heteroduplex formation was detected by formation of lower molecular weight bands upon AltR treatment, indicating that all of the vectors were effective at targeting each of the viral genes (e.g. Topo1, Topo2, RH1, RH2, Pol1, and Pol2).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Embodiments

The following embodiments are intended to be illustrative and not to be limiting in any way.

Embodiment 1. A method for inhibiting infection of or reducing replication of a virus in an animal in need thereof, comprising introducing to a cell of said animal a nuclease comprising a gene-binding moiety, wherein said gene binding moiety is configured to bind at least one essential gene of said virus, or any combination thereof.

Embodiment 2. The method of embodiment 1, wherein said virus belongs to the family Asfarviridae.

Embodiment 3. The method of embodiment 1 or embodiment 2, wherein said at least one essential gene of said virus encodes DNA polymerase or a fragment thereof, Topoisomerase II or a fragment thereof, RNA helicase or a fragment thereof, or a multigene family (MGF) family member or a fragment thereof.

Embodiment 4. The method of any one of embodiments 1-3, wherein said at least one essential gene encodes DNA polymerase or a fragment thereof, wherein the DNA polymerase is G1211R or a fragment thereof.

Embodiment 5. The method of any one of embodiments 1-4, wherein said at least one essential gene encodes Topoisomerase II or a fragment thereof, wherein the Topoisomerase II is p1192R or a fragment thereof.

Embodiment 6. The method of any one of embodiments 1-5, wherein said at least one essential gene encodes RNA helicase or a fragment thereof, wherein the RNA helicase is QP509L, A859L, F105L, B92L, D1133LK, or Q706L.

Embodiment 7. The method of any one of embodiments 1-6, wherein said MGF family member belongs to the MGF-100, MGF-110, MGF-300, MGF-360, or MGF-505 families.

Embodiment 8. The method of embodiment 6, wherein said gene-binding moiety is configured to bind more than one gene within a single MGF family.

Embodiment 9. The method of embodiment 7 or 8, wherein the MGF-110 family member is MGF-110-L.

Embodiment 10. The method of any one of embodiments 1-9, wherein said animal is a mammal.

Embodiment 11. The method of embodiment 10, wherein said mammal is a porcine mammal.

Embodiment 12. The method of embodiment 11, wherein said porcine mammal is Sus scrofa, Sus ahenobarbus, Sus barbatus, Sus cebrifons, Sus celebensis, Sus oliveri, Sus philippensis, or Sus verrucosus.

Embodiment 13. The method of any one of embodiments 1-12, wherein said virus belongs to the genus Asfivirus.

Embodiment 14. The method of embodiment 13, wherein said virus is African swine fever virus (ASFV).

Embodiment 15. The method of any one of embodiments 1-14, wherein said gene-binding moiety is configured to bind a plurality of different portions of said one or more genes of said virus.

Embodiment 16. The method of any one of embodiments 1-15, wherein said gene-binding moiety is configured to bind a combination of at least two, at least three, or all four of DNA polymerase, Topoisomerase II, RNA helicase, an MGF family member, or any combination thereof.

Embodiment 17. The method of any one of embodiments 1-16, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

Embodiment 18. The method of any one of embodiments 1-17, wherein said nuclease is configured to bind at least 5 consecutive nucleotides at least one sequence selected from SEQ ID NOs: 1-10, 11-34, 61-69, any of the sequences in Table 3, any of the sequences in Table 4, any of the sequences in Table 3, or any of the genes in Tables 1-2 or a variant having at least 80%, 90%, 95%, or 99% identity thereto.

Embodiment 19. The method of any one of embodiments 1-18, wherein said nuclease is a programmable nuclease comprising a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

Embodiment 20. The method of embodiment 18 or 19, wherein said gene-binding moiety of said nuclease comprises a heterologous RNA polynucleotide configured to hybridize to said one or more genes of said virus.

Embodiment 21. The method of embodiment 20, wherein said heterologous RNA polynucleotide comprises at least one, at least two, or at least three targeting sequences, wherein said targeting sequence comprises at least 17 consecutive nucleotides of at least one sequence selected from SEQ ID NOs: 25-36 or any of the sequences in Table 4 or a variant having at least 80%, 90%, 95%, or 99% identity thereto.

Embodiment 22. The method of any one of embodiments 1-21, wherein introducing a nuclease comprising a gene-binding moiety to said cell of said animal comprises contacting said cell with said nuclease.

Embodiment 23. The method of embodiment 22, wherein said nuclease comprises a ribonucleoprotein complex comprising a Cas polypeptide and at least one, at least two, or at least three heterologous RNA polynucleotides configured to hybridize to said one or more genes of said virus.

Embodiment 24. The method of any one of embodiments 1-23, wherein introducing a nuclease comprising a gene-binding moiety to said cell of said animal comprises contacting said cell with an mRNA comprising a sequence encoding said nuclease.

Embodiment 25. The method of embodiment 24, wherein said nuclease comprises a Cas polypeptide, wherein introducing a nuclease comprising a gene-binding moiety to said cell of said animal further comprises contacting said cell with at least one, at least two, or at least three heterologous RNA polynucleotides configured to hybridize to said one or more genes of said virus.

Embodiment 26. The method of embodiment 25, wherein said mRNA and said heterologous RNA polynucleotide are separate RNAs.

Embodiment 27. The method of any one of embodiments 1-26, wherein introducing a nuclease comprising a gene-binding moiety to said cell of said animal comprises contacting said cell with a vector comprising a sequence encoding said nuclease.

Embodiment 28. The method of embodiment 27, wherein said nuclease comprises a Cas polypeptide, wherein said vector further encodes at least one, at least two, or at least three heterologous RNA polynucleotides configured to hybridize to said one or more genes of said virus.

Embodiment 29. The method of embodiment 27 or 28, wherein said vector is a plasmid, a minicircle, or a viral vector.

Embodiment 30. The method of embodiment 29, wherein said vector is a viral vector, wherein said viral vector is a retroviral vector, an adenoviral vector, an adeno-associated viral vector (AAV), a lentiviral vector, a pox vector, a parvoviral vector, a measles viral vector, betaarterivirus vector, pseudorabies vector, or a herpes simplex virus vector (HSV).

Embodiment 31. The method of embodiment 30, wherein said vector is a lentiviral vector.

Embodiment 32. The method of any one of embodiments 24-31, wherein said sequence encoding said nuclease is codon-optimized for expression in said animal.

Embodiment 33. The method of any one of embodiments 1-32, wherein said introducing occurs in vivo, ex vivo, or in vitro.

Embodiment 34. The method of any one of embodiments 1-33, wherein said nuclease cleaves viral genomic DNA encoding said one or more genes of said virus within said cell of said animal.

Embodiment 35. The method of any one of embodiments 1-34, wherein said nuclease cleaves mRNA transcribed from DNA encoding said one or more genes of said virus within said cell of said animal.

Embodiment 36. The method of any one of embodiments 1-35, wherein said method results in prevention or delay of mortality of said animal upon infection with said virus belonging to the family Asfarviridae.

Embodiment 37. The method of any one of embodiments 1-36, wherein said method results in reduced mortality of said animal upon infection with said virus belonging to the family Asfarviridae.

Embodiment 38. The method of any one of embodiments 1-37, wherein introducing to a cell of said animal said nuclease comprises injecting said animal with, administering nasally to said animal, or administering orally to said animal said nuclease or a vector encoding said nuclease.

Embodiment 39. A vector comprising a sequence encoding at least one programmable nuclease configured to bind at least one essential viral gene.

Embodiment 40. The vector of embodiment 39, wherein the essential viral gene is of a virus from the family Asfarviridae.

Embodiment 41. The vector of embodiment 39 or 40, wherein said at least one essential gene of said virus encodes DNA polymerase or a fragment thereof, Topoisomerase II or a fragment thereof, RNA helicase or a fragment thereof, or a multigene family (MGF) family member or a fragment thereof.

Embodiment 42. The vector of any one of embodiments 39-41, wherein said at least one essential gene encodes DNA polymerase or a fragment thereof, wherein said DNA polymerase is G1211R or a fragment thereof.

Embodiment 43. The vector of any one of embodiments 39-42, wherein said at least one essential gene encodes Topoisomerase II or a fragment thereof, wherein said Topoisomerase II is p1192R or a fragment thereof.

Embodiment 44. The vector of any one of embodiments 39-43, wherein said at least one essential gene encodes RNA helicase or a fragment thereof, wherein said RNA helicase is QP509L, A859L, F105L, B92L, D1133LK, or Q706L.

Embodiment 45. The vector of any one of embodiments 39-44, wherein said at least one essential gene an MGF family member or a fragment thereof, wherein said MGF family member belongs to the MGF-100, MGF-110, MGF-300, MGF-360, or MGF-505 families.

Embodiment 46. The vector of any one of embodiments 39-45, wherein said gene-binding moiety is configured to bind more than one gene within a single MGF family.

Embodiment 47. The vector of embodiment 39 or 40, wherein said vector is a plasmid, a minicircle, or a viral vector.

Embodiment 48. The vector of embodiment 39 or 40, wherein said vector is a viral vector, wherein said viral vector is a retroviral vector, an adenoviral vector, an adeno-associated viral vector (AAV), a lentiviral vector, a pox vector, a parvoviral vector, a measles viral vector, a betaarterivirus vector, a pseudorabies vector or a herpes simplex virus vector (HSV).

Embodiment 49. The vector of any one of embodiments 39-48, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

Embodiment 50. The vector of any one of embodiments 39-49, wherein said programmable nuclease is configured to bind a plurality of different portions of said one or more genes of said virus.

Embodiment 51. The vector of any one of embodiments 39-50, wherein said nuclease is configured to bind at least 5 consecutive nucleotides at least one sequence selected from SEQ ID NOs: 1-10, 11-34, 61-69, any of the sequences in Table 3, any of the sequences in Table 4, any of the sequences in Table 3, or any of the genes in Tables 1-2 or a variant having at least 80%, 90%, 95%, or 99% identity thereto.

Embodiment 52. The vector of any one of embodiments 39-51, wherein said programmable nuclease comprises a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

Embodiment 53. The vector of embodiment 52, wherein said vector further comprises a second sequence encoding at least one, at least two, or at least three heterologous RNA polynucleotides configured to hybridize to said one or more genes of said virus.

Embodiment 54. The vector of embodiment 53, wherein said heterologous RNA polynucleotide comprises at least one, at least two, or at least three targeting sequences, wherein said targeting sequence comprises at least 17 consecutive nucleotides of at least one sequence selected from SEQ ID NOs: 25-36, a variant having at least 80%, 90%, 95%, or 99% identity thereto, or a variant substantially identical thereto.

Embodiment 55. The vector of any one of embodiments 53-54, wherein said sequence encoding said heterologous RNA polynucleotide is operably linked to a sequence comprising a U6 or an ASFV p30 promoter.

Embodiment 56. The vector of any one of embodiments 53-55, wherein said sequence encoding said heterologous RNA polynucleotide is operably linked to a sequence comprising at least 43 consecutive nucleotides of an ASFV p30 promoter, a variant having at least 80%, at least 90%, at least 95%, at least 99% identity thereto, or a variant substantially identical thereto.

Embodiment 57. The vector of any one of embodiments 39-56, wherein said programmable nuclease is operably linked to a sequence comprising a CMV promoter or an ASFV p72 promoter.

Embodiment 58. The vector of any one of embodiments 39-57, wherein said programmable nuclease is operably linked to a sequence comprising at least 43 consecutive nucleotides of an ASFV p72 promoter, a variant having at least 80%, at least 90%, at least 95%, at least 99% identity thereto, or a variant substantially identical thereto.

Embodiment 59. The vector of any one of embodiments 39-58, wherein said sequence encoding said programmable nuclease is codon-optimized for expression in said animal.

Embodiment 60. The vector of embodiments 59, wherein said animal is a mammal.

Embodiment 61. The vector of embodiments 60, wherein said animal is a mammal and said mammal is a porcine mammal.

Embodiment 62. A pharmaceutically-acceptable composition, comprising the vector of any one of embodiments 39-61 and a pharmaceutically-acceptable excipient.

What is claimed is:

1. A method for inhibiting infection of or reducing replication of a virus in an animal in need thereof, comprising introducing to a cell of said animal a nuclease comprising a gene-binding moiety, wherein said gene binding moiety is configured to bind at least one essential gene of said virus, or any combination thereof, wherein said virus belongs to the family Asfarviridae.

2. The method of claim 1, wherein said at least one essential gene of said virus encodes DNA polymerase or a fragment thereof, Topoisomerase II or a fragment thereof, RNA helicase or a fragment thereof, or a multigene family (MGF) family member or a fragment thereof.

3. The method of claim 2, wherein said at least one essential gene encodes DNA polymerase or a fragment thereof, wherein the DNA polymerase is G1211R or a fragment thereof.

4. The method of claim 2, wherein said at least one essential gene encodes Topoisomerase II or a fragment thereof, wherein the Topoisomerase II is p1192R or a fragment thereof.

5. The method of claim 2, wherein said at least one essential gene encodes RNA helicase or a fragment thereof, wherein the RNA helicase is QP509L, A859L, F105L, B92L, D1133LK, or Q706L.

6. The method of claim 2, wherein said MGF family member or a fragment thereof belongs to the MGF-100, MGF-110, MGF-300, MGF-360, or MGF-505 families.

7. The method of claim 6, wherein said gene-binding moiety is configured to bind more than one gene within a single MGF family.

8. The method of claim 6, wherein the MGF-110 family member is MGF-110-L.

9. The method of claim 1, wherein said animal is a mammal.

10. The method of claim 9, wherein said mammal is a porcine mammal.

11. The method of claim 10, wherein said porcine mammal is Sus scrofa, Sus ahenobarbus, Sus barbatus, Sus cebrifons, Sus celebensis, Sus oliveri, Sus philippensis, or Sus verrucosus.

12. The method of claim 1, wherein said virus belongs to the genus Asfivirus.

13. The method of claim 12, wherein said virus is African swine fever virus (ASFV).

14. The method of claim 1, wherein said gene-binding moiety is configured to bind a plurality of different portions of said at least one essential gene of said virus.

15. The method of claim 1, wherein said gene-binding moiety is configured to bind a combination of at least two, at least three, or all four of DNA polymerase, Topoisomerase II, RNA helicase, an MGF family member, or any combination thereof.

16. The method of claim 1, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

17. The method of claim 1, wherein said nuclease is configured to bind at least 5 consecutive nucleotides of at least one sequence selected from SEQ ID NOs: 1-10, 11-34, 61-69, any of the sequences in Table 3, any of the sequences in Table 4, any of the sequences in Table 3, or any of the genes in Tables 1-2 or a variant having at least 80%, 90%, 95%, or 99% identity thereto.

18. The method of claim 1, wherein said nuclease is a programmable nuclease comprising a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

19. The method of claim 17, wherein said gene-binding moiety of said nuclease comprises a heterologous RNA polynucleotide configured to hybridize to said at least one essential gene of said virus.

20. The method of claim 19, wherein said heterologous RNA polynucleotide comprises at least one, at least two, or at least three targeting sequences, wherein said targeting sequence comprises at least 17 consecutive nucleotides of at least one sequence selected from SEQ ID NOs: 25-36 or a variant having at least 80%, 90%, 95%, or 99% identity thereto.

21. The method of claim 1, wherein introducing a nuclease comprising a gene-binding moiety to said cell of said animal comprises contacting said cell with said nuclease.

22. The method of claim 21, wherein said nuclease comprises a ribonucleoprotein complex comprising a Cas polypeptide and at least one, at least two, or at least three heterologous RNA polynucleotides configured to hybridize to said at least one essential gene of said virus.

23. The method of claim 1, wherein introducing a nuclease comprising a gene-binding moiety to said cell of said animal comprises contacting said cell with an mRNA comprising a sequence encoding said nuclease.

24. The method of claim 23, wherein said nuclease comprises a Cas polypeptide, wherein introducing a nuclease comprising a gene-binding moiety to said cell of said animal further comprises contacting said cell with at least one, at least two, or at least three heterologous RNA polynucleotides configured to hybridize to said at least one essential gene of said virus.

25. The method of claim 24, wherein said mRNA and said heterologous RNA polynucleotide are separate RNAs.

26. The method of claim 1, wherein introducing a nuclease comprising a gene-binding moiety to said cell of said animal comprises contacting said cell with a vector comprising a sequence encoding said nuclease.

27. The method of claim 26, wherein said nuclease comprises a Cas polypeptide, wherein said vector further encodes at least one, at least two, or at least three heterologous RNA polynucleotides configured to hybridize to said one or more genes of said virus.

28. The method of claim 26, wherein said vector is a plasmid, a minicircle, or a viral vector.

29. The method of claim 28, wherein said vector is a viral vector, wherein said viral vector is a retroviral vector, an adenoviral vector, an adeno-associated viral vector (AAV), a lentiviral vector, a pox vector, a parvoviral vector, a measles viral vector, betaarterivirus vector, pseudorabies vector, or a herpes simplex virus vector (HSV).

30. The method of claim 29, wherein said vector is a lentiviral vector.

31. The method of claim 23, wherein said sequence encoding said nuclease is codon-optimized for expression in said animal.

32. The method of claim 1, wherein said introducing occurs in vivo, ex vivo, or in vitro.

33. The method of claim 1, wherein said nuclease cleaves viral genomic DNA encoding said at least one essential gene of said virus within said cell of said animal.

34. The method of claim 1, wherein said nuclease cleaves mRNA transcribed from DNA encoding said at least one essential gene of said virus within said cell of said animal.

35. The method of claim 1, wherein said method results in prevention or delay of mortality of said animal upon infection with said virus belonging to the family Asfarviridae.

36. The method of claim 1, wherein said method results in reduced mortality of said animal upon infection with said virus belonging to the family Asfarviridae.

37. The method of claim 1, wherein introducing to a cell of said animal said nuclease comprises injecting said animal with said nuclease or a vector encoding said nuclease.

38. A vector comprising a sequence encoding at least one programmable nuclease configured to bind at least one essential viral gene of a virus from the family Asfarviridae.

39. The vector of claim 38, wherein said at least one essential gene of said virus encodes DNA polymerase or a fragment thereof, Topoisomerase II or a fragment thereof, RNA helicase or a fragment thereof, or a multigene family (MGF) family member or a fragment thereof.

40. The vector of claim 39, wherein said at least one essential gene encodes DNA polymerase or a fragment thereof, wherein said DNA polymerase is G1211R or a fragment thereof.

41. The vector of claim 38, wherein said at least one essential gene encodes Topoisomerase II or a fragment thereof, wherein said Topoisomerase II is p1192R or a fragment thereof.

42. The vector of claim 38, wherein said at least one essential gene encodes RNA helicase or a fragment thereof, wherein said RNA helicase is QP509L, A859L, F105L, B92L, D1133LK, or Q706L.

43. The vector of claim 39, wherein said at least one essential gene an MGF family member or a fragment thereof, wherein said MGF family member belongs to the MGF-100, MGF-110, MGF-300, MGF-360, or MGF-505 families.

44. The vector of claim 38, wherein said gene-binding moiety is configured to bind more than one gene within a single MGF family.

45. The vector of claim 38, wherein said vector is a plasmid, a minicircle, or a viral vector.

46. The vector of claim 45, wherein said vector is a viral vector, wherein said viral vector is a retroviral vector, an adenoviral vector, an adeno-associated viral vector (AAV), a lentiviral vector, a pox vector, a parvoviral vector, a measles viral vector, a betaarterivirus vector, a pseudorabies vector or a herpes simplex virus vector (HSV).

47. The vector of claim 38, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

48. The vector of claim 38, wherein said programmable nuclease is configured to bind a plurality of different portions of said at least one essential gene of said virus.

49. The vector of claim 38, wherein said nuclease is configured to bind at least 5 consecutive nucleotides at least one sequence selected from SEQ ID NOs: 1-10, 11-34, 61-69, any of the sequences in Table 3, any of the sequences in Table 4, any of the sequences in Table 3, or any of the genes in Tables 1-2 or a variant having at least 80%, 90%, 95%, or 99% identity thereto.

50. The vector of claim 38, wherein said programmable nuclease comprises a CRISPR-associated (Cas) polypeptide, wherein said Cas polypeptide is a type I CRISPR-associated (Cas) polypeptide, a type II CRISPR-associated (Cas) polypeptide, a type III CRISPR-associated (Cas) polypeptide, a type IV CRISPR-associated (Cas) polypeptide, a type V CRISPR-associated (Cas) polypeptide, a type VI CRISPR-associated (Cas) polypeptide.

51. The vector of claim 50, wherein said vector further comprises a second sequence encoding at least one, at least two, or at least three heterologous RNA polynucleotides configured to hybridize to said at least one essential gene of said virus.

52. The vector of claim 51, wherein said heterologous RNA polynucleotide comprises at least one, at least two, or at least three targeting sequences, wherein said targeting sequence comprises at least 17 consecutive nucleotides of at least one sequence selected from SEQ ID NOs: 11-34, 61-69, any of the sequences in Table 4, a variant having at least 80%, 90%, 95%, or 99% identity thereto, or a variant substantially identical thereto.

53. The vector of claim 51, wherein said sequence encoding said heterologous RNA polynucleotide is operably linked to a sequence comprising a U6 or an ASFV p30 promoter.

54. The vector of claim 51, wherein said sequence encoding said heterologous RNA polynucleotide is operably linked to a sequence comprising at least 43 consecutive nucleotides of an ASFV p30 promoter, a variant having at least 80%, at least 90%, at least 95%, at least 99% identity thereto, or a variant substantially identical thereto.

55. The vector of claim 38, wherein said programmable nuclease is operably linked to a sequence comprising a CMV promoter or an ASFV p72 promoter.

56. The vector of claim 38, wherein said programmable nuclease is operably linked to a sequence comprising at least 43 consecutive nucleotides of an ASFV p72 promoter, a variant having at least 80%, at least 90%, at least 95%, at least 99% identity thereto, or a variant substantially identical thereto.

57. The vector claim 38, wherein said sequence encoding said programmable nuclease is codon-optimized for expression in said animal.

58. The vector of claim 57, wherein said animal is a mammal.

59. The vector of claim 58, wherein said animal is a mammal and said mammal is a porcine mammal.

60. A pharmaceutically-acceptable composition, comprising the vector of claim 38 and a pharmaceutically-acceptable excipient.
